(19) United States
(12) Patent Application Publication
(10) Pub. No.: US 2003/0044399 A1
(43) Pub. Date: Mar. 6, 2003

(54) Method of treatment

(75) Inventors: Scott Kevin Thompson, Phoenixville, PA (US); David Graham Tew, Little Houghton (GB); Daniel Frank Veber, Ambler, PA (US)

Correspondence Address: GLAXOSMITHKLINE, Corporate Intellectual Property - UW2220, P.O. Box 1539, King of Prussia, PA, 19406-0939, US

(73) Assignee: SmithKline Beecham Corporation

(21) Appl. No.: 10/074,639

(22) Filed: Feb. 13, 2002

Related U.S. Application Data
(63) Continuation of application No. 09/653,815, filed on Sep. 1, 2000.
Application No. 09/653,815 is a continuation-in-part of application No. 09/593,845, filed on Jun. 14, 2000.
Application No. 09/593,845 is a continuation-in-part of application No. PCT/US99/30730, filed on Dec. 21, 1999.
(60) Provisional application No. 60/113,636, filed on Dec. 23, 1998.
(60) Provisional application No. 60/164,581, filed on Nov. 10, 1999.

(51) Int. Cl. A61K 38/43

(52) U.S. Cl. 424/94.1

(57) ABSTRACT

The present invention relates to methods of treating parasitic diseases which are mediated by cysteine proteases by administration of 4-amino-azepan-3-one protease inhibitors. In particular, the present invention relates to a method of treating malaria by inhibiting the cysteine protease falcipain.

### FIELD OF THE INVENTION

The present invention relates to methods for treating parasitic diseases using 4-amino-azepan-3-one protease inhibitors. In particular, the present methods serve to inhibit cysteine proteases of the papain superfamily. Thus, the present invention is useful for treating parasitic diseases which are mediated by the activity of such proteases. In particular, the present invention relates to treating malaria by inhibiting falcipain.

### BACKGROUND OF THE INVENTION

Infection with Plasmodium falciparum, the most virulent human malaria pathogen, infects over 280 million people and is estimated to be responsible for over 1 million deaths annually (Gibbons, A. Science 1992, 256, 1135; Walsh, J. A. Ann. N. Y. Acad. Sci. 1989, 569, 1135). The Plasmodium falciparum parasite has a 48 hour life cycle within host erythrocytes that is responsible for all of the clinical manifestations of falciparum malaria. During this cycle, the erythrocyte is invaded by a merozoite, then the intracellular parasite develops from a ring stage into a more metabolically active trophozoite, divides asexually and becomes a schizont, and finally ruptures the host erythrocyte, releasing daughter merozoites that invade other erythrocytes to reinitiate the cycle. During the trophozoite stage, hemoglobin from the host erythrocyte is degraded for use as the parasites principal source of amino acids.

Rosenthal and coworkers have identified a 28 kD trophozoite cysteine protease (TCP or falcipain) from malaria parasites that mediates host hemoglobin degradation (Rosenthal, P. J.; McKerrow, J. H.; Aikawa, M.; Nagasawa, H.; Leech, J. H. J. Clin. Invest 1988, 82, 1560) and is expressed only at the trophozoite stage (Rosenthal, P. J.; Kim, J. H.; McKerrow, J. H.; Leech, J. H. J. Exp. Med. 1987, 166, 816). Inhibition of this enzyme results in a blocking of hemoglobin degradation and killing of cultured parasites (Rosenthal, P. J.; Wollish, W. S.; Palmer, J. T.; Rasnick, D. J. Clin. Invest. 1991, 88, 1467; Li, R.; Kenyon, G. L.; Cohen, F. E.; Chen, X.; Gong, B.; Dominguez, J. N.; Davidson, E.; Kurzban, G.; Miller, R. E.; Nuzum, E. O.; Rosenthal. P. J.; McKerrow, J. H. J. Med. Chem. 1995, 38, 5031). In a mouse model of infection with P. vinckei, the analogous murine malarial parasite, treatment with cysteine protease inhibitors resulted in a long-term curative effect (>75 days) in 80% of animals (Rosenthal, P. J.; Lee, G. K.; Smith R. E. J. Clin. Invest. 1993, 91, 1052). Thus, a selective inhibitor of falcipain may be an effective anti-malarial therapy either in conjunction with or as a replacement for the quinoline-derived drugs.

In addition to Plasmodium falciparum, other parasites utilize cysteine proteases in their life cycle. These include Trypanosoma cruzi, Trypanosoma Brucei [trypanosomiasis (African sleeping sickness, Chagas disease)], Leishmania mexicana, Leishmania pifanoi, Leishmania major (Ieishmaniasis), Schistosoma mansoni (schistosomiasis), Onchocerca volvuilus [onchocerciasis (river blindness)] Brugia pahangi, Entamoeba histolytica, Giardia lambia, the helminths, Haemonchus contortus and Fasciola hepatica, as well as helminths of the genera Spirometra, Trichitiella, Necator and Ascaris, and protozoa of the genera Cryptosporidium, Eimeria, Toxoplasma and Naegleria (McKerrow, J. H. (1995) in Perspect. Drug Dis. Des. 2, eds., Craik, C. S., Debouck, C., pp. 437-444; Robertson, C. D., Coombs, G. H., North, M. J., Mottram, J. C. (1996) in Perspect. Drug Dis. Des. 6, eds., McKerrow, J. H. and James, M. N. G., pp. 99-118).

It has now been discovered that certain 4-amino-azepan-3-ones are protease inhibitors, most particularly inhibitors of falcipain, and that these compounds are useful for treating parasitic diseases, particularly malaria.

### SUMMARY OF THE INVENTION

An object of the present invention is to provide methods of treatment which serve to inhibit cysteine proteases, and particularly cysteine proteases of the papain superfamily. The present methods are useful for treating parasitic diseases which may be therapeutically modified by altering the activity of such proteases. In particular, the present invention relates to treating malaria by inhibiting falcipain.

Accordingly, in the first aspect, this invention provides a method of treating parasitic diseases in which the disease pathology may be therapeutically modified by inhibiting proteases, such as cysteine proteases, using 4-amino-azepan-3-ones of Formula I.

In particular, these compounds are used in the present method to treat parasitic diseases by inhibiting cysteine proteases of the papain superfamily. Most particularly, the present invention provides a method of treating malaria by the inhibition of falcipain with such compounds.

### DETAILED DESCRIPTION OF THE INVENTION

The present invention provides a method for treating parasitic diseases which may be therapeutically modified by altering the activity of cysteine proteases by administering to a patient in need thereof, particularly an animal, more particularly a mammal, most particularly a human being, one or more compounds of Formula I:

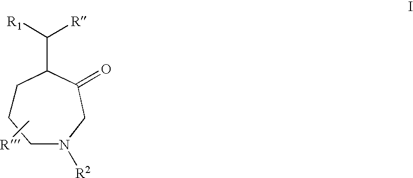

Wherein:

R¹ is selected from the group consisting of:

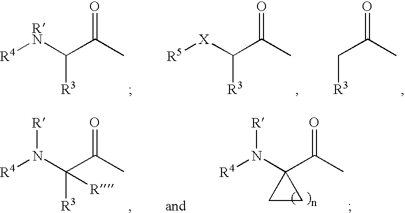

R² is selected from the group consisting of: H, C₁₋₆alkyl, C₃₋₆cycloalkyl-C₀₋₆alkyl, Ar—C₀₋₆alkyl, Het-C₀₋₆alkyl, R⁹C(O)—, R⁹C(S)—, R⁹SO₂—, R⁹OC(O)—,

R⁹R¹¹NC(O)—, R⁹R¹¹NC(S)—,

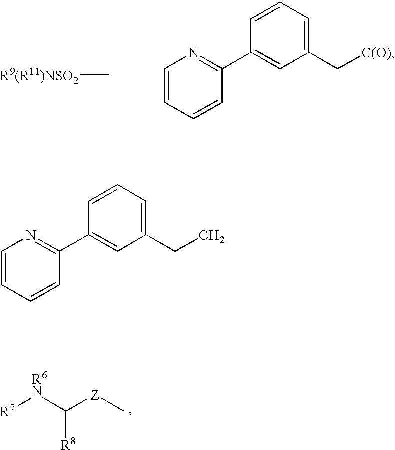

and R⁹SO₂R¹¹NC(O)—;

R³ is selected from the group consisting of: H, C₁₋₆alkyl, C₃₋₆cycloalkyl-C₀₋₆alkyl, C₂₋₆alkenyl, C₂₋₆alkynyl, HetC₀₋₆alkyl and ArC₀₋₆alkyl;

R³ and R′ may be connected to form a pyrrolidine, piperidine or morpholine ring;

R⁴ is selected from the group consisting of: H, C₁₋₆alkyl, C₃₋₆cycloalkyl-C₀₋₆alkyl, Ar—C₀₋₆alkyl, Het-C₀₋₆alkyl, R⁵C(O)—, R⁵C(S)—, R⁵SO₂—, R⁵OC(O)—, R⁵R¹²NC(O)—, and R⁵R¹²NC(S)—;

R⁵ is selected from the group consisting of: H, C₁₋₆alkyl, C₂₋₆alkenyl, C₂₋₆alkynyl, C₃₋₆cycloalkyl-C₀₋₆alkyl, Ar—C₀₋₆alkyl and Het-C₀₋₆alkyl;

R⁶ is selected from the group consisting of: H, C₁₋₆alkyl, Ar—C₀₋₆alkyl, and Het-C₀₋₆alkyl;

R⁷ is selected from the group consisting of: H, C₁₋₆alkyl, C₃₋₆cycloalkyl-C₀₋₆alkyl, Ar—C₀₋₆alkyl, Het-C₀₋₆alkyl, R¹⁰C(O)—, R¹⁰C(S)—, R¹⁰SO₂—, R¹⁰OC(O)—, R¹⁰R¹³NC(O)—, and R¹⁰R¹³NC(S)—;

R⁸ is selected from the group consisting of: H, C₁₋₆alkyl, C₂₋₆alkenyl, C₂₋₆alkynyl, HetC₀₋₆alkyl and ArC₀₋₆alkyl;

R⁹ is selected from the group consisting of: C₁₋₆alkyl, C₃₋₆cycloalkyl-C₀₋₆alkyl, Ar—C₀₋₆alkyl and Het-C₀₋₆alkyl;

R¹⁰ is selected from the group consisting of: C₁₋₆alkyl, C₃₋₆cycloalkyl-C₀₋₆alkyl, Ar—C₀₋₆alkyl and Het-C₀₋₆alkyl;

R¹¹ is selected from the group consisting of: H, C₁₋₆alkyl, Ar—C₀₋₆alkyl, and Het-C₀₋₆alkyl;

R¹² is selected from the group consisting of: H, C₁₋₆alkyl, Ar—C₀₋₆alkyl, and Het-C₀₋₆alkyl;

R¹³ is selected from the group consisting of: H, C₁₋₆alkyl, Ar—C₀₋₆alkyl, and Het-C₀₋₆alkyl;

R′ is selected from the group consisting of: H, C₁₋₆alkyl, Ar—C₀₋₆alkyl, and Het-C₀₋₆alkyl;

R″ is selected from the group consisting of: H, C₁₋₆alkyl, Ar—C₀₋₆alkyl, or Het-C₀₋₆alkyl;

R′″ is selected from the group consisting of: H, C₁₋₆alkyl, C₃₋₆cycloalkyl-C₀₋₆alkyl, Ar—C₀₋₆alkyl, and Het-C₀₋₆alkyl;

R″″ is selected from the group consisting of: C₁₋₆alkyl, C₃₋₆cycloalkyl-C₀₋₆alkyl C₂₋₆alkenyl, C₂₋₆alkynyl, HetC₀₋₆alkyl and ArC₀₋₆alkyl;

X is selected from the group consisting of: CH₂, S, and O;

Z is selected from the group consisting of: C(O) and CH₂;

n is an integer from 1 to 5;

And pharmaceutically acceptable salts, hydrates and solvates thereof.

R¹ is preferably

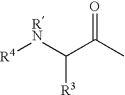

In compounds of Formula I. When R¹ is

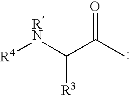

R³ is selected from the group consisting of: H, C₁₋₆alkyl, C₃₋₆cycloalkyl-C₀₋₆alkyl, C₂₋₆alkenyl, C₂₋₆alkynyl, Het-C₀₋₆alkyl and Ar—C₀₋₆alkyl;

R³ is preferably selected from the group consisting of: H, C₃₋₆cycloalkyl-C₀₋₆alkyl, C₂₋₆alkenyl, Ar—C₀₋₆alkyl, and C₁₋₆alkyl;

R³ is more preferably selected from the group consisting of:

H, methyl, ethyl, n-propyl, prop-2-yl, n-butyl, isobutyl, but-2-yl, cyclopropylmethyl, cyclohexylmethyl, 2-methanesulfinyl-ethyl, 1-hydroxyethyl, toluyl, naphthalen-2-ylmethyl, benzyloxymethyl, and hydroxymethyl.

R³ is even more preferably selected from the group consisting of: toluyl, isobutyl and cyclohexylmethyl.

R³ is most preferably isobutyl.

R⁴ is selected from the group consisting of: H, C₁₋₆alkyl, C₃₋₆cycloalkyl-C₀₋₆alkyl, Ar—C₀₋₆alkyl, Het-C₀₋₆alkyl, R⁵C(O)—, R⁵C(S)—, R⁵SO₂—, R⁵OC(O)—, R⁵R¹³NC(O)—, and R⁵R¹³NC(S)—.

R⁴ is preferably selected from the group consisting of: R⁵OC(O)—, R⁵C(O)— and R⁵SO₂—.

R⁴ is most preferably R⁵C(O)—.

In some embodiments, R⁴ is preferably methanesulfonyl.

R⁵ is selected from the group consisting of: H, C₁₋₆alkyl, C₂₋₆alkenyl, C₂₋₆alkynyl, C₃₋₆cycloalkyl-C₀₋₆alkyl, Ar—C₀₋₆alkyl or Het-C₀₋₆alkyl.

Preferably R⁵ is selected from the group consisting of: C₁₋₆alkyl, Ar—C₀₋₆alkyl and Het-C₀₋₆alkyl.

More preferably, and especially when R⁴ is R⁵C(O)—, R⁵ is selected from the group consisting of:

methyl, especially halogenated methyl, more especially trifluoromethyl , especially C₁₋₆alkoxy substituted methyl, more especially phenoxy-methyl , 4-fluoro-phenoxy-methyl, especially heterocycle substituted methyl, more especially 2-thiophenyl-methyl

ethyl, especially piperidin-1-yl-ethyl;

butyl, especially aryl substituted butyl, more especially 4-(4-methoxy)phenyl-butyl;

isopentyl;

cyclohexyl;

pentanonyl, especially 4-pentanonyl;

butenyl, especially aryl substituted butenyl, more especially 4,4-bis(4-methoxyphenyl)-but-3-enyl;

acetyl;

phenyl, especially phenyl substituted with one or more halogens, more especially 3,4-dichlorophenyl and 4-fluorophenyl, especially phenyl substituted with one or more aryloxy or C₁₋₆alkoxy groups, more especially 3,4-dimethoxy-phenyl, 3-benzyloxy-4-methoxy-phenyl, especially phenyl substituted with one or more C₁₋₆alkyl sulfonyl groups, more especially 4-methanesulfonyl-phenyl;

benzyl;

naphthalenyl, especially naphthylen-2-yl;

benzo[1,3]dioxolyl, especially benzo[1,3]dioxol-5-yl;

furanyl, especially furan-2-yl, especially substituted furanyl, such as 5-nitro-furan-2-yl, 5-(4-nitrophenyl)-furan-2-yl, 5-(3-trifluoromethyl-phenyl)-furan-2-yl, more especially halogen substituted furanyl, even more especially 5-bromo-furan-2-yl, more especially aryl substituted furanyl, even more especially 5-(4-chloro-phenyl)-furan-2-yl, more especially C₁₋₆alkyl substituted furanyl, even more especially 3-methyl-furan-2-yl, 4-methyl-furan-2-yl, 2,5-dimethyl-furan-2-yl, and 2,4-dimethyl-furan-3-yl;

tetrahydrofuranyl, tetrahydrofuran-2-yl;

benzofuranyl, especially benzofuran-2-yl, and substituted benzofuranyl, more especially 5-(2-pipergzin-4-carboxylic acid tert-butyl ester-ethoxy) benzofuran-2-yl, 5-(2-morpholino-4-yl-ethoxy)-benzofuran-2-yl, 5-(2-piperazin-1-yl-ethoxy)benzofuran-2-yl, 5-(2-cyclohexyl-ethoxy)-benzofuran-2-yl; especially C₁₋₆alkoxy substituted benzofuranyl, more especially 7-methoxy-benzofuran-2-yl, 5-methoxy-benzofuran-2-yl, 5,6-dimethoxy-benzofuran-2-yl, especially halogen substituted benzofuranyl, more especially 5-fluoro-benzofuran-2-yl, 5,6-difluoro-benzofuran-2-yl, especially C₁₋₆alkyl substituted benzofuranyl, most especially 3-methyl-benzofuran-2-yl, 3,5-dimethyl-benzofuran-2-yl, and 3-ethyl-benzofuran-2-yl; also 5-fluoro-3-methyl-benzofuran-2-yl, 6-fluoro-3-methyl-benzofuran-2-yl, 5-methoxy-3-methyl-benzofuran-2-yl, 4-methoxy-3-methyl-benzofuran-2-yl, and 6-methoxy-3-methyl-benzofuran-2-yl;

naphtho[2,1-b]-furanyl, especially naphtho[2,1-b]-furan-2-yl, alkyl substituted naphtho[2,1-b]-furanyl, especially 1methyl-naphtho[2,1-b]-furan-2-yl;

benzo[b]thiophenyl, especially benzo[b]thiophen-2-yl; especially C₁₋₆alkoxy substituted benzo[b]thiophenyl, more especially 5,6-dimethoxy-benzo[b]thiophen-2-yl;

quinolinyl, especially quinolin-2-yl, quinolin-3-yl, quinolin-4-yl, quinolin-6-yl, and quinolin-8-yl;

quinoxalinyl, especially quinoxalin-2-yl;

1,8 naphthyridinyl, especially 1,8 naphthyridin-2-yl;

indolyl, especially indol-2-yl, especially indol-6-yl, indol-5-yl, especially C₁₋₆alkyl substituted indolyl, more especially N-methyl-indol-2-yl;

pyridinyl, especially pyridin-2-yl, pyridin-3-yl, pyridin-5-yl, especially C₁₋₆alkyl substituted pyridinyl, more especially 2-methyl-pyridin-5-yl, and oxy-pyridinyl, especially 1-oxy-pyridin-2-yland 1-oxy-pyridin-3-yl;

furo[3,2-b]-pyridinyl, especially furo[3,2-b]-pyridin-2-yl, C₁₋₆alkyl substituted furo[3,2-b]-pyridinyl, especially 3-methyl-furo[3,2-b]-pyridin-2-yl;

thiophenyl, especially thiophen-3-yl, also thiophen-2-yl, especially C₁₋₆alkyl substituted thiophenyl, more especially 5-methyl-thiophen-2-yland 5-methyl-thiophen-3-yl, especially halogen substituted thiophenyl, more especially 4,5-dibromo-thiophen-2-yl;

thieno[3,2-b]thiophene, especially thieno[3,2-b]thiophene-2-yl, more especially C₁₋₆alkyl substituted thieno[3,2-b]thiophene-2-yl, more especially 5-tert-butyl-3-methyl-thieno[3,2-b]thiophene-2-yl;

isoxazolyl, especially isoxazol-4-yl, especially C₁₋₆alkyl substituted isoxazolyl, more especially 3,5-dimethyl-isoxazol-4-yl;

oxazolyl, especially oxazol-4-yl, more especially 5-methyl-2-phenyl oxazol-4-yl, 2-phenyl-5-trifluoromethyl-oxazol-4-yl; and

1H-benzoimidazolyl, especially 1H-benzoimidazol-5-yl.

When R⁴ is R⁵SO₂, R⁵ is preferably pyridin-2-yl or 1-oxo-pyridin-2-yl.

R′ is selected from the group consisting of: H, C₁₋₆alkyl, Ar—C₀₋₆alkyl, and Het-C₀₋₆alkyl.

Preferably R′ is selected from the group consisting of: H and naphthalen-2-yl-methyl.

Most preferably R′ is H.

R″ is selected from the group consisting of: H, C₁₋₆alkyl, Ar—C₀₋₆alkyl, and Het-C₀₋₆alkyl.

Most preferably R″ is H.

R′″ is selected from the group consisting of: H, C₁₋₆alkyl, C₃₋₆cycloalkyl-C₀₋₆alkyl, and Het-C₀₋₆alkyl.

R′″ is preferably selected from the group consisting of: H and C₁₋₆alkyl.

R′″ is more preferably selected from the group consisting of: H, methyl and 6,6-dimethyl.

When R′″ is methyl, methyl is preferably selected from the group consisting of: 6-methyl and 7-methyl.

Even more preferably R′″ is selected from the group consisting of: H, 6-methyl and 7-methyl, most preferably 7-methyl.

In compounds of Formula I, when R¹ is

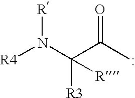

R³ is selected from the group consisting of: C₁₋₆alkyl, C₃₋₆cycloalkyl-C₀₋₆alkyl, C₂₋₆alkenyl, C₂₋₆alkynyl, Het-C₀₋₆alkyl and Ar—C₀₋₆alkyl.

R³ is preferably C₁₋₆alkyl.

R³ is more preferably selected from the group consisting of methyl, ethyl, n-propyl, n-butyl, isobutyl, t-butyl, cyclohexylmethyl, and toluyl.

R″″ is selected from the group consisting of: C₁₋₆alkyl, C₃₋₆cycloalkyl-C₀₋₆alkyl, C₂₋₆alkenyl, C₂₋₆alkynyl, HetC₀₋₆alkyl and ArC₀₋₆alkyl;

R″″ is preferably C₁₋₆alkyl;

R″″ is more preferably selected from the group consisting of methyl, ethyl, n-propyl, n-butyl, isobutyl and t-butyl.

R″″ is most preferably methyl.

In such compounds, R′, R″, R′″, R⁴, and R⁵ are as described above wherein

R¹ is

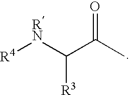

In compounds of Formula I, when R¹ is

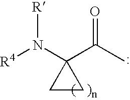

n is preferably an integer of from 1 to 5; and

R′, R″, R′″, R⁴, and R⁵ are as described above wherein

R¹ is

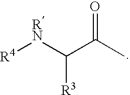

n is most preferably 3.

The ring may be unsubstituted or substituted with one or more of C₁₋₆alkyl, C₃₋₆cycloalkyl-C₀₋₆alkyl, C₂₋₆alkenyl, C₂₋₆alkynyl, HetC₀₋₆alkyl, ArC₀₋₆alkyl, or halogen.

The ring is preferably unsubstituted.

In compounds of Formula I, R² is selected from the group consisting of: H, C₁₋₆alkyl, C₃₋₆cycloalkyl-C₀₋₆alkyl, Ar—C₀₋₆alkyl, Het-C₀₋₆alkyl, R⁹C(O)—, R⁹C(S)—, R⁹SO₂—, R⁹OC(O)—, R⁹R¹¹NC(O)—, R⁹R¹¹NC(S)—, R⁹R¹¹NSO₂—,

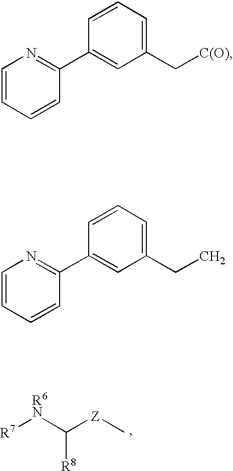

and R⁹SO₂R¹¹NC(O)—.

More preferably R² is selected from the group consisting of: Ar—C₀₋₆alkyl, R⁹C(O)—, R⁹SO₂, R⁹R¹¹NC(O)—, and

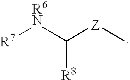

Even more preferably, R² is selected from the group consisting of: Ar—C₀₋₆alkyl, R⁹C(O)—, and R⁹SO₂.

Most preferably R² is R⁹SO₂.

In such embodiments:

R⁶ is selected from the group consisting of: H, C₁₋₆alkyl, Ar—C₀₋₆alkyl, or Het-C₀₋₆alkyl, preferably H.

R⁷ is selected from the group consisting of: H, C₁₋₆alkyl, C₃₋₆cycloalkyl-C₀₋₆alkyl, Ar—C₀₋₆alkyl, Het-C₀₋₆alkyl, R¹⁰C(O)—, R¹⁰C(S)—, R¹⁰SO₂—, R¹⁰OC(O)—, R¹⁰R¹⁴NC(O)—, R¹⁰R¹⁴NC(S)—, R⁷ is preferably R¹⁰OC(O).

R⁸ is selected from the group consisting of: H, C₁₋₆alkyl, C₂₋₆alkenyl, C₂₋₆alkynyl, HetC₀₋₆alkyl and ArC₀₋₆alkyl; preferably C₁₋₆alkyl, more preferably isobutyl.

R⁹ is selected from the group consisting of: C₁₋₆alkyl, C₃₋₆cycloalkyl-C₀₋₆alkyl, Ar—C₀₋₆alkyl, and Het-C₀₋₆alkyl.

R⁹ is preferably selected from the group consisting of: C₁₋₆alkyl, Ar—C₀₋₆alkyl, and Het-C₀₋₆alkyl.

More preferably, R⁹ is selected from the group consisting of:

methyl;

ethyl, especially C₁₋₆alkyl-substituted ethyl, more especially 2-cyclohexyl-ethyl;

propyl;

butyl, especially C₁₋₆butyl, more especially 3-methylbutyl;

tert-butyl, particularly when R² is R⁹OC(O);

isopentyl;

phenyl, especially halogen substituted phenyl, more especially 3,4-dichlorophenyl, 4-bromophenyl, 2-fluorophenyl, 3-fluorophenyl, 4-fluorophenyl, 2-chlorophenyl, 3-chlorophenyl, 4-chlorophenyl, especially C₁₋₆alkoxy phenyl, more especially 3-methoxyphenyl, 4-methoxyphenyl, 3,4-dimethoxyphenyl, especially cyanophenyl, more especially 2-cyanophenyl; especially C₁₋₆alkyl substituted phenyl, more especially 4-ethyl-phenyl, 2-methyl phenyl, 4-methyl phenyl, especially C₁₋₆alkyl sulfonyl substituted phenyl, more especially 4-methanesulfonyl phenyl, and 2-methanesulfonyl phenyl;

toluyl, especially Het-substituted toluyl, more especially 3-(pyridin-2-yl)toluyl;

naphthylene, especially naphthyl-2-ene;

benzoic acid, especially 2-benzoic acid;

benzo[1,3]dioxolyl, especially benzo[1,3]dioxol-5-yl;

benzo[1,2,5]oxadiazolyl, especially benzo[1,2,5]oxadiazol-4-yl;

pyridinyl, especially pyridin-2-yl, pyridin-3-yl, especially 1-oxy-pyridinyl, more especially 1-oxy-pyridin-2-yl, 1-oxy-pyridin-3-yl; especially C₁₋₆alkylpyridinyl, more especially 3-methyl-pyridin-2-yl, 6-methyl-pyridin-2-yl;

thiophenyl, especially thiophenyl-2-yl;

thiazolyl, especially thiazol-2-yl;

1H-imidazolyl, especially 1H-imidazol-2-yl, 1H-imidazol-4-yl, more especially C₁₋₆alkyl substituted imidazolyl, even more especially 1-methyl-1H-imidazol-2-yl, 1-methyl-1H-imidazol-4-yl, and 1,2-dimethyl-1H-imidazol-4-yl;

triazolyl, especially 1H-[1,2,4]triazolyl, more especially 1H-[1,2,4]triazol-3-yl, especially C₁₋₆alkyl substituted 1H-[1,2,4]triazolyl, more especially 5-methyl-1H-[1,2,4]triazol-3-yl; and

isoxazolyl, especially isoxazol-4-yl, especially C₁₋₆alkyl substituted isoxazolyl, more especially 3,5-dimethyl-isoxazol-4-yl.

When R² is R⁹SO₂, R⁹ is most preferably selected from the group consisting of: pyridin-2-yl and 1-oxy-pyridin-2-yl.

When R² is R⁹SO₂R¹¹NC(O)—, R⁹ is preferably Ar—C₀₋₆alkyl, more preferably Ar, most preferably substituted phenyl such as 2-methyl phenyl, 4-methyl phenyl, 2-chloro phenyl, and 4-fluoro phenyl.

When R² is R⁹C(O)—, R⁹ is preferably selected from the group consisting of C₁₋₆alkyl, C₃₋₆cycloalkyl-C₀₋₆alkyl, and Het-C₀₋₆alkyl, more preferably 1-oxy-pyridin-2-yl, cyclohexyl ethyl, and 3-methyl butyl.

R¹¹ is selected from the group consisting of: H, C₁₋₆alkyl, Ar—C₀₋₆alkyl, and Het-C₀₋₆alkyl.

When R² is R⁹SO₂R¹¹NC(O)—, R¹¹ is preferably H.

When R² is Ar—C₀₋₆alkyl, R² is preferably phenyl, especially substituted phenyl, more especially halogen substituted phenyl, even more especially 2-fluorobenzyl.

When R² is C₁₋₆alkyl, R² is preferably selected from 1-propyl, 1-butyl, and 1-pentyl.

When R² is Het-C₀₋₆alkyl, Het-C₀₋₆alkyl is preferably Het-methyl, and Het in Het-methyl is preferably selected from the group consisting of:

pyridinyl, especially pyridin-2-yl, especially C₁₋₆alkylpyridinyl, more especially 6-methyl-pyridin-2-yl;

thiophenyl, especially thiophene-2-yl, more especially thiophen-2-yl or benzo[b]thiophen-2-yl;

thiazolyl, especially thiazol-4-yl such as 1-(2-morpholin-4-yl-thiazol-4-yl), and 1-(isothiazol-3-yl);

1H-imidazolyl, especially 1H-imidazol-2-yl, 1H-imidazol-4-yl, especially C₁₋₆alkyl substituted imidazolyl, more especially 1-methyl-1H-imidazol-2yl;

triazolyl, especially 3H-[1,2,3]triazolyl, more especially 3H-[1,2,3]triazol-4-yl, especially C₁₋₆alkyl substituted 3H-[1,2,3]triazolyl, more especially 3-phenyl-3H-[1,2,3]triazolyl-4-yl;

quinolinyl, especially quinolin-2-yl, quinolin-2-yl;

furanyl, especially furan-2-yl, especially substituted furanyl, such as 5-ethyl-furan-2-yl;

thieno[3,2-b]thiophene, especially thieno[3,2-b]thiophene-2-yl, especially C₁₋₆alkyl substituted thieno[3,2-b]thiophenyl, especially 3,4-dimethyl-thieno[3,2-b]thiophene-2-yl.

R² is also preferably:

H;

toluyl;

aryl substituted ethyl, especially 2-phenyl ethyl, 2-[3-(pyridin-2-yl) phenyl]ethyl.

Compounds of Formula I where R″ and R′″ are both H are preferred. Also preferred are such compounds wherein R′″ is selected from the group consisting of: 6-methyl and 7-methyl, preferably 7-methyl.

More preferred are compounds of Formula I wherein:

R¹ is

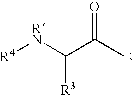

R² is selected from the group consisting of: Ar—C₀₋₆alkyl, R⁹C(O)—, R⁹SO₂, R⁹R¹¹NC(O)—, and

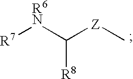

R³ is selected from the group consisting of: H, C₁₋₆alkyl, C₃₋₆cycloalkyl-C₀₋₆alkyl and Ar—C₀₋₆alkyl;

R⁴ is selected from the group consisting of: R⁵OC(O)—, R⁵C(O)— and R⁵SO₂—;

R⁵is selected from the group consisting of: C₁₋₆alkyl, Ar—C₀₋₆alkyl and Het-C₀₋₆alkyl;

R⁶ is H;

R⁷ is R¹⁰OC(O);

R⁸ is C₁₋₆alkyl;

R⁹ is selected from the group consisting of: C₁₋₆alkyl, Ar—C₀₋₆alkyl and Het-C₀₋₆alkyl;

R¹⁰ is selected from the group consisting of: C₁₋₆alkyl, Ar—C₀₋₆alkyl and Het-C₀₋₆alkyl;

R′ is H;

R″ is H;

R′″ is H; and

Z is selected from the group consisting of: C(O) and CH₂.

Also preferred are such compounds wherein R′″ is selected from the group consisting of: 6-methyl and 7-methyl, preferably 7-methyl.

Even more preferred are such compounds of Formula I wherein R² is selected from the group consisting of: Ar—C₀₋₆alkyl, R⁹C(O)—, R⁹SO₂.

Yet more preferred are compounds of Formula I wherein:

R¹ is

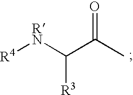

R² is selected from the group consisting of: Ar—C₀₋₆alkyl, R⁹C(O)—and R⁹SO₂;

R³ is selected from the group consisting of: H, methyl, ethyl, n-propyl, prop-2-yl, n-butyl, isobutyl, but-2-yl, cyclopropylmethyl, cyclohexylmethyl, 2-methanesulfinyl-ethyl, 1-hydroxyethyl, toluyl, naphthalen-2-ylmethyl, benzyloxymethyl, and hydroxymethyl;

R⁴ is R⁵C(O)—;

R⁵ is selected from the group consisting of:

methyl, especially halogenated methyl, more especially trifluoromethyl , especially C₁₋₆alkoxy substituted methyl, more especially phenoxy-methyl , 4-fluoro-phenoxy-methyl, especially heterocycle substituted methyl, more especially 2-thiophenyl-methyl;

ethyl, especially piperidin-1-yl-ethyl;

butyl, especially aryl substituted butyl, more especially 4-(4-methoxy)phenyl-butyl;

isopentyl;

cyclohexyl;

pentanonyl, especially 4-pentanonyl;

butenyl, especially aryl substituted butenyl, more especially 4,4-bis(4-methoxyphenyl)-but-3-enyl;

acetyl;

phenyl, especially phenyl substituted with one or more halogens, more especially 3,4-dichlorophenyl and 4-fluorophenyl, especially phenyl substituted with one or more aryloxy or C₁₋₆alkoxy groups, more especially 3,4-dimethoxy-phenyl, 3-benzyloxy-4-methoxy-phenyl, especially phenyl substituted with one or more C₁₋₆alkyl sulfonyl groups, more especially 4-methanesulfonyl-phenyl;

benzyl;

naphthalenyl, especially naphthylen-2-yl;

benzo[1,3]dioxolyl, especially benzo[1,3]dioxol-5-yl;

furanyl, especially furan-2-yl, especially substituted furanyl, such as 5-nitro-furan-2-yl, 5-(4-nitrophenyl)-furan-2-yl, 5-(3-trifluoromethyl-phenyl)-furan-2-yl, more especially halogen substituted furanyl, even more especially 5-bromo-furan-2-yl, more especially aryl substituted furanyl, even more especially 5-(4-chloro-phenyl)-furan-2-yl, more especially C₁₋₆alkyl substituted furanyl, even more especially 3-methyl-furan-2-yl, 4-methyl-furan-2-yl, 2,5-dimethyl-furan-2-yl, and 2,4-dimethyl-furan-3-yl;

tetrahydrofuranyl, especially tetrahydrofuran-2-yl;

benzofuranyl, especially benzofuran-2-yl, and substituted benzofuranyl, more especially 5-(2-piperazin-4-carboxylic acid tert-butyl ester-ethoxy) benzofuran-2-yl, 5-(2-morpholino-4-yl-ethoxy)-benzofuran-2-yl, 5-(2-piperazin-1-yl-ethoxy)benzofuran-2-yl, 5-(2-cyclohexyl-ethoxy)-benzofuran-2-yl; especially C₁₋₆alkoxy substituted benzofuranyl, more especially 7-methoxy-benzofuran-2-yl, 5-methoxy-benzofuran-2-yl, 5,6-dimethoxy-benzofuran-2-yl, especially halogen substituted benzofuranyl, more especially 5-fluoro-benzofuran-2-yl, 5,6-difluoro-benzofuran-2-yl, especially C₁₋₆alkyl substituted benzofuranyl, most especially 3-methyl-benzofuran-2-yl, 3,5-dimethyl-benzofuran-2-yl, and 3-ethyl-benzofuran-2-yl; also 5-fluoro-3-methyl-benzofuran-2-yl, 6-fluoro-3-methyl-benzofuran-2-yl, 5-methoxy-3-methyl-benzofuran-2-yl, 4-methoxy-3-methyl-benzofuran-2-yl, and 6-methoxy-3-methyl-benzofuran-2-yl;

naphtho[2,1-b]-furanyl, especially naphtho[2,1-b]-furan-2-yl, alkyl substituted naphtho[2,1-b]-furanyl, especially 1-methyl-naphtho[2,1-b]-furan-2-yl;

benzo[b]thiophenyl, especially benzo[b]thiophen-2-yl; especially C₁₋₆alkoxy substituted benzo[b]thiophenyl, more especially 5,6-dimethoxy-benzo[b]thiophen-2-yl;

quinolinyl, especially quinolin-2-yl, quinolin-3-yl, quinolin-4-yl, quinolin-6-yl, and quinolin-8-yl;

quinoxalinyl, especially quinoxalin-2-yl;

1,8 naphthyridinyl, especially 1,8 naphthyridin-2-yl;

indolyl, especially indol-2-yl, especially indol-6-yl, indol-5-yl, especially C₁₋₆alkyl substituted indolyl, more especially N-methyl-indol-2-yl;

pyridinyl, especially pyridin-2-yl, pyridin-3-yl, pyridin-5-yl, especially C₁₋₆alkyl substituted pyridinyl, more especially 2-methyl-pyridin-5-yl, and oxy-pyridinyl, especially 1-oxy-pyridin-2-yland 1-oxy-pyridin-3-yl;;

furo[3,2-b]-pyridinyl, especially furo[3,2-b]-pyridin-2-yl, CI₁₋₆alkyl substituted furo[3,2-b]-pyridinyl, especially 3-methyl-furo[3,2-b]-pyridin-2-yl;

thiophenyl, especially thiophen-3-yl, also thiophen-2-yl, especially C₁₋₆alkyl substituted thiophenyl, more especially 5-methyl-thiophen-2-yland 5-methyl-thiophen-3-yl, especially halogen substituted thiophenyl, more especially 4,5-dibromo-thiophen-2-yl;

thieno[3,2-b]thiophene, especially thieno[3,2-b]thiophene-2-yl, more especially C₁₋₆alkyl substituted thieno[3,2-b]thiophene-2-yl, more especially 5-tert-butyl-3-methyl-thieno[3,2-b]thiophene-2-yl;

isoxazolyl, especially isoxazol-4-yl, especially C₁₋₆alkyl substituted isoxazolyl, more especially 3,5-dimethyl-isoxazol-4-yl;

oxazolyl, especially oxazol-4-yl, more especially 5-methyl-2-phenyl oxazol-4-yl, 2-phenyl-5-trifluoromethyl-oxazol-4-yl; and

1H-benzoimidazolyl, especially 1H-benzoimidazol-5-yl.

R⁹ is selected from the group consisting of:

methyl;

ethyl, especially C₁₋₆alkyl-substituted ethyl, more especially 2-cyclohexyl-ethyl;

propyl;

butyl, especially C₁₋₆butyl, more especially 3-methylbutyl;

tert-butyl, particularly when R² is R⁹OC(O);

isopentyl;

phenyl, especially halogen substituted phenyl, more especially 3,4-dichlorophenyl, 4-bromophenyl, 2-fluorophenyl, 3-fluorophenyl, 4-fluorophenyl, 2-chlorophenyl, 3-chlorophenyl, 4-chlorophenyl, especially C₁₋₆alkoxy phenyl, more especially 3-methoxyphenyl, 4-methoxyphenyl, 3,4-dimethoxyphenyl, especially cyanophenyl, more especially 2-cyanophenyl; especially C₁₋₆alkyl substituted phenyl, more especially 4-ethyl-phenyl, 2-methyl phenyl, 4-methyl phenyl, especially C₁₋₆alkyl sulfonyl substituted phenyl, more especially 4-methanesulfonyl phenyl, and 2-methanesulfonyl phenyl;

toluyl, especially Het-substituted toluyl, more especially 3-(pyridin-2-yl)toluyl;

naphthylene, especially naphthyl-2-ene;

benzoic acid, especially 2-benzoic acid;

benzo[1,3]dioxolyl, especially benzo[1,3]dioxol-5-yl;

benzo[1,2,5]oxadiazolyl, especially benzo[1,2,5]oxadiazol-4-yl;

pyridinyl, especially pyridin-2-yl, pyridin-3-yl, especially 1-oxy-pyridinyl, more especially 1-oxy-pyridin-2-yl, 1-oxy-pyridin-3-yl; especially C₁₋₆alkylpyridinyl, more especially 3-methyl-pyridin-2-yl, 6-methyl-pyridin-2-yl:

thiophenyl, especially thiophenyl-2-yl;

thiazolyl, especially thiazol-2-yl;

1H-imidazolyl, especially 1H-imidazol-2-yl, 1H-imidazol-4-yl, more especially C₁₋₆alkyl substituted imidazolyl, even more especially 1-methyl-1H-imidazol-2-yl, 1-methyl-1H-imidazol-4-yl, and 1,2-dimethyl-1H-imidazol-4-yl;

triazolyl, especially 1H-[1,2,4]triazolyl, more especially 1H-[1,2,4]triazol-3-yl, especially C₁₋₆alkyl substituted 1H-[1,2,4]triazolyl, more especially 5-methyl-1H-[1,2,4]triazol-3-yl; and

isoxazolyl, especially isoxazol-4-yl, especially C₁₋₆alkyl substituted isoxazolyl, more especially 3,5-dimethyl-isoxazol-4-yl;

R′ is H;

R″ is H; and

R′″ is H.

Also preferred are such compounds wherein R′″ is selected from the group consisting of: 6-methyl and 7-methyl, preferably 7-methyl.

The following compounds are preferred for use in the present methods of treatment:

Chemical Name

Benzo[1,3]dioxole-5-carboxylic acid [(S)-1-(1-benzyl-3-oxoazepan-4-ylcarbamoyl)-3-methyl-butyl]amide;

Quinoline-2-carboxylic acid [(S)-1-(1-benzyl-3-oxo-azepan-4-ylcarbamoyl)-3-methyl-butyl]amide;

5-(2-Morpholino-4-yl-ethoxy)-benzofuran-2-carboxylic acid ((S)-3-methyl-1-{3-oxo-1-[2-(3-pyridin-2-yl-phenyl)acetyl]-azepan-4-ylcarbamoyl}-butyl)amide;

5-(2-Morpholino-4-yl-ethoxy)-benzofuran-2-carboxylic acid ((S)-3-methyl-1-{3-oxo-1-[2-(3-pyridin-2-yl-phenyl)acetyl]-azepan-4-ylcarbamoyl}-butyl)amide;

4-((S)-4-Methyl-2-{[5-(2-morpholino-4-yl-ethoxy)-benzofuran-2-carbonyl]amino}-pentanoylamino)-3-oxo-azepane-1-carboxylic acid phenylamide;

5-(2-Morpholin-4-yl-ethoxy)-benzofuran-2-carboxylic acid [(S)-1-(1-benzenesulfonyl-3-oxo-azepan-4-ylcarbamoyl)-3-methyl-butyl]amide;

5-(2-Morpholin-4-yl-ethoxy)-benzofuran-2-carboxylic acid [(S)-1-(1-benzenesulfonyl-3-oxo-azepan-4-ylcarbamoyl)-3-methyl-butyl]amide;

5-(2-Pyrrolidin-1-yl-ethoxy)-benzofuran-2-carboxylic acid [(S)-1-(1-benzenesulfonyl-3-oxo-azepan-4-ylcarbamoyl)-3-methyl-butyl]amide;

5-(2-Piperidin-1-yl-ethoxy)-benzofuran-2-carboxylic acid [(S)-1-(1-benzenesulfonyl-3-oxo-azepan-4-ylcarbamoyl)-3-methyl-butyl]amide;

Naphthlene-2-carboxylic acid ((S)-3-methyl-1-{3-oxo-1-[2-(3-pyridin-2-yl-phenyl)ethyl-]-azepan-4-ylcarbamoyl}-butyl)amide;

1H-Indole-2-carboxylic acid [(S)-1-(1-benzenesulfonyl-3-oxo-azepan-4-ylcarbamoyl)-3-methyl-butyl]amide;

Benzofuran-2-carboylic acid [(S)-1-(1-benzenesulfonyl-3-oxo-azepan-4-ylcarbamoyl)-3-methyl-butyl]amide,

5-(2-Pyrrolidin-1-yl-ethoxy)-benzofuran-2-carboxylic acid [(S)-1-(1-benzenesulfonyl-3-oxo-azepan-4-ylcarbamoyl)-3-methyl-butyl]amide;

5-(2-Piperidin-1-yl-ethoxy)-benzofuran-2-carboxylic acid [(S)-1-(1-benzenesulfonyl-3-oxo-azepan-4-ylcarbamoyl)-3-methyl-butyl]amide;

5-(2-Morpholino-4-yl-ethoxy)-benzofuran-2-carboxylic acid {(S)-3-methyl-1-[1-(4-methy-pentanoyl)-3-oxo-azepan-4-ylcarbamoyl]-butyl}-amide;

Benzofuran-2-carboxylic acid {(S)-3-methyl-1-[3-oxo-1-(pyridine-2-sulfonyl)-azepan-4-ylcarbamoyl]-butyl}-amide;

Naphthalene-2-carboxylic acid {(S)-3-methyl-1-[3-oxo-1-(pyridine-2-sulfonyl)-azepan-4-ylcarbamoyl]-butyl}-amide;

5-(2-Morpholino-4-yl-ethoxy)-benzofuran-2-carboxylic acid {(S)-3-methyl-1-[3-oxo-1-(pyridine-2-sulfonyl)-azepan-4-ylcarbamoyl]-butyl}-amide;

5-(2-Morpholino-4-yl-ethoxy)-benzofuran-2-carboxylic acid {(S)-3-methyl-1-[3-oxo-1-(pyridine-2-sulfonyl)-azepan-4-ylcarbamoyl]-butyl}-amide;

5-(2-Morpholino-4-yl-ethoxy)-benzofuran-2-carboxylic acid {(S)-3-methyl-1-[3-oxo-1-(pyridine-2-sulfonyl)-azepan-4ylcarbamoyl]-butyl}-amide;

Benzofuran-2-carboxylic acid {(S)-3-methyl-1-[3-oxo-1-(pyridine-2-sulfonyl)-azepan-4-ylcarbamoyl]-butyl}-amide;

4-[2-(2-{(S)-3-Methyl-1-[3-oxo-1-(pyridine-2-sulfonyl)-azepan-4-ylcarbamoy]-butylcarbamoyl}-benzofuran-5-yloxy)-ethyl]-piperazine-1-carboxylic acid tert-butyl ester;

5-(2-Piperizin-1-yl-ethoxy)-benzofuran-2-carboxylic acid {(S)-3-methyl-1-[3-oxo-1-(pyridine-2-sulfonyl)-azepan-4-ylcarbamoyl]-3-butyl}-amide;

Quinoline-2-carboxylic acid {(S)-3-methyl-1-[3-oxo-1-(pyridine-2-sulfonyl)-azepan-4-ylcarbamoyl]-butyl}amide;

Quinoline-6-carboxylic acid {(S)-3-methyl-1-[3-oxo-1-(pyridine-2-sulfonyl)-azepan-4-ylcarbamoyl]-butyl}amide;

Quinoline-4-carboxylic acid {(S)-3-methyl-1-[3-oxo-1-(pyridine-2-sulfonyl)-azepan-4-ylcarbamoyl]-butyl}amide;

Quinoline-3-carboxylic acid {(S)-3-methyl-1-[3-oxo-1-(pyridine-2-sulfonyl)-azepan-4-ylcarbamoyl]-butyl}amide;

Isoquinoline-3-carboxylic acid {(S)-3-methyl-1-[3-oxo-1-(pyridine-2-sulfonyl)-azepan-4-ylcarbamoyl]-butyl}amide;

Isoquinoline-1-carboxylic acid {(S)-3-methyl-1-[3-oxo-1-(pyridine-2-sulfonyl)-azepan-4-ylcarbamoyl]-butyl}amide;

Quinoxaline-2-carboxylic acid {(S)-3-methyl-1-[3-oxo-1-(pyridine-2-sulfonyl)-azepan-4-ylcarbamoyl]-butyl}amide;

Benzo[b]thiophene-2-carboxylic acid {(S)-3-methyl-1-[3-oxo-1-(pyridine-2-sulfonyl)-azepan-4-ylcarbamoyl]-butyl}amide;

1H-Indole-2-carboxylic acid {(S)-3-methyl-1-[3-oxo-1-(pyridine-2-sulfonyl)-azepan-4-ylcarbamoyl]-butyl}amide;

5-Methoxy-benzofuran-2-carboxylic acid {(S)-3-methyl-1-[3-oxo-1-(pyridine-2-sulfonyl)-azepan-4-ylcarbamoyl]-butyl} amide;

5-Bromo-furan-2-carboxylic acid {(S)-3-methyl-1-[3-oxo-1-(pyridine-2-sulfonyl)-azepan-4-ylcarbamoyl]-butyl)}amide;

5-Nitro-furan-2-carboxylic acid {(S)-3-methyl-1-[3-oxo-1-(pyridine-2-sulfonyl)-azepan-4-ylcarbamoyl]-butyl}amide;

5-(4-Nitro-phenyl)-furan-2-carboxylic acid {(S)-3-methyl-1-[3-oxo-1-(pyridine-2-sulfonyl)-azepan-4-ylcarbamoyl]-butyl}amide;

(S)-2-[2-(4-Fluoro-phenoxy)-acetylamino]-4-methyl-pentanoic acid [3-oxo-(pyridine-2-sulfonyl)-azepan-4-yl]-amide;

Benzofuran-2-carboxylic acid {(S)-3-methyl-1-[3-oxo-1-(thiophene-2-sulfonyl)-azepan-4-ylcarbamoyl]-butyl}-amide;

5,6-Dimethoxy-benzofuran-2-carboxylic acid {(S)-3-methyl-1-[3-oxo-1-(1-methyl-1H-imidazole-4-sulfonyl)-azepan-4-ylcarbamoyl]-butyl}amide;

Benzofuran-2-carboxylic acid {(S)-3-methyl-1-[1-(1-methyl-1H-imidazole-3-sulfonyl)-3-oxo-azepan-4-ylcarbamoyl]-butyl}amide;

Benzofuran-2-carboxylic acid {(S)-3-methyl-1-[1-(1H-imidazole-2-sulfonyl)-3-oxo-azepan-4-ylcarbamoyl]-butyl}amide;

Benzofuran-2-carboxylic acid {(S)-3-methyl-1-[1-(1-methyl-1H-imidazole-4-sulfonyl)-3-oxo-azepan-4-ylcarbamoyl]-butyl}amide;

5-(4-Oxy-morpholino-4-yl-ethoxy)-benzofuran-2-carboxylic acid {(S)-3-methyl-1-[3-oxo-1-(pyridine-2-sulfonyl)-azepan-4-ylcarbamoyl]-butyl}amide;

5-Hydroxy-benzofuran-2-carboxylic acid {(S)-3-methyl-1-[1-(1-methyl-1H-imidazole-4-sulfonyl)-3-oxo-azepan-4-ylcarbamoyl]-butyl}amide;

Benzofuran-2-carboxylic acid {(S)-3-methyl-1-[3-oxo-1-(1-oxy-pyridine-2-sulfonyl)-azepan-4-ylcarbamoyl)]-3-methyl-butyl}-amide;

Benzo[b]thiophene-2-carboxylic acid {(S)-3-methyl-1-[3-oxo-1-(1-oxy-pyridine-2-sulfonyl)-azepan-4-ylcarbamoyl]-butyl}amide;

5-Bromo-furan-2-carboxylic acid {(S)-3-methyl-1-[3-oxo-1-(1-oxy-pyridine-2-sulfonyl)-azepan-4-ylcarbamoyl]-butyl}amide;

5,6-Dimethoxy-benzofuran-2-carboxylic acid {(S)-3-methyl-1-[3-oxo-1-(1-oxy-pyridine-2-sulfonyl)-azepan-4-ylcarbamoyl]-butyl}amide;

Benzofuran-2-carboxylic acid {(S)-3-methyl-1-[3-oxo-1-(1-oxy-pyridine-2-sulfonyl)-azepan-4-ylcarbamoyl)]-3-methyl-butyl}-amide;

5-Bromo-furan-2-carboxylic acid {(S)-3-methyl-1-[3-oxo-1-(1-oxy-pyridine-2-sulfonyl)-azepan-4-ylcarbamoyl]-butyl}amide;

Benzo[b]thiophene-2-carboxylic acid {(S)-3-methyl-1-[3-oxo-1-(1-oxy-pyridine-2-sulfonyl)-azepan-4-ylcarbamoyl]-butyl}amide;

5,6-Dimethoxy-benzofuran-2-carboxylic acid {(S)-3-methyl-1-[3-oxo-1-(1-oxy-pyridine-2-sulfonyl)-azepan-4-ylcarbamoyl]-butyl}amide;

5-Methoxy-benzofuran-2-carboxylic acid {(S)-3-methyl-1-[3-oxo-1-(1-oxy-pyridine-2-sulfonyl)-azepan-4-ylcarbamoyl]-butyl}amide;

1H-Indole-5-carboxylic acid {(S)-3-methyl-1-[3-oxo-1-(1-oxy-pyridine-2-sulfonyl)-azepan-4-ylcarbamoyl]-butyl}amide;

Benzo[1,3]dioxole-5-carboxylic acid {(S)-3-methyl-1-[3-oxo-1-(1-oxy-pyridine-2-sulfonyl)-azepan-4-ylcarbamoyl]-butyl}amide;

5-(2-Morpholin-4-yl-ethoxy)-benzofuran-2-carboxylic acid {(S)-3-methyl-1-[3-oxo-(1-oxy-pyridine2-sulfonyl)-azepan-4-ylcarbamoyl]-buty}-amide;

3-Methyl-benzofuran-2-carboxylic acid {(S)-3-methyl-1-[3-oxo-1-(1-oxy-pyridine-2-sulfonyl)-azepan-4-ylcarbamoyl]-butyl}amide;

1H-Indole-6-carboxylic acid {(S)-3-methyl-1-[3-oxo-1-(pyridine-2-sulfonyl)-azepan-4-ylcarbamoyl]-butyl}amide;

Benzo[1,3]dioxole-5-carboxylic acid {(S)-3-methyl-1-[3-oxo-1-(pyridine-2-sulfonyl)-azepan-4-ylcarbamoyl]-butyl}amide;

3,4-Dihydro-2H-benzo[b][1,4]dioxepine-7-carboxylic acid {(S)-3-methyl-1-[3-oxo-1-(1-oxy-pyridine-2-sulfonyl)-azepan-4-ylcarbamoyl]butyl}amide;

4,5-Dibromo-thiophene-2-carboxylic acid {(S)-3-methyl-1-[3-oxo-1-(1-oxy-pyridine-2-sulfonyl)-azepan-4-ylcarbamoyl]-butyl}amide;

Thieno[3,2-b]thiophene-2-carboxylic acid {(S)-3-methyl-1-[3-oxo-1-(1-oxy-pyridine-2-sulfonyl)-azepan-4-ylcarbamoyl]-butyl}amide;

5-Methoxy-benzofuran-2-carboxylic acid {(S)-3-methyl-1-[3-oxo-1-(1-oxy-pyridine-2-sulfonyl)-azepan-4-ylcarbamoyl]-butyl}amide;

1H-Indole-5-carboxylic acid {(S)-3-methyl-1-[3-oxo-1-(1-oxy-pyridine-2-sulfonyl)-azepan-4-ylcarbamoyl]-butyl}amide;

5-(4-Chloro-phenyl)-furan-2-carboxylic acid {(S)-3-methyl-1-[3-oxo-1-(pyridine-2-sulfonyl)-azepan-4-ylcarbamoyl]-butyl}amide;

5-(3-Trifluoromethyl-phenyl)-furan-2-carboxylic acid {(S)-3-methyl-1-[3-oxo-1-(pyridine-2-sulfonyl)-azepan-4-ylcarbamoyl]-butyl}amide;

Benzo[1,3]dioxole-5-carboxylic acid {(S)-1-[1-(4-fluoro-benzenesulfonyl)-3-oxo-azepan-4-ylcarbamoyl]-3methyl-butyl}-amide;

5-Bromo-furan-2-carboxylic acid {(S)-3-methyl-1-[3-oxo-1-(pyridine-2-sulfonyl)-azepan-4-ylcarbamoyl]-butyl}amide;

5-Methoxy-benzofuran-2-carboxylic acid {(S)-3-methyl-1-[3-oxo-1-(pyridine-2-sulflotyl)-azepan-4-ylcarbamoyl]-butyl}amide;

3-Methyl-benzofuran-2-carboxylic acid {(S)-3-methyl-1-[3-oxo-1-(pyridine-2-sulfonyl)-azepan-4-ylcarbamoyl]-butyl}amide;

Thieno[3,2-b]thiophene-2-carboxylic acid {(S)-3-methyl-1-[3-oxo-1-(pyridine-2-sulfonyl)-azepan-4-ylcarbamoyl]-butyl}amide;

2-Phenyl-5-trifluoromethyl-oxazole-4-carboxylic acid {(S)-3-methyl-1-[3-oxo-1-(pyridine-2-sulfonyl)-azepan4-ylcarbamoyl]-butyl}amide;

Benzo[1,3]dioxole-5-carboxylic acid {(S)-1-[-(4-methoxy-benzenesulfonyl)-3-oxo-azepan-4-ylcarbamoyl]-3-methyl-butyl}-amide;

Benzofuran-2-carboxylic acid {(S)-1-[1-(4-methoxy-benzenesulfonyl)-3-oxo-azepan-4-ylcarbamoyl]-3-methyl-butyl}-amide;

Furan-2-carboxylic acid {(S)-3-methyl-1-[3-oxo-1-(1-oxy-pyridine-2-sulfonyl)-azepan-4-ylcarbamoyl]-butyl}amide;

Benzo[1,3]dioxole-5-carboxylic acid {(S)-3-methyl-1-[3-oxo-1-(1-oxy-pyridine-2-sulfonyl)-azepan-4-ylcarbamoyl]-butyl}amide;

4-Fluoro-{(S)-3-methyl-1-[3-oxo-1-(1-oxy-pyridine-2-sulfonyl)-azepan-4-carbamoyl]-butyl}-benzamide;

3,4-Dihydro-2H-benzo[b][1,4]dioxepine-7-carboxylic acid {(S)-3-methyl-1-[3-oxo-1-(1-oxy-pyridine-2-sulfonyl)-azepan-4-ylcarbamoyl]butyl}amide;

5-Methyl-thiophene-2-carboxylic acid {(S)-3-methyl-1-[3-oxo-1-( -oxy-pyridine-2-sulfonyl)-azepan-4-ylcarbamoyl]-butyl}amide;

(S)-2-(3-Benzyl-ureido)-4-methyl-pentanoic acid [3-oxo-1-(pyridine-2-sulfonyl)-azepan-4-yl]-amide;

5-Methoxy-benzofuran-2-carboxylic acid [(S)-1-(1-methanesulfonyl-3-oxo-azepan-4-ylcarbamoyl)-3-methyl-butyl]-amide;

Furan-2-carboxylic acid ({(S)-1-[1-(4-methoxy-benzenesulfonyl)-3-oxo-azepan-4-ylcarbamoyl]-3-methyl-butylcarbamoyl}-methyl)-amide;

Quinoline-2-carboxylic acid {[(S)-1-[1-(4-fluoro-benzenesulfonyl)-3-oxo-azepan-4-ylcarbamoyl]-3-methyl-butyl}-amide;

1-Methyl-1H-indole-2-carboxylic acid {[(S)-1-[1-(4-fluoro-benzenesulfonyl)-3-oxo-azepan-4-ylcarbamoyl]-3-methyl-butyl}-amide;

5-Methoxy-benzofuran-2-carboxylic acid {[(S)-1-[1-(4-fluoro-benzenesulfonyl)-3-oxo-azepan-4-ylcarbamoyl]-3-methyl-butyl}-amide;

Quinoxaline-2-carboxylic acid {[(S)-1-[1-(4-fluoro-benzenesulfonyl)-3-oxo-azepan-4-ylcarbamoyl]-3-methyl-butyl}-amide;

Benzo[b]thiophene-2-carboxylic acid {(S)-3-methyl-1-[3-oxo-1-(pyridine-2-sulfonyl)-azepan-4-ylcarbamoyl]-butyl}amide;

Benzofuran-2-carboxylic acid-{(S)-1-[1-(3-chloro-benzenesulfonyl)-3-oxo-azepan-4-ylcarbamoyl]-3-methyl-butyl}-amide;

5-Methoxy-benzofuran-2-carboxylic acid-{(S)-1-[1-(3-chloro-benzenesulfonyl)-3-oxo-azepan-4-ylcarbamoyl]-3-methyl-butyl}-amide;

3-Methyl-benzofuran-2-carboxylic acid-{(S)-1-[1-(3-chloro-benzenesulfonyl)-3-oxo-azepan-4-ylcarbamoyl]-3-methyl-butyl}-amide;

Benzofuran-2-carboxylic acid-{(S)-1-[1-(2-fluoro-benzenesulfonyl)-3-oxo-azepan-4-ylcarbamoyl]-3-methyl-butyl}-amide;

5-Methoxy-benzofuran-2-carboxylic acid-{(S)-1-[1-(2-fluoro-benzenesulfonyl)-3-oxo-azepan-4-ylcarbamoyl]-3-methyl-butyl}-amide;

7-Methoxy-benzofuran-2-carboxylic acid-{(S)-1-[1-(2-fluoro-benzenesulfonyl)-3-oxo-azepan-4-ylcarbamoyl]-3-methyl-butyl}-amide;

3-methylbenzofuran-2-carboxylic acid-{(S)-1-[1-(2-fluoro-benzenesulfonyl)-3-oxo-azepan-4-ylcarbamoyl]-3-methyl-butyl}-amide;

Benzo[b]thiophene-2-carboxylic acid-{(S)-1-[1-(2-fluoro-benzenesulfonyl)-3-oxo-azepan-4-ylcarbamoyl]-3-methyl-butyl}-amide;

Quinoxaline-2-carboxylic acid-{(S)-1-[1-(2-fluoro-benzenesulfonyl)-3-oxo-azepan-4-ylcarbamoyl]-3-methyl-butyl}-amide;

3-Methyl-benzofuran-2-carboxylic acid {(S)-3-methyl-1-[3-oxo-1-(pyridine-2-sulfonyl)-azepan-4-ylcarbamoyl]-butyl}amide;

Thieno[3,2-b]thiophene-2-carboxylic acid {(S)-3-methyl-1-[3-oxo-1-(pyridine-2-sulfonyl)-azepan-4-ylcarbamoyl]-butyl}amide;

3-Methyl-benzofuran-2-carboxylic acid {(S)-3-methyl-1-[3-oxo-1-(1-oxy-pyridine-2-sulfonyl)-azepan-4-ylcarbamoyl]-butyl}amide;

Benzofuran-2-carboxylic acid {(S)-3-methyl-1-[(2,2′,4-tridueterio)-3-oxo-1-(pyridine-2-sulfonyl)-azepan-4-ylcarbamoyl]-butyl}amide;

Quinoxaline-2-carboxylic acid {(S)-3-methyl-1-[3-oxo-1-(1-oxy-pyridine-2-sulfonyl)-azepan-4-ylcarbamoyl]-butyl}amide;

Benzofuran-2-carboxylic acid {(S)-2-cyclohexyl-1-[3-oxo-1-(pyridine-2-sulfonyl)-azepan-4-ylcarbamoyl]-ethyl}-amide;

Benzofuran-2-carboxylic acid {(S)-1-[3-oxo-1-(pyridine-2-sulfonyl)-azepan-4-ylcarbamoyl]-pentyl}-amide;

Benzofuran-2-carboxylic acid {(S)-1-[3-oxo-1-(pyridine-2-sulfonyl)-azepan-4-ylcarbamoyl]-2-phenyl-ethyl}-amide;

2-Phenyl-5-trifluoromethyl-oxazole-4-carboxylic acid {(S)-3-methyl-1-[3-oxo-1-(pyridine-2-sulfonyl)-azepan-4-ylcarbamoyl]-butyl}amide;

5-Methyl-2-phenyl-oxazole-4-carboxylic acid {(S)-3-methyl-1-[3-oxo-1-(pyridine-2-sulfonyl)-azepan-4-ylcarbamoyl]-butyl}amide;

3,4-Dimethoxy-N—{(S)-1-[1-(4-methoxy-benzenesulfonyl)-3-oxo-azepan-4-ylcarbamoyl]-3-methyl-butyl}-benzamide;

Benzo[b]thiophene-2-carboxylic acid-{(S)-1-[1-(4-fluoro-benzenesulfonyl)-3-oxo-azepan-4-yl carbamoyl]-3-methyl-butyl}-amide;

Benzofuran-2-carboxylic acid-{(S)-1-[1-(4-fluoro-benzenesulfonyl)-3-oxo-azepan-4-yl carbamoyl]-3-methyl-butyl}-amide;

N—{(S)-1-{1-(4-Fluoro-benzenesulfonyl)-3-oxo-azepan-4-ylcarbamoyl}-3-methyl-butyl}-3,4-dimethoxy-benzamide;

Benzo[b]thiophene-2-carboxylic acid-{(S)-1-(1-methanesulfonyl-3-oxo-azepan-4-yl carbamoyl)-3-methyl-butyl]-amide;

Benzofuran-2-carboxylic acid-{(S)-1-(1-methanesulfonyl-3-oxo-azepan-4-yl carbamoyl)-3-methyl-butyl]-amide;

N—[(S)-1-(1-Methanesulfonyl)-3-oxo-azepan-4-ylcarbamoyl}-3-methyl-butyl}-3,4-dimethoxy-benzamide;

N—{(S)-1-{1-(2-Cyano-benzenesulfonyl)-3-oxo-azepan-4-ylcarbamoyl}-3-methyl-butyl}-4-methanesulfonyl-benzamide;

Benzofuran-2-carboxylic acid {(S)-1-[1-(2-cyano-benzenesulfonyl)-3-oxo-azepan-4-yl carbamoyl]-3-methyl-butyl}-amide;

5-(2-Morpholin-4-yl-ethoxy)-benzofuran-2-carboxylic acid {(S)-3-methyl-1-[3-oxo-(1-oxy-pyridine2-sulfonyl)-azepan-4-ylcarbamoyl]-1-buty}-amide;

5-Methyl-2-phenyl-oxazole-4-carboxylic acid {(S)-3-methyl-1-[3-oxo-1-(1-oxy-pyridine-2-sulfonyl)-azepan-4-ylcarbamoy ]-butyl}amide;

6-Methyl-N—{(S)-3-methyl-1-[3-oxo-1-(1-oxy-pyridine-2-sulfonyl)-azepan-4-ylcarbamoyl]-butyl}-nicotinamide;

5-(3-Trifluoromethyl-phenyl)-furan-2-carboxylic acid {(S)-3-methyl-1-[3-oxo-1-(1-oxy-pyridine-2-sulfonyl)-azepan-4-ylcarbamoyl]-butyl}amide;

N—{(S)-1-{(1-(2-cyano-benzenesulfonyl)-3-oxo-azepan -4-ylcarbamoyl}-3-methyl-butyl}-3,4-dimethoxy-benzamide;

4-Methansulfonyl-N—{(S)-1-[4-fluoro-benzenesulfonyl)-3-oxo-azepan-4-carbamoyl]-3-methyl-butyl-benzamide;

(S)-2-[5-(4-Methoxy-phenyl)-pentanoylamnio]-4-methyl-pentanoic acid [3-oxo-1-(pyridine-2-sulfonyl)-azepan-4-yl]-amide;

(S)-2-[2-(3-Benzyloxy-4-methoxy-phenyl)-acetylamnio]-4-methylpentanoic acid [3-oxo-1-(pyridine-2-sulfonyl)-azepan-4-yl]-amide;

5-Methoxybenzofuran-2-carboxylic acid {(S)-3-methyl-1-[3-oxo-1-(thiazole-2-sulfonyl)-azepan-4-ylcarbamoyl]-butyl}amide;

7-Methoxybenzofuran-2-carboxylic acid {(S)-3-methyl-1-[3-oxo-1-(thiazole-2-sulfonyl)-azepan-4-ylcarbamoyl]-butyl}amide;

3-Methylbenzofuran-2-carboxylic acid {(S)-3-methyl-1-[3-oxo-1-(thiazole-2-sulfonyl)-azepan-4-ylcarbamoyl]-butyl}amide;

Benzo[b]thiophene-2-carboxylic acid {(S)-3-methyl-1-[3-oxo-1-(thiazole-2-sulfonyl)-azepan-4-ylcarbamoyl]-butyl}amide;

1-Methyl-1H-indole-2-carboxylic acid {(S)-3-methyl-1-[3-oxo-1-(thiazole-2-sulfonyl)-azepan-4-ylcarbamoyl]-butyl}amide;

Quinoxaline-2-carboxylic acid {(S)-3-methyl-1-[3-oxo-1-(thiazole-2-sulfonyl)-azepan-4-ylcarbamoyl]-butyl}amide;

Benzofuran-2-carboxylic acid {(S)-3-methyl-1-[3-oxo-1-(thiazole-2-sulfonyl)-azepan-4-ylcarbamoyl]-butyl)}amide;

Benzofuran-2-carboxylic acid {(S)-1-[1-(4-chloro-benzenesulfonyl)-3-oxo-azepan-4-ylcarbamoyl]-3-methyl-butyl}-amide;

5-Methoxy-benzofuran-2-carboxylic acid-{(S)-1-[1-(4-chloro-benzenesulfonyl)-3-oxo-azepan-4-ylcarbamoyl]-3-methyl-butyl}-amide;

7-Methoxy-benzofuran-2-carboxylic acid-{(S)-1-[1-(4-chloro-benzenesulfonyl)-3-oxo-azepan-4-ylcarbamoyl]-3-methyl-butyl}-amide;

3-Methyl-benzofuran-2-carboxylic acid-{(S)-1-[1-(4-chloro-benzenesulfonyl)-3-oxo-azepan-4-ylcarbamoyl]-3-methyl-butyl}-amide;

Benzo[b]thiophene-2-carboxylic acid-{(S)-1-[1-(4-chloro-benzenesulfonyl)-3-oxo-azepan-4-ylcarbamoyl]-3-methyl-butyl}-amide;

Benzofuran-2-carboxylic acid-{(S)-1-[1-(3-methoxy-benzenesulfonyl)-3-oxo-azepan-4-ylcarbamoyl]-3-methyl-butyl}-amide;

5-Methoxy-benzofuran-2-carboxylic acid-{(S)-1-[1-(3-methoxy-benzenesulfonyl)-3-oxo-azepan-4-ylcarbamoyl]-3-methyl-butyl}-amide;

7-Methoxy-benzofuran-2-carboxylic acid-{(S)-1-[1-(3-methoxy-benzenesulfonyl)-3-oxo-azepan-4-ylcarbamoyl]-3-methyl-butyl}-amide;

3-Methyl-benzofuran-2-carboxylic acid-{(S)-1-[1-(3-methoxy-benzenesulfonyl)-3-oxo-azepan-4-ylcarbamoyl]-3-methyl-butyl}-amide;

Benzo[b]thiophene-2-carboxylic acid-{(S)-1-[1-(3-methoxy-benzenesulfonyl)-3-oxo-azepan-4-ylcarbamoyl]-3-methyl-butyl}-amide;

1-Methyl-1H-indole-2-carboxylic acid-{(S)-1-[1-(3-methoxy-benzenesulfonyl)-3-oxo-azepan-4-ylcarbamoyl]-3-methyl-butyl}-amide;

Benzofuran-2-carboxylic acid-{(S)-3-methyl-1-[3-oxo-1-(thiophene-2-sulfonyl)-azepan-4-ylcarbamoyl]-butyl}-amide;

5-Methoxy-benzofuran-2-carboxylic acid-{(S)-3-methyl-1-[3-oxo-1-(thiophene-2-sulfonyl)-azepan-4-ylcarbamoyl3-butyl}-amide;

7-Methoxy-benzofuran-2-carboxylic acid-{(S)-3-methyl-1-[3-oxo-1-(thiophene-2-sulfonyl)-azepan-4-ylcarbamoyl]-butyl}-amide;

3-Methyl-benzofuran-2-carboxylic acid-{(S)-3-methyl-1-[3-oxo-1-(thiophene-2-sulfonyl)-azepan-4-ylcarbamoyl]-butyl}-amide;

Benzo[b]thiophene-2-carboxylic acid-{(S)-3-methyl-1-[3-oxo-1-(thiophene-2-sulfonyl)-azepan-4-ylcarbamoyl]-butyl}-amide;

Quinoxaline-2-carboxylic acid-{(S)-3-methyl-1-[3-oxo-1-(thiophene-2-sulfonyl)-azepan-4-ylcarbamoyl]-butyl}-amide;

1-Methyl-1-H-indole-2-carboxylic acid-{(S)-3-methyl-1-[3-oxo-1-(thiophene-2-sulfonyl)-azepan-4-ylcarbamoyl]-butyl}-amide;

5,6-Difluoro-benzofuran-2-carboxylic acid {(S)-3-methyl-1-[1-(pyridine-2-sulfonyl)-3-oxo-azepan-4-ylcarbamoyl]-buty}amide;

5,6-Difluoro-benzofuran-2-carboxylic acid {(S)-3-methyl-1-[1-(pyridine-2-sulfonyl)-3-oxo-azepan-4-ylcarbamoyl]-butyl}amide;

Quinoline-2-carboxylic acid {(S)-3-methyl-1-[3-oxo-1-(pyridine-2-sulfonyl)-azepan-4-ylcarbamoyl]-butyl}amide;

Quinoline-6-carboxylic acid {(S)-3-methyl-1-[3-oxo-1-(pyridine-2-sulfonyl)-azepan-4-ylcarbamoyl]-butyl}amide;

Quinoline-4-carboxylic acid {(S)-3-methyl-1-[3-oxo-1-(pyridine-2-sulfonyl)-azepan-4-ylcarbamoyl]-butyl}amide;

Isoquinoline-1-carboxylic acid {(S)-3-methyl-1-[3-oxo-1-(pyridine-2-sulfonyl)-azepan-4-ylcarbamoyl]-butyl}amide;

Naphthalene-2-carboxylic acid {(S)-3-methyl-1-[3-oxo-1-(pyridine-2-sulfonyl)-azepan-4-ylcarbamoyl]-butyl}-amide;

Quinoline-3-carboxylic acid {(S)-3-methyl-1-[3-oxo-1-(pyridine-2-sulfonyl)-azepan-4-ylcarbamoyl]-butyl}amide;

5,6-Dimethoxy-benzo[b]thiophene-2-carboxylic acid {(S)-3-methyl-1-[1-(6-methyl-pyridine-2-sulfonyl)3-oxo-azepan-4-ylcarbamoyl]-butyl}amide;

(R)-1-Benzyl-5-oxo-pyrrolidine-2-carboxylic acid {(S)-3-methyl-1-{ 3-oxo-(pyridine-2-sulfonyl)-azepan-4-ylcarbamoyl]-butyl}amide;

Benzofuran-2-carboxylic acid {(S)-2-naphthalen-2-yl-1-[3-oxo-1-(pyridine-2-sulfonyl)-azepan-4-ylcarbamoyl)-ethyl]-amide;

Thieno[3,2-b]thiophene-2-carboxylic acid {(S)-3-methyl-1-[1-(3-methyl-pyridine-2-sulfonyl)-3-oxo-azepan-4-ylcarbamoyl]-butyl}amide;

3-Methyl-benzofuran-2-carboxylic acid {(S)-3-methyl-1-[1-(3-methyl-pyridine-2-sulfonyl)-3-oxo-azepan-4-ylcarbamoyl]-butyl}amide;

5-Methoxy-benzofuran-2-carboxylic acid {(S)-3-methyl-1-[1-(3-methyl-pyridine-2-sulfonyl)-3-oxo-azepan-4-ylcarbamoyl]-butyl}amide;

5,6-Difluoro-benzofuran-2-carboxylic acid {(S)-3-methyl-1-[3-oxo-1-(1-oxy-pyridine-2sulfonyl)-azepan-4-ylcarbamoyl]-butyl}amide;

7-Methoxy-benzofuran-2-carboxylic acid {(S)-3-methyl-1-[1-(6-methyl-pyridine-2-sulfonyl)-3-oxo-azepan-4-ylcarbamoyl]-butyl}amide;

5,6-Dimethoxy-benzo[b]thiophene-2-carboxylic acid {(S)-3-methyl-1-[1-(6-methyl-pyridine-2-sulfonyl)3-oxo-azepan-4-ylcarbamoyl]-butyl}amide;

5-Fluoro-benzofuran-2-carboxylic acid {(S)-3-methyl-1-[3-oxo-1-(pyridine-2-sulfonyl)-azepan-4-ylcarbamoyl]-butyl}-amide;

Benzofuran-2-carboxylic acid {(S)-3-methyl-1-[6-methyl-3-oxo-1-(pyridine-sulfonyl)-azepan-4-ylcarbamoyl]-butyl}-amide;

5-Fluoro-benzofuran-2-carboxylic acid {(S)-3-methyl-1-[3-oxo-1-(pyridine-2-sulfonyl)-azepan-4-ylcarbamoyl]-butyl}-amide;

5-(3-Trifluoromethyl-phenyl)-furan-2-carboxylic acid (S)-2-cyclohexyl-1-[3-oxo-1-(1-oxy-pyridine-2-sulfonyl)-azepan-4-ylcarbamoyl]-ethyl}-amide;

5,6-Dimethoxy-benzofuran-2-carboxylic acid {(S)-2-cyclohexyl-1-[3-oxo-1-(1-oxy-pyridine-2-sulfonyl)-azepan-4-ylcarbamoyl]-ethyl}-amide;

5-Methoxy-benzofuran-2-carboxylic acid {(S)-3-methyl-1-[1-(3-methyl-pyridine-2-sulfonyl)-3-oxo-azepan-4-ylcarbamoyl]-butyl}amide;

Benzofuran-2-carboyylic acid {(S)-3-methyl-1-[1-(6-methyl-pyridine-2-sulfonyl)-3-oxo-azepan-4-ylcarbamoyl]3-butyl}amide;

Benzofuran-2-carboxylic acid {(S)-3-methyl-1-[6-methyl-3-oxo-1-(pyridine-sulfonyl)-azepan-4-ylcarbamoyl]-butyl}-amide;

Quinoline-8-carboxylic acid {(S)-2-naphthalen-2-yl-1-[3-oxo-1-(pyridine-2-sulfonyl)-azepan-4-ylcarbamoyl)-ethyl]-amide;

Naphthalene-1-carboxylic acid {(S)-2-naphthalen-2-yl-1-[3-oxo-1-(pyridine-2-sulfonyl)-azepan-4-ylcarbamoyl)-ethyl]-amide;

Quinoline-8-carboxylic acid {(S)-1-[3-oxo-1-(pyridine-2-sulfonyl)-azepan-4-ylcarbamoyl]-2-phenyl-ethyl}-amide;

Naphthyridine-2-carboxylic acid {(S)-3-methyl-1-[3-oxo-1-(pyridine-2-sulfonyl)-azepan-4-ylcarbamoyl]-butyl}-amide;

Naphthalene-1-carboxylic acid {(S)-1-[3-oxo-1-(pyridine-2-sulfonyl)-azepan-4-ylcarbamoyl]-2-phenyl-ethyl}-amide;

Benzofuran-2-carboxylic acid {(S)-3-methyl-1-[1-(2-methyl-furan-3-sulfonyl)-3-oxo-azepan-4-ylcarbamoyl]-butyl}-amide;

Quinoline-2-carboxylic acid {(S)-1-[3-oxo-1-(pyridine-2-sulfonyl)-azepan-4-ylcarbamoyl]-2-phenyl-ethyl}-amide;

Benzofuran-2-carboxylic acid {(S)-3-methyl-1-[(4S ,7S)-7-methyl-3-oxo-1-(pyridine-2-sulfonyl)-azepan-4-ylcarbamoyl]-butyl}-amide;

Benzofuran-2-carboxylic acid {(S)-3-methyl-1-[(4R,7R)-7-methyl-3-oxo-1-(pyridine-2-sulfonyl)-azepan-4-ylcarbamoyl]-butyl}-amide;

Benzofuran-2-carboxylic acid {(S)-1-[-(3-fluoro-benzensulfonyl)-3-oxo-azepan-4-ylcarbamoyl]-3-methyl-1-butyl}-amide;

Naphthalene-1-carboxylic acid {(S)-3-methyl-1-[3-oxo-1-(pyridine-2-sulfonyl)-azepan-4-ylcarbamoyl]-butyl}-amide;

Quinoline-5-carboxylic acid {(S)-3-methyl-1-[3-oxo-1-(pyridine-2-sulfonyl)-azepan-4-ylcarbamoyl]-butyl}-amide;

5-(3-Trifluoromethyl-phenyl)-furan-2-carboxylic acid ((S)-3-methyl-1-{3-oxo-1-[1-(1-oxy-pyridin-2-yl)-methanoyl]-azepan-4-ylcarbamoyl}-butyl)-amide;

Quinoline-8-carboxylic acid {(S)-2-naphthalen-2-yl-1-[3-oxo-1-(pyridine-2-sulfonyl)-azepan-4-ylcarbamoyl]-ethyl}-amide;

Naphthalene-1-carboxylic acid {(S)-2-naphthalen-2-yl-1-[3-oxo-1-(pyridine-2-sulfonyl)-azepan-4-ylcarbamoyl]-ethyl}-amide;

Quinoline-8-carboxylic acid ((S)-1-[3-oxo-1-(pyridine-2-sulfonyl)-azepan-4-ylcarbamoyl]-2-phenyl-ethyl}-amide;

Naphthalene-1-carboxylic acid {(S)-1-[3-oxo-1-(pyridine-2-sulfonyl)-azepan-4-ylcarbamoyl]-2-phenyl-ethyl}-amide;

5-Fluoro-benzofuran-2-carboxylic acid {(S)-3-methyl-1-[3-oxo-1-(1-oxy-pyridine-2-sulfonyl)-azepan-4-ylcarbamoyl]-butyl}-amide;

5-Fluoro-3-methyl-benzofuran-2-carboxylic acid {(S)-3-methyl-1-[3-oxo-1-(1-oxy-pyridine-2-sulfonyl)-azepan-4-ylcarbamoyl]-butyl}-amide;

6-Fluoro-3-methyl-benzofuran-2-carboxylic acid {(S)-3-methyl-1-[3-oxo-1-(1-oxy-pyridine-2-sulfonyl)-azepan-4-ylcarbamoyl]-butyl}-amide;

5-Fluoro-benzofuran-2-carboxylic acid {(S)-3-methyl-1-[3-oxo-1-(1-oxy-pyridine-2-sulfonyl)-azepan-4-ylcarbamoyl]-butyl}-amide;

5-Fluoro-3-methyl-benzofuran-2-carboxylic acid ((S)-3-methyl-1-[3-oxo-1-(1-oxy-pyridine-2-sulfonyl)-azepan-4-ylcarbamoyl]-butyl}-amide;

6-Fluoro-3-methyl-benzofuran-2-carboxylic acid {(S)-3-methyl-1-[3-oxo-1-(1-oxy-pyridine-2-sulfonyl)-azepan-4-ylcarbamoyl]-butyl}-amide;

Benzo[b]thiophene-2-carboxylic acid {(S)-3-methyl-1-[6-methyl-3-oxo-1-(pyridine-2-sulfonyl)-azepan-4ylcarbamoyl]-butyl}-amide;

5-Methoxy-benzofuran-2-carboxylic acid {(S)-3-methyl-1-[6-methyl-3-oxo-1-(pyridine-2-sulfonyl)-azepan-4-ylcarbamoyl]-butyl}-amide;

3-Methyl-benzofuran-2-carboxylic acid {(S)-3-methyl-1-[6-methyl-3-oxo-1-(pyridine-2-sulfonyl)-azepan-4-ylcarbamoyl]-butyl}-amide;

Thieno[3,2-b]thiophene-2-carboxylic acid {(S)-3-methyl-l -[6-methyl-3-oxo-1-(pyridine-2-sulfonyl)-azepan-4-ylcarbamoyl]-butyl}-amide;

3,5-Dimethyl-benzofuran-2-carboxylic acid ((S)-3-methyl-1-[3-oxo-1-(1-oxy-pyridine-2-sulfonyl)-azepan-4-ylcarbamoyl]-butyl}-amide;

3-Ethyl-benzofuran-2-carboxylic acid {(S)-3-methyl-1-[3-oxo-1-(1-oxy-pyridine-2-sulfonyl)-azepan-4-ylcarbamoyl]-butyl}-amide;

4-Methoxy-3-methyl-benzofuran-2-carboxylic acid {(S)-3-methyl-1-[3-oxo-1-(1-oxy-pyridine-2-sulfonyl)-azepan-4-ylcarbamoyl]-butyl}-amide;

6-Methoxy-3-methyl-benzofuran-2-carboxylic acid {(S)-3-methyl-1-[3-oxo-1-(1-oxy-pyridine-2-sulfonyl)-azepan-4-ylcarbamoyl]-butyl}-amide;

5-Methoxy-3-methyl-benzofuran-2-carboxylic acid {(S)-3-methyl-1-[3-oxo-1-(1-oxy-pyridine-2-sulfonyl)-azepan-4-ylcarbamoyl]-butyl}-amide;

3,5-Dimethyl-benzofuran-2-carboxylic acid {(S)-3-methyl-1-[3-oxo-1-(1-oxy-pyridine-2-sulfonyl)-azepan-4-ylcarbamoyl]-butyl}-amide;

3-Ethyl-benzofuran-2-carboxylic acid {(S)-3-methyl-1-[3-oxo-1-(1-oxy-pyridine-2-sulfonyl)-azepan-4-ylcarbamoyl]-butyl}-amide;

4-Methoxy-3-methyl-benzofuran-2-carboxylic acid {(S)-3-methyl-1-[3-oxo-1-(1-oxy-pyridine-2-sulfonyl)-azepan-4-ylcarbamoyl]-butyl}-amide;

4-Methoxy-3-methyl-benzofuran-2-carboxylic acid {(S)-3-methyl-1-[3-oxo-1-(1-oxy-pyridine-2-sulfonyl)-azepan-4-ylcarbamoyl]-butyl}-amide;

1-methyl-naphtho[2,1-b]-furan-2-carboxylic acid {(S)-3-methyl-1-[3-oxo-1-(1-oxy-pyridine-2-sulfonyl)-azepan-4-ylcarbamoyl]-butyl}-amide;

6-Methoxy-3-methyl-benzofuran-2-carboxylic acid {(S)-3-methyl-1-[3-oxo-1-(1-oxy-pyridine-2-sulfonyl)-azepan-4-ylcarbamoyl]-butyl}-amide;

Benzofuran-2-carboxylic acid {(S)-3-methyl-1-[3-oxo-1-quinolin-2-ylmethyl-azepan-4-ylcarbamoyl]-butyl}-amide;

3-Methyl-benzofuran-2-carboxylic acid [(S)-3-methyl-1-[3-oxo-1-quinolin-2-ylmethyl-azepan-4-ylcarbamoyl]-butyl}-amide;

Benzo[b]thiophene-2-carboxylic acid {(S)-3-methyl-1-[3-oxo-1-quinolin-2-ylmethyl-azepan-4-ylcarbamoyl]-butyl}-amide;

Benzo[b]thiophene-2-carboxylic acid {(S)-1-[1-(2-fluoro-phenylcarbamoyl)-3-oxo-azepan-4-ylcarbamoyl]-3-methyl-butyl}-amide;

3-Methyl-benzofuran-2-carboxylic acid {(S)-1-[1-(2-fluoro-phenylcarbamoyl)-3-oxo-azepan-4-ylcarbamoyl]-3-methyl-butyl}-amide;

Quinoxaline-2-carboxylic acid {(S)-1-[1-(2-fluoro-phenylcarbamoyl)-3-oxo-azepan-4-ylcarbamoyl]-3-methyl-butyl}-amide;

Thieno[3,2-b]thiophene-2-carboxylic acid {(S)-1-[1-(2-fluoro-phenylcarbamoyl)-3-oxo-azepan-4-ylcarbamoyl]-3-methyl-butyl}-amide;

Quinoline-2-carboxylic acid {(S)-1-[1-(2-fluoro-phenylcarbamoyl)-3-oxo-azepan-4-ylcarbamoyl]-3-methyl-butyl}-amide;

4-Methyl-2-carboxylic acid {(S)-1-[1-(2-fluoro-phenylcarbamoyl)-3-oxo-azepan-4-ylcarbamoyl]-3-methyl-butyl}-amide;

5-Methoxy-benzofuran-2-carboxylic acid {(S)-1-[1-(2-fluoro-phenylcarbamoyl)-3-oxo-azepan-4-ylcarbamoyl]-3-methyl-butyl}-amide; and

4-Methyl-furan-2-carboxylic acid {(S)-1-[1-(2-fluoro-phenylcarbamoyl)-3-oxo-azepan-4-ylcarbamoyl]-3-methyl-butyl}-amide.

Specific representative compounds which are useful in the present methods are set forth in Examples 1-222.

The present invention includes deuterated analogs of the inventive compounds. A representative synthetic route for the deuterated compounds of the present invention is set forth in Scheme 7, below. The deuterated compounds of the present invention exhibit superior chiral stability compared to the protonated isomer.

### Definitions

The compounds used in the present invention includes all hydrates, solvates, complexes and prodrugs of the compounds of this invention. Prodrugs are any covalently bonded compounds which release the active parent drug according to Formula I in vivo. If a chiral center or another form of an isomeric center is present in a compound of the present invention, all forms of such isomer or isomers, including enantiomers and diastereomers, are intended to be covered herein. Compounds containing a chiral center may be used as a racemic mixture, an enantiomerically enriched mixture, or the racemic mixture may be separated using well-known techniques and an individual enantiomer may be used alone. In cases in which compounds have unsaturated carbon-carbon double bonds, both the cis (Z) and trans (E) isomers are within the scope of this invention. In cases wherein compounds may exist in tautomeric forms, such as keto-enol tautomers, each tautomeric form is contemplated as being included within this invention whether existing in equilibrium or predominantly in one form.

The meaning of any substituent at any one occurrence in Formula I or any subformula thereof is independent of its meaning, or any other substituent's meaning, at any other occurrence, unless specified otherwise.

Abbreviations and symbols commonly used in the peptide and chemical arts are used herein to describe the compounds of the present invention. In general, the amino acid abbreviations follow the IUPAC-IUB Joint Commission on Biochemical Nomenclature as described in Eur. J. Biochem., 158, 9 (1984).

“Proteases” are enzymes that catalyze the cleavage of amide bonds of peptides and proteins by nucleophilic substitution at the amide bond, ultimately resulting in hydrolysis. Such proteases include: cysteine proteases, serine proteases, aspartic proteases, and metalloproteases. The compounds of the present invention are capable of binding more strongly to the enzyme than the substrate and in general are not subject to cleavage after enzyme catalyzed attack by the nucleophile. They therefore competitively prevent proteases from recognizing and hydrolyzing natural substrates and thereby act as inhibitors.

The term “amino acid” as used herein refers to the D- or L-isomers of alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine and valine. “C₁₋₆alkyl” as applied herein is meant to include substituted and unsubstituted methyl, ethyl, n-propyl, isopropyl, n-butyl, isobutyl and t-butyl, pentyl, n-pentyl, isopentyl, neopentyl and hexyl and the simple aliphatic isomers thereof. C₁₋₆alkyl may be optionally substituted by a moiety selected from the group consisting of: OR¹⁴, C(O)R¹⁴, SR¹⁴, S(O)R¹⁴, NR¹⁴₂, R¹⁴NC(O)OR⁵, CO₂R¹⁴, CO₂NR¹⁴₂, N(C═NH)NH₂, Het, C₃₋₆cycloalkyl, and Ar; where R⁵ is selected from the group consisting of: H, C₁₋₆alkyl, C₂₋₆alkenyl, C₂₋₆alkynyl, C₃₋₆cycloalkyl-C₀₋₆alkyl, Ar—C₀₋₆alkyl and Het-C₀₋₆alkyl; and R¹⁴ is selected from the group consisting of: H, C₁₋₆alkyl, Ar—C₀₋₆alkyl, and Het-C₀₋₆alkyl;

“C₃₋₆cycloalkyl” as applied herein is meant to include substituted and unsubstituted cyclopropane, cyclobutane, cyclopentane and cyclohexane.

“C₂₋₆ alkenyl” as applied herein means an alkyl group of 2 to 6 carbons wherein a carbon-carbon single bond is replaced by a carbon-carbon double bond. C₂₋₆alkenyl includes ethylene, 1-propene, 2-propene, 1-butene, 2-butene, isobutene and the several isomeric pentenes and hexenes. Both cis and trans isomers are included.

“C₂₋₆alkynyl” means an alkyl group of 2 to 6 carbons wherein one carbon-carbon single bond is replaced by a carbon-carbon triple bond. C₂₋₆ alkynyl includes acetylene, 1-propyne, 2-propyne, 1-butyne, 2-butyne, 3-butyne and the simple isomers of pentyne and hexyne.

“Halogen” means F, Cl, Br, and I.

“Ar” or “aryl” means phenyl or naphthyl, optionally substituted by one or more of Ph-C₀₋₆alkyl; Het-C₀₋₆alkyl; C₁₋₆alkoxy; Ph-C₀₋₆alkoxy; Het-C₀₋₆alkoxy; OH, (CH₂)₁₋₆NR¹⁵R¹⁶; O(CH₂)₁₋₆NR¹⁵R¹⁶; C₁₋₆alkyl, OR¹⁷, N(R¹⁷)₂, SR¹⁷, CF₃, NO₂, CN, CO₂R¹⁷, CON(R¹⁷), F, Cl, Br or I; where R¹⁵ and R¹⁶ are H, C₁₋₆alkyl, Ph—C₀₋₆alkyl, naphthyl-C₀₋₆alkyl or Het-C₀₋₆alkyl; and R¹⁷ is phenyl, naphthyl, or C₁₋₆alkyl.

As used herein “Het” or “heterocyclic” represents a stable 5- to 7-membered monocyclic, a stable 7- to 10-membered bicyclic, or a stable 11- to 18-membered tricyclic heterocyclic ring which is either saturated or unsaturated, and which consists of carbon atoms and from one to three heteroatoms selected from the group consisting of N,O and S, and wherein the nitrogen and sulfur heteroatoms may optionally be oxidized, and the nitrogen heteroatom may optionally be quaternized, and including any bicyclic group in which any of the above-defined heterocyclic rings is fused to a benzene ring. The heterocyclic ring may be attached at any heteroatom or carbon atom which results in the creation of a stable structure, and may optionally be substituted with one or two moieties selected from C₀₋₆Ar, Cl6alkyl, OR¹⁷, N(R¹⁷)₂, SR¹⁷, CF₃, NO₂, CN, CO₂R¹⁷, CON(R¹⁷), F, Cl, Br and I, where R¹⁷ is phenyl, naphthyl, or C₁₋₆alkyl. Examples of such heterocycles include piperidinyl, piperazinyl, 2-oxopiperazinyl, 2-oxopiperidinyl, 2-oxopyrrolodinyl, 2-oxoazepinyl, azepinyl, pyrrolyl, 4-piperidonyl, pyrrolidinyl, pyrazolyl, pyrazolidinyl, imidazolyl, pyridinyl, 1-oxo-pyridinyl, pyrazinyl, oxazolidinyl, oxazolinyl, oxazolyl, isoxazolyl, morpholinyl, thiazolidinyl, thiazolinyl, thiazolyl, quinuclidinyl, indolyl, quinolinyl, quinoxalinyl, isoquinolinyl, benzimidazolyl, benzopyranyl, benzoxazolyl, furanyl, benzofuranyl, thiophenyl, benzo[b]thiophenyl, thieno[3,2-b]thiophenyl, benzo[1,3]dioxolyl, 1,8 naphthyridinyl, pyranyl, tetrahydrofuranyl, tetrahydropyranyl, thienyl, benzoxazolyl, thiamorpholinyl sulfoxide, thiamorpholinyl sulfone, and oxadiazolyl, as well as triazolyl, thiadiazolyl, oxadiazolyl, isothiazolyl, imidazolyl, pyridazinyl, pyrimidinyl, triazinyl and tetrazinyl which are available by routine chemical synthesis and are stable. The term heteroatom as applied herein refers to oxygen, nitrogen and sulfur.

Here and throughout this application the term C₀ denotes the absence of the substituent group immediately following; for instance, in the moiety ArC₀₋₆alkyl, when C is 0, the substituent is Ar, e.g., phenyl. Conversely, when the moiety ArC₀₋₆alkyl is identified as a specific aromatic group, e.g., phenyl, it is understood that the value of C is 0.

Certain radical groups are abbreviated herein. t-Bu refers to the tertiary butyl radical, Boc refers to the t-butyloxycarbonyl radical, Fmoc refers to the fluorenylmethoxycarbonyl radical, Ph refers to the phenyl radical, Cbz refers to the benzyloxycarbonyl radical.

Certain reagents are abbreviated herein. m-CPBA refers to 3-chloroperoxybenzoic acid, EDC refers to N-ethyl-N′(dimethylaminopropyl)-carbodiimide, DMF refers to dimethyl formamide, DMSO refers to dimethyl sulfoxide, TEA refers to triethylamine, TFA refers to trifluoroacetic acid, and THF refers to tetrahydrofuran.

### Methods of Preparation

Compounds of the general formula I may be prepared in a fashion analogous to that outlined in Schemes 1, 2 and 3. Alkylation of tert-butyl N-allylcarbamate (1) with a base such as sodium hydride and 5-bromo-1-pentene provides the diene 2. Treatment of 2 with either 2,6-diisopropylphenylimido neophylidene molybenum bis(tert-butoxide) or bis(tricyclohexylphosphine)benzylidine ruthenium (IV) dichloride olefin metathesis catalysts developed by Grubbs provides the azepine 3. Epoxidation of 3 with standard oxidizing agents common to the art such as m-CPBA provide the epoxide 4. Nucleophilic epoxide ring opening may be effected with a reagent such as sodium azide to provide the azido alcohol (not shown) which may be reduced to the amino alcohol 5 under conditions common to the art such as 1,3-propanedithioln and triethylamine in methanol or with hydrogen gas in the presence of a catalyst such as palladium on carbon. Acylation of 5 with an acid such as Cbz-leucine in the presence of a coupling agent such as EDC followed by removal of the BOC protecting group under acidic conditions provides the amine salt 6. Coupling of 6 with Cbz-leucine was effected with a coupling agent such as EDC provides the intermediate alcohol (not shown) which was oxidized with an oxidant such as pyridine sulfur trioxide complex in DMSO and triethylamine to provide the ketone 7.

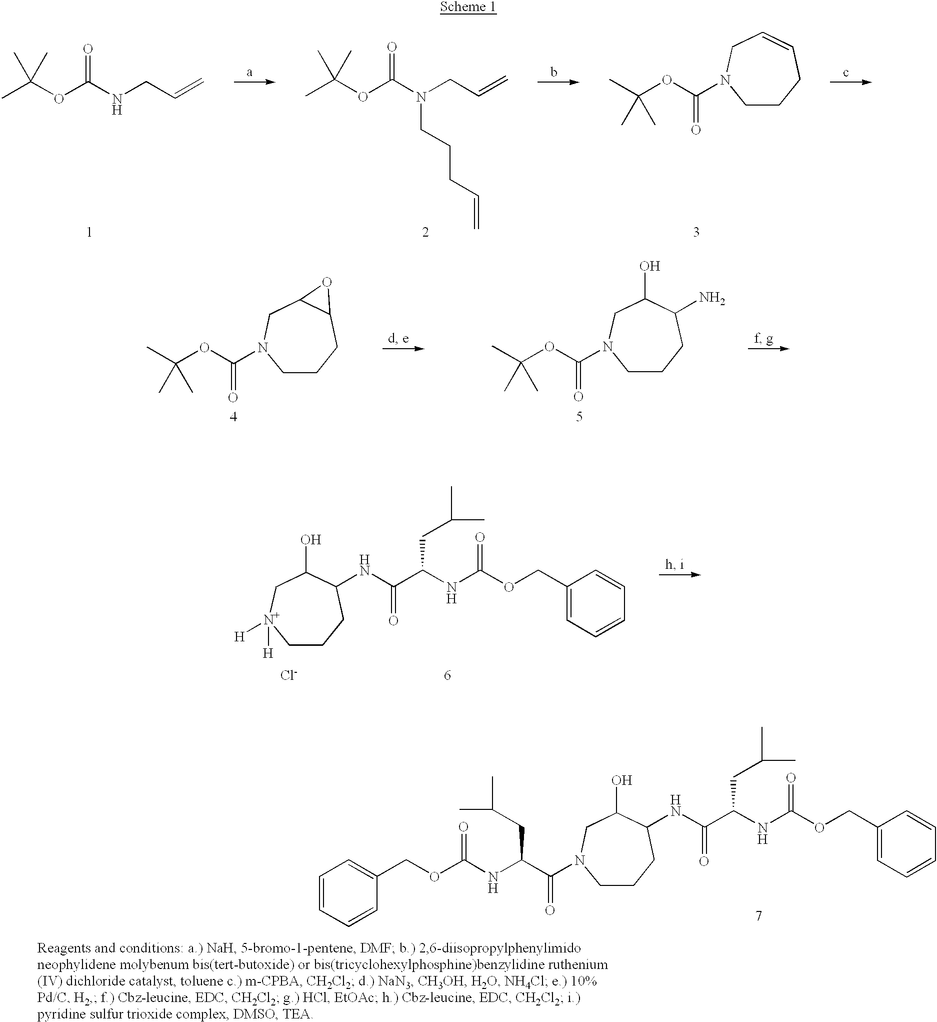

Compounds of the general formula I wherein R1 and R2 are amides may be prepared in the general fashion outlined in Scheme 2. Alkylation of N-Cbz allyl amine (8) with a base such as sodium hydride and 5-bromo-1-pentene provides the diene 9. Treatment of 9 with bis(tricyclohexylphosphine)benzylidine ruthenium(IV)dichloride olefin metathesis catalyst developed by Grubbs provides the azepine 10. Epoxidation of 10 with standard oxidizing agents common to the art such as m-CPBA provide the epoxide 11. Nucleophilic epoxide ring opening may be effected with a reagent such as sodium azide to provide the azido alcohol (not shown) which may be reduced to the amino alcohol 12 with a reducing agent such as propanedithiol in the presence of triethylamine. Acylation of 12 with N-Boc-leucine and a coupling agent such as EDC followed by removal of the Cbz protecting group under hydrogenolysis conditions provides the amine 13. Coupling of 13 with a carboxylic acid was effected with a coupling agent such as EDC followed by removal of the acid labile N-Boc protecting group with an acid such as HC or TFA provides intermediate 14. Acylation of 14 maybe effected with a carboxylic acid in the presence of a coupling agent common to the art such as EDC to give the intermediate alcohol (not shown) which is oxidized with an oxidant such as pyridine sulfur trioxide complex in DMSO and triethylamine to provide the ketone 15.

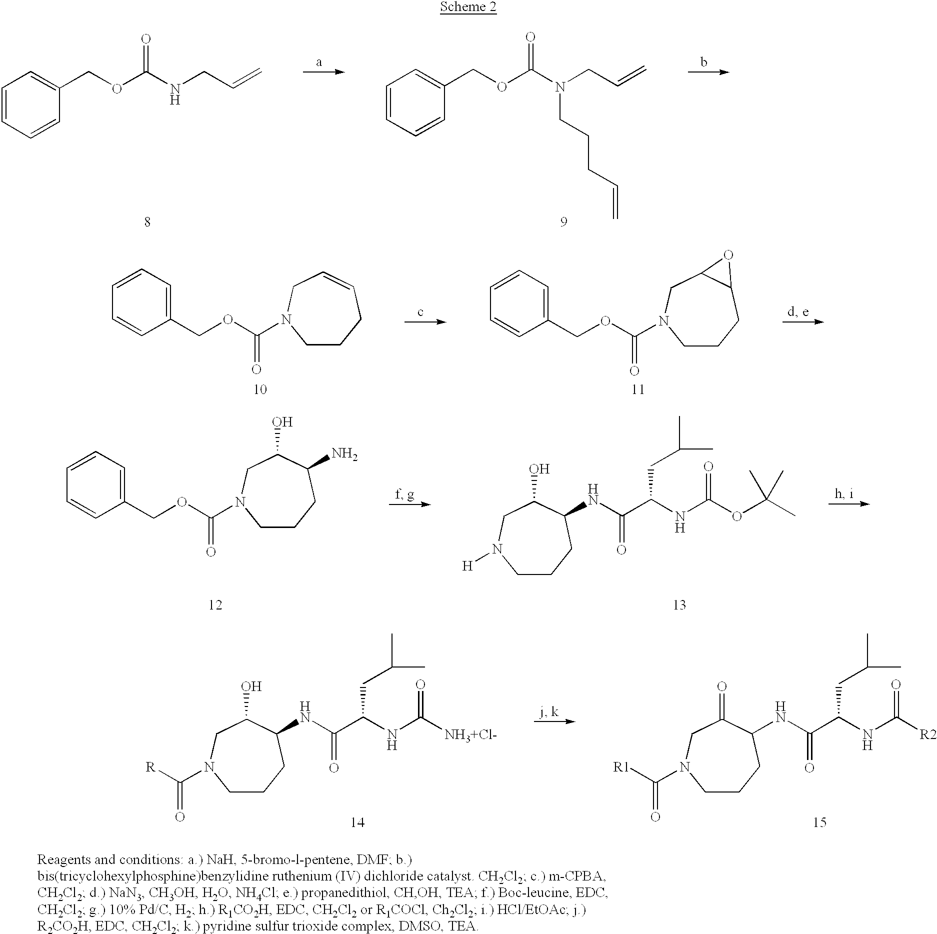

Compounds of the general formula I wherein R₂ is an alkyl, urea or sulphonamide group and R₁ is an amide may be prepared in the general fashion outlined in Scheme 3. Reductive amination of 13 may be effected by treatment with an aldehyde followed by a reducing agent such as sodium triacetoxyborohydride. Subsequent deprotection of the N-Boc group under acidic conditions provides the amine salt 16. Coupling of 16 with an acid chloride or with a carboxylic acid in the presence of a coupling agent common to the art such as EDC followed by oxidation of the intermediate alcohol (not shown) with an oxidant such as pyridine sulfurtrioxide complex provides the ketone 17. Alternatively, treatment of amine 13 with an isocyanate followed by deprotection of the N-Boc group provides the amine salt 18. Acylation and oxidation provides the ketone 19. Further derivatization of amine 13 may be effected by treatment with a sulphonyl chloride followed by deprotection of the N-Boc group to provide the amine salt 20. Acylation and oxidation provides the ketone 21.

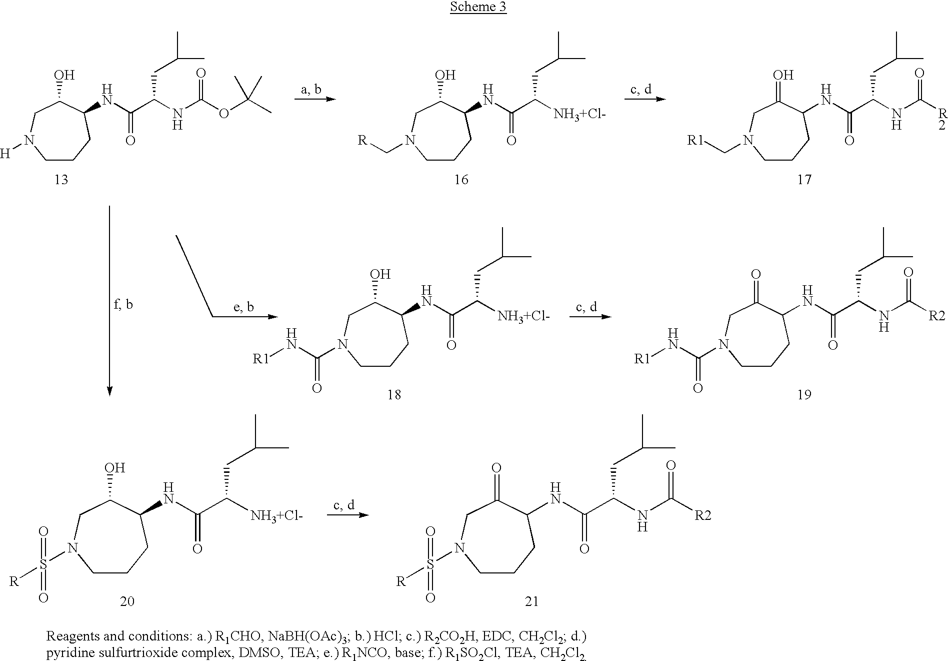

Compounds of the general formula I may be prepared in a fashion analogous to that outlined in Schemes 4, 5, 6, and 7

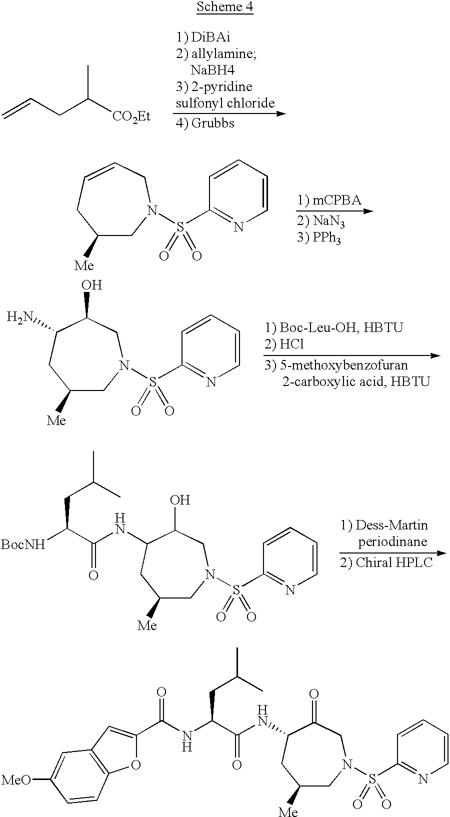

2-Methyl-pent-4-enoic acid ethyl ester is converted to a N-2-pyridinesulfonyl-azapine by reduction to the aldehyde, reductive amination with allylamine, sulfonylation with 2-pyridyl sulfonyl chloride, and olefin metathesis with Grubbs' catalyst. Epoxidation with mCPBA affords a mixture of epoxides that are separable by column chromatography. The syn epoxide is converted into an amino alcohol by opening with sodium azide followed by reduction with triphenylphosphine. Acylation of the free amine with Boc-leucine and a coupling reagent such as HBTU or EDC, followed by deprotection of the Boc group with HCl, and acylation with a variety of aromatic carboxylic acids and coupling reagents such as HBTU or EDC gives the intermediate alcohols. Final oxidation with Dess-Martin periodinane and HPLC affords the desired ketones.

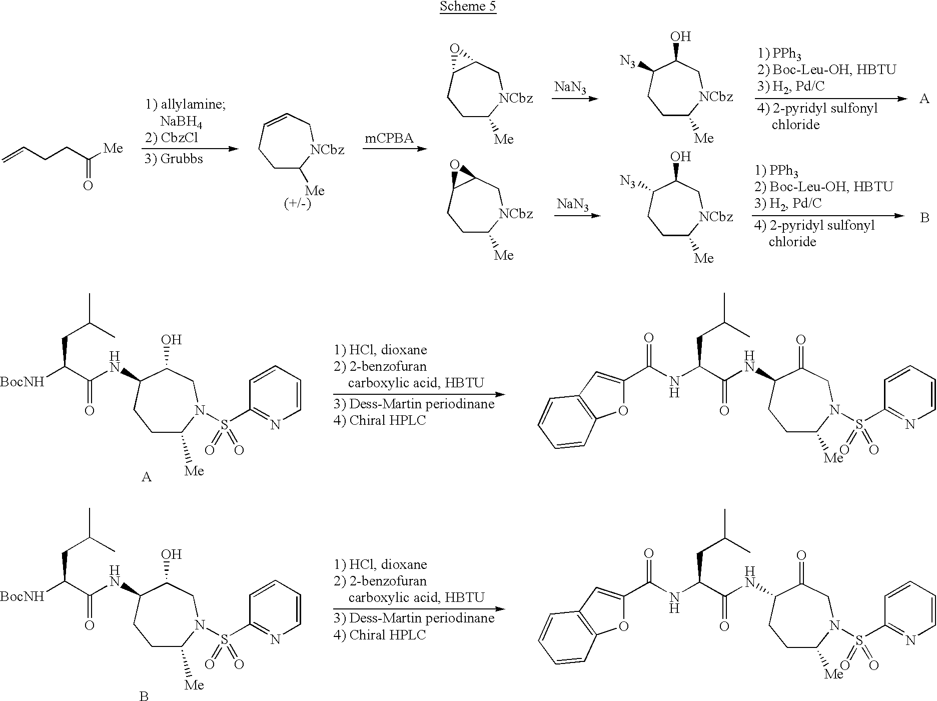

5-Hexen-2-one is converted to a N-carbobenzyloxy-azapine by reductive amination with allylamine, protection with carbobenzyloxychloride, and olefin metathesis with Grubbs' catalyst. Epoxidation with mCPBA affords a mixture of epoxides that are separable by column chromatography. Each epoxide is converted into an amino alcohol by opening with sodium azide followed by reduction with triphenylphosphine. Acylation of the free amine with Boc-leucine and a coupling reagent such as HBTU or EDC, followed by deprotection of the Cbz group by hydrogenolysis provides the secondary amines which are in turn sulfonylated with 2-pyridine sulfonylchloride. Deprotection of the Boc groups with HCI and acylation with a variety of aromatic carboxylic acids and coupling reagents such as HBTU or EDC gives the interrnediate alcohols. Final oxidation with Dess-Martin periodinane and HPLC affords the desired ketones.

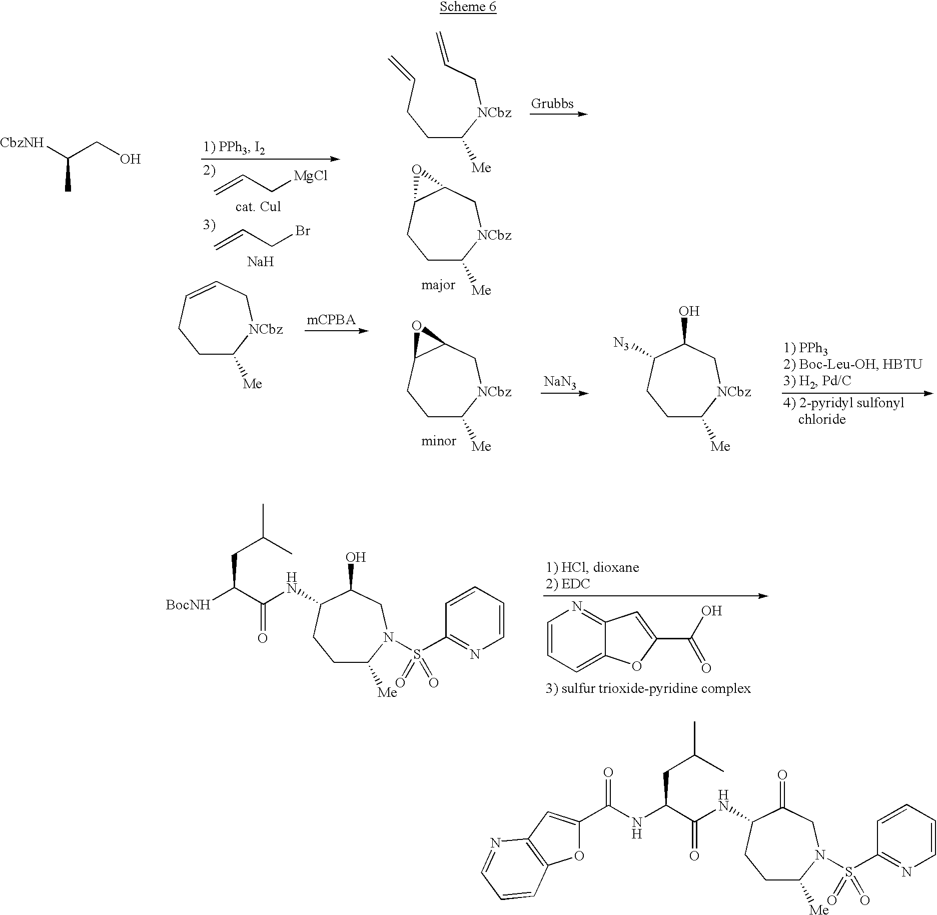

Carbobenyzloxy-D-alaninol (Cbz-D-alaninol)is first converted to an iodide, then is reacted with allyl Grignard with a copper (I) catalyst or a similar allyl organometallic reagent. The amine is then alkylated with allyl iodide. Grubbs' catalyst is then used to form the azapine ring by ring closing metathesis. Epoxidation of the alkene followed by separation of the diastereomers followed by opening of the epoxide of the minor component with sodium azide provides the intermediate azido alcohol. Reduction of the azide followed by acylation of the amine with Boc-leucine followed by deprotection of the Cbz gives the intermediate secondary amine, which is then sulfonylated with an sulfonyl chloride. Deprotection of the Boc group followed by acylation and final oxidation of the secondary alcohol to the ketone provides the desired products.

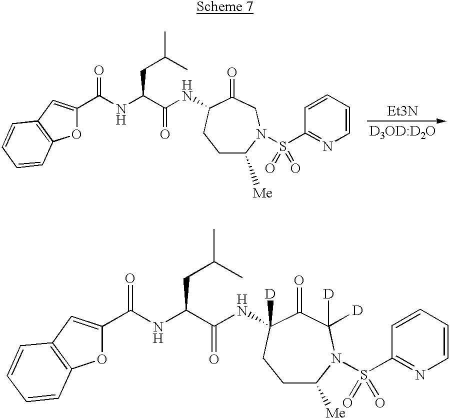

Deuterated inhibitors can be prepared from the parent inhibitors such as benzofuran-2-carboxylic acid {(S)-3-methyl-1-[(4S ,7R)-7-methyl-3-oxo-1-(pyridine-2-sulfonyl)-azepan-4-ylcarbamoyl]-butyl}-amide by treating with a base such as triethyl amine and stirring for several days in a deteurated protic solvent such as CD₃OD: D₂O.

### Utility of the Invention

In general, the present invention provides a method of inhibiting cysteine proteases of the papain superfamily by administering to a patient in need thereof, particularly an animal, more particularly a mammal, most particularly a human being, one or more of the compounds of Formula I.

The present invention also provides a method of treating a parasitic disease mediated by a cysteine protease by administering to a patient in need thereof, particularly an animal, more particularly a mammal, most particularly a human being, one or more of the compounds of Formula I.

Parasites known to utilize cysteine proteases in their life cycle include Plasmoditim falciparim (malaria), Trypanosoma cruzi, Trypanosoma Brucei [trypanosomiasis (African sleeping sickness, Chagas disease)], Leishmania mexicana, Leishmania pifanoi, Leishmania major (leishmaniasis), Schistosoma mansoni (schistosomiasis), Onchocerca volvulus [onchocerciasis (river blindness)] Brugia pahangi, Entamoeba histolytica, Giardia lambia, the helminths, Haemonchus contortus and Fasciola hepatica, as well as helminths of the genera Spirometra, Trichinella, Necator and Ascaris, and protozoa of the genera Cryptosporidium, Eimeria, Toxoplasma and Naegleria. The present method provides treatment of diseases caused by infection by these parasites by inhibiting, cysteine proteases of the papain superfamily by administering to a patient in need thereof, particularly an animal, more particularly a mammal, most particularly a human being, one or more of the compounds of Formula I.

As demonstrated in Table I below, the compounds of Formula I used in the inventive method are especially effective in inhibition of one or more of the following parasitic proteases: falcipain (P. falciparum), cruzain (T. cruzi), rhodazain (T. brucei rhodesiensi), leishmania L (Leishmania spp.), leishmania B (Leishmania spp.), and schistosoma B (S. mansoni).

More particularly, the present invention provides a method of treating diseases selected from a group consisting of: malaria, trypanosomiasis (African sleeping sickness, Chagas disease), leishmaniasis, schistosomiasis, onchocerciasis (river blindness) and giardiasis by administering to a patient in need thereof, particularly an animal, more particularly a mammal, most particularly a human being, one or more of the compounds of Formula I.

Most particularly, the present invention provides a method of treating malaria, caused by infection with Plasmodium falciparum, by the inhibition of falcipain by administering to a patient in need thereof, particularly an animal, more particularly a mammal, most particularly a human being, one or more of the above-listed compounds.

The present method may be practiced by administering the above-listed compounds alone or in combination with other therapeutically effective compounds, including but not limited to quinoline-derived drugs.

Certain radical groups are abbreviated herein. t-Bu refers to the tertiary butyl radical, Boc refers to the t-butyloxycarbonyl radical, Fmoc refers to the fluorenylmethoxycarbonyl radical, Ph refers to the phenyl radical, Cbz refers to the benzyloxycarbonyl radical.

The present invention includes all esters, hydrates, solvates, complexes and prodrugs of the above-listed compounds useful in the inventive method. Prodrugs are any covalently bonded compounds which release the active parent drug in vivo. If a chiral-center or another form of an isomeric center is present in a compound of the present invention, all forms of such isomer or isomers, including enantiomers and diastereomers, are intended to be covered herein. Inventive compounds containing a chiral center may be used as a racemic mixture, an enantiomerically enriched mixture, or the racemic mixture may be separated using well-known techniques and an individual enantiomer may be used alone. In cases in which compounds have unsaturated carbon-carbon double bonds, both the cis (Z) and trans (E) isomers are within the scope of this invention. In cases wherein compounds may exist in tautomeric forms, such as keto-enol tautomers, each tautomeric form is contemplated as being included within this invention whether existing in equilibrium or predominantly in one form.

The methods of treatment of this invention also use pharmaceutical compositions which comprise one or more of compounds of Formula I and a pharmaceutically acceptable carrier, diluent or excipient. Accordingly, the compounds of Formula I may be used in the manufacture of a medicament useful in the practice of the present methods of treatment. Pharmaceutical compositions of compounds of Formula I prepared as hereinbefore described may be formulated as solutions or lyophilized powders for parenteral administration. Powders may be reconstituted by addition of a suitable diluent or other pharmaceutically acceptable carrier prior to use. The liquid formulation may be a buffered, isotonic, aqueous solution. Examples of suitable diluents are normal isotonic saline solution, standard 5% dextrose in water, or buffered sodium or ammonium acetate solution. Such formulation is especially suitable for parenteral administration, but may also be used for oral administration or contained in a metered dose inhaler or nebulizer for insulation. It may be desirable to add excipients such as polyvinylpyrrolidone, gelatin, hydroxy cellulose, acacia, polyethylene glycol, mannitol, sodium chloride, or sodium citrate.

Alternately, these compounds may be encapsulated, tableted, or prepared in an emulsion or syrup for oral administration. Pharmaceutically acceptable solid or liquid carriers may be added to enhance or stabilize the composition, or to facilitate preparation of the composition. Solid carriers include starch, lactose, calcium sulfate dihydrate, terra alba, magnesium stearate or stearic acid, talc, pectin, acacia, agar or gelatin. Liquid carriers include syrup, peanut oil, olive oil, saline and water. The carrier may also include a sustained release material such as glyceryl monostearate or glyceryl distearate, alone or with a wax. The amount of solid carrier varies but, preferably, will be between about 20 mg to about 1 g per dosage unit. The pharmaceutical preparations are made following the conventional techniques of pharmacy involving milling, mixing, granulating, and compressing, when necessary, for tablet forms; or milling, mixing and filling for hard gelatin capsule forms. When a liquid carrier is used, the preparation will be in the form of a syrup, elixir, emulsion or an aqueous or non-aqueous suspension. Such a liquid formulation may be administered directly or filled into a soft gelatin capsule.

For rectal administration, the compounds of Formula I may also be combined with excipients such as cocoa butter, glycerin, gelatin or polyethylene glycols and molded into a suppository.

In accordance with this invention, an effective amount of one or more compounds of Formula I is administered to inhibit the protease implicated with a particular condition or disease. Of course, this dosage amount will further be modified according to the type of administration of the compound. For example, for acute therapy, parenteral administration of an effective amount of a compound of Formula I is preferred. An intravenous infusion of the compound in 5% dextrose in water or normal saline, or a similar formulation with suitable excipients, is most effective, although an intramuscular bolus injection is also useful. Typically, the parenteral dose will be about 0.01 to about 100 mg/kg; preferably between 0.1 and 20 mg/kg, in a manner to maintain the concentration of drug in the plasma at a concentration effective to inhibit the parasitic protease, e.g. falcipain in the case of treatment of malaria. The compounds are administered one to four times daily at a level to achieve a total daily dose of about 0.4 to about 400 mg/kg/day. The precise amount of an inventive compound which is therapeutically effective, and the route by which such compound is best administered, is readily determined by one of ordinary skill in the art by comparing the blood level of the agent to the concentration required to have a therapeutic effect.

The compound may be administered in the form of a prodrug which, in general, is designed to enhance absorption and is cleaved in vivo to form the active component. Efficacious levels may also be achieved by administration of pharmaceutically active metabolites or bioisosteres of the compound. Prodrugs of compounds of the present invention may be prepared by any suitable method.

The compounds used in this method may also be administered orally to the patient, in a manner such that the concentration of drug is sufficient to inhibit cysteine proteases, e.g. falcipain in the case of treatment of malaria, or to achieve any other therapeutic indication as disclosed herein. Typically, a pharmaceutical composition containing the compound is administered at an oral dose of between about 0.1 to about 50 mg/kg in a manner consistent with the condition of the patient. Preferably the oral dose would be about 0.1 to about 50 mg/kg given 1-2 times/day.

No unacceptable toxicological effects are expected when compounds of the present invention are administered in accordance with the present method of treatment.

### Biological Assays

The compounds used in the present method may be tested in one of several biological assays to determine the concentration of a compound which is required to have a given pharmacological effect. For example, an assay for determining parasitic cysteine protease catalytic activity and an assay to determine the amount of cysteine protease inhibition by a compound of the inventive method are provided.

### Determination of cysteine protease catalytic activity

Standard assay conditions for the determination of kinetic constants used 10 uM fluorogenic peptide substrate, Cbz-Phe-Arg-AMC for the cysteine proteases from Leishmania spp and Schistosoma Mansoni, Cbz-Leu-Arg-AMC for the cysteine proteases from Plasmodium falciparum and Trypanosoma brucei rhodesiensi, and Ac-Lys-Glu-Lys-Leu-Arg-AMC (4 uM final substrate concentration) for the cysteine protease from Trypoanosoma cruzi, and were determined in 100 mM Na acetate at pH 5.5 containing 5 mM cysteine. Stock substrate solutions were prepared at concentrations of 10 MM in DMSO with 10 uM (4 uM for Ac-Lys-Glu-Lys-Leu-Arg-AMC) final substrate concentration in the assays. The final DMSO concentration was 2% and the final volume was 100 uL. All assays were conducted at ambient temperature. Product progress curves were generated over 20 to 30 minutes following formation of AMC product.

### Inhibition Studies

Potential inhibitors were evaluated using the progress curve method. Assays were carried out in the presence of variable concentrations of test compound. Reactions were initiated by addition of enzyme to buffered solutions of inhibitor and substrate. Data analysis was conducted according to one of two procedures depending on the appearance of the progress curves in the presence of inhibitors. For those compounds whose progress curves were linear, apparent inhibition constants (K_{i,app}) were calculated according to equation 1 (Brandt et al., Biochemitsry, 1989, 28, 140):

v=VₘA/[Kₐ(1+I/K_{i,app})+A] (1)

Where v is the velocity of the reaction with maximal velocity Vₘ, A is the concentration of substrate with Michaelis constant of Kₐ, and I is the concentration of inhibitor.

For those compounds whose progress curves showed downward curvature characteristic of time-dependent inhibition, the data from individual sets was analyzed to give k_{obs} according to equation 2:

[AMC]=vₛₛt+(v₀−vₛₛ)[1−exp (−k_{obs}t)]/k_{obs} (2)

Where [AMC] is the concentration of product formed over time t, v₀ is the initial reaction velocity and vₛₛ is the final steady state rate. Values for k_{obs} were then analyzed as a linear function of inhibitor concentration to generate an apparent second order rate constant (k_{obs}/inhibitor concentration or k_{obs}/[I]) describing the time-dependent inhibition. A complete discussion of this kinetic treatment has been fully described (Morrison et al., Adv. Enzymol. Relat. Areas Mol. Biol., 1988, 61, 201).

Exemplary inhibition data for the compounds used in the present method collected in accordance with the above-described procedure are listed in Table I.

The data in Table I demonstrate that the compounds of the present invention are efficacious inhibitors of the cysteine protease of one or more of the parasites selected from the group consisting of: Leishmania spp, Schistosoma Mansoni, Plasmodium falciparim, Trypanosoma brucei rhodesiensi, and Trypoanosoma cruzi, and thus, if administered according to the present method, may be therapeutically effective in treating malaria and other parasitic diseases identified herein above in animals, particularly mammals, most particularly human beings.

### EXAMPLES

In the following synthetic examples, unless otherwise indicated, all of the starting materials were obtained from commercial sources. Without further elaboration, it is believed that one skilled in the art can, using the preceding description, utilize the present invention to its fullest extent. These Examples are given to illustrate the invention, not to limit its scope.

Flash column chromatography was performed using silica gel 60 (Merck Art 9385). ¹H NMR (300 MHz) spectra were measured in CDCl₃ solutions and were determined on a Varian 300 instrument utilizing a Varian UNITYplus300 operating software. Chemical shifts are reported in parts per million (ppm) downfield from tetramethylsilane as the internal standard, and coupling constants are given in Hertz. The following abbreviations are used for spin multiplicity: br=broad, s=singlet, d=doublet, t=triplet, q=quartet, m=multiplet, cm=complex multiplet. Infrared (IR) spectra were recorded on a Perkin-Elmer 1600 series FTIR spectrometer and are reported in wave numbers (cm-1).

### Example 1

### Preparation of benzo[1,3]dioxole-5-carboxylic acid [(S)-1-(1-benzyl-3-oxoazepan-4-ylcarbamoyl)-3-methyl-butyl]amide

a) allyl-pent4-enyl-carbamic acid benzyl ester

To a suspension of NaH (1.83 g, 76.33 mmol of 90% NaH) in DMF was added benzyl allyl-carbamic acid benzyl ester (7.3 g, 38.2 mmol) in a dropwise fashion. The mixture was stirred at room temperature for approximately 10 minutes whereupon 5-bromo-1-pentene (6.78 mL, 57.24 mmol) was added in a dropwise fashion. The reaction was heated to 40° C. for approximately 4 hours whereupon the reaction was partitioned between dichloromethane and water. The organic layer was washed with water (2×'s), brine, dried (MgSO₄), filtered and concentrated. Column chromatography of the residue (10% ethyl acetate:hexanes) provided 10.3 grams of the title compound as an oil. MS (ESI): 260 (M+H⁺).

b) 2,3,4,7-tetrahydro-azepine-1-carboxylic acid benzyl ester

To a solution of the compound of Example 1(a) (50 g) in dichloromethane was added bis(tricyclohexylphosphine)benzylidine ruthenium (IV) dichloride (5.0 g). The reaction was heated to reflux until complete as determined by TLC analysis. The reaction was concentrated in vacuo. Column chromatography of the residue (50% dichloromethane:hexanes) gave 35 g of the title compound. MS (ESI): 232 (M+H⁺).

c) 8-oxa-3-aza-bicyclo[5.1.0]octane-3-carboxylic acid benzyl ester

To a solution of the compound of Example 1 (b) (3.0 g 13.0 mmol) in CH₂Cl₂ was added m-CPBA (6.7 g, 39.2 mmol). The mixture was stirred overnight at room temperature whereupon it was partitioned between CH₂Cl₂ and staurated K₂CO₃. The organic layer was washed with sat. NaHCO₃, water, brine, dried (MgSQ₄), filtered and concentrated to give 3.08 g of the title compound as an oil. MS (ESI): 248 (M+H⁺), 270 (M+Na⁺).

d) 4-azido-3-hydroxy-azepane-1-carboxylic acid benzyl ester

To a solution of the compound of Example 1(c) (2.0 g, 8.1 mmol) in methanol:water (8:1 solution) was added NH₄Cl (1.29 g, 24.3 mmol) and sodium azide (1.58 g, 24.30 mmol), The reaction was heated to 40° C. until complete consumption of the starting epoxide was observed by TLC analysis. The majority of the solvent was removed in vacuo and the remaining solution was partitioned between ethyl acetate and pH 4 buffer. The organic layer was washed with sat. NaHCO₃, water, brine dried (MgSO₄), filtered and concentrated. Column chromatography (20% ethyl acetate:hexanes) of the residue provided 1.3 g of the title compound. MS (ESI): 291 (M+H⁺) plus 0.14 g of trans-4-hydroxy-3-azido-hexahydro-1H-azepine

e) 4-amino-3-hydroxy-azepane-1-carboxylic acid benzyl ester

To a solution of the azido alcohol of Example 1(d) (1.1 g, 3.79 mmol) in methanol was added triethylamine (1.5 mL, 11.37 mmol) and 1,3-propanedithiol (1.1 mL, 11.37 mL). The reaction was stirred until complete consumption of the starting material was observed by TLC analysis whereupon the reaction was concentrated in vacuo. Column chromatography of the residue (20% methanol:dichloromethane) provided 0.72 g of the title compound. MS (ESI): 265 (M+H⁺).

f) 4-((S)-2-tert-butoxycarbonylamino-4-methyl-pentanoylamino)-3-hydroxy-azepan-1-carboxylic acid benzyl ester

To a solution of the amino alcohol of Example 1(e) (720 mg, 2.72 mmol) in CH₂Cl₂ was added EDC (521 mg), HOBt (368 mg) and N-Boc-leucine (630 mg). The reaction was maintained at room temperature until complete consumption of the starting material was observed by TLC analysis. The reaction was diluted with ethyl acetate and washed with 1N HCl, sat. K₂CO₃, water, brine, dried (MgSO₄), filtered and concentrated. Column chromatography of the residue (3% methanol:dichloromethane) gave 1.0 g of the title compound. MS (ESI): 478 (M+H⁺).

g) [(S)-1-(3-hydroxy-azepan-4-ylcarbamoyl)-3-methyl-butyl]-carbamic acid tert butyl ester

To a solution of the compound of Example 1(f) (1.0 g) and 10% Pd/C (catalytic) in ethyl acetate:methanol (2:1 solution) was affixed a balloon of hydrogen. The reaction was stirred until complete consumption of the starting material was observed by TLC analysis. The reaction was filtered to remove the catalyst and the filtrate was concentrated in vacuo to provide 0.82 g of the title compound. MS (ESI): 344 (M+H⁺).

h) [(S)-1-(1-benzyl-3 hydroxy-azepan-4-ylcarbamoyl)-3-methyl-butyl]-carbamic acid tert butyl ester

To a solution of the compound of Example 1(g) (0.69 g, 2.01 mmol) in CH₂Cl₂ was added benzaldehyde (0.32 mL, 3.01 mmol) followed by sodium triacetoxyborohydride (0.85 g, 4.02 mmol). The reaction was stirred until complete as determined by TLC analysis whereupon several drops of water were added to the reaction to destroy the excess sodium triacetoxyborohydride. The mixture was diluted with ethyl acetate washed with sat. NaHCO₃, water, brine, dried (Na₂SO₄), filtered and concentrated. Column chromatography of the residue (5% methanol:dichloromethane) gave 800 mg of the title compound. MS (ESI): 434 (M+H⁺).

i) (S)-2-amino4-methyl-pentanoic acid (1-benzyl-3-hydroxy-azepan-4-yl)-amide

To a solution of the compound of Example 1(h) (800 mg) in methanol (15 mL) was added 4M HCl in dioxane (15 mL). The reaction was stirred at room temperature overnight whereupon it was concentrated in vacuo to give 800 mg of the title compound. MS (ES): 334 (M+H+).

j) benzo[1,3]dioxole-5-carboxylic acid [(S)-1-(1-benzyl-3-hydroxy-azepan-4-ylcarbamoyl)-3-methyl-butyl]-amide

To a solution of the amine salt of Example 1(i) (200 mg, 0.49 mmol) in CH₂Cl₂ was added triethylamine (0.17 mL, 1.22 mmol), EDC (103.5 mg, 0.54 mmol), HOBt (73 mg, 0.54 mmol) and benzo[1,3]dioxole-5-carboxylic acid (90 mg, 0.54 mmol). The reaction was stirred until complete by TLC analysis. The reaction was diluted with ethyl acetate and washed with sat. NaHCO₃, water, brine, dried (Na₂SO₄), filtered and concentrated. Column chromatography of the residue (5% methanol:dichloromethane) gave 0.14 g of the title compound. MS (ESI): 482 (M+H⁺).

k) benzo[1,3]dioxole-5-carboxylic acid [(S)-1-(1-benzyl-3-oxoazepan-4-ylcarbamoyl)-3-methyl-butyl]amide

To a solution of the alcohol of Example 1(j) (130 mg, 0.21 mmol) in DMSO was added TEA (0.17 mL) and pyridine sulfur trioxide complex (96 mg, 0.61 mmol). The reaction was stirred at room temperature for approximately 2 hours whereupon it was partitioned between ethyl acetate and water. The organic layer was washed with brine, dried (MgSO₄), filtered and concentrated. Column chromatography of the residue (5% CH₃OH:CH₂Cl₂) provided 100 mg of the title compound as a mixture of diastereomers. Separation of the diastereomers by HPLC (Whelk-O1; ethanol/hexanes) provided the title compound. MS (ESI): 480.3 (M+H⁺).

### Example 2

### Preparation of Quinoline-2-carboxylic acid [(S)-1-(1-benzyl-3-oxo-azepan-4-ylcarbamoyl)-3-methyl-butyl]amide

Following the procedure of Example 1(a)-1(k), except substituting quinoline-2-carboxylic acid for benzo[1,3]dioxole-5-carboxylic acid in step (j), the title compound was prepared. MS (ESI): 492.4 (M+H⁺).

### Example 3

### Preparation of 5-(2-morpholino-4-yl-ethoxy)-benzofuran-2-carboxylic acid ((S)-3-methyl-1-{3-oxo-1-[2-(3-pyridin-2-yl-phenyl)acetyl]-azepan-4-ylcarbamoyl}-butyl)amide

a) methyl 3-(trifluoromethylsulfonyloxy)phenylacetate

To an oven-dried flask under argon atmosphere containing sodium hydride (2.54 g, 60% dispersion in mineral oil, 63.5 mmol) was added anhydrous pentane (20 mL). The slurry was allowed to stir for 5 min, allowed to settle, most of the pentane was removed, and anhydrous THF (40 mL) was added. To this suspension was added a solution of methyl 3-hydroxyphenylacetate (9.99 g, 60.1 mmol) in anhydrous THF (20 mL) and the reaction was allowed to stir at room temperature for 20 min. To this mixture was then added a solution of N-phenyltrifluoromethanesulfonimide (22.53 g, 63.1 mmol)) in anhydrous THF (40 mL) and the reaction was allowed to stir at room temperature until TLC analysis indicated the complete consumption of starting material (1.5 h). The reaction was quenched by the addition of H₂O (10 mL), concentrated to one half original volume, then diluted with CHCl₃ (200 mL) and washed with H₂O. The aqueous layer was washed with fresh CHCl₃ (50 mL), the combined organic layers were washed with 10% Na₂CO₃, water, and saturated brine, then dried (MgSO₄), filtered and concentrated. Column chromatography of the residue (silica gel, 5:95 EtOAc:hexanes, then 10:90 EtOAc: hexanes) gave 17.47 g of the title compound. ¹H NMR (400 MHz, CDCl₃) 7.42 (m, 1H), 7.31-7.19 (m, 3H), 3.72 (s, 3H), 3.68 (s, 2H).

b) methyl 3-(2-pyridyl)phenylacetate

To a solution of the compound of Example 3(a) (6.86 g, 23.0 mmol) in anhydrous dioxane (100 mL) was added 2-pyridyltributylstannane (8.89 g, 24.1 mmol), LiCl (2.94 g, 69.3 mmol), 2,6-di-tert-butyl-4-methylphenol (a few crystals), and Pd(PPh₃)₄ (632.1 mg, 0.55 mmol). The reaction was protected from light with foil and heated at reflux overnight. The reaction was allowed to cool to room temperature and was concentrated. Column chromatography of the residue (silica gel, 1:3 EtOAc:hexanes, then 1:2 EtOAc:hexanes) gave 3.85 g of the title compound. MS (ESI): 228.1 (M+H)⁺.

c) 3-(2-pyridyl)phenylacetic acid

To a solution of the compound of Example 3(b) (3.8 g, 16.7 mmol) in THF (50 mL) was added a solution of LiOH.H₂O (780.2 mg, 18.6 mmol) in water (10 mL). The reaction was allowed to at room temperature until TLC analysis indicated the complete consumption of starting material (2 h). The reaction mixture was concentrated to remove THF, then neutralized to pH 7 by the addition of 1N HCl, diluted with brine (50 mL), and washed with CHCl₃ (100 mL) The aqueous layer was readjusted back to pH 7 by the addition on 1N NaOH and washed with fresh CHCl₃ (100 mL). After repeating this procedure once more, the organic layers were combined, dried (MgSO₄), filtered and concentrated to give 3.79 g of the title compound. MS (ESI): 214.3 (M+H)⁺.

d) ((S)-3-methyl-1{3-hydroxy-1-[2-(3-pyridin-2-yl-phenyl)-ethanoyl]-azepan-4-ylcarbamoyl}-butyl)-carbamic acid tert-butyl ester

To a solution of the compound of Example 1(g) (0.5 g, 1.46 mmol) in CH₂Cl₂ was added EDC (307 mg, 1.60 mmol), HOBt (216 mg, 1.60 mmol) the compound of Example 3(c) (341 mg, 1.60 mmol). The reaction was stirred at room temperature until complete as determined by TLC analysis. Workup and column chromatography (2% methanol:dichlorome,thane) provided the title compound. MS (ESI): 539 (M+H⁺).

e) ethyl 5-hydroxybenzofuran-2-carboxylate

To a mixture of aluminum chloride (6.3 g, 47.7 mmol) and ethanethiol (4.5 g, 72.9 mmol) in dichloromethane (81 mL) at 0° C. was added ethyl 5-methoxybenzofuran-2-carboxylate (3.0 g, 13.6 mmol). After stirring for 16 h at room temperature, the mixture was poured into water, acidified with 3N HCl and extracted with dichloromethane (2×). The organic layers were combined, washed with saturated brine, dried (MgSO₄), filtered and concentrated. The residue was purified by column chromatography (silica gel, ethyl acetatelhexane) to yield the title compound as a white solid (2.16 g, 77%). ¹H NMR (400 MHz, CDCl₃) δ 7.45 (m, 2H), 7.08 (m, 1H), 7.02 (m, 1H), 5.35 (s b, 1H), 4.44 (q, 2H), 1.42 (t, 3H).

f) ethyl 5-[2-(4-morpholino)ethoxy]benzofuran-2-carboxylate

To a solution of the compound of Example 3(e) (0.200 g 0.971 mmol), 4-(2-hydroxyethyl)morpholine (0.165 g, 1.26 mmol), and triphenylphosphine (0.331 g, 1.26 mmol) in THF (4 mL) at 0° C. was added dropwise diisopropylazodicarboxylate (0.254 g, 1.26 mmol). After stirring at room temperature for 16 h, the solution was concentrated and purified by column chromatography (silica gel, ethyl acetatelhexane) to yield the title compound as a white solid (0.235 g, 76%). ¹H NMR (400 MHz, CDCl₃) δ 7.48 (m, 2H), 7.07 (m, 2H), 4.43 (q, 2H), 4.14 (m, 2H), 3.76 (m, 4H), 2.86 (m, 2H), 2.61 (m 4H). 1.40 (t, 3H).

g) 5-[2-(4-morpholino)ethoxy]benzofuran-2-carboxylic acid

To a stirring solution of the compound of Example 3(f) (0.235 g, 0.74 mmol) in THF (4.0 mL) and water (4.0 mL) was added lithium hydroxide monohydrate (0.035 g, 0.81 mmol). After stirring at reflux for 16 h, the solution was concentrated and the residue was dissolved in water and acidified with 1eq 1N HCl. The mixture was frozen and placed on a lyophilizer for 16 h to yield the title compound as an off-white solid (0.150 g, 70%). MS (ESI): 292.1 (M+H)⁺.

h) 5-(2-morpholino-4-yl-ethoxy)-benzofuran-2-carboxylic acid ((S)-3-methyl-1-{3-oxo-1-[2-(3-pyridin-2-yl-phenyl)acetyl]-azepan-4-ylcarbamoyl}-butyl)amide

Following the procedure of Example 1(i)-1(k), except substituting ((S)-3-methyl-1{3-hydroxy-1-[2-(3-pyridin-2-yl-phenyl)-ethanoyl]-azepan-4-ylcarbamoyl}-butyl)-carbamic acid tert-butyl ester for [(S)-1-(1-benzyl-3-hydroxy-azepan-4-ylcarbamoyl)-3-methyl-butyl]-carbamic acid tert butyl ester in step (i) and 5-[2-(4-morpholino)ethoxy]benzofuran-2-carboxylic acid for benzo[1,3]dioxole-5-carboxylic acid in step (j), the title compound was prepared. MS (ESI): 710.3 (M+H⁺).

### Example 4

### Preparation of 5-(2-morpholino-4-yl-ethoxy)-benzofuran-2-carboxylic acid ((S)-3-methyl-1-{3-oxo-1-[2-(3-pyridin-2-yl-phenyl)acetyl]-azepan-4-ylcarbamoyl{-butyl)amide

The title compound was isolated as the second eluting compound from the HPLC purification in Example 3(h). MS (ESI): 710.3 (M+H⁺).

### Example 5

### Preparation of 4-((S)-4-Methyl-2-{[5-(2-morpholino-4-yl-ethoxy)-benzofuran-2-carbonyl]amino}-pentanoylamino)-3-oxo-azepane-1-carboxylic acid phenylamide

a) [(S)-1-(3-hydroxy-1-phenylcarbamoyl-azepan4-ylcarbamoyl)-3-methyl-butyl]-carbamic acid tert-butyl ester

To a solution of the compound of Example 1(g) (0.5 g, 1.46 mmol) in dichloromethane (20 mL) was added phenyl isocyanate (0.24 mL, 2.18 mmol). The reaction was stirred at room temperature until complete as determined by TLC analysis. Workup and column chromatography (5% methanol:dichloromethane) provided 578 mg of the title compound. MS (ESI): 463 (M+H⁺).

b) 4-((S)-4-methyl-2-{[5-(2-morpholino-4-yl-ethoxy)-benzofuran-2-carbonyl]amino}-pentanoylamino)-3-oxo-azepane-1-carboxylic acid phenylamide

Following the procedure of Example 1(i)-1(k), except substituting [(S)-1-(3-Hydroxy-1-phenylcarbamoyl-azepan-4-ylcarbamoyl)-3-methyl-butyl]-carbamic acid tert-butyl ester for [(S)-1-(1-benzyl-3-hydroxy-azepan-4-ylcarbamoyl)-3-methyl-butyl]-carbamic acid tert butyl ester in step (i) and 5-[2-(4-morpholino)ethoxy]benzofuran-2-carboxylic acid for benzo[1,3]dioxole-5-carboxylic acid in step (j), the title compound was prepared. MS (ESI): 634 (M+H⁺).

### Example 6

### Preparation of 5-(2-Morpholin-4-yl-ethoxy)-benzofuran-2-carboxylic acid [(S)-1-(1-benzenesulfonyl-3-oxo-azepan-4-ylcarbamoyl)-3-methyl-butyl]amide

a) [(S)-1-(3-hydroxy-1-phenylsulfonyl-azepan-4-ylcarbamoyl)-3-methyl-butyl]-carbamic acid tert-butyl ester

To a solution of the compound of Example 1(g) (0.5 g, 1.46 mmol) in dichloromethane was added triethylamine (0.4 mL, 2.92 mmol) followed by benzenesulfonyl chloride (0.28 mL, 2.18 mmol). The reaction was stirred at room temperature until complete as determined by TLC analysis. Workup and column chromatography (10% methanol:dichloromethane) provided 450 mg of the title compound. MS (ESI): 484 (M+H⁺).

b) 5-(2-morpholin-4-yl-ethoxy)-benzofuran-2-carboxylic acid [(S)-1-(1-benzenesulfonyl-3-oxo-azepan-4-ylcarbamoyl)-3-methyl-butyl]amide

Following the procedure of Example 1(i)-1(k), except substituting [(S)-1-(3-hydroxy-1-phenylsulfonyl-azepan-4-ylcarbamoyl)-3-methyl-butyl]-carbamic acid tert-butyl ester for [(S)-1-(1-benzyl-3-hydroxy-azepan-4-ylcarbamoyl)-3-methyl-butyl]-carbamic acid tert butyl ester in step (i), and 5-[2-(4-morpholino)ethoxy]benzofuran-2-carboxylic acid for benzo[1,3]dioxole-5-carboxylic acid in step (j), the title compound was prepared. MS (ESI): 655 (M+H⁺).

### Example 7

### Preparation of 5-(2-Morpholin-4-yl-ethoxy)-benzofuran-2-carboxylic acid [(S)-1-(1-benzenesulfonyl-3-oxo-azepan-4-ylcarbamoyl)-3-methyl-butyl]amide

The title compound was isolated as the second eluting compound from the HPLC purification in Example 6(b). MS (ESI): 655 (M+H⁺).

### Example 8

### Preparation of 5-(2-Pyrrolidin-1-yl-ethoxy)-benzofuran-2-carboxylic acid [(S)-1-(1-benzenesulfonyl-3-oxo-azepan-4-ylcarbamoyl)-3-methyl-butyl]amide

Following the procedure of Example 6(a)-6(b), except substituting 5-(2-pyrrolidin-1-yl-ethoxy)benzofuran-2-carboxylic acid for 5-(2-morpholin-4-yl-ethoxy)-benzofuran-2-carboxylic acid in step (b), the title compound was prepared. MS (ESI): 639 (M+H⁺).

### Example 9

### Preparation of 5-(2-Piperidin-1-yl-ethoxy)-benzofuran-2-carboxylic acid [(S)-1-(1-benzenesulfonyl-3-oxo-azepan-4-ylcarbamoyl)-3-methyl-butyl]amide

Following the procedure of Example 6(a)-6(b), except substituting 5-(2-piperidin-1-yl-ethoxy)benzofuran-2-carboxylic acid for 5-(2-morpholin-4-yl-ethoxy)-benzofuran-2-carboxylic acid in step (b), the title compound was prepared. MS (ESI): 653 (M+H⁺).

### Example 10

### Preparation of Naphthlene-2-carboxylic acid ((S)-3-methyl-1-{3-oxo-1-[2-(3-pyridin-2-yl-phenyl)ethyl]-azepan-4-ylcarbamoyl}-butyl)amide

Following the procedure of Example 1(a)-1(k), except substituting 5-(2-piperidin-1-yl-ethoxy)benzofurfan-2-carboxylic acid for benzaldehyde in step (h), and naphthalene-2-carboxylic acid for benzo[1,3]dioxole-5-carboxylic acid in step (j), the title compound was prepared. MS (ESI): 557 (M+H⁺).

### Example 11

### Preparation of 1H-Indole-2-carboxylic acid [(S)-1-(1-benzenesulfonyl-3-oxo-azepan-4-ylcarbamoyl)-3-methyl-butyl]amide

Following the procedure of Example 6(a)-6(b), except substituting indole-2-carboxylic acid for 5-(2-morpholin-4-yl-ethoxy)-benzofuran-2-carboxylic acid in step (b), the title compound was prepared. MS (ESI): 525 (M+H⁺).

### Example 12

### Preparation of Benzofuran-2-carboxylic acid [(S)-1-(1-benzenesulfonyl-3-oxo-azepan-4-ylcarbamoyl)-3-methyl-butyl]amide

Following the procedure of Example 6(a)-6(b), except substituting benzofuran-2-carboxylic acid for 5-(2-morpholin-4-yl-ethoxy)-benzofuran-2-carboxylic acid in step (b), the title compound was prepared. ¹H NMR (CDCl₃): δ 1.0 ( m, 6H), 1.5-2.1 ( m, 5H), 2.2 (m, 2H), 2.6 (m, 1H), 3.5 (d, 1H). 4.1 (m, 1H), 4.7 ( m, 2H), 5.0 ( m, 1H), 7.2-7.2 (m, 10H).

### Example 13

### Preparation of 5-(2-Pyrrolidin-1-yl-ethoxy)-benzofuran-2-carboxylic acid [(S)-1-(1-benzenesulfonyl-3-oxo-azepan-4-ylcarbamoyl)-3-methyl-butyl]amide

The title compound was isolated as the second eluting compound from the HPLC purification in Example 8. MS (ESI): 639 (M+H⁺).

### Example 14

### Preparation of 5-(2-Piperidin-1-yl-ethoxy)-benzofuran-2-carboxylic acid [(S)-1-(1-benzenesulfonyl-3-oxo-azepan-4-ylcarbamoyl)-3-methyl-butyl]amide

The title compound was isolated as the first eluting compound from the HPLC purification in Example 9. MS (ESI): 653 (M+H⁺).

### Example 15

### Preparation of 5-(2-Morpholino-4-yl-ethoxy)-benzofuran-2-carboxylic acid {(S)-3-methyl-1-[1-(4-methyl-2entanoyl)-3-oxo-azepan-4-ylcarbamoyl]-butyl}-amide

Following the procedure of Example 3(a)-3(h), except substituting isocaproic acid for 3-(2-Pyridyl)phenylacetic acid in step (d), the title compound was prepared. MS (ESI): 613 (M+H⁺).

### Example 16

### Preparation of Benzofuran-2-carboxylic acid }(S)-3-methyl-1-[3-oxo-1-(pyridine-2-sulfonyl)-azepan-4-ylcarbamoyl]-butyl}-amide

Following the procedure of Example 6(a)-6(b), except substituting pyridine-2-sulfonyl chloride for benzenesulfonyl chloride in step (a) and benzofuran-2-carboxylic acid for 5-(2-morpholin-4-yl-ethoxy)-benzofuran-2-carboxylic acid in step (b), the title compound was prepared. MS (ESI): 527 (M+H⁺).

### Example 17

### Preparation of Naphthalene-2-carboxylic acid {(S)-3-methyl-1-[3-oxo-1-(pyridine-2-sulfonyl)-azepan-4-ylcarbamoyl]butyl}-amide

Following the procedure of Example 6(a)-6(b), except substituting pyridine-2-sulfonyl chloride for benzenesulfonyl chloride in step (a) and naphthalne-2-carboxylic acid for 5-(2-morpholin-4-yl-ethoxy)-benzofuran-2-carboxylic acid in step (b), the title compound was prepared. MS (ESI): 537 (M+H⁺).

### Example 18

### Preparation of 5-(2-Morpholino-4-yl-ethoxy)-benzofuran-2-carboxylic acid {(S)-3-methyl-1-[3-oxo-1-(pyridine-2-sulfonyl)-azepan-4-ylcarbamoyl]-butyl}1-amide

Following the procedure of Example 6(a)-6(b), except substituting pyridine-2-sulfonyl chloride for benzenesulfonyl chloride in step (a), the title compound was prepared. MS (ESI): 656 (M+H⁺).

### Example 19

### Preparation of 5-(2-Morpholino-4-yl-ethoxy)-benzofuran-2-carboxylic acid {(S)-3-methyl-1-[3-oxo-1-(pyridine-2-sulfonyl)-azepan-4-ylcarbamoyl]-butyl}-amide

The title compound was isolated as the first eluting compound from the HPLC purification in Example 18. MS (ESI): 656 (M+H⁺).

### Example 20

### Preparation of 5-(2-Morpholino-4-yl-ethoxy)-benzofuran-2-carboxylic acid {(S)-3-methyl-1-[3-oxo-1-(pyridine-2-sulfonyl)-azepan-4-ylcarbamoyl]-butyl}-amide

The title compound was isolated as the second eluting compound from the HPLC purification in Example 18. MS (ESI): 656 (M+H⁺).

### Example 21

### Preparation of Benzofuran-2-carboxylic acid {(S)-3-methyl-1-[3-oxo-1-(pyridine-2-sulfonyl)-azepan-4-ylcarbamoyl]-butyl}-amide

The title compound was isolated as the first eluting compound from the HPLC purification in Example 16. MS (ESI): 527 (M+H⁺).

### Example 22

Preparation of 4-[2-(2-{(S)-3-Methyl-1[3-oxo-1-(pridine-2-sulfonyl)-azepan-4-ylcarbamoyl]-butylcarbamoyl}-benzofuran-5-yloxy)-ethyl]-piperazine-1-carboxylic acid tert-butyl ester

Following the procedure of Example 6(a)-6(b), except substituting pyridine-2-sulfonyl chloride for benzenesulfonyl chloride in step (a) and 4-[2-(2-carboxy-benzofuran-5-yloxy)-ethyl]-piperazine-1-carboxylic acid tert-butyl ester for 5-(2-morpholin-4-yl-ethoxy)-benzofuran-2-carboxylic acid in step (b), the title compound was prepared. MS (ESI): 757 (M+H⁺).

### Example 23

### Preparation of 5-(2-Piperizin-1-yl-ethoxy)-benzofuran-2-carboxylic acid {(S)-3-methyl-1-[3-oxo-1-(pyridine-2-sulfonyl)-azepan-4-ylcarbamoyl]-3-butyl}-amide

The compound of Example 22 (0.02 g) was dissolved in 4M HCl in dioxane. The reaction was stirred until complete whereupon it was concentrated to provide the title compound. MS (ESI): 655 (M+H⁺).

### Example 24

### Preparation of Quinoline-2-carboxylic acid {(S)-3-methyl-1-[3-oxo-1-(pyridine-2-sulfonyl)-azepan-4-ylcarbamoyl]-butyl}amide

Following the procedure of Example 6(a)-6(b), except substituting pyridine-2-sulfonyl chloride for benzenesulfonyl chloride in step (a) and quinoline-2-carboxylic acid for 5-(2-morpholin-4-yl-ethoxy)-benzofuran-2-carboxylic acid in step (b), the title compound was prepared. MS (ESI): 538 (M+H⁺).

### Example 25

### Preparation of Quinoline-6-carboxylic acid {(S)-3-methyl-1-[3-oxo-1-(pyridine-2-sulfonyl)-azepan-4-ylcarbamoyl]-butyl}amide

Following the procedure of Example 6(a)-6(b), except substituting pyridine-2-sulfonyl chloride for benzenesulfonyl chloride in step (a) and quinoline-6-carboxylic acid tert-butyl ester for 5-(2-morpholin-4-yl-ethoxy)-benzofuran-2-carboxylic acid in step (b), the title compound was prepared. MS (ESI): 538 (M+H⁺).

### Example 26

### Preparation of Quinoline-4-carboxylic acid {(S)-3-methyl-1-[3-oxo-1-(pyridine-2-sulfonyl)-azepan-4-ylcarbamoyl]-butyl}amide

Following the procedure of Example 6(a)-6(b), except substituting pyridine-2-sulfonyl chloride for benzenesulfonyl chloride in step (a) and quinoline-4-carboxylic acid for 5-(2-morpholin-4yl-ethoxy)-benzofuran-2-carboxylic acid in step (b), the title compound was prepared. MS (ESI): 538 (M+H⁺).

### Example 27

### Preparation of Quinoline-3-carboxylic acid {(S)-3-methyl-1-3-oxo-1-(pyridine-2-sulfonyl)-azepan-4-ylcarbamoyl]-butyl}amide

Following the procedure of Example 6(a)-6(b), except substituting pyridine-2-sulfonyl chloride for benzenesulfonyl chloride in step (a) and quinoline-3-carboxylic acid tert-butyl ester for 5-(2-morpholin4-yl-ethoxy)-benzofuran-2-carboxylic acid in step (b), the title compound was prepared. MS (ESI): 538 (M+H⁺).

### Example 28

### Preparation of Isoquinoline-3-carboxylic acid {(S)-3-methyl-1-[3-oxo-1-(pyridine-2-sulfonyl)-azepan-4-ylcarbamoyl]-butyl}amide

Following the procedure of Example 6(a)-6(b), except substituting pyridine-2-sulfonyl chloride for benzenesulfonyl chloride in step (a) and isoquinoline-3-carboxylic acid for 5-(2-morpholin4-yl-ethoxy)-benzofuran-2-carboxylic acid in step (b), the title compound was prepared. MS (ESI): 538 (M+H⁺).

### Example 29

### Preparation of Isoquinoline-1-carboxylic acid {(S)-3-methyl-1-[3-oxo-1-(pyridine-2-sulfonyl)-azepan-4-ylcarbamoyl]-butyl}amide

Following the procedure of Example 6(a)-6(b), except substituting pyridine-2-sulfonyl chloride for benzenesulfonyl chloride in step (a) and isoquinoline-1-carboxylic acid for 5-(2-morpholin-4-yl-ethoxy)-benzofuran-2-carboxylic acid in step (b), the title compound was prepared. MS (ESI): 538 (M+H⁺).

### Example 30

### Preparation of Quinoxaline-2-carboxylic acid {(S)-3-methyl-1-[3-oxo-1-(pyridine-2-sulfonyl)-azepan-4-ylcarbamoyl]-butyl}amide

Following the procedure of Example 6(a)-6(b), except substituting pyridine-2-sulfonyl chloride for benzenesulfonyl chloride in step (a) and quinoline-2-carboxylic acid for 5-(2-morpholin-4-yl-ethoxy)-benzofuran-2-carboxylic acid in step (b), the title compound was prepared. MS (ESI): 539 (M+H⁺).

### Example 31

### Preparation of Benzo[b]thiophene-2-carboxylic acid {(S)-3-methyl-1-[3-oxo-1-(pyridine-2-sulfonyl)-azepan-4-ylcarbamoyl]-butyl}amide

Following the procedure of Example 6(a)-6(b), except substituting pyridine-2-sulfonyl chloride for benzenesulfonyl chloride in step (a) and benzo[b]thiophene-2-carboxylic acid for 5-(2-morpholin-4-yl-ethoxy)-benzofuran-2-carboxylic acid in step (b), the title compound was prepared. MS (ESI): 545 (M+H⁺).

### Example 32

### Preparation of 1H-Indole-2-carboxylic acid {(S)-3-methyl-1-[3-oxo-1-(pyridine-2-sulfonyl)-azepan-4-ylcarbamoyl]-butyl}amide

Following the procedure of Example 6(a)-6(b), except substituting pyridine-2-sulfonyl chloride forbenzenesulfonyl chloride in step (a) and indole-2-carboxylic acid for 5-(2-morpholin-4-yl-ethoxy)-benzofuran-2-carboxylic acid in step (b), the title compound was prepared. MS (ESI): 526 (M+H⁺).

### Example 33

### Preparation of 5-Methoxy-benzofuran-2-carboxylic acid {(S)-3-methyl-1-[3-oxo-1-(pyridine-2-sulfonyl)-azepan-4-ylcarbamoyl]-butyl}amide

Following the procedure of Example 6(a)-6(b), except substituting pyridine-2-sulfonyl chloride for benzenesulfonyl chloride in step (a) and 5-methoxybenzofuran-2-carboxylic acid for 5-(2-morpholin-4-yl-ethoxy)-benzofuran-2-carboxylic acid in step (b), the title compound was prepared. MS (ESI): 557 (M+H⁺).

### Example 34

### Preparation of 5-Bromo-furan-2-carboxylic acid {(S)-3-methyl-1-[3-oxo-1-(pyridine-2-sulfonyl)-azepan-4-ylcarbamoyl]-butyl}amide

Following the procedure of Example 6(a)-6(b), except substituting pyridine-2-sulfonyl chloride for benzenesulfonyl chloride in step (a) and 5-bromofuran-2-carboxylic acid for 5-(2-morpholin-4-yl-ethoxy)-benzofuran-2-carboxylic acid in step (b), the title compound was prepared. MS (ESI): 555 (M+H⁺).

### Example 35

### Preparation of 5-Nitro-furan-2-carboxylic acid {(S)-3-methyl-1-[3-oxo-1-(pyridine-2-sulfonyl)-azepan-4-ylcarbamoyl]-butyl}amide

Following the procedure of Example 6(a)-6(b), except substituting pyridine-2-sulfonyl chloride for-benzenesulfonyl chloride in step (a) and 5-nitrofuran-2-carboxylic acid for 5-(2-morpholin-4-yl-ethoxy)-benzofuran-2-carboxylic acid in step (b), the title compound was prepared. MS (ESI): 522 (M+H⁺).

### Example 36

### Preparation of 5-(4-Nitro-phenyl)-furan-2-carboxylic acid {(S)-3-methyl-1-[3-oxo-1-(pyridine-2-sulfonyl)-azepan-4-ylcarbamoyl]-butyl}amide

Following the procedure of Example 6(a)-6(b), except substituting pyridine-2-sulfonyl chloride for benzenesulfonyl chloride in step (a) and 5-(4-nitrophenyl)furan-2-carboxylic acid for 5-(2-morpholin-4-yl-ethoxy)-benzofuran-2-carboxylic acid in step (b), the title compound was prepared. MS (ESI): 598 (M+H⁺).

### Example 37

### Preparation of (S)-2-[2-(4-Fluoro-phenoxy)-acetylamino]-4-methyl-pentanoic acid [3-oxo-(pyridine-2-sulfonyl)-azepan-4-yl]-amide

Following the procedure of Example 6(a)-6(b), except substituting pyridine-2-sulfonyl chloride for benzenesulfonyl chloride in step (a) and 2-(4-fluorophenoxy)acetic acid for 5-(2-morpholin-4-yl-ethoxy)-benzofuran-2-carboxylic acid in step (b), the title compound was prepared. MS (ESI): 535 (M+H⁺).

### Example 38

### Preparation of Benzofuran-2-carboxylic acid {(S)-3-methyl-1-[3-oxo-1-(thiophene-2-sulfonyl)-azepan-4-ylcarbamoyl]-butyl}-amide

Following the procedure of Example 6(a)-6(b), except substituting thiophene-2-sulfonyl chloride fortbenzenesulfonyl chloride in step (a) and benzofuran-2-carboxylic acid for 5-(2-morpholin-4-yl-ethoxy)-benzofuran-2-carboxylic acid in step (b), the title compound was prepared. MS (ESI): 532 (M+H⁺).

### Example 39

Preparation of 5,6-Dimethoxy-benzofuran-2-carboxylic acid {(S)-3-methyl-1-[3-oxo-1-(1-methyl-1H-imidazole-4-sulfonyl)-azepan-4-ylcarbamoyl]-butyl}amide

a) 2-hydroxy-4,5-dimethoxybenzaldehyde

To a stirring solution of 2-benzyloxy-4,5-dimethoxybenzaldehyde (1.0 g, 3.67 mmol) in ethyl acetate (25 mL) was added 10% palladium on carbon (0.50 g). The mixture was stirred under a hydrogen atmosphere for 4 h, then filtered through Celite. The filtrate was concentrated to yield the title compound as a pale yellow solid (0.632 g, 95%). ¹H NMR (400 MHz, CDCl₃) δ 11.41 (s, 1H), 9.72 (s, 1H), 6.89 (s, 1H), 6.48 (s, 1H), 3.91 (s, 3H), 3.88 (s, 3H).

b) 4,5-dimethoxy-2-ethoxycarbonylmethoxybenzaldehyde

To a stirring solution of the compound of Example 39(a) (0.628 g, 3.4 mmol), and ethyl bromoacetate (0.575 g, 3.4 mmol) in acetone (150 mL) was added K₂CO₃ (0.715 g, 5.2 mmol). After stirring at reflux for 4 h, the mixture was partitioned between ethyl acetate and water. The organic layer was washed with brine, dried (MgSO₄), filtered and concentrated. The residue was purified by column chromatography (silica gel, ethyl acetate/hexane) to yield the title compound as a colorless oil (0.758 g, 82%). ¹H NMR (400 MHz, CDCl₃) δ 10.39 (s, 1H), 7.30 (s, 1H), 6.41 (s, 1H), 4.72 (s, 2H), 4.22 (q, 2H), 3.90 (s, 3H), 3.83 (s, 3H), 1.26 (t, 3H).

c) ethyl 5,6-dimethoxybenzofuran-2-carboxylate

A mixture of the compound of Example 39(b) (0.758 g, 2.8 mmol) and potassium carbonate (0.975 g, 7.1 mmol) was stirred at 80° C. in DMF (20 mL) for 5 h. The mixture was cooled and partitioned between ethyl acetate and water. The organic layer was washed with water and satruated brine then dried (MgSO₄), filtered and concentrated. The residue was purified by column chromatography (silica gel, ethyl acetate/hexane) to yield the title compound as a white solid (0.405 g, 58%). ¹H NMR (400 MHz, CDCl₃) δ 7.45 (s, 1H), 7.10 (s, 1H), 7.04 (s, 1H), 4.41 (q, 2H), 3.93 (s, 3H), 3.91 (s, 3H), 1.41 (t, 3H).

d) 5,6-dimethoxybenzofuran-2-carboxylic acid

Following the procedure of Example 3(g), except substituting ethyl 5,6-dimethoxybenzofuran-2-carboxylate for ethyl 5-[2-(4-morpholino)ethoxy]benzofuran-2-carboxylate, the title compound was prepared as a white solid (0.263 g, 73%). ¹H NMR (400 MHz, CDCl₃) δ 7.40 (s, 1H), 7.03 (s, 1H), 7.01 (s, 1H), 3.90 (s, 3H), 3.88 (s, 3H).

e) 5,6-Dimethoxy-benzofuran-2-carboxylic acid {(S)-3-methyl-1-[3-oxo-1-(1-methyl-1H-imidazole-4-sulfonyl)-azepan-4-ylcarbamoyl]-butyl}amide

Following the procedure of Example 6(a)-6(b), except substituting 1-methylimidazole-4-sulfonyl chloride for benzenesulfonyl chloride in step (a) and 5,6-dimethoxybenzofuran-2-carboxylic acid for 5-(2-morpholin4-yl-ethoxy)-benzofuran-2-carboxylic acid in step (b), the title compound was prepared. MS (ESI): 590 (M+H⁺).

### Example 40

### Preparation of Benzofuran-2-carboxylic acid {(S)-3-methyl-1-[1-(1-methyl-1H-imidazole-3-sulfonyl)-3-oxo-azepan-4-ylcarbamoyl]-butyl}amide

Following the procedure of Example 6(a)-6(b), except substituting 1-methylimidazole-2-sulfonyl chloride for benzenesulfonyl chloride in step (a) and benzofuran-2-carboxylic acid for 5-(2-morpholin-4-yl-ethoxy)-benzofuran-2-carboxylic acid in step (b), the title compound was prepared. MS (ESI): 530 (M+H⁺).

### Example 41

### Preparation of Benzofuran-2-carboxylic acid {(S)-3-methyl-1-[1-(1H-imidazole-2-sulfonyl)-3-oxo-azepan-4-ylcarbamoyl]-butyl}amide

Following the procedure of Example 6(a)-6(b), except substituting imidazole-2-sulfonyl chloride forbenzenesulfonyl chloride in step (a) and benzofuran-2-carboxylic acid for 5-(2-morpholin-4-yl-ethoxy)-benzofuran-2-carboxylic acid in step (b), the title compound was prepared. MS (ESI): 516 (M+H⁺).

### Example 42

### Preparation of Benzofuran-2-carboxylic acid {(S)-3-methyl-1-[1-(1-methyl-1H-imidazole-4-sulfonyl)-3-oxo-azepan-4-ylcarbamoyl]-butyl}amide

Following the procedure of Example 6(a)-6(b), except substituting 1-methylimidazole-4-sulfonyl chloride for benzenesulfonyl chloride in step (a) and benzofuran-2-carboxylic acid for 5-(2-morpholin4yl-ethoxy)-benzofuran-2-carboxylic acid in step (b), the title compound was prepared. MS (ESI): 530 (M+H⁺).

### Example 43

### Preparation of 5-(4-Oxy-morpholino-4-yl-ethoxy)-benzofuran-2-carboxylic acid {(S)-3-methyl-1-[3-oxo-1-(pyridine-2-sulfonyl)-azepan-4-ylcarbamoyl]-butyl}amide

To a solution of the compound of Example 18 (0.01 g) in dichloromethane (2 mL) was added m-CPBA (0.008 g). The reaction was stirred overnight. Workup and column chromatography (30% methanol:dichloromethane) provided the title compound. MS (ESI): 671 (M+H⁺).

### Example 44

### Preparation of 5-Hydroxy-benzofuran-2-carboxylic acid {(S)-3-methyl-1-[1-(1-methyl-1H-imidazole-4-sulfonyl)-3-oxo-azepan-4-ylcarbamoyl]-butyl}amide

Following the procedure of Example 6(a)-6(b), except substituting 1-methylimidazole-4-sulfonyl chloride for benzenesulfonyl chloride in step (a) and 5-hydroxybenzofuran-2-carboxylic acid for 5-(2-morpholin-4-yl-ethoxy)-benzofuran-2-carboxylic acid in step (b), the title compound was prepared. MS (ESI): 546 (M+H⁺).

### Example 45

### Preparation of Benzofuran-2-carboxylic acid {(S)-3-methyl-1-[3-oxo-1-(1-oxy-pyridine-2-sulfonyl)-azepan-4-ylcarbamoyl)]-3-methyl-butyl}-amide

Following the procedure of Example 6(a)-6(b), except substituting 1-oxypyridine-2-sulfonyl chloride for benzenesulfonyl chloride in step (a) and benzofuran-2-carboxylic acid for 5-(2-morpholin-4-yl-ethoxy)-benzofuran-2-carboxylic acid in step (b), the title compound was prepared. MS (ESI): 542 (M+H⁺).

### Example 46

### Preparation of Benzo[b]thiophene-2-carboxylic acid {(S)-3-methyl-1-[3-oxo-1-(1-oxy-pyridine-2-sulfonyl)-azepan-4-ylcarbamoyl]-butyl}amide

Following the procedure of Example 6(a)-6(b), except substituting 1-oxypyridine-2-sulfonyl chloride for benzenesulfonyl chloride in step (a) and benzo[b]thiophene-2-carboxylic acid for 5-(2-morpholin-4-yl-ethoxy)-benzofuran-2-carboxylic acid in step (b), the title compound was prepared. MS (ESI): 558 (M+H⁺).

### Example 47

### Preparation of 5-Bromo-furan-2-carboxylic acid {(S)-3-methyl-1-[3-oxo-1-1-oxy-pyridine-2-sulfonyl)-azepan-4-ylcarbamoyl]-butyl}amide

Following the procedure of Example 6(a)-6(b), except substituting 1-oxypyridine-2-sulfonyl chloride forfbenzenesulfonyl chloride in step (a) and 5-bromofuran-2-carboxylic acid for 5-(2-morpholin-4-yl-ethoxy)-benzofuran-2-carboxylic acid in step (b), the title compound was prepared. MS (ESI): 572 (M+H⁺).

### Example 48

### Preparation of 5,6-Dimethoxy-benzofuran-2-carboxylic acid {(S)-3-methyl-1-[3-oxo-1-(1-oxy-pyridine-2-sulfonyl)-azepan-4-ylcarbamoyl]-butyl}amide

Following the procedure of Example 6(a)-6(b), except substituting 1-oxypyridine-2-sulfonyl chloride for benzenesulfonyl chloride in step (a) and 5,6-dimethoxybenzofuran-2-carboxylic acid for 5-(2-morpholin-4-yl-ethoxy)-benzofuran-2-carboxylic acid in step (b), the title compound was prepared. MS (ESI): 602 (M+H⁺).

### Example 49

### Preparation of Benzofuran-2carboxylic acid {(S)-3-methyl-1-[3-oxo-1-(1-oxy-pyridine-2-sulfonyl)-azepan-4-ylcarbamoyl)]-3-methyl-butyl}-amide

The title compound was isolated as the first eluting compound from the HPLC purification in Example 45. MS (ESI): 542 (M+H⁺).

### Example 50

Preparation of 5-Bromo-furan-2-carboxylic acid {(S)-3-methyl-1-[3-oxo-1-(1-oxy-pyridine-2-sulfonyl)-azepan-4-ylcarbamoyl]-butyl}amide

The title compound was isolated as the first eluting compound from the HPLC purification in Example 47. MS (ESI): 572 (M+H⁺).

### Example 51

### Preparation of Benzofblthiophene-2-carboxylic acid {(S)-3-methyl-1-[3-oxo-1-(1-oxy-pyridine-2-sulfonyl)-azepan-4-ylcarbamoyl]-butyl}amide

The title compound was isolated as the first eluting compound from the HPLC purification in Example 46. MS (ESI): 558 (M+H⁺).

### Example 52

### Preparation of 5,6-Dimethoxy-benzofuran-2-carboxylic acid {(S)-3-methyl-1-[3-oxo-1-(1-oxy-pyridine-2-sulfonyl)-azepan-4-ylcarbamoyl]-butyl}amide

The title compound was isolated as the first eluting compound from the HPLC purification in Example 48. MS (ESI): 602 (M+H⁺).

### Example 53

### Preparation of 5-Methoxy-benzofuran-2-carboxylic acid {(S)-3-methyl-1[3-oxo-1-(1-oxy-pyridine-2-sulfonyl)-azepan-4-ylcarbamoyl]-butyl}amide

Following the procedure of Example 6(a)-6(b), except substituting 1-oxypyridine-2-sulfonyl chloride for benzenesulfonyl chloride in step (a) and 5-methoxybenzofuran-2-carboxylic acid for 5-(2-morpholin-4-yl-ethoxy)-benzofuran-2carboxylic acid in step (b), the title compound was prepared. MS (ESI): 573 (M+H⁺).

### Example 54

### Preparation of 1H-Indole-5-carboxylic acid {(S)-3-methyl-1-[3-oxo-1-(1-oxy-pyridine-2-sulfonyl)-azepan-4-ylcarbamoyl]-butyl}amide

Following the procedure of Example 6(a)-6(b), except substituting 1-oxypyridine-2-sulfonyl chloride for benzenesulfonyl chloride in step (a) and indole-5-carboxylic acid for 5-(2-morpholin-4-yl-ethoxy)-benzofuran-2carboxylic acid in step (b), the title compound was prepared. MS (ESI): 542 (M+H⁺).

### Example 55

### Preparation of Benzo[1,3]dioxole-5-carboxylic acid {(S)-3-methyl-1-[3-oxo-1-(1-oxy-pyridine-2-sulfonyl)-azepan-4-ylcarbamoyl]-butyl}amide

Following the procedure of Example 6(a)-6(b), except substituting 1-oxypyridine-2-sulfonyl chloride for benzenesulfonyl chloride in step (a) and benzo[1,3]dioxole-5-carboxylic acid for 5-(2-morpholin-4-yl-ethoxy)-benzofuran-2-carboxylic acid in step (b), the title compound was prepared. MS (ESI): 547 (M+H⁺).

### Example 56

### Preparation of 5-(2-Morpholin-4-yl-ethoxy)-benzofuran-2-carboxylic acid {(S)-3-methyl-1-[3-oxo-(1-oxy-pyridine2-sulfonyl)-azepan-4-ylcarbamoyl]-buty}-amide

Following the procedure of Example 6(a)-6(b), except substituting 1-oxypyridine-2-sulfonyl chloride for benzenesulfonyl chloride in step (a), the title compound was prepared. MS (ESI): 672 (M+H⁺).

### Example 57

### Preparation of 3-Methyl-benzofuran-2-carboxylic acid {(S)-3-methyl-1-[3-oxo-1-(1-oxy-pyridine-2-sulfonyl)-azepan-4-ylcarbamoyl]-butyl}amide

Following the procedure of Example 6(a)-6(b), except substituting 1-oxypyridine-2-sulfonyl chloride for benzenesulfonyl chloride in step (a) and 3-methylbenzofuran-2-carboxylic acid for 5-(2-morpholin-4-yl-ethoxy)-benzofuran-2-carboxylic acid in step (b), the title compound was prepared. MS (ESI): 557 (M+H⁺).

### Example 58

### Preparation of 1H-Indole-6-carboxylic acid {(S)-3-methyl-1-[3-oxo-1-(pyridine-2-sulfonyl)-azepan-4-ylcarbamoyl]-butyl}amide

Following the procedure of Example 6(a)-6(b), except substituting pyridine-2-sulfonyl chloride for benzenesulfonyl chloride in step (a) and indole-6-carboxylic acid for 5-(2-morpholin-4-yl-ethoxy)-benzofuran-2-carboxylic acid in step (b), the title compound was prepared. MS (ESI): 526 (M+H⁺).

### Example 59

### Preparation of Benzo[1,3]dioxole-5-carboxylic acid {(S)-3-methyl-1-[3-oxo-1-(pyridine-2-sulfonyl)-azepan-4-ylcarbamoyl]-butyl}amide

Following the procedure of Example 6(a)-6(b), except substituting pyridine-2-sulfonyl chloride for benzenesulfonyl chloride in step (a) and indole-5-carboxylic acid for benzo[1,3]dioxole-5-carboxylic acid in step (b), the title compound was prepared. MS (ESI): 531 (M+H⁺).

### Example 60

### Preparation of 3,4-Dihydro-2H-benzo[b][1,4]dioxepine-7-carboxylic acid {(S)-3-methyl-1-[3-oxo-1-(1-oxy-pyridine-2-sulfonyl)-azepan-4-ylcarbamoyl]butyl}amide

Following the procedure of Example 6(a)-6(b), except substituting 1-oxypyridine-2-sulfonyl chloride for benzenesulfonyl chloride in step (a) and 3,4-dihydro-2H-benzo[b][1,4]dioxepine-7-carboxylic acid for 5-(2-morpholin-4-yl-ethoxy)-benzofuran-2-carboxylic acid in step (b), the title compound was prepared. MS (ESI): 575 (M+H⁺).

### Example 61

### Preparation of 4,5-Dibromo-thiophene-2-carboxylic acid {(S)-3-methyl-1-[3-oxo-1-(1-oxy-pyridine-2-sulfonyl)-azepan-4-ylcarbamoyl]-butyl}amide

Following the procedure of Example 6(a)-6(b), except substituting 1-oxypyridine-2-sulfonyl chloride for benzenesulfonyl chloride in step (a) and 4,5-dibromothiophene-2-carboxylic acid for 5-(2-morpholin4-yl-ethoxy)-benzofuran-2-carboxylic acid in step (b), the title compound was prepared. MS (ESI): 665 (M+H⁺).

### Example 62

### Preparation of Thieno[3,2-b]thiophene-2-carboxylic acid {(S)-3-methyl-1-[3-oxo-1-(1-oxy-pyridine-2-sulfonyl)-azepan-4-ylcarbamoyl]-butyl}amide

Following the procedure of Example 6(a)-6(b), except substituting 1-oxypyridine-2-sulfonyl chloride for benzenesulfonyl chloride in step (a) and thieno[3,2-b]thiophene-2-carboxylic acid for 5-(2-morpholin-4-yl-ethoxy)-benzofuran-2-carboxylic acid in step (b), the title compound was prepared. MS (ESI): 565 (M+H⁺).

### Example 63

### Preparation of 5-Methoxy-benzofuran-2-carboxylic acid {(S)-3-methyl-1-[3-oxo-1-(1-oxy-pyridine-2-sulfonyl)-azepan-4-ylcarbamoyl]-butyl}amide

The title compound was isolated as the first eluting compound from the HPLC purification in Example 53. MS (ESI): 573 (M+H⁺).

### Example 64

### Preparation of 1H-Indole-5-carboxylic acid {(S)-3-methyl-1-[3-oxo-1-(1-oxy-pyridine-2-sulfonyl)-azepan-4-ylcarbamoyl]-butyl}amide

The title compound was isolated as the first eluting compound from the HPLC purification in Example 54. MS (ESI): 542 (M+H⁺).

### Example 65

### Preparation of 5-(4-Chloro-phenyl)-furan-2-carboxylic acid {(S)-3-methyl-1-[3-oxo-1-(pyridine-2-sulfonyl)-azepan-4-ylcarbamoyl]-butyl}amide

Following the procedure of Example 6(a)-6(b), except substituting pyridine-2-sulfonyl chloride for benzenesulfonyl chloride in step (a) and 5-(4-chloro-phenyl)-furan-2-carboxylic acid for benzo[1,3]dioxole-5-carboxylic acid in step (b), the title compound was prepared. MS (ESI): 587 (M+H⁺).

### Example 66

### Preparation of 5-(3-Trifluoromethyl-phenyl)-furan-2-carboxylic acid {(S)-3-methyl-1-[3-oxo-1-(pyridine-2-sulfonyl)-azepan-4-ylcarbamoyl]-butyl}amide

Following the procedure of Example 6(a)-6(b), except substituting pyridine-2-sulfonyl chloride for benzenesulfonyl chloride in step (a) and 5-(3-trifluoromethyl-phenyl)-furan-2-carboxylic acid for benzo[1,3]dioxole-5-carboxylic acid in step (b), the title compound was prepared. MS (ESI): 621 (M+H⁺).

### Example 67

### Preparation of Benzo[1,3]dioxole-5-carboxylic acid {(S)-1-[1-(4-fluoro-benzenesulfonyl)-3-oxo-azepan-4-ylcarbamoyl]-3methyl-butyl}-amide

Following the procedure of Example 6(a)-6(b), except substituting 4-fluorobenzenesulfonyl chloride for benzenesulfonyl chloride in step (a), the title compound was prepared. MS (ESI): 548 (M+H⁺).

### Example 68

### Preparation of 5-Bromo-furan-2-carboxylic acid {(S)-3-methyl-1-[3-oxo-1-(pyridine-2-sulfonyl)-azepan-4-ylcarbamoyl]-butyl}amide

The title compound was isolated as the first eluting compound from the HPLC purification in Example 34. MS (ESI): 555 (M+H⁺).

### Example 69

### Preparation of 5-Methoxy-benzofuran-2-carboxylic acid {(S)-3-methyl-1-[3-oxo-1-(pyridine-2-sulfonyl)-azepan-4-ylcarbamoyl]-butyl}amide

The title compound was isolated as the first eluting compound from the HPLC purification in Example 33. MS (ESI): 557 (M+H⁺).

### Example 70

### Preparation of 3-Methyl-benzofuran-2-carboxylic acid {(S)-3-methyl-1-[3-oxo-1-(pyridine-2-sulfonyl)-azepan-4-ylcarbamoyl]-butyl}amide

Following the procedure of Example 6(a)-6(b), except substituting pyridine-2-sulfonyl chloride for benzenesulfonyl chloride in step (a) and 3-methylbenzofuran-2-carboxylic acid for benzo[1,3]dioxole-5-carboxylic acid in step (b), the title compound was prepared. MS (ESI): 541 (M+H⁺).

### Example 71

### Preparation of Thieno[3,2-b]thiophene-2-carboxylic acid {(S)-3-methyl-1-[3-oxo-1-(pyridine-2-sulfonyl)-azepan-4-ylcarbamoyl]-butyl}amide

Following the procedure of Example 6(a)-6(b), except substituting pyridine-2-sulfonyl chloride for benzenesulfonyl chloride in step (a) and thieno[3,2-b]thiophene-2-carboxylic acid for benzo[1,3]dioxole-5-carboxylic acid in step (b), the title compound was prepared. MS (ESI): 549 (M+H⁺).

### Example 72

### Preparation of 2-Phenyl-5-trifluoromethyl-oxazole-4-carboxylic acid {(S)-3-methyl-1-[3-oxo-1-(pyridine-2-sulfonyl)-azepan-4-ylcarbamoyl]-butyl}amide

Following the procedure of Example 6(a)-6(b), except substituting pyridine-2-sulfonyl chloride for benzenesulfonyl chloride in step (a) and 2-Phenyl-5-trifluoromethyl-oxazole-4-carboxylic acid for benzo[1,3]dioxole-5-carboxylic acid in step (b), the title compound was prepared. MS (ESI): 622 (M+H⁺).

### Example 73

### Preparation of Benzo[1,3]dioxole-5-carboxylic acid {(S)-1-[1-(4-methoxy-benzenesulfonyl)-3-oxo-azepan-4-ylcarbamoyl]-3-methyl-butyl}-amide

Following the procedure of Example 6(a)-6(b), except substituting 4-methoxybenzenesulfonyl chloride for benzenesulfonyl chloride in step (a), the title compound was prepared. MS (ESI): 560 (M+H⁺).

### Example 74

Preparation of Benzofuran-2-carboxylic acid {(S)-1-[1-(4-methoxy-benzenesulfonyl)-3-oxo-azepan-4-ylcarbamoyl]-3-methyl-butyl}-amide

Following the procedure of Example 6(a)-6(b), except substituting 4-methoxybenzenesulfonyl chloride for benzenesulfonyl chloride in step (a) and benzofuran-2-carboxylic acid for benzo[1,3]dioxole-5-carboxylic acid in step (b), the title compound was prepared. MS (ESI): 556 (M+H⁺).

### Example 75

### Preparation of Furan-2-carboxylic acid {(S)-3-methyl-1-[3-oxo-1-(1-oxy-pyridine-2-sulfonyl)-azepan-4-ylcarbamoyl]-butyl}amide

Following the procedure of Example 6(a)-6(b), except substituting 1-oxypyridine-2-sulfonyl chloride for benzenesulfonyl chloride in step (a) and furan-2-carboxylic acid for 5-(2-morpholin-4-yl-ethoxy)-benzofuran-2-carboxylic acid in step (b), the title compound was prepared. MS (ESI): 493 (M+H⁺).

### Example 76

### Preparation of Benzo[1,3]dioxole-5-carboxylic acid {(S)-3-methyl-1-[3-oxo-1-(1-oxy-pyridine-2-sulfonyl)-azepan-4-ylcarbamoyl]-buty{amide

The title compound was isolated as the first eluting compound from the HPLC purification in Example 55. MS (ESI): 547 (M+H⁺).

### Example 77

### Preparation of 4-Fluoro-{(S)-3-methyl-1-[3-oxo-1-(1-oxy-pyridine-2-sulfonyl)-azepan-4-carbamoyl]-butyl}-benzamide

Following the procedure of Example 6(a)-6(b), except substituting 1-oxypyridine-2-sulfonyl chloride for benzenesulfonyl chloride in step (a) and 4-fluorobenzoic acid for 5-(2-morpholin-4-yl-ethoxy)-benzofuran-2-carboxylic acid in step (b), the title compound was prepared. MS (ESI): 521 (M+H⁺).

### Example 78

### Preparation of 3,4-Dihydro-2H-benzo[b][1,4]dioxepine-7-carboxylic acid {(S)-3-methyl-1-[3-oxo-1-(1-oxy-pyridine-2-sulfonyl)-azepan-4-ylcarbamoyl]-butyl}amide

The title compound was isolated as the first eluting, compound from the HPLC purification in Example 60. MS (ESI): 575 (M+H⁺).

### Example 79

### Preparation of 5-Methyl-thiophene-2-carboxylic acid {(S)-3-methyl-1-[3-oxo-1-(1-oxy-1pyridine-2-sulfonyl)-azepan-4-ylcarbamoyl]-butyl}amide

Following the procedure of Example 6(a)-6(b), except substituting 1-oxypyridine-2-sulfonyl chloride for benzenesulfonyl chloride in step (a) and 5-methylthiophene-2-carboxylic acid for 5-(2-morpholin4-yl-ethoxy)-benzofuran-2-carboxylic acid in step (b), the title compound was prepared. MS (ESI): 523 (M+H⁺).

### Example 80

### Preparation of (S)-2-(3-Benzyl-ureido)-4-methyl-pentanoic acid [3-oxo-carboxylic -(pyridine-2-sulfonyl)azepan4-yl]-amide

a) (S)-2-amino-4-methyl-pentanoic acid [3-hydroxy-1-(pyridine-2-sulfonyl)-azepan-4-yl]-amide

Following the procedure of Example 6(a) except substituting 2-pyridinesulfonyl chloride for benzenesulfonyl chloride, the title compound was prepared. MS (ESI): 385 M+H⁺).

b) (S)-2-(3-Benzyl-ureido)-4-methyl-pentanoic acid [3-hydroxy-1-(pyridine-2-sulfonyl)-azepan-4-yl]-amide

To a solution of the compound of Example 80(a) (0.25 g) in dichloromethane was added triethylamine (0.17 mL) and benzyl isocyanate (0.088 g). The reaction was stirred until complete. Workup and column chromatography (5% methanol:dichloromethane) provided the title compound (0.12 g).

c) (S)-2-(3-Benzyl-ureido)-4-methyl-pentanoic acid [3-oxo-1-(pyridine-2-sulfonyl)-azepan-4-yl]-amide

Following the procedure of Example 1 (k) except substituting (S)-2-(3-Benzyl-ureido)4-methyl-pentanoic acid [3-hydroxy-1-(pyridine-2-sulfonyl)-azepan-4-yl]-amide for benzo[1,3]dioxole-5-carboxylic acid [(S)-i-(1-benzyl-3-hydroxy-azepan-4-ylcarbamoyl)-3-methyl-butyl]-amide, the title compound was prepared. MS (ESI): 516 (M+H⁺).

### Example 81

### Preparation of 5-Methoxy-benzofuran-2-carboxylic acid [(S)-1-(1-methanesulfonyl-3-oxo-azepan-4-ylcarbamoyl)-3-methyl-butyl]-amide

Following the procedure of Example 6(a)-6(b), except substituting methanesulfonyl chloride for benzenequlfonyl chloride in step (a) and 5-methoxybnzofuran-2-carboxylic acid for 5-(2-morpholin-4-yl-ethoxy)-benzofuran-2-carboxylic acid in step (b), the title compound was prepared. MS (ESI): 494 (M+H⁺).

### Example 82

### Preparation of Furan-2-carboxylic acid ({(S)-1-[1-(4-methoxy-benzenesulfonyl-3-oxo-azepan-4-ylcarbamoyl]-3-methyl-butylcarbamoyl}-methyl)-amide

Following the procedure of Example 6(a)-6(b), except substituting 4-methoxybenzenesulfonyl chloride for benzenesulfonyl chloride in step (a) and N-(2-furan-carbonyl)-glycine for 5-(2-morpholin-4-yl-ethoxy)-benzofuran-2-carboxylic acid in step (b), the title compound was prepared. MS (ESI): 563 (M+H⁺).

### Example 83

### Preparation of Quinoline-2-carboxylic acid {[(S)-1-[1-(4-fluoro-benzenesulfonyl)-3-oxo-azepan-4-ylcarbamoyl]-3-methyl-butyl}-amide

Following the procedure of Example 6(a)-6(b), except substituting 4-fluorobenzenesulfonyl chloride for benzenesulfonyl chloride in step (a) and quinoline-2-carboxylic acid for 5-(2-morpholin-4-yl-ethoxy)-benzofuran-2-carboxylic acid in step (b), the title compound was prepared. MS (ESI): 555 (M+H⁺).

### Example 84

### Preparation of 1-Methyl-1H-indole-2-carboxylic acid {[(S)-1-[1-(4-fluoro-benzenesulfonyl)-3-oxo-azepan-4-ylcarbamoyl]-3-methyl-butyl}-amide

Following the procedure of Example 6(a)-6(b), except substituting 4-fluorobenzenesulfonyl chloride for benzenesulfonyl chloride in step (a) and 1-methylindole-2-carboxylic acid for 5-(2-morpholin-4-yl-ethoxy)-benzofuran-2-carboxylic acid in step (b), the title compound was prepared. MS (ESI): 557 (M+H⁺).

### Example 85

### Preparation of 5-Methoxy-benzofuran-2-carboxylic acid {[(S)-1-[1-(4-fluoro-benzenesulfonyl)-3-oxo-azepan-4-ylcarbamoyl]-3-methyl-butyl}-amide

Following the procedure of Example 6(a)-6(b), except substituting 4-fluorobenzenesulfonyl chloride for benzenesulfonyl chloride in step (a) and 5-methoxybenzofuran-2-carboxylic acid for 5-(2-morpholin-4-yl-ethoxy)-benzofuran-2-carboxylic acid in step (b), the title compound was prepared. MS (ESI): 574 (M+H⁺).

### Example 86

### Preparation of Quinoxaline-2-carboxylic acid {[(S)-1-[l -(4-fluoro-benzenesulfonyl)-3-oxo-azepan-4-ylcarbamoyl]-3-methyl-butyl}-amide

Following the procedure of Example 6(a)-6(b), except substituting 4-fluorobenzenesulfonyl chloride for benzenesulfonyl chloride in step (a) and 5-quinoixaline-2-carboxylic acid for 5-(2-morpholin4-yl-ethoxy)-benzofuran-2-carboxylic acid in step (b), the title compound was prepared. MS (ESI): 556 (M+H⁺).

### Example 87

### Preparation of Benzo[b]thiophene-2-carboxylic acid {(S)-3-methyl-1-[3-oxo-1-(pyridine-2-sulfonyl)-azepan-4-ylcarbamoyl]-butyl}amide

Following the procedure of Example 6(a)-6(b), except substituting pyridine-2-sulfonyl chloride for benzenesulfonyl chloride in step (a) and benzo[b]thiophene-2-carboxylic acid for benzo[1,3]dioxole-5-carboxylic acid in step (b), the title compound was prepared. MS (ESI): 543 (M+H⁺).

### Example 88

### Preparation of Benzofuran-2-carboxylic acid-{(S)-1-[1-(3-chloro-benzenesulfonyl)-3-oxo-azepan-4ylcarbamoyl]-3-methyl-butyl}-amide

a) {(S)-1-[1-(3-chloro-benzenesulfonyl)-3-hydroxy-azepan-4-ylcarbamoyl]-3-methyl-butyl}-carbamic acid tert-butyl ester

To a solution of the compound of Example 1(g) (2.50 g, 7.29 mmol) in dichloromethane (100 ml) was added P-NMM (4.0 g) and 3-chlorobenzenesulfonyl chloride (1.85 g, 8.75 mmol). After shaking at room temperature overnight, the solution was filtered. The filtrate was concentrated to yield the title compound as white solid (3.13 g, 83.3%). MS (ESI): 539.8 (M+Na)⁺.

b) (S)-2-amino-4-methyl-pentanoic acid [1-(3-chloro-benzenesulfonyl)-3-hydroxy-azepan-4-yl]-amide

To a stirring solution of the compound of Example 88(a) (1.0 g, 1.93 mmol) in methnol (10 ml) was added HCl (4M in dioxane) (10 ml). After stirring at room temperature for 3 hr the solution was concentrated to provide a white solid. To a solution of the white solid (0.68 g, 1.50 mmol, 78%) in methnol (37 ml) was added P-CO₃ (2.85 g, 2.63 mmol/g). After shaking for 2 hr, the solution was filtered and concentrated to yield the title compound as white solid (0.59 g, 1.42 mmol, 95%). MS (ESI): 417.9 (M+H)⁺.

c) Benzofuran-2-carboxylic acid-{(S)-1-[1-(3-chloro-benzenesulfonyl)-3-hydroxy-azepan-4-ylcarbamoyl]-3-methyl-butyl}-amide

To a solution of the compound of Example 88(b) (0.14 g, 0.33 mmol) in dichloromethane (20 mL) was added benzofuran-2-carboxylic acid (0.81 g, 0.50 mmol), 1-hydroxybenzotriazole (0.77 g, 0.57 mmol), and P-EDC (0.67 g, 1 mmol/g) in dichloromethane (10 mL). After shaking at room temperature overnight, the solution was treated with trisamnine resin (0.45 g, 3.75 mmol/g). After shaking for another 2 hr, the solution was filtered and concentrated to yield the title compound as a white solid (122 ml, 65%). MS (ESI): 562.2 (M+H)⁺.

d) Benzofuran-2-carboxylic acid-{(S)-1-[1-(3-chloro-benzenesulfonyl)-3-oxo-azepan-4-ylcarbamoyl]-3-methyl-butyl}-amide

To a stirring solution of the compound of Example 88(c) (122 mg, 0.22 mmol) in dichloromethane (4 mL) was added Dess-Martin reagent (185 mg, 0.44 mmol). After stirring at room temperature for 2 h, solutions of sodium thiosulfate (2 mL of 10% in water) and saturated aqueous sodium bicarbonate (2 mL) were added simultaneously to the solution. The aqueous layer was extracted with dichloromethane (2×). The organic phases were combined, washed with saturated brine, dried (MgSO₄), filtered and concentrated. The residue was purified by HPLC (Whelk-O1; ethanol/hexanes) to yield the title compound as a white solid (62.7 mg, 52%). MS (ESI): 560.2 (M+H)⁺.

### Example 89

### Preparation of 5-Methoxy-benzofuran-2-carboxylic acid-{(S)-1-[1-(3-chloro-benzenesulfonyl)-3-oxo-azepan-4-ylcarbamoyl]-3-methyl-butyl}-amide

Following the procedure of Example 88(a)-88(d), except substituting 5-methoxybenzofuran-2-carboxylic acid for benzofuran-2-carboxylic acid in step (c), the title compound was prepared. MS (ESI): 590 (M+H⁺).

### Example 90

### Preparation of 3-Methyl-benzofuran-2-carboxylic acid-{(S)-1-[1-(3-chloro-benzenesulfonyl)-3-oxo-azepan-4-ylcarbamoyl]-3-methyl-butyl}-amide

Following the procedure of Example 88(a)-88(d), except substituting 3-methylbenzofuran-2-carboxylic acid for benzofuran-2-carboxylic acid in step (c), the title compound was prepared. MS (ESI): 574 (M+H⁺).

### Example 91

### Preparation of Benzofuran-2-carboxylic acid-{(S)-1-[1-(2-fluoro-benzenesulfonyl )-3-oxo-azepan-4-ylcarbamoyl]-3-methyl-butyl 1-amide

Following the procedure of Example 88(a)-88(d), except substituting 2-fluorobenzenesulfonyl chloride for 3-chlorobenzenesulfonyl chloride in step (a), the title compound was prepared. MS (ESI): 544 (M+H⁺).

### Example 92

### Preparation of 5-Methoxy-benzofuran-2-carboxylic acid-{(S)-1-[1-(2-fluoro-benzenesulfonyl)-3-oxo-azepan-4-ylcarbamoyl]-3-methyl-butyl}-amide

Following the procedure of Example 88(a)-88(d), except substituting 2-fluorobenzenesulfonyl chloride for 3-chlorobenzenesulfonyl chloride in step (a) and 5-methoxybenzofuran-2-carboxylic acid for benzofuran-2-carboxylic acid in step (c), the title compound was prepared. MS (ESI): 574 (M+H⁺).

### Example 93

### Preparation of 7-Methoxy-benzofuran-2-carboxylic acid-{(S)-1-[1-(2-fluoro-benzenesulfonyl)-3-oxo-azepan-4-ylcarbamoyl]-3-methyl-butyl}-amide

Following the procedure of Example 88(a)-88(d), except substituting 2-fluorobenzenesulfonyl chloride for 3-chlorobenzenesulfonyl chloride in step (a) and 7-methoxybenzofuran-2-carboxylic acid for benzofuran-2-carboxylic acid in step (c), the title compound was prepared. MS (ESI): 574 (M+H⁺).

### Example 94

### Preparation of 3-methylbenzofuran-2-carboxylic acid-{(S)-1-[1-(2-fluoro-benzenesulfonyl)-3-oxo-azepan-4-ylcarbamoyl]-3-methyl-butyl}-amide

Following the procedure of Example 88(a)-88(d), except substituting 2-fluorobenzenesulfonyl chloride for 3-chlorobenzenesulfonyl chloride in step (a) and 3-methylbenzofuran-2-carboxylic acid for benzofuran-2-carboxylic acid in step (c), the title compound was prepared. MS (ESI): 558 (M+H⁺).

### Example 95

### Preparation of Benzo[b]thiophene-2-carboxylic acid-{(S)-1-[1-(2-fluoro-benzenesulfonyl)-3-oxo-azepan-4-ylcarbamoyl]-3-methyl-butyl}-amide

Following the procedure of Example 88(a)-88(d), except substituting 2-fluorobenzenesulfonyl chloride for 3-chlorobenzenesulfonyl chloride in step (a) and benzo[b]thiophene-2-carboxylic acid for benzofuran-2-carboxylic acid in step (c), the title compound was prepared. MS (ES[): 560 (M+H⁺).

### Example 96

### Preparation of Quinoxaline-2-carboxylic acid-{(S)-1-[1-(2-fluoro-benzenesulonyl)-3-oxo-azepan-4-ylcarbamoyl]-3-methyl-butyl}-amide

Following the procedure of Example 88(a)-88(d), except substituting 2-fluorobenzenesulfonyl chloride for 3-chlorobenzenesulfonyl chloride in step (a) and quinoxaline-2-carboxylic acid for benzofuran-2-carboxylic acid in step (c), the title compound was prepared. MS (ESI): 556 (M+H⁺).

### Example 97

### Preparation of 3-Methyl-benzofuran-2-carboxylic acid {(S)-3-methyl-1-[3-oxo-1-(pyridine-2-sulfonyl)-azepan-4-ylcarbamoyl]-butyl}amide

The title compound was isolated as the first eluting compound from the HPLC purification in Example 70. MS (ESI): 541 (M+H⁺).

### Example 98

### Preparation of Thieno[3.2-b]thiophene-2-carboxylic acid {(S)-3-methyl-1-[3-oxo-1-(pyridine-2-sulfonyl)-azepan-4-ylcarbamoyl]-butyl}amide

The title compound was isolated as the first eluting compound from the HPLC purification in Example 71. MS (ESI): 549 (M+H⁺).

### Example 99

### Preparation of 3-Methyl-benzofuran-2-carboxylic acid {(S)-3-methyl-1-[3-oxo-1-(1-oxy-pyridine-2-sulfonyl)-azepan-4-ylcarbamoyl]-butyl}amide

The title compound was isolated as the first eluting compound from the HPLC purification in Example 57. MS (ESI): 557 (M+H⁺).

### Example 100

### Preparation of Benzofuran-2-carboxylic acid {(S)-3-methyl-1-[(2,2′,4-tridueterio)-3-oxo-1-(pyridine-2-sulfonyl)-azepan-4-ylcarbamoyl]-butyl}amide

To a solution of the compound of Example 16 (0.03 g) in D₂O:CD₃OD (0.4:4 mL) was added triethylamine (0.04 mL). The reaction was heated to reflux for 2 hours whereupon it was concentrated and dried under vacuum. The residue was the redissolved in the same mixture and heated to reflux overnight. The reaction was concentrated and the residue purified by column chromatography (5% methanol:dichloromethane) to provide the title compound (0.02 g). Separation of the diastereomers by HPLC (Whelk-O1; ethanol/hexanes) provided the title compound. MS (ESI): 530 (M+H⁺).

### Example 101

### Preparation of Quinoxaline-2-carboxylic acid {(S)-3-methyl-1-[3-oxo-1-(1-oxy-pyridine-2-sulfonyl)-azepan-4-ylcarbamoyl]-butyl}amide

Following the procedure of Example 6(a)-6(b), except substituting 1-oxypyridine-2-sulfonyl chloride for benzenesulfonyl chloride in step (a) and quinoxaline-2-carboxylic acid for 5-(2-morpholin-4-yl-ethoxy)-benzofuran-2-carboxylic acid in step (b), the title compound was prepared. MS (ESI): 555 (M+H⁺).

### Example 102

### Preparation of Benzofuran-2-carboxylic acid {(S)-2-cyclohexyl-1-[3-oxo-1-(pyridine-2-sulfonyl)-azepan-4-ylcarbamoyl]-ethyl}-amide

a) 4-tert-Butoxycarbonylamino-3-hydroxy-azepane-1-carboxylicacid benzyl ester

To a stirring solution of compound of Example 1(e) (1.04 g, 3.92 mmol) in THF was added di-tert-butyldicarbonate (0.864 g). After stirring at room temperature for 30 minutes, the reaction mixture was diluted with diethylether and extracted with saturated NaHCO₃ The organic layer was dried over anhydrous Na₂SO₄, filtered, concentrated, and purified by silica gel column to give the title compound as a yellow oil (0.963 g, 2.64 mmol, 67%). MS ESI): 365.0 (M+H)⁺.

b) (3-Hydroxy-azepan-4-yl)-carbamic acid tert-butyl ester

To a solution of compound of Example 102(a) (0.963 g, 2.64 mmol) in ethyl acetate (16 ml) was added 10% palladium on carbon (500 mg). After stirring the solution at room temperature for 48 hours, the mixture was filtered through celite. The filterate was concentrated to yield the title compound ( 0.529 g, 2.29 mmol, 87%). MS (ESI): 231.9 (M+H)⁺.

c) [3-Hydroxy-1-(pyridine-2-sulfonyl)-azepan-4-yl]-carbamic acid tert-butyl ester

To a solution of the compound of Example 102(b) (0.53, 2.29 mmol) in dichloromethane (20 ml) was added triethylamine (232 mg) and pyridine-2-sulfonyl chloride (410 mg, 2.32 mmol). After stirring at room temperature for 30 minutes, the mixture was washed with saturated NaHCO₃ The organic layer was dried, filtered, concentrated and purified on a silica gel column to give the title compound as a solid (0.58 g, 1.57 mmol, 68%). MS (ESI): 373.0 (M+H)⁺.

d) 4-Amino-1-(pryidine-2-sulfonyl)-azepan-3-ol

To a stirring solution of the compound of Example 102(c) (0.583 g, 1.57 mmol) in ethyl acetate (0.5 ml) was added HCl (4M in dioxane, 3.9 ml). After stirring the reaction mixture for 30 minutes at room temperature, the mixture was concentrated to yield a white solid. The solid was treated with NaOH and then extracted with ethylacetate. The organic layer was dried, filtered, and concentrated to yield a yellow solid (0.35 g, 1.28 mmol, 81%). MS (ESI): 272.9 (M+H)⁺.

e) (S)-1-[3-Hydroxy-1-(pryidine-2-sulfonyl)-azepan-4-ylcarbamoyl]-2-meth-butyl}-carbamic acid tert-butyl ester

To a solution of the compound of example 102(d) (19 mg, 0.070 mmol) in CH₁Cl₂ was added N-tert-butoxycarbonyl-L-cyclohexylalanine (28.5 mg, 0.10 mmol), 1-hydroxybenzotriazole (16.1 mg, 0.12 mmol), and P-EDC (140 mg, 0.14 mmol ) in dichloromethane. After shaking at room temperature overnight, the mixture was treated with PS-Trisamnine. After shaking for another 2 hours. the mixture was filtered and concentrated to yield the title compound as a solid. MS (ESI): 525.0 (M+H)⁺.

f) (S)-2-Amino-3-methyl-penatanoic acid [3-hydroxy-1-(pyridine-2-sulfonyl)-azepan-4-yl]-amide

To a stirring solution of the compound of example 102(e) (37 mg, 0.07 mmol) in dicloromethane (0.50 ml) was added HCl (4M in dioxane) (0.165 ml). After stirring at room temperature for 30 minutes, the mixture was concentrated, giving a white solid. The white solid was azeotroped with toluene then treated with MP-carbonate (0.35 mmol) in methanol. After four hours of shaking, the mixture was filtered and concentrated to give the title compound as a solid. MS (ESI): 425.0 (M+H)⁺.

g) Benzofuran-2-carboxylic acid {(S)-2-methyl-1-[3-hydroxy-1-(pyridine-2-sulfonyl)-azepan-4-ylcarbamoyl]-butyl}-amide

To a solution of the compound of example 102(f) (30 mg, 0.070 mmol) in ichloromethane was added benzofuran-2-carboxylic acid (17.0 mg, 0.106 mmol), 1-hydroxybenzotriazole (16.1 mg, 0.12 mmol), and P-EDC (140 mg, 0.14 mmol )in dichloromethane. After shaking at room temperature overnight, the mixture was treated with PS-Trisamine. After shaking for another 2 hours, the mixture was filtered and concentrated to yield the title compound as a solid. MS (ESI): 569.0 (M+H)⁺.

h) Benzofuran-2-carboxylic acid {(S)-2-methyl-1-[3-oxo-1-(pyridine-2-sulfonyl)-azepan-4-ylcarbamoyl]-butyl}-amide

To a stirring solution of the compound of example 102(g) (40 mg, 0.07 mmol) in dichloromethane (0.5 ml) was added Dess-Martin reagent (45 mg, 0.105 mmol). After stirring for 30 minutes, solutions of sodium thiosulfate (10% in water, 0.50 ml) and saturated aqueous sodium bicarbonate (0.50 ml) were added simultaneously to the. reaction. The mixture was then extracted with dichloromethane (2 times). The organic layer was dried, filtered, and concentrated. The residue was purified by HPLC (Whelk-O1; ethanol/hexanes) to yield title compound as a white solid. MS (ESI): 567.0 (M+H)⁺.

### Example 103

### Preparation of Benzofuran-2-carboxylic acid{(S)-1-[3-oxo-1-(pyridine-2-sulfonyl)-azepan-4-ylcarbamoyl]-pentyl}-amide

Following the procedure of Example 102(a)-102(h), except substituting N-Boc-norleucine for N-tert-butoxycarbonyl-L-cyclohexylalanine in step (e), the title compound was prepared. MS (ESI): 527 (M+H⁺).

### Example 104

### Preparation of Benzofuran-2-carboxylic acid {(S)-1-[3-oxo-1-(pyridine-2-sulfonyl)-azepan-4-ylcarbamoyl]-2-phenyl-ethyl}-amide

Following the procedure of Example 102(a)-102(h), except substituting N-tert-butoxycarbonyl-L-phenylalanine for N-Boc-cyclohexylalanine in step (e), the title compound was prepared. MS (ESI): 561 (M+H⁺).

### Example 105

### Preparation of 2-Phenyl-5-trifluoromethyl-oxazole-4-carboxylic acid {(S)-3-methyl-1-[3-oxo-1-(pyridine-2-sulfonyl)-azepan-4-ylcarbamoyl]-butyl}amide

The title compound was isolated as the first eluting compound from the HPLC purification in Example 72. MS (ESI): 622 (M+H⁺).

### Example 106

### Preparation of 5-Methyl-2-phenyl-oxazole-4-carboxylic acid {(S)-3-methyl-1-[3-oxo-1-(pyridine-2-sulfonyl)-azepan-4-ylcarbamoyl]-butyl}amide

Following the procedure of Example 6(a)-6(b), except substituting pyridine-2-sulfonyl chloride for benzenesulfonyl chloride in step (a) and 5-Methyl-2-phenyloxazole-4-carboxylic acid for benzo[1,3]dioxole-5-carboxylic acid in step (b), the title compound was prepared. MS (ESI): 568 (M+H⁺).

### Example 107

### Preparation of 3,4-Dimethoxy-N—{(S)-1-[1-(4-methoxy-benzenesulfonyl)-3-oxo-azepan-4-ylcarbamoyl]-3-methyl-butyl}-benzamide

Following the procedure of Example 6(a)-6(b), except substituting 4-methoxybenzenesulfonyl chloride for benzenesulfonyl chloride in step (a) and 3,4-dimethoxybenzoic acid for benzo[1,3]dioxole-5-carboxylic acid in step (b), the title compound was prepared. MS (ESI): 576 (M+H⁺).

### Example 108

### Preparation of Benzo[b]thiophene-2-carboxylic acid-{(S)-1-[1-(4-fluoro-benzenesulfonyl)-3-oxo-azepan-4-yl carbamoyl]-3-methyl-butyl}-amide

Following the procedure of Example 6(a)-6(b), except substituting 4-fluorobenzenesulfonyl chloride for benzenesulfonyl chloride in step (a) and benzo[b]thiophene-2-carboxylic acid for 5-(2-morpholin4-yl-ethoxy)-benzofuran-2-carboxylic acid in step (b), the title compound was prepared. MS (ESI): 560 (M+H⁺).

### Example 109

### Preparation of Benzofuran-2-carboxylic acid-{(S)-1-[1-(4-fluoro-benzenesulfonyl)-3-oxo-azepan-4-yl carbamoyl[-3-methyl-butyl}-amide

Following the procedure of Example 6(a)-6(b), except substituting 4-fluorobenzenesulfonyl chloride for benzenesulfonyl chloride in step (a) and benzofuran-2-carboxylic acid for 5-(2-morpholin-4-yl-ethoxy)-benzofuran-2-carboxylic acid in step (b), the title compound was prepared. MS (ESI): 544 (M+H⁺).

### Example 110

### Preparation of N—{(S)-1-[1-(4-Fluoro-benzenesulfonyl)-3-oxo-azepan-4-ylcarbamoyl}-3-methyl-butyl}-3,4-dimethoxy-benzamide

Following the procedure of Example 6(a)-6(b), except substituting 4-fluorobenzenesulfonyl chloride for benzenesulfonyl chloride in step (a) and 3,4-dimethoxybenzoic acid for 5-(2-morpholin-4-yl-ethoxy)-benzofuran-2-carboxylic acid in step (b), the title compound was prepared. MS (ESI): 546 (M+H⁺).

### Example 111

### Preparation of Benzo[b]thiophene-2-carboxylic acid-{(S)-1-(1-methanesulfonyl-3-oxo-azepan-4-yl carbamoyl)-3-methyl-butyl]-amide

Following the procedure of Example 6(a)-6(b), except substituting methanesulfonyl chloride for benzenesulfonyl chloride in step (a) and benzo[b]thiophene-2-carboxylic acid for 5-(2-morpholin-4-yl-ethoxy)-benzofuran-2-carboxylic acid in step (b), the title compound was prepared. MS (ESI): 480 (M+H⁺).

### Example 112

### Preparation of Benzofuran-2-carboxylic acid-{(S)-1-(1-methanesulfonyl-3-oxo-azepan-4-yl carbamoyl)-3-methyl-butyl]-amide

Following the procedure of Example 6(a)-6(b), except substituting methanesulfonyl chloride for benzenesulfonyl chloride in step (a) and benzofuran-2-carboxylic acid for 5-(2-morpholin-4-yl-ethoxy)-benzofuran-2-carboxylic acid in step (b), the title compound was prepared. MS (ESI): 464 (M+H⁺).

### Example 113

### Preparation of N—[(S)-1-(1-Methanesulfonyl)-3-oxo-azepan-4-ylcarbamoyl}-3-methyl-butyl}-3,4-dimethoxy-benzamide

Following the procedure of Example 6(a)-6(b), except substituting methanesulfonyl chloride for benzenesulfonyl chloride in step (a) and 3,4-dimethoxybenzoic acid for 5-(2-morpholin-4-yl-ethoxy)-benzofuran-2-carboxylic acid in step (b), the title compound was prepared. MS (ESI): 484 (M+H⁺).

### Example 114

### Preparation of N—{(S)-1-[1-(2-Cyano-benzenesulfonyl)-3-oxo-azepan-4-ylcarbamoyl}-3-methyl-butyl 1-4-methanesulfonyl-benzamide

Following the procedure of Example 6(a)-6(b), except substituting 2-cyanobenzenesulfonyl chloride for benzenesulfonyl chloride in step (a) and 4-methanesulfonylbenzoic acid for 5-(2-morpholin-4-yl-ethoxy)-benzofuran-2-carboxylic acid in step (b), the title compound was prepared. MS (ESI): 589 (M+H⁺).

### Example 115

### Preparation of Benzofuran-2-carboxylic acid {(S)-1-1-(2-cyano-benzenesulfonyl)-3-oxo-azepan-4-yl carbamoyl[-3-methyl-butyl}-amide

Following the procedure of Example 6(a)-6(b), except substituting 2-cyanobenzenesulfonyl chloride for benzenesulfonyl chloride in step (a) and benzofuran-2-carboxylic acid for 5-(2-morpholin4-yl-ethoxy)-benzofuran-2-carboxylic acid in step (b), the title compound was prepared. MS (ESI): 551 (M+H⁺).

### Example 116

### Preparation of 5-(2-Morpholin-4-yl-ethoxy)-benzofuran-2-carboxylic acid {(S)-3-methyl-1-[3-oxo-(1-oxy-pyridine2-sulfonyl)-azepan-4-ylcarbamoyl]-buty}-amide

The title compound was isolated as the first eluting compound from the HPLC purification in Example 56. MS (ESI): 672 (M+H⁺).

### Example 117

### Preparation of 5-Methyl-2-phenyl-oxazole-4-carboxylic acid {(S)-3-methyl-1-[3-oxo-1-(1-oxy-pyridine-2-sulfonyl)-azepan-4-ylcarbamoyl]-butyl}amide

Following the procedure of Example 6(a)-6(b), except substituting 1-oxypyridine-2-sulfonyl chloride for benzenesulfonyl chloride in step (a) and 5-Methyl-2-phenyloxazole-4-carboxylic acid for benzo[1,3]dioxole-5-carboxylic acid in step (b), the title compound was prepared. MS (ESI): 584 (M+H⁺).

### Example 118

### Preparation of 6-Methyl-N—(S)-3-methyl-1-3-oxo-1-(1-oxy-pyridine-2-sulfonyl)-azepan-4-ylcarbamoyl]-butyl}-nicotinamide

Following the procedure of Example 6(a)-6(b), except substituting 1-oxypyridine-2-sulfonyl chloride for benzenesulfonyl chloride in step (a) and 6-methylnicotinic acid for benzo[1,3]dioxole-5-carboxylic acid in step (b), the title compound was prepared. MS (ESI): 518 (M+H⁺).

### Example 119

Preparation of 5-(3-Trifluoromethyl-phenyl)-furan-2-carboxylic acid {(S)-3-methyl-1-[3-oxo-1-(1-oxy-pyridine-2-sulfonyl)-azepan-4-ylcarbamoyl]-butyl}amide

Following the procedure of Example 6(a)-6(b), except substituting 1-oxypyridine-2-sulfonyl chloride forbenzenesulfonyl chloride in step (a) and 5-(3-trifluoromethyl-phenyl)-furan-2-carboxylic acid for benzo[1,3]dioxole-5-carboxylic acid in step (b), the title compound was prepared. MS (ESI): 637 (M+H⁺).

### Example 120

### Preparation of N—{(S)1-[(1-(2-cyano-benzenesulfonyl)-3-oxo-azepan-4-ylcarbamoyl]-3-methyl-butyl}-3,4-dimethoxy-benzamide

Following the procedure of Example 6(a)-6(b), except substituting 2-cyanobenzenesulfonyl chloride for benzenesulfonyl chloride in step (a) and 3,4-dimethoxybenzoic acid for benzo[1,3]dioxole-5-carboxylic acid in step (b), the title compound was prepared. MS (ESI): 571 (M+H⁺).

### Example 121

### Preparation of 4-Methansulfonyl-N—{(S)-1-[4-fluoro-benzenesulfonyl)-3-oxo-azepan-4-carbamoyl]-3-methyl-butyl-benzamide

Following the procedure of Example 6(a)-6(b), except substituting 4-fluorobenzenesulfonyl chloride for benzenesulfonyl chloride in step (a) and 4-methanesulfonylbenzoic acid for benzo[1,3]dioxole-5-carboxylic acid in step (b), the title compound was prepared. MS (ESI): 582 (M+H⁺).

### Example 122

### Preparation of (S)-2-[5-(4-Methoxy-phenyl)-pentanoylamniol-4-methyl-pentanoic acid [3-oxo-1-(pyridine-2-sulfonyl)-azepan-4-yl[-amide

Following the procedure of Example 6(a)-6(b), except substituting pyridine-2-sulfonyl chloride for benzenesulfonyl chloride in step (a) and 5-(4-methoxyphenyl)pentanoic acid for benzo[1,3]dioxole-5-carboxylic acid in step (b), the title compound was prepared. MS (ESI): 573 (M+H⁺).

### Example 123

### Preparation of (S)-2-[2-(3-Benzyloxy-4-methoxy-phenyl)-acetylamnio-4-methylpentanoic acid [3-oxo-1-(pyridine-2-sulfonyl)-azepan-4-yl]-amide

Following the procedure of Example 6(a)-6(b), except substituting pyridine-2-sulfonyl chloride for benzenesulfonyl chloride in step (a) and 2-(3-Benzyloxy-4-methoxy-phenyl)acetic acid for benzo[1,3]dioxole-5-carboxylic acid in step (b), the title compound was prepared. MS (ESI): 637 (M+H⁺).

### Example 124

### Preparation of 5-Methoxybenzofuran-2-carboxylic acid {(S)-3-methyl-1-[3-oxo-1-(thiazole-2-sulfonyl)-azepan-4-ylcarbamoyl]-buty}amide

Following the procedure of Example 88(a)-88(d), except substituting thiazole-2-sulfonyl chloride for 3-chlorobenzenesulfonyl chloride in step (a) and 5-methoxybenzofuran-2-carboxylic acid for benzofuran-2-carboxylic acid in step (c), the title compound was prepared. MS (ESI): 563 (M+H⁺).

### Example 125

### Preparation of 7-Methoxybenzofuran-2-carboxylic acid {(S)-3-methyl-1-3-oxo-1-(thiazole-2-sulfonyl)-azepan-4-ylcarbamoyl]-butyl}amide

Following the procedure of Example 88(a)-88(d), except substituting thiazole-2-sulfonyl chloride for 3-chlorobenzenesulfonyl chloride in step (a) and 7-methoxybenzofuran-2-carboxylic acid for benzofuran-2-carboxylic acid in step (c), the title compound was prepared. MS (ESI): 563 (M+H⁺).

### Example 126

### Preparation of 3-Methylbenzofuran-2-carboxylic acid {(S)-3-methyl-1-[3-oxo-1-(thiazole-2-sulfonyl)-azepan-4-ylcarbamoyl]-butyl}amide

Following the procedure of Example 88(a)-88(d), except substituting thiazole-2-sulfonyl chloride for 3-chlorobenzenesulfonyl chloride in step (a) and 3-methylbenzofuran-2-carboxylic acid for benzofuran-2-carboxylic acid in step (c), the title compound was prepared. MS (ESI): 547 (M+H⁺).

### Example 127

### Preparation of Benzo[b]thiophene-2-carboxylic acid {(S)-3-methyl-1-[3-oxo-1-(thiazole-2-sulfonyl)-azepan-4-ylcarbamoyl]-butyl}amide

Following the procedure of Example 88(a)-88(d), except substituting thiazole-2-sulfonyl chloride for 3-chlorobenzenesulfonyl chloride in step (a) and benzo[b]thiophene-2-carboxylic acid for benzofuran-2-carboxylic acid in step (c), the title compound was prepared. MS (ESI): 549 (M+H⁺).

### Example 128

### Preparation of 1-Methyl-1H-indole-2-carboxylic acid{(S)-3-methyl-1-[3-oxo-1-(thiazole-2-sulfonyl)-azepan-4-ylcarbamoyl]-butyl}amide

Following the procedure of Example 88(a)-88(d), except substituting thiazole-2-sulfonyl chloride for 3-chlorobenzenesulfonyl chloride in step (a) and 1-methylindole-2-carboxylic acid for benzofuran-2-carboxylic acid in step (c), the title compound was prepared. MS (ESI): 563 (M+H⁺).

### Example 129

### Preparation of Quinoxaline-2-carboxylic acid {(S)-3-methyl-1-[3-oxo-1-(thiazole-2-sulfonyl)-azepan-4-ylcarbamoyl]-butyl}amide

Following the procedure of Example 88(a)-88(d), except substituting thiazole-2-sulfonyl chloride for 3-chlorobenzenesulfonyl chloride in step (a) and quinoxaline-2-carboxylic acid for benzofuran-2-carboxylic acid in step (c), the title compound was prepared. MS (ESI): 545 (M+H⁺).

### Example 130

### Preparation of Benzofuran-2-carboxylic acid {(S)-3-methyl-1-[3-oxo-1-(thiazole-2-sulfonyl)-azepan-4-ylcarbamoyl]-butyl}amide

Following the procedure of Example 88(a)-88(d), except substituting thiazole-2-sulfonyl chloride for 3-chlorobenzenesulfonyl chloride in step (a), the title compound was prepared. MS (ESI): 533 (M+H⁺).

### Example 131

### Preparation of Benzofuran-2-carboxylic acid {(S)-1-[1-(4-chloro-benzenesulfonyl)-3-oxo-azepan-4-yl carbamoyl]-3-methyl-butyl}-amide

Following the procedure of Example 88(a)-88(d), except substituting 4-chlorobenzenesulfonyl chloride for 3-chlorobenzenesulfonyl chloride in step (a), the title compound was prepared. MS (ESI): 561 (M+H⁺).

### Example 132

### Preparation of 5-Methoxy-benzofuran-2-carboxylic acid {(S)-1-[1-(4-chloro-benzenesulfonyl)-3-oxo-azepan-4-ylcarbamoyl]-3-methyl-butyl}-amide

Following the procedure of Example 88(a)-88(d), except substituting 4-chlorobenzenesulfonyl chloride for 3-chlorobenzenesulfonyl chloride in step (a) and 5-methoxybenzofuran-2-carboxylic acid for benzofuran-2-carboxylic acid in step (c), the title compound was prepared. MS (ESI): 590 (M+H⁺).

### Example 133

### Preparation of 7-Methoxy-benzofuran-2-carboxylic acid-{(S)-1-[1-(4-chloro-benzenesulfonyl)-3-oxo-azepan-4-ylcarbamoyl]-3-methyl-butyl}-amide

Following the procedure of Example 88(a)-88(d), except substituting 4-chlorobenzenesulfonyl chloride for 3-chlorobenzenesulfonyl chloride in step (a) and 7-methoxybenzofuran-2-carboxylic acid for benzofuran-2-carboxylic acid in step (c), the title compound was prepared. MS (ESI): 590 (M+H⁺).

### Example 134

### Preparation of 3-Methyl-benzofuran-2-carboxylic acid-{(S)-1-[1-(4-chloro-benzenesulfonyl)-3-oxo-azepan-4-ylcarbamoyl]-3-methyl-butyl}-amide

Following the procedure of Example 88(a)-88(d), except substituting 4-chlorobenzenesulfonyl chloride for 3-chlorobenzenesulfonyl chloride in step (a) and 3-methylbenzofuran-1-carboxylic acid for benzofuran-2-carboxylic acid in step (c), the title compound was prepared. MS (ESI): 574 (M+H⁺).

### Example 135

### Preparation of Benzo[b]thiophene-2-carboxylic acid-{(S)-1-[1-(4-chloro-benzenesulfonyl)-3-oxo-azepan-4-ylcarbamoyl]-3-methyl-butyl}-amide

Following the procedure of Example 88(a)-88(d), except substituting 4-chlorobenzenesulfonyl chloride for 3-chlorobenzenesulfonyl chloride in step (a) and benzo[b]thiophene-2-carboxylic acid for benzofuran-2-carboxylic acid in step (c), the title compound was prepared. MS (ESI): 576 (M+H⁺).

### Example 136

### Preparation of Benzofuran-2-carboxylic acid-{(S)-1-[1-(3-methoxy-benzenesulfonyl)-3-oxo-azepan-4-ylcarbamoyl]-3-methyl-butyl}-amide

Following the procedure of Example 88(a)-88(d), except substituting 3-methoxybenzenesulfonyl chloride for 3-chlorobenzenesulfonyl chloride in step (a), the title compound was prepared. MS (ESI): 536 (M+Na⁺).

### Example 137

### Preparation of 5-Methoxy-benzofuran-2-carboxylic acid-{(S)-1-[1-(3-methoxy-benzenesulfonyl)-3-oxo-azepan-4-ylcarbamoyl]-3-methyl-butyl}-amide

Following the procedure of Example 88(a)-88(d), except substituting 3-methoxy benzenesulfonyl chloride for 3-chlorobenzenesulfonyl chloride in step (a) and 5-methoxybenzofuran-2-carboxylic acid for benzofuran-2-carboxylic acid in step (c), the title compound was prepared. MS (ESI): 586 (M+H⁺).

### Example 138

### Preparation of 7-Methoxy-benzofuran-2-carboxylic acid-{(S)-1-[1-(3-methoxy-benzenesulfonyl)-3-oxo-azepan-4-ylcarbamoyl]-3-methyl-butyl}-amide

Following the procedure of Example 88(a)-88(d), except substituting 3-methoxy benzenesulfonyl chloride for 3-chlorobenzenesulfonyl chloride in step (a) and 7-methoxybenzofuran-2-carboxylic acid for benzofuran-2-carboxylic acid in step (c), the title compound was prepared. MS (ESI): 586 (M+H⁺).

### Example 139

### Preparation of 3-Methyl-benzofuran-2-carboxylic acid-{(S)-1-[1-(3-methoxy-benzenesulfonyl)-3-oxo-azepan-4-ylcarbamoyl]-3-methyl-butyl}-amide

Following the procedure of Example 88(a)-88(d), except substituting 3-methoxy benzenesulfonyl chloride for 3-chlorobenzenesulfonyl chloride in step (a) and 3-methylbenzofuran-2-carboxylic acid for benzofuran-2-carboxylic acid in step (c), the title compound was prepared. MS (ESI): 570 (M+H⁺).

### Example 140

### Preparation of Benzo[b]thiophene-2-carboxylic acid-{(S)-1-[1-(3-methoxy-benzenesulfonyl)-3-oxo-azepan-4-ylcarbamoyl]-3-methyl-butyl}-amide

Following the procedure of Example 88(a)-88(d), except substituting 3-methoxy benzenesulfonyl chloride for 3-chlorobenzenesulfonyl chloride in step (a) and benzo[b]thiophene-2-carboxylic acid for benzofuran-2-carboxylic acid in step (c), the title compound was prepared. MS (ESI): 572 (M+H⁺).

### Example 141

### Preparation of 1-Methyl-1H-indole-2-carboxylic acid-{(S)-1-[1-(3-methoxy-benzenesulfonyl)-3-oxo-azepan-4-ylcarbamoyl]-3-methyl-butyl}-amide

Following the procedure of Example 88(a)-88(d), except substituting 3-methoxy benzenesulfonyl chloride for 3-chlorobenzenesulfonyl chloride in step (a) and 1-methylindole-2-carboxylic acid for benzofuran-2-carboxylic acid in step (c), the title compound was prepared. MS (ESI): 569 (M+H⁺).

### Example 142

### Preparation of Benzofuran-2-carboxylic acid-{(S)-3-methyl-1-[3-oxo-1-(thiophene-2-sulfonyl)-azepan-4-ylcarbamoyl]-butyl}-amide

Following the procedure of Example 88(a)-88(d), except substituting thiophene-2-sulfonyl chloride for 3-chlorobenzenesulfonyl chloride in step (a), the title compound was prepared. MS (ESI): 532 (M+H⁺).

### Example 143

### Preparation of 5-Methoxy-benzofuran-2-carboxylic acid-{(S)-3-methyl-1-[3-oxo-1-(thiophene-2-sulfonyl)-azepan-4-ylcarbamoyl]-butyl}-amide

Following the procedure of Example 88(a)-88(d), except substituting thiophene-2-sulfonyl chloride for 3-chlorobenzenesulfonyl chloride in step (a) and 5-methoxybenzofuran-2-carboxylic acid for benzofuran-2-carboxylic acid in step (c), the title compound was prepared. MS (ESI): 562 (M+H⁺).

### Example 144

### Preparation of 7-Methoxy-benzofuran-2-carboxylic acid-{(S)-3-methyl-1-[3-oxo-1-(thiophene-2-sulfonyl)-azepan-4-ylcarbamoyl]-butyl}-amide

Following the procedure of Example 88(a)-88(d), except substituting thiophene-2-sulfonyl chloride for 3-chlorobenzenesulfonyl chloride in step (a) and 7-methoxybenzofuran-2-carboxylic acid for benzofuran-2-carboxylic acid in step (c), the title compound was prepared. MS (ESI): 562 (M+H⁺).

### Example 145

### Preparation of 3-Methyl-benzofuran-2-carboxylic acid-{(S)-3-methyl-1-[3-oxo-1-(thiophene-2-sulfonyl)-azepan-4-ylcarbamoyl]-butyl}-amide

Following the procedure of Example 88(a)-88(d), except substituting thiophene-2-sulfonyl chloride for 3-chlorobenzenesulfonyl chloride in step (a) and 3-methylbenzofuran-2-carboxylic acid for benzofuran-2-carboxylic acid in step (c), the title compound was prepared MS (ESI): 546 (M+H⁺).

### Example 146

### Preparation of Benzo[b]thiophene -2-carboxylic acid-{(S)-3-methyl-1-[3-oxo-1-(thiophene-2-sulfonyl)-azepan-4-ylcarbamoyl]-butyl}-amide

Following the procedure of Example 88(a)-88(d), except substituting thiophene-2-sulfonyl chloride for 3-chlorobenzenesulfonyl chloride in step (a) and benzo[b]thiophene-2-carboxylic acid for benzofuran-2-carboxylic acid in step (c), the title compound was prepared. MS (ESI): 548 (M+H⁺).

### Example 147

### Preparation of Quinoxaline-2-carboxylic acid-{(S)-3-methyl-1-[3-oxo-1-(thiophene-2-sulfonyl)-azepan-4-ylcarbamoyl]-butyl}-amide

Following the procedure of Example 88(a)-88(d), except substituting thiophene-2-sulfonyl chloride for 3-chlorobenzenesulfonyl chloride in step (a) and quinoxaline-2-carboxylic acid for benzofuran-2-carboxylic acid in step (c), the title compound was prepared. MS (ESI): 544 (M+H⁺).

### Example 148

### Preparation of 1-Methyl-1-H-indole-2-carboxylic acid-{(S)-3-methyl-1-[3-oxo-1-(thiophene-2-sulfonyl)-azepan-4-ylcarbamoyl]-butyl}-amide

Following the procedure of Example 88(a)-88(d), except substituting thiophene-2-sulfonyl chloride for 3-chlorobenzenesulfonyl chloride in step (a) and 1-methylindole-2-carboxylic acid for benzofuran-2-carboxylic acid in step (c), the title compound was prepared. MS (ESI): 545 (M+H⁺).

### Example 149

### Preparation of 5,6-Difluoro-benzofuran-2-carboxylic acid {(S)-3-methyl-1-[1-(pyridine-2-sulfonyl)-3-oxo-azepan-4-ylcarbamoyl]-butyl}amide

Following the procedure of Example 6(a)-6(b), except substituting pyridine-2-sulfonyl chloride fortbenzenesulfonyl chloride in step (a) and 5,6-difluorobenzofuran-2-carboxylic acid for benzo[1,3]dioxole-5-carboxylic acid in step (b), the title compound was prepared. MS (ESI): 562 (M+H⁺).

### Example 150

### Preparation of 5,6-Difluoro-benzofuran-2-carboxylic acid {(S)-3-methyl-1-[1-(pyridine-2-sulfonyl)-3-oxo-azepan-4-ylcarbamoyl]-butyl}amide

The title compound was isolated as the first eluting compound from the HPLC purification in Example 149. MS (ESI): 562 (M+H⁺).

### Example 151

### Preparation of Quinoline-2-carboxylic acid {(S)-3-methyl-1-[3-oxo-1-(pyridine-2-sulfonyl)-azepan-4-ylcarbamoyl]-butyl}amide

The title compound was isolated as the first eluting compound from the HPLC purification in Example 24. MS (ESI): 538 (M+H⁺).

### Example 152

### Preparation of Quinoline-6-carboxylic acid {(S)-3-methyl-1-[3-oxo-1-(pyridine-2-sulfonyl)-azepan-4-ylcarbamoyl]-butyl}amide

The title compound was isolated as the first eluting compound from the HPLC purification in Example 25. MS (ESI): 538 (M+H⁺).

### Example 153

### Preparation of Quinoline-4-carboxylic acid {(S)-3-methyl-1-[3-oxo-1-(pyridine-2-sulfonyl)-azepan-4-ylcarbamoyl]-butyl}amide

The title compound was isolated as the first eluting compound from the HPLC purification in Example 26. MS (ESI): 538 (M+H⁺).

### Example 154

### Preparation of Isoguinoline-1-carboxylic acid {(S)-3-methyl-1-[3-oxo-1-(pyridine-2-sulfonyl)-azepan-4-ylcarbamoyl]-butyl}amide

The title compound was isolated as the first eluting compound from the HPLC purification in Example 29. MS (ESI): 538 (M+H⁺).

### Example 155

### Preparation of Naphthalene-2-carboxylic acid {(S)-3-methyl-1-[3-oxo-1-(pyridine-2-sulfonyl)-azepan-4-ylcarbamoyl]-butyl}-amide

The title compound was isolated as the first eluting compound from the HPLC purification in Example 17. MS (ESI): 537 (M+H⁺).

### Example 156

### Preparation of Quinoline-3-carboxylic acid {(S)-3-methyl-1-[3-oxo-1-(pyridine-2-sulfonyl)-azepan-4-ylcarbamoyl]-butyl}amide

The title compound was isolated as the first eluting compound from the HPLC purification in Example 27. MS (ESI): 538 (M+H⁺).

### Example 157

### Preparation of 5,6-Dimethoxy-benzo[b]thiophene-2-carboxylic acid {(S)-3-methyl-1-[1-(6-methyl-pyridine-2-sulfonyl)3-oxo-azepan-4-ylcarbamoyl]-butyl}amide

Following the procedure of Example 6(a)-6(b), except substituting pyridine-2-sulfonyl chloride for benzenesulfonyl chloride in step (a) and 5,6-dimethoxybenzo[b]thiophene-2-carboxylic acid for benzo[1,3]dioxole-5-carboxylic acid in step (b), the title compound was prepared. MS (ESI): 603 (M+H⁺).

### Example 158

### Preparation of (R)-1-Benzyl-5-oxo-pyrrolidine-2-carboxylic acid {(S)-3-methyl-1-]3-oxo-(pyridine-2-sulfonyl)-azepan-4-ylcarbamoyl]-butyl}amide

Following the procedure of Example 6(a)-6(b), except substituting pyridine-2-sulfonyl chloride for benzenesulfonyl chloride in step (a) and (R)-1-benzyl-5-oxo-pyrrolidine-2-carboxylic acid for benzo[1,3]dioxole-5-carboxylic acid in step (b), the title compound was prepared. MS (ESI): 584 (M+H⁺).

### Example 159

### Preparation of Benzofuran-2-carboxylic acid {(S)-2-naphthalen-2-yl-1-[3-oxo-1-(pyridine-2-sulfonyl)-azepan-4-ylcarbamoyl)-ethyl]-amide

Following the procedure of Example 102(a)-102(h), except substituting N-tert-butoxycarbonyl-L-2-naphthylalanine for N-Boc-cyclohexylalanine in step (e), the title compound was prepared. MS (ESI): 611 (M+H⁺).

### Example 160

### Preparation of Thieno [3.2-b]thiophene-2-carboxylic acid {(S)-3-methyl-1-[1-(3-methyl-pyridine-2-sulfonyl)-3-oxo-azepan-4-ylcarbamoyl]-butyl}amide

Following the procedure of Example 6(a)-6(b), except substituting 3-methylpyridine-2-sulfonyl chloride for benzenesulfonyl chloride in step (a) and thieno[3,2-b]thiophene-2-carboxylic acid for benzo[1,3]dioxole-5-carboxylic acid in step (b), the title compound was prepared. MS (ESI): 563 (M+H⁺).

### Example 161

### Preparation of 3-Methyl-benzofuran-2-carboxylic acid {(S)-3-methyl-1-[1-(3-methyl-pyridine-2-sulfonyl)-3-oxo-azepan-4-ylcarbamoyl]-butyl}amide

Following the procedure of Example 6(a)-6(b), except substituting 3-methylpyridine-2-sulfonyl chloride for benzenesulfonyl chloride in step (a) and 3-methylbenzofuran-2-carboxylic acid for benzo[1,3]dioxole-5-carboxylic acid in step (b), the title compound was prepared. MS (ESI): 555 (M+H⁺).

### Example 162

### Preparation of 5-Methoxy-benzofuran-2-carboxylic acid {(S)-3-methyl-1-[1-(3-methyl-pyridine-2-sulfonyl)-3-oxo-azepan-4-ylcarbamoyl]-butyl}amide

Following the procedure of Example 6(a)-6(b), except substituting 3-methylpyridine-2-sulfonyl chloride for benzenesulfonyl chloride in step (a) and 5-methoxybenzofuran-2-carboxylic acid for benzo[1,3]dioxole-5-carboxylic acid in step (b), the title compound was prepared. MS (ESI): 571 (M+H⁺).

### Example 163

### Preparation of 5,6-Difluoro-benzofuran-2-carboxylic acid {(S)-3-methyl-1-[3-oxo-1-(1-oxy-pyridine-2-sulfonyl)-azepan-4-ylcarbamoyl]-butyl}amide

Following the procedure of Example 6(a)-6(b), except substituting 1-oxypyridine-2-sulfonyl chloride fortbenzenesulfonyl chloride in step (a) and 5,6-difluorobenzofuran-2-carboxylic acid for benzo[1,3]dioxole-5-carboxylic acid in step (b), the title compound was prepared. MS (ESI): 579 (M+H⁺).

### Example 164

### Preparation of 7-Methoxy-benzofuran-2-carboxylic acid {(S)-3-methyl-1-[1-(6-methyl-pyridine-2-sulfonyl)-3-oxo-azepan-4-ylcarbamoyl]-butyl}amide

Following the procedure of Example 6(a)-6(b), except substituting pyridine-2-sulfonyl chloride for benzenesulfonyl chloride in step (a) and 7-methoxybenzofuran-2-carboxylic acid for benzo[1,3]dioxole-5-carboxylic acid in step (b), the title compound was prepared. MS (ESI): 557 (M+H⁺).

### Example 165

### Preparation of 5,6-Dimethoxy-benzo[b]thiophene-2-carboxylic acid {(S)-3-methyl-1-[1-(6-methyl-pyridine-2-sulfonyl)3-oxo-azepan-4-ylcarbamoyl]-butyl}amide

The title compound was isolated as the first eluting compound from the HPLC purification in Example 157. MS (ESI): 603 (M+H⁺).

### Example 166

### Preparation of 5-Fluoro-benzofuran-2-carboxylic acid {(S)-3-methyl-1-[3-oxo-1-(pyridine-2-sulfonyl)-azepan-4-ylcarbamoyl]-butyl}-amide

Following the procedure of Example 6(a)-6(b), except substituting pyridine-2-sulfonyl chloride for benzenesulfonyl chloride in step (a) and 5-fluorobenzofuran-2-carboxylic acid for benzo[1,3]dioxole-5-carboxylic acid in step (b), the title compound was prepared. MS (ESI): 545 (M+H⁺).

### Example 167

### Preparation of Benzofuran-2-carboxylic acid {(S)-3-methyl-1-[6-methyl-3-oxo-1-(pyridine-sulfonyl)-azepan-4-ylcarbamoyl]-butyl}-amide

a) [(S)-1-(3-hydroxy-6-methyl-azepan-4-ylcarbamoyl)-3-methyl-butyl]-carbamic acid tert butyl ester

Following the procedure of Example 1(a)-1(g), except substituting 5-bromo-4-methyl-1-pentene for 5-bromo-1-pentene in step (a), the title compound was prepared. MS (ESI): 358 (M+H⁺).

b) Benzofuran-2-carboxylic acid {(S)-3-methyl-1-[6-methyl-3-oxo-1-(pyridine-sulfonyl)-azepan-4-ylcarbamoyl]-butyl}-amide

Following the procedure of Example 6(a)-6(b), except substituting pyridine-2-sulfonyl chloride for benzenesulfonyl chloride and [(S)-1-(3-hydroxy-6-methyl-azepan 4-ylcarbamoyl)-3-methyl-butyl]-carbamic acid tert butyl ester for [(S)-1-(3-hydroxy-azepan-4-ylcarbamoyl)-3-methyl-butyl]-carbamic acid tert butyl ester in step (a), and benzofuran-2-carboxylic acid for benzo[1,3]dioxole-5-carboxylic acid in step (b), the title compound was prepared. MS (ESI): 541 (M+H⁺).

### Example 168

### Preparation of 5-Fluoro-benzofuran-2-carboxylic acid {(S)-3-methyl-1-[3-oxo-1-(pyridine-2-sulfonyl)-azepan-4-ylcarbamoyl]-butyl}-amide

The title compound was isolated as the first eluting compound from the HPLC purification in Example 166. MS (ESI): 545 (M+H⁺).

### Example 169

### Preparation of 5-(3-Trifluoromethyl-phenyl)-furan-2-carboxylic acid {(S)-2-cyclohexyl-1-[3-oxo-1-(1-oxy-pyridine-2-sulfonyl)-azepan-4-ylcarbamoyl]-ethyl}-amide

a) [(S)-1-(3-hydroxy-azepan-4-ylcarbamoyl)-2-cyclohexyl-ethyl]-carbamic acid tert butyl ester

Following the procedure of Example 1(a)-1(g), except substituting N-tert-butoxycarbonyl-L-cyclohexylalanine for N-tert-butoxycarbonyl-L-leucine in step (f), the title compound was prepared. MS (ESI): 384 (M+H⁺).

b) 5-(3-Trifluoromethyl-phenyl)-furan-2-carboxylic acid {(S)-2-cyclohexyl-1-[3-oxo-1-(1-oxy-pyridine-2-sulfonyl)-azepan-4-ylcarbamoyl]-ethyl}-amide

Following the procedure of Example 6(a)-6(b), except substituting 1-oxypyridine-2-sulfonyl chloride for benzenesulfonyl chloride and [(S)-1-(3-hydroxy-azepan-4-ylcarbamoyl)-2-cyclohexyl-ethyl]-carbamic acid tert butyl ester for [(S)-1-(3-hydroxy-azepan-4-ylcarbamoyl)-3-methyl-butyl]-carbamic acid tert butyl ester in step (a), and 5-(3-trifluoromethyl-phenyl)-furan-2-carboxylic acid for benzo[1,3]dioxole-5-carboxylic acid in step (b), the title compound was prepared. MS (ESI): 677 (M+H⁺).

### Example 170

### Preparation of 5,6-Dimethoxy-benzofuran-2-carboxylic acid {(S)-2-cyclohexyl-1-[3-oxo-1-(1-oxy-pyridine-2-sulfonyl)-azepan-4-ylcarbamoyl]-ethyl}-amide

Following the procedure of Example 6(a)-6(b), except substituting1-oxypyridine-2-sulfonyl chloride for benzenesulfonyl chloride and [(S)-1-(3-hydroxy-azepan-4-ylcarbamoyl)-2-cyclohexyl-ethyl]-carbamic acid tert butyl ester for [(S)-1-(3-hydroxy-azepan-4-ylcarbamoyl)-3-methyl-butyl]-carbamic acid tert butyl ester in step (a), and 5,6-dimethoxybenzofuran-2-carboxylic acid for benzo[1,3]dioxole-5-carboxylic acid in step (b), the title compound was prepared. MS (ESI): 643 (M+H⁺).

### Example 171

### Preparation of 5-Methoxy-benzofuran-2-carboxylic acid {(S)-3-methyl-1-[1-(3-methyl-pyridine-2-sulfonyl)-3-oxo-azepan-4-ylcarbamoyl]-butyl}amide

The title compound was isolated as the first eluting, compound from the HPLC purification in Example 162. MS (ESI): 571 (M+H⁺).

### Example 172

### Preparation of Benzofuran-2-carboxylic acid {(S)-3-methyl-1-[1-(6-methyl-pyridine-2-sulfonyl)-3-oxo-azepan-4-ylcarbamoyl]-butyl}amide

Following the procedure of Example 6(a)-6(b), except substituting 3-methylpyridine-2-sulfonyl chloride for benzenesulfonyl chloride in step (a) and benzofuran-2-carboxylic acid for benzo[1,3]dioxole-5-carboxylic acid in step (b), the title compound was prepared. MS (ESI):. 541 (M+H⁺).

### Example 173

### Preparation of Benzofuran-2-carboxylic acid {(S)-3-methyl-1-[6-methyl-3-oxo-1-(pyridine-sulfonyl)-azepan-4-ylcarbamoyl]-butyl}-amide

The title compound was isolated as the first eluting compound from the HPLC purification in Example 167. MS (ESI): 541 (M+H⁺).

### Example 174

### Preparation of Quinoline-8-carboxylic acid {(S)-2-naphthalen-2-yl-1-[3-oxo-1-(pyridine-2-sulfonyl)-azepan-4-ylcarbamoyl)-ethyl]-amide

a) [(S)-1-(3-hydroxy-azepan-4-ylcarbamoyl)-2-(2-naphthyl)-ethyl]-carbamic acid tert butyl ester

Following the procedure of Example 1(a)-1(g), except substituting N-tert-butoxycarbonyl-L-2-naphthylalanine for N-tert-butoxycarbonyl-L-leucine in step (f), the title compound was prepared. MS (ESI): 428 (M+H⁺).

b) Quinoline-8-carboxylic acid {(S)-2-naphthalen-2-yl-1-[3-oxo-1-(pyridine-2-sulfonyl)-azepan-4-ylcarbamoyl)-ethyl]-amide

Following the procedure of Example 6(a)-6(b), except substituting pyridine-2-sulfonyl chloride for benzenesulfonyl chloride and [(S)-1-(3-hydroxy-azepan-4-ylcarbamoyl)-2-(2-naphthyl)-ethyl]-carbamic acid tert butyl ester for [(S)-1-(3-hydroxy-azepan-4-ylcarbamoyl)-3-methyl-butyl]-carbamic acid tert butyl ester in step (a), and quinoline-8-carboxylic acid for benzo[1,3]dioxole-5-carboxylic acid in step (b), the title compound was prepared. MS (ESI): 622 (M+H⁺).

### Example 175

### Preparation of Naphthalene-1-carboxylic acid {(S)-2-naphthalen-2-yl-1-[3-oxo-1-(pyridine-2-sulfonyl)-azepan-4-ylcarbamoyl)-ethyl]-amide

Following the procedure of Example 6(a)-6(b), except substituting pyridine-2-sulfonyl chloride forbenzenesulfonyl chloride and [(S)-1-(3-hydroxy-azepan-4-ylcarbamoyl)-2-(2-naphthyl)-ethyl]-carbamic acid tert butyl ester for [(S)-1-(3-hydroxy-azepan-4-ylcarbamoyl)-3-methyl-butyl]-carbamic acid tert butyl ester in step (a), and naphthalene-1-carboxylic acid for benzo[1,3]dioxole-5-carboxylic acid in step (b), the title compound was prepared. MS (ESI): 621 (M+H⁺).

### Example 176

### Preparation of Quinoline-8-carboxylic acid {(S)-1-[3-oxo-1-(pyridine-2-sulfonyl)-azepan-4-ylcarbamoyl]-2-phenyl-ethyl}-amide

a) [(S)-1-(3-hydroxy-azepan-4-ylcarbamoyl)-2-phenyl-ethyl]-carbamic acid tert butyl ester

Following the procedure of Example 1(a)-1(g), except substituting N-tert-butoxycarbonyl-L-phenylalanine for N-tert-butoxycarbonyl-L-leucine in step (f), the title compound was prepared. MS (ESI): 378 (M+H⁺).

b) Quinoline-8-carboxylic acid {(S)-2-naphthalen-2-yl-1-[3-oxo-1-(pyridine-2-sulfonyl)-azepan-4-ylcarbamoyl)-ethyl]-amide

Following the procedure of Example 6(a)-6(b), except substituting pyridine-2-sulfonyl chloride for benzenesulfonyl chloride and [(S)-1-(3-hydroxy-azepan-4-ylcarbamoyl)-2-phenyl-ethyl]-carbamic acid tert butyl ester for [(S)-1-(3-hydroxy-azepan-4-ylcarbamoyl)-3-methyl-butyl]-carbamic acid tert butyl ester in step (a), and quinoline-8-carboxylic acid for benzo[1,3]dioxole-5-carboxylic acid in step (b), the title compound was prepared. MS (ESI): 572 (M+H⁺).

### Example 177

### Preparation of Naphthyridine-2-carboxylic acid {(S)-3-methyl-1-[3-oxo-1-(pyridine-2-sulfonyl)-azepan-4-ylcarbamoyl]-butyl}-amide

Following the procedure of Example 6(a)-6(b), except substituting pyridine-2-sulfonyl chloride forfbenzenesulfonyl chloride in step (a) and naphthyridine-2-carboxylic acid for benzo[1,3]dioxole-5-carboxylic acid in step (b), the title compound was prepared. MS (ESI): 539 (M+H⁺).

### Example 178

### Preparation of Naphthalene-1-carboxylic acid {(S)-1-[3-oxo-1-(pyridine-2-sulfonyl)-azepan-4-ylcarbamoyl]-2-phenyl-ethyl}-amide

Following the procedure of Example 6(a)-6(b), except substituting pyridine-2-sulfonyl chloride for benzenesulfonyl chloride and [(S)-1-(3-hydroxy-azepan4-ylcarbamoyl)-2-phenyl-ethyl]-carbamic acid tert butyl ester for [(S)-1-(3-hydroxy-azepan4-ylcarbamoyl)-3-methyl-butyl]-carbamic acid tert butyl ester in step (a), and naphthalene-1-carboxylic acid for benzo[1,3]dioxole-5-carboxylic acid in step (b), the title compound was prepared. MS (ESI): 571 (M+H⁺).

### Example 179

### Preparation of Benzofuran-2-carboxylic acid {(S)-3-methyl-1-[-(2-methyl-furan-3-sulfonyl)-3-oxo-azepan-4-ylcarbamoyl]-butyl}-amide

Following the procedure of Example 6(a)-6(b). except substituting 2-methylfuran-3-sulfonyl chloride for benzenesulfonyl chloride in step (a) and benzofuran-2-carboxylic acid for benzo[1,3]dioxole-5-carboxylic acid in step (b), the title compound was prepared. MS (ESI): 530 (M+H⁺).

### Example 180

### Preparation of Quinoline-2-carboxylic acid {(S)-1-[3-oxo-1-(pyridine-2-sulfonyl)-azepan-4-ylcarbamoyl]-2-phenyl-ethyl}-amide

Following the procedure of Example 6(a)-6(b), except substituting pyridine-2-sulfonyl chloride for benzenesulfonyl chloride and [(S)-1-(3-hydroxy-azepan-4-ylcarbamoyl)-2-phenyl-ethyl]-carbamic acid tert butyl ester for [(S)-1-(3-hydroxy-azepan-4-ylcarbamoyl)-3-methyl-butyl]arbamic acid tert butyl ester in step (a), and quinoline-2-carboxylic acid for benzo(1,3]dioxole-5-carboxylic acid in step (b), the title compound was prepared. MS (ESI): 572 (M+H⁺).

### Example 181

### Preparation of Benzofuran-2-carboxylic acid {(S)-3-methyl-1-[(4S,7S)-7-methyl-3-oxo-1-(pyridine-2-sulfonyl)-azepan-4-ylcarbamoyl]-butyl}-amide

a) allyl-(1-methyl-pent-4-enylidene)-amine

Hex-5-en-2-one (9.8 g, 11.6 ml, 100 mmol) was added to a stirred solution of allylamine (8.55 mmol, 11.25 ml, 150 mmol), 4 Angstrom molecular sieves (52 g), and p-toluene sulfonic acid (10 mg) in CH₂Cl₂ (200 ml) and was stirred overnight. The reaction mixture was concentrated in vacuo by rotary evaporation and was used in the next reaction without further purification (13 g, 95%). MS (ESI): 137.9 (M+H⁺).

b) allyl-(1-methyl-pent-4-enyl)-amine

Sodium borohydride (2.7 g, 71 mmol) was added portionwise to a stirred solution of the compound of Example 181(a) (6.5 g, 47 mmol) in MeOH (100 ml) at 0 C. The reaction mixture was stirred for 30 minutes, then warmed to RT. Approximately 90 ml of MeOH was removed from the reaction mixture by rotary evaporation, then the reaction mixture was diluted with ether (200 ml), then extracted with water then brine. The combined organics were dried with MgSO₄, filtered, concentrated in vacuo by rotary evaporation to give a pale yellow liquid that was used in the next reaction without further purification (5.2 g, 80%).

c) allyl-(1-methyl-pent-4-enyl)-carbamic acid benzyl ester

Carbobenzyloxy chloride (9.56 g, 8 ml) was added dropwise to a stirred solution of the compound of Example 181(b) (7 g, 50 mmol), triethylamine (5.5 g, 8.0 ml, 57.5 mmol) in CH₂Cl₂ (100 ml) at 0 C. The reaction mixture was warmed to RT, then was stirred for 2 h. The reaction mixture was diluted with CH₂Cl₂ (100 ml), then was extracted with water, then brine. The combined organics were dried with MgSO₄, filtered, concentrated in vacuo by rotary evaporation, then was chromatographed (silica gel, 4% EtOAc/hexanes) to give the title compound (8.9 g, 65% yield). MS (ESI): 274.2 (M+H⁺).

d) 2-methyl-2,3,4,7-tetrahydro-azepine-1-carboxylic acid benzyl ester

The compound of Example 181(c) (1.036 g, 3.8 mmol) was dissolved in CH₂Cl₂ (10 ml) and a stream of argon gas was bubbled into the reaction mixture for 10 minutes. Then bis(tricyclohexylphosphine)benzylidine ruthenium(IV) dichloride (Strem Chemicals, Grubbs' catalyst, 22 mg, 0.027 mmol) was added and the reaction mixture was refluxed for 2 h. Additional bis(tricyclohexylphosphine)benzylidine ruthenium(IV) dichloride (11 mg, 0.014 mmol) was added and the reaction mixtrue was refluxed for an additional 1.5 hours. The reaction was cooled to RT under argon overnight, then was concentrated in vacuo by rotary evaporation, then was chromatographed (silica gel, 5% EtOAc/hexanes) to give the title compound (0.83 g, 89%). MS (ESI): 246.2 (M+H⁺).

e) (1S,4R,7R)-4-Methyl-8-oxa-3-aza-bicyclo[5.1.0]octane-3-carboxylic acid benzyl ester

m-Chloro-perbenzoic acid (1.05 g, 57-86% pure) was added to a solution of the compound of Example 181(d) (0.83 g, 3.34 mmol) in CH₂Cl₂ at 0 degrees C. The reaction mixture was stirred for half an hour, then was warmed to RT. Additional m-chloro-perbenzoic acid (0.3 g, 57-86% pure) was added and the reaction was stirred 2 h. The reaction mixture was concentrated in vacuo by rotary evaporation, then 80 ml of 9:1 hexanes/EtOAc was added and the reaction mixture was filtered. The filtrate was concentrated in vacuo by rotary evaporation, then was chromatoaraphed (silica gel, 20% EtOAc:hexanes) to give racemic (1S,4R,7S)-4-methyl-8-oxa-3-aza-bicyclo[5.1.0]octane-3-carboxylic acid benzyl ester (0.44 g, 50%) and the title compound as a racemic mixture of the title compound (0.15 g, 17% yield). MS (ESI): 262.0 (M+H⁺).

f) (2R,5S,6S)-5-Azido-6-hydroxy-2-methyl-azepane-1-carboxylic acid benzyl ester

Sodium azide (0.56 g, 8.62 mmol) was added to a solution of the compound of Example 181(e) (0.75 g, 2.87 mmol) and ammonium chloride (0.46 g, 8.62 mmol) in MeOH (5 ml) and H₂O (0.5 ml), then was refluxed for 6 h. The reaction mixture was concentrated in vacuo by rotary evaporation, then was diluted with water (5 ml) and extracted with EtOAc (10 ml). The organic layer was then extracted with water, brine, dried with MgSO₄, filtered, concentrated, in vacuo by rotary evaporation, and chromatographed (silica gel, 20% EtOAc/hexanes) to yield the title compound (0.7 g, 80%). MS (ESI): 305.2 (M+H⁺).

g) (2R,5S,6S)-5-Amino-6-hydroxy-2-methyl-azepane-1-carboxylic acid benzyl ester Triphenylphosphine (1.94 g, 7.4 mmol) was added to a solution of the compound of Example 181(f) (1.5 g, 4.93 mmol) in THF (185 ml) and H₂O (0.7 ml), then was heated to 45 degrees C overnight. The reaction mixture was then diluted with toluene (100 ml×2) and was azeotroped in vacuo by rotary evaporation twice. The resulting oil was dissolved in MeOH and HCl in Et₂O and the resulting salt was collected following filtration and was used in the next reaction without further purification (1.4 g, 90%).

h) (2R,5S,6S)-5-((S)-2-tert-Butoxycarbonylamino-4-methyl-pentanoylamino)-6-hydroxy-2-methyl-azepane-1-carboxylic acid benzyl ester and (2S,5R,6R)-5-((S)-2-tert -Butoxycarbonylamino-4-methyl-pentanoylamino)-6-hydroxy-2-methyl-azepane-1-carboxylic acid benzyl ester

1-(3-Dimethylaminopropyl)-3-ethylcarbodiimide (0.33 g, 1.73 mmol) was added to a solution of Boc-leucine-hydrate (0.43 g, 1.7 mmol), diisopropylethylamine (0.22 g, 0.3 ml, 1.7 mmol), hydroxybenztriazole (0.25 g, 1.85 mmol), and the compound of Example 181(g) (0.5 g, 1.6 mmol) in DMF (10 ml). The reaction was stirred overnight at RT then was diluted with EtOAc (100 ml), washed with H₂O (3×50 ml), brine (50 ml), dried with magnesium sulfate, filtered, concentrated in vacuo by rotary evaporation, and chromatographed (silica gel, 50% EtOAc/hexanes) to yield the title compound (0.78 g, 100%). MS (ESI): 492.0 (M+H⁺).

i) [(S)-1-((3S,4S,7R)-3-Hydroxy-7-methyl-azepan-4-ylcarbamoyl)-3-methyl-butyl]-carbamic acid tert-butyl ester and [(S)-1-((3R,4R,7S)-3-Hydroxy-7-methyl-azepan-4-ylcarbamoyl)-3-methyl-butyl]-carbamic acid tert-butyl ester

The compound of Example 181(h) (0.77 g, 1.57 mmol) was dissolved in EtOAc (27.5 ml), MeOH (5.5 ml). Then 10% Pd/C (0.39 g) was added and the reaction was stirred overnight under a balloon filled with hydrogen gas. The reaction mixture was filtered through Celite, concentrated in vacuo by rotary evaporation and was used in the next reaction without further purification (0.56 g). MS (ESI): 358.1 (M+H⁺).

j) [(S)-1-((3S,4S ,7R)-1-Benzenesulfonyl-3-hydroxy-7-methyl-azepan-4-ylcarbamoyl)-3-methyl-butyl]-carbamic acid tert-butyl ester and [(S)-1-((3R,4R,7S)-1-Benzenesulfonyl-3-hydroxy-7-methyl-azepan-4-ylcarbamoyl)-3-methyl-butyl]-carbamic acid tert-butyl ester

2-Pyridine sulfonyl chloride (0.6 g, 3.4 mmol) was added to a solution of the compound of Example 181(i) (1.0 g, 2.8 mmol), N-methyl morpholine (0.45 ml, 4.1 mmol) in CH₂Cl₂ (35 ml) and was stirred at RT overnight. The reaction mixture was diluted with EtOAc (100 ml), washed with H₂O, brine, dried with magnesium sulfate, filtered, concentrated in vacuo by rotary evaporation, and chromatographed (silica gel, 2.5% MeOH/CH₂Cl₂) to yield the title compound (0.9 g, 64%). MS (ESI): 499.0 (M+H⁺).

k) (S)-2-Amino-4-methyl-pentanoic acid ((3S,4S,7R)-1-(2-pyridine)-sulfonyl-3-hydroxy-7-methyl-azepan4-yl)-amide and (S)-2-Amino-4-methyl-pentanoic acid ((3R,4R,7S)-1-(2-pyridine)-sulfonyl-3-hydroxy-7-methyl-azepan-4-yl)-amide

HCl in dioxane (4.0 M, 15 ml) was added to a stirred solution of the compound of Example 181(j) (0.9 g, 1.8 mmol) in MeOH (15 ml). The reaction mixture was stirred for 2 h at RT, then was concentrated in vacuo by rotary evaporation and was used in the next reaction without further purification (0.85 g).

1) Benzofuran-2-carboxylic acid {(S)-1-[(3S,4S,7R)-3-hydroxy-7-methyl-1-(pyridine-2sulfonyl)-azepan-4-ylcarbamoyl]-3-methyl-butyl}-amide and benzofuran-2-carboxylic acid {(S)-1-[(3R,4R,7S)-3-hydroxy-7-methyl-1-(pyridine-2-sulfonyl)-azepan-4-ylcarbamoyl]-3-methyl-butyl}-amide

1-(3-Dimethylaminopropyl)-3-ethylcarbodiimide (0.35 g, 1.85 mmol) was added to a solution of 2-benzofuran-carboxylic acid (0.3 g, 1.85 mmol), the compound of Example 181(k) (0.85 g 1.8 mmol), diisopropylethylamine (0.48 g, 0.65 ml, 3.7 mmol), hydroxybenztriazole (0.25 g, 1.85 mmol) in DMF (10 ml) and was stirred at RT overnight. The reaction mixture was then warmed to RT and was stirred overnight. The reaction mixture was diluted with EtOAc (100 ml), washed with H₂O, brine, dried with magnesium sulfate, filtered, concentrated in vacuo by rotary evaporation, and chromatographed (silica gel, 2.5% MeOH/CH₂Cl₂) to yield the title compound (0.8 g, 82%). MS (ESI): 542.98 (M+H⁺).

m) Benzofuran-2-carboxylic acid {(S)-3-methyl-1-[(4S,7S)-7-methyl-3-oxo-1-(pyridine-2-sulfonyl)-azepan-4-ylcarbamoyl]-butyl}-amide

Dess-Martin periodinane (1.0 g, 2.36 mmol) was added to a solution of The compound of Example 181(l) (0.8 g, 1.48 mmol) in CH₂Cl₂ (20 ml) and was stirred at RT for 45 minutes. The solution was washed with 10% NaHCO₃ and brine. Purification by column chromatography (60% ethyl acetate/hexanes) followed by HPLC (Whelk-Ol; ethanol/hexanes) gave the title compound as a mixture of diasteromers (0.75 g, 94%). MS (ESI): 541 (M+H⁺).

### Example 182

### Preparation of Benzofuran-2-carboxylic acid {(S)-3-methyl-1-[(4R,7R)-7-methyl-3-oxo-1-(pyridine-2-sulfonyl)-azepan-4-ylcarbamoyl]-butyl}-amide

The title compound was isolated as the second eluting compound from the HPLC purification in Example 181. MS (ESI): 541 (M+H⁺).

### Example 183

### Preparation of Benzofuran-2-carboxylic acid {(S)-1-[-(3-fluoro-benzensulfonyl)-3-oxo-azepan-4-ylcarbamoyl]-3-methyl-1-butyl}-amide

Following the procedure of Example 88(a)-88(d), except substituting 3-fluoobenzenesulfonyl chloride for 3-chlorobenzenesulfonyl chloride in step (a), the title compound was prepared. MS (ESI): 543 (M+H⁺).

### Example 184

### Preparation of Naphthalene-1-carboxylic acid {(S)-3-methyl-1-[3-oxo-1-(pyridine-2-sulfonyl)-azepan-4-ylcarbamoyl]-butyl}-amide

Following the procedure of Example 6(a)-6(b), except substituting pyridine-2-sulfonyl chloride for benzenesulfonyl chloride in step (a) and naphthalene-1-carboxylic acid for benzo[1,3]dioxole-5-carboxylic acid in step (b), the title compound was prepared. MS (ESI): 537 (M+H⁺).

### Example 185

### Preparation of Quinoline-5-carboxylic acid {(S)-3-methyl-1-[3-oxo-1-(pyridine-2-sulfonyl)-azepan-4-ylcarbamoyl]-butyl}-amide

Following the procedure of Example 6(a)-6(b), except substituting pyridine-2-sulfonyl chloride for benzenesulfonyl chloride in step (a) and quinoline-5-carboxylic acid for benzo[1,3]dioxole-5-carboxylic acid in step (b), the title compound was prepared. MS (ESI): 538 (M+H⁺).

### Example 186

### Preparation of 5-(3-Trifluoromethyl-phenyl)-furan-2-carboxylic acid ((S)-3-methyl-1-{3-oxo-1[-1-(1-oxy-pyridin-2-yl)-methanoyl]-azepan-4-ylcarbamoyl}-butyl)-amide

Following the procedure of Example 3(d) and 3(h), except substituting 1-oxypicolinic acid for 3-(2-pyridyl)phenylacetic acid in step (d) and 5-(3-trifluoromethyl-phenyl)-furan-2-carboxylic acid for benzo[1,3]dioxole-5-carboxylic acid in step (b), the title compound was prepared. MS (ESI): 601 (M+H⁺).

### Example 187

### Preparation of Quinoline-8-carboxylic acid {(S)-2-naphthalen-2-yl-1-[3-oxo-1-(pyridine-2-sulfonyl)-azepan-4-ylcarbamoyl]-ethyl}-amide

The title compound was isolated as the first eluting compound from the HPLC purification in Example 174. MS (ESI): 622 (M+H⁺).

### Example 188

### Preparation of Naphthalene-1-carboxylic acid {(S)-2-naphthalen-2-yl-1-[3-oxo-1-(pyridine-2-sulfonyl)-azepan-4-ylcarbamoyl]-ethyl}-amide

The title compound was isolated as the first eluting compound from the HPLC purification in Example 175. MS (ESI): 621 (M+H⁺).

### Example 189

### Preparation of Quinoline-8-carboxylic acid {(S)-1-[3-oxo-1-(pyridine-2-sulfonyl)-azepan-4-ylcarbamoyl]-2-phenyl-ethyl}-amide

The title compound was isolated as the first eluting compound from the HPLC purification in Example 176. MS (ESI): 572 (M+H⁺).

### Example 190

### Preparation of Naphthalene-1-carboxylic acid {(S)-1-[3-oxo-1-(pyridine-2-sulfonyl)-azepan-4-ylcarbamoyl]-2-phenyl-ethyl}-amide

The title compound was isolated as the first eluting compound from the HPLC purification in Example 178. MS (ESI): 571 (M+H⁺).

### Example 191

### Preparation of 5-Fluoro-benzofuran-2-carboxylic acid {(S)-3-methyl-1-[3-oxo-1-(1-ox -pyridine-2-sulfonyl)-azepan-4-ylcarbamoyl]-butyl}-amide

Following the procedure of Example 6(a)-6(b), except substituting 1-oxypyridine-2-sulfonyl chloride forbenzenesulfonyl chloride in step (a) and 5-fluorobenzofuran-2-carboxylic acid for benzo[1,3]dioxole-5-carboxylic acid in step (b), the title compound was prepared. MS (ESI): 561 (M+H⁺).

### Example 192

### Preparation of 5-Fluoro-3-methyl-benzofuran-2-carboxylic acid {(S)-3-methyl-1-[3-oxo-1-(1-oxy-pyridine-2-sulfonyl)-azepan-4-ylcarbamoyl]-butyl}-amide

Following the procedure of Example 6(a)-6(b), except substituting 1-oxypyridine-2-sulfonyl chloride for benzenesulfonyl chloride in step (a) and 5-fluoro-3-methylbenzofuran-2-carboxylic acid for benzo[1,3]dioxole-5-carboxylic acid in step (b), the title compound was prepared. MS (ESI): 575 (M+H⁺).

### Example 193

### Preparation of 6-Fluoro-3-methyl-benzofuran-2-carboxylic acid {(S)-3-methyl-1-[3-oxo-1-(1-oxy-pyridine-2-sulfonyl)-azepan-4-ylcarbamoyl]-butyl}-amide

Following the procedure of Example 6(a)-6(b), except substituting1-oxypyridine-2-sulfonyl chloride for benzenesulfonyl chloride in step (a) and 6-fluoro-3-methylbenzofuran-2-carboxylic acid for benzo[1,3]dioxole-5-carboxylic acid in step (b), the title compound was prepared. MS (ESI): 575 (M+H⁺).

### Example 194

### Preparation of 5-Fluoro-benzofuran-2-carboxylic acid {(S)-3-methyl-1-[3-oxo-1-(1-oxy-pyridine-2-sulfonyl)-azepan-4-ylcarbamoyl]-butyl}-amide

The title compound was isolated as the first eluting compound from the HPLC purification in Example 191. MS (ESI): 561 (M+H⁺).

### Example 195

### Preparation of 5-Fluoro-3-methyl-benzofuran-2-carboxylic acid {(S)-3-methyl-1-[3-oxo-1-(1-oxy-pyridine-2-sulfonyl)-azepan-4-ylcarbamoyl]-butyl}-amide

The title compound was isolated as the first eluting compound from the HPLC purification in Example 192. MS (ESI): 575 (M+H⁺).

### Example 196

### Preparation of 6-Fluoro-3-methyl-benzofuran-2-carboxylic acid {(S)-3-methyl-1-[3-oxo-1-(1-oxy-pyridine-2-sulfonyl)-azepan-4-ylcarbamoyl]-butyl}-amide

The title compound was isolated as the first eluting compound from the HPLC purification in Example 193. MS (ESI): 575 (M+H⁺).

### Example 197

### Preparation of Benzo[b]thiophene-2-carboxylic acid {(S)-3-methyl-1-[6-methyl-3-oxo-1-(pyridine-2-sulfonyl)-azepan-4-ylcarbamoyl]-butyl}-amide

Following the procedure of Example 6(a)-6(b), except substituting pyridine-2-sulfonyl chloride for benzenesulfonyl chloride and [(S)-1-(3-hydroxy-6-methyl-azepan-4-ylcarbamoyl)-3-methyl-butyl]-carbamic acid tert butyl ester for [(S)-1-(3-hydroxy-azepan-4-ylcarbamoyl)-3-methyl-butyl]-carbamic acid tert butyl ester in step (a), and benzo[b]thiophene-2-carboxylic acid for benzo[1,3]dioxole-5-carboxylic acid in step (b), the title compound was prepared. MS (ESI): 557 (M+H⁺).

### Example 198

### Preparation of 5-Methoxy-benzofuran-2-carboxylic acid {(S)-3-methyl-1-[6-methyl-3-oxo-1-(pyridine-2-sulfonyl l)-azepan-4-ylcarbamoyl]-butyl}-amide

Following the procedure of Example 6(a)-6(b), except substituting pyridine-2-sulfonyl chloride for benzenesulfonyl chloride and [(S)-1-(3-hydroxy-6-methyl-azepan-4-ylcarbamoyl)-3-methyl-butyl]-carbamic acid tert butyl ester for [(S)-1-(3-hydroxy-azepan-4-ylcarbamoyl)-3-methyl-butyl]-carbamic acid tert butyl ester in step (a), and 5-methoxybenzofuran-2-carboxylic acid for benzo[1,3]dioxole-5-carboxylic acid in step (b), the title compound was prepared. MS (ESI): 571 (M+H⁺).

### Example 199

### Preparation of 3-Methyl-benzofuran-2-carboxylic acid {(S)-3-methyl-1-[6-methyl-3-oxo-1-(pyridine-2-sulfonyl)-azepan-4-ylcarbamoyl]-butyl}-amide

Following the procedure of Example 6(a)-6(b), except substituting pyridine-2-sulfonyl chloride for benzenesulfonyl chloride and [(S)-1-(3-hydroxy-6-methyl-azepan-4-ylcarbamoyl)-3-methyl-butyl]-carbamic acid tert butyl ester for [(S)-1-(3-hydroxy-azepan-4-ylcarbamoyl)-3-methyl-butyl]-carbamic acid tert butyl ester in step (a), and 3-methylbenzofuran-2-carboxylic acid for benzo[1,3]dioxole-5-carboxylic acid in step (b), the title compound was prepared. MS (ESI): 555 (M+H⁺).

### Example 200

### Preparation of Thieno[3,2-]thiophene-2-carboxylic acid {(S)-3-methyl-1-[6-methyl-3-oxo-1-(pyridine-2-sulfonyl)-azepan-4-ylcarbamoyl]-butyl}-amide

Following the procedure of Example 6(a)-6(b), except substituting pyridine-2-sulfonyl chloride forbenzenesulfonyl chloride and [(S)-1-(3-hydroxy-6-methyl-azepan-4-ylcarbamoyl)-3-methyl-butyl]-carbamic acid tert butyl ester for [(S)-1-(3-hydroxy-azepan-4-ylcarbamoyl)-3-methyl-butyl]-carbamic acid tert butyl ester in step (a), and thieno[3,2-b]thiophene-2-carboxylic acid for benzo[1,3]dioxole-5-carboxylic acid in step (b), the title compound was prepared. MS (ESI): 563 (M+H⁺).

### Example 201

### Preparation of 3,5-Dimethyl-benzofuran-2-carboxylic acid {(S)-3-methyl-1-[3-oxo-1-(1-oxy-pyridine-2-sulfonyl)-azepan-4-ylcarbamoyl]-butyl}-amide

Following the procedure of Example 6(a)-6(b), except substituting 1-oxypyridine-2-sulfonyl chloride for benzenesulfonyl chloride in step (a) and 3,5-dimethylbenzofuran-2-carboxylic acid for benzo[1,3]dioxole-5-carboxylic acid in step (b), the title compound was prepared. MS (ESI): 571 (M+H⁺).

### Example 202

### Preparation of 3-Ethyl-benzofuran-2-carboxylic acid {(S)-3-methyl-1-[3-oxo-1-(1-oxy-pyridine-2-sulfonyl)-azepan-4-ylcarbamoyl]-butyl}-amide

Following the procedure of Example 6(a)-6(b), except substituting 1-oxypyridine-2-sulfonyl chloride for benzenesulfonyl chloride in step (a) and 3-ethylbenzofuran-2-carboxylic acid for benzo[1,3]dioxole-5-carboxylic acid in step (b), the title compound was prepared. MS (ESI): 571 (M+H⁺).

### Example 203

### Preparation of 4-Methoxy-3-methyl-benzofuran-2-carboxylic acid {(S)-3-methyl-1-[3-oxo-1-(1-oxy-pyridine-2-sulfonyl)-azepan-4-ylcarbamoyl]-butyl}-amide

Following the procedure of Example 6(a)-6(b), except substituting 1-oxypyridine-2-sulfonyl chloride for benzenesulfonyl chloride in step (a) and 4-methoxy-3-methylbenzofuran-2-carboxylic acid for benzo[1,3]dioxole-5-carboxylic acid in step (b), the title compound was prepared. MS (ESI): 587 (M+H⁺).

### Example 204

### Preparation of 6-Methoxy-3-methyl-benzofuran-2-carboxylic acid {(S)-3-methyl-1-[3-oxo-1-(1-oxy-pyridine-2-sulfonyl)-azepan-4-ylcarbamoyl]-butyl}-amide

Following the procedure of Example 6(a)-6(b), except substituting 1-oxypyridine-2-sulfonyl chloride for benzenesulfonyl chloride in step (a) and 6-methoxy-3-methylbenzofuran-2-carboxylic acid for benzo[1,3]dioxole-5-carboxylic acid in step (b), the title compound was prepared. MS (ESI): 587 (M+H⁺).

### Example 205

### Preparation of 5-Methoxy-3-methyl-benzofuran-2-carboxylic acid {(S)-3-methyl-1-[3-oxo-1-(1-oxy-pyridine-2-sulfonyl)-azepan-4-ylcarbamoyl]-butyl}-amide

Following the procedure of Example 6(a)-6(b), except substituting 1-oxypyridine-2-sulfonyl chloride for benzenesulfonyl chloride in step (a) and 5-methoxy-3-methylbenzofuran-2-carboxylic acid for benzo[1,3]dioxole-5-carboxylic acid in step (b), the title compound was prepared. MS (ESI): 587 (M+H⁺).

### Example 206

### Preparation of 3.5-Dimethyl-benzofuran-2-carboxylic acid {(S)-3-methyl-1-[3-oxo-1-(1-oxy-pyridine-2-sulfonyl)-azepan-4-ylcarbamoyl]-butyl}-amide

The title compound was isolated as the first eluting compound from the HPLC purification in Example 201. MS (ESI): 571 (M+H⁺).

### Example 207

### Preparation of 3-Ethyl-benzofuran-2-carboxylic acid {(S)-3-methyl-1-[3-oxo-1-(1-oxy-pyridine-2-sulfonyl)-azepan-4-ylcarbamoyl]-butyl}-amide

The title compound was isolated as the first eluting compound from the HPLC purification in Example 202. MS (ESI): 571 (M+H⁺).

### Example 208

### Preparation of 4-Methoxy-3-methyl-benzofuran-2-carboxylic acid {(S)-3-methyl-1-[3-oxo-1-(1-oxy-pyridine-2-sulfonyl)-azepan-4-ylcarbamoyl]-butyl}-amide

The title compound was isolated as the first eluting compound from the HPLC purification in Example 203. MS (ESI): 587 (M+H⁺).

### Example 209

### Preparation of 4-Methoxy-3-methyl-benzofuran-2-carboxylic acid {(S)-3-methyl-1-[3-oxo-1-(1-oxy-pyridine-2-sulfonyl)-azepan-4-ylcarbamoyl]-butyl}-amide

The title compound was isolated as the second eluting compound from the HPLC purification in Example 203. MS (ESI): 587 (M+H⁺).

### Example 210

### Preparation of 1-methyl-naphtho[2,1-b]-furan-2-carboxylic acid {(S)-3-methyl-1-[3oxo-1-(1-oxy-pyridine-2-sulfonyl)-azepan-4-ylcarbamoyl]-butyl}-amide

Following the procedure of Example 6(a)-6(b), except substituting1-oxypyridine-2-sulfonyl chloride forbenzenesulfonyl chloride in step (a) and 1-methyl-naphtho[2,1-b]-furan-2-carboxylic acid for benzo[1,3]dioxole-5-carboxylic acid in step (b), the title compound was prepared. MS (ESI): 607 (M+H⁺).

### Example 211

### Preparation of 6-Methoxy-3-methyl-benzofuran-2-carboxylic acid {(S)-3-methyl-1-[3-oxo-1-(1-oxy-pyridine-2-sulfonyl)-azepan-4-ylcarbamoyl]-butyl}-amide

The title compound was isolated as the first eluting compound from the HPLC purification in Example 204. MS (ESI): 587 (M+H⁺).

### Example 212

### Preparation of Benzofuran-2-carboxylic acid [(S)-3-methyl-1-[3-oxo-1-quinolin-2-ylmethyl-azepan-4-ylcarbamoyl]-butyl}-amide

Following the procedure of Example 1(a)-1(k), except substituting quinoline-2-carboxaldehyde for benzaldehyde in step (h) and benzofuran-2-carboxylic acid for benzo[1,3]dioxole-5-carboxylic acid in step (j), the title compound was prepared. MS (ESI): 527 (M+H⁺).

### Example 213

### Preparation of 3-Methyl-benzofuran-2-carboxylic acid [(S)-3-methyl-1-[3-oxo-1-quinolin-2-ylmethyl-azepan-4-ylcarbamoyl]-butyl}-amide

Following the procedure of Example 1(a)-1(k), except substituting quinoline-2-carboxaldehyde for benzaldehyde in step (h) and 3-methylbenzofuran-2-carboxylic acid for benzo[1,3]dioxole-5-carboxylic acid in step (j), the title compound was prepared. MS (ESI): 541 (M+H⁺).

### Example 214

### Preparation of Benzo[b]thiophene-2-carboxylic acid {(S)-3-methyl-1-[3-oxo-1-quinolin-2-ylmethyl-azepan-4-ylcarbamoyl]-butyl}-amide

Following the procedure of Example 1(a)-1(k), except substituting quinoline-2-carboxaldehyde for benzaldehyde in step (h) and benzo[b]thiophene-2-carboxylic acid for benzo[1,3]dioxole-5-carboxylic acid in step (j), the title compound was prepared. MS (ESI): 543 (M+H⁺).

### Example 215

### Preparation of Benzo[b]thiophene-2-carboxylic acid {(S)-1-[1-(2-fluoro-phenylcarbamoyl)-3-oxo-azepan-4-ylcarbamoyl]-3-methyl-butyl}-amide

a) [(S)-1-(3-hydroxy-1-(2-fluorophenylcarbamoyl-azepan-4-ylcarbamoyl)-3-methyl-butyl]-carbamic acid tert-butyl ester

Following the procedure of Example 5(a), except subsituting 2-fluorophenyl isocyanate for phenyl isocyanate, the title compound was prepared. MS (ESI): 482 (M+H⁺).

b) Benzo[b]thiophene-2-carboxylic acid {(S)-1-[-(2-fluoro-phenylcarbamoyl)-3-oxo-azepan-4-ylcarbamoyl]-3-methyl-butyl}-amide

Following the procedure of Example 1(i)-1(k), except substituting [(S)-1-(3-hydroxy-1-(2-fluorophenylcarbamoyl)-azepan-4-ylcarbamoyl]-3-methyl-butyl]-carbamic acid tert-butyl ester for [(S)-1-(1-benzyl-3-hydroxy-azepan-4-ylcarbamoyl)-3-methyl-butyl]-carbamic acid tert butyl ester in step (i) and benzo[b]thiophene-2-carboxylic acid for benzo[1,3]dioxole-5carboxylic acid in step (j), the title compound was prepared. MS (ESI): 539 (M+H⁺).

### Example 216

### Preparation of 3-Methyl-benzofuran-2-carboxylic acid {(S)-1-[1-(2-fluoro-phenylcarbamoyl)-3-oxo-azepan-4-ylcarbamoyl]-3-methyl-butyl}-amide

Following the procedure of Example 1(i)-1(k), except substituting [(S)-1-(3-hydroxy-1-(2-fluorophenylcarbamoyl)-azepan-4-ylcarbamoyl)-3-methyl-butyl]-carbamic acid tert-butyl ester for [(S)-1-(1-benzyl-3-hydroxy-azepan-4-ylcarbamoyl)-3-methyl-butyl]-carbaric acid tert butyl ester in step (i) and 3-methylbenzofuran-2-carboxylic acid for benzo[1,3]dioxole-5-carboxylic acid in step (j), the title compound was prepared. MS (ESI): 537 (M+H⁺).

### Example 217

### Preparation of Quinoxaline-2-carboxylic acid {(S)-1-[1-(2-fluoro-phenylcarbamol)-3-oxo-azepan-4-ylcarbamoyl]-3-methyl-butyl}-amide

Following the procedure of Example 1(i)-1(k), except substituting [(S)-1-(3-hydroxy-1-(2-fluorophenylcarbamoyl)-azepan-4-ylcarbamoyl)-3-methylbutyl]-carbamic acid tert-butyl ester for [(S)-1-(1-benzyl-3-hydroxy-azepan-4-ylcarbamoyl)-3-methyl-butyl]-carbamic acid tert butyl ester in step (i) and quinoxaline-2-carboxylic acid for benzo[1,3]dioxole-5-carboxylic acid in step (j), the title compound was prepared. MS (ESI): 535 (M+H⁺).

### Example 218

### Preparation of Thieno[3,2-b]thiophene-2-carboxylic acid {(S)-1-[1-(2-fluoro-phenylcarbamoyl)-3-oxo-azepan-4-ylcarbamoyl]-3-methyl-butyl}-amide

Following the procedure of Example 1(i)-1(k), except substituting [(S)-1-(3-hydroxy-1-(2-fluorophenylcarbamoyl)-azepan-4-ylcarbamoyl)-3methyl-butyl]-carbamic acid tert-butyl ester for [(S)-1-(1-benzyl-3-hydroxy-azepan4-ylcarbamoyl)-3-methyl-butyl]-carbamic acid tert butyl ester in step (i) and thieno[3,2-b]thiophene-2-carboxylic acid for benzo[1,3]dioxole-5-carboxylic acid in step (j), the title compound was prepared. MS (ESI): 545 (M+H⁺).

### Example 219

### Preparation of Quinoline-2-carboxylic acid {(S)-1-[1-(2-fluoro-phenylcarbamoyl)-3-oxo-azepan-4-ylcarbamoyl]-3-methyl-butyl}-amide

Following the procedure of Example 1(i)-1(k), except substituting [(S)-1-(3-hydroxy-1-(2-fluorophenylcarbamoyl)-azepan-4-ylcarbamoyl)-3-methyl-butyl]-carbamic acid tert-butyl ester for [(S)-1-(1-benzyl-3-hydroxy-azepan4-ylcarbamoyl)-3-methyl-butyl]-carbamic acid tert butyl ester in step (i) and quinoline-2-carboxylic acid for benzo[1,3]dioxole-5-carboxylic acid in step (j), the title compound was prepared. MS (ESI): 534 (M+H⁺).

### Example 220

### Preparation of 4-Methyl-2-carboxylic acid {(S)-1-[-(2-fluoro-phenylcarbamoyl)-3-oxo-azepan-4-ylcarbamoyl]-3-methyl-butyl}-amide

Following the procedure of Example 1(i)-1(k), except substituting [(S)-1-(3-hydroxy-1-(2-fluorophenylcarbamoyl)-azepan-4-ylcarbamoyl]-3-methyl-butyl]-carbamic acid tert-butyl ester for [(S)-1-(1-benzyl-3-hydroxy-azepan-4-ylcarbamoyl)-3-methyl-butyl]-carbamic acid tert butyl ester in step (i) and 4-methylthiophene-2-carboxylic acid for benzo[1,3]dioxole-5-carboxylic acid in step (j), the title compound was prepared. MS (ESI): 503 (M+H⁺).

### Example 221

### Preparation of 5-Methoxy-benzofuran-2-carboxylic acid {(S)-1-[1-(2-fluoro-phenylcarbamoyl)-3-oxo-azepan-4-ylcarbamoyl]-3-methyl-butyl}-amide

Following the procedure of Example 1(i)-1(k), except substituting [(S)-1-(3-hydroxy-1-(2-fluorophenylcarbamoyl)-azepan-4-ylcarbamoyl)-3-methyl-butyl]-carbamic acid tert-butyl ester for [(S)-1-(1-benzyl-3-hydroxy-azepan-4-ylcarbamoyl)-3-methyl-butyl]-carbamic acid tert butyl ester in step (i) and 5-methoxybenzofuran-2-carboxylic acid for benzo[1,3]dioxole-5-carboxylic acid in step (j), the title compound was prepared. MS (ESI): 553 (M+H⁺).

### Example 222

### Preparation of 4-Methyl-furan-2-carboxylic acid {(S)-1-[1-(2-fluoro-phenylcarbamoyl)-3-oxo-azepan-4-ylcarbamoyl]-3-methyl-butyl}-amide

Following the procedure of Example 1(i)-1(k), except substituting [(S)-1-(3-hydroxy-1-(2-fluorophenylcarbamoyl)-azepan-4-ylcarbamoyl)-3-methyl-butyl]-carbamic acid tert-butyl ester for [(S)-1-(1-benzyl-3-hydroxy-azepan-4-ylcarbamoyl)-3-methyl-butyl]-carbamic acid tert butyl ester in step (i) and 4-methylfuran-2-carboxylic acid for benzo[1,3]dioxole-5-carboxylic acid in step (j), the title compound was prepared. MS (ESI): 487 (M+H⁺).

The above specification and Examples fully disclose how to make and use the compounds of the present invention. However, the present invention is not limited to the particular embodiments described hereinabove, but includes all modifications thereof within the scope of the following claims. The various references to journals, patents and other publications which are cited herein comprise the state of the art and are incorporated herein by reference as though fully set forth.

| | | Falcipain | Cruzain | Rhodazain | Leishmania | Leishmania | Schisto | Schisto |
|---|---|---|---|---|---|---|---|---|
| Example | Structure | 2 Ki (nM) | Ki nM | Ki (nM) | L Ki nM | B Ki nM | B 1 Ki nM | B 2 Ki nM |
| 1 | 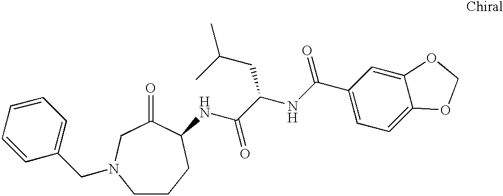 | | 58 | | | | | |
| 2 | 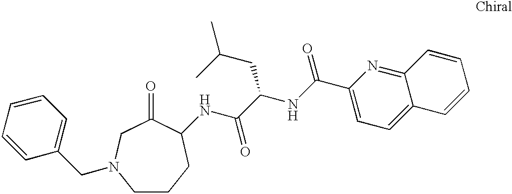 | | 44 | | | | | |
| 3 | 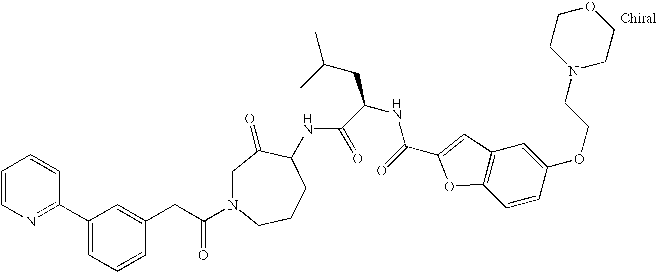 | 3 | | | | | | |
| 4 | 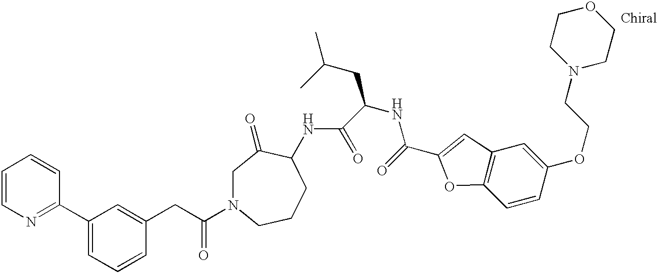 | | | | | | 100 | 320 |
| 5 | 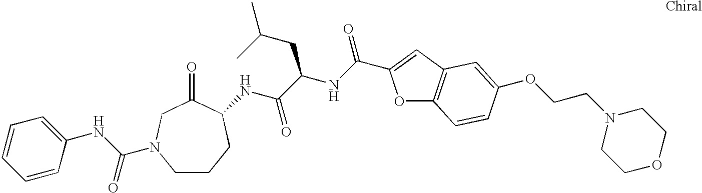 | 34 | | | | | | |
| 6 | 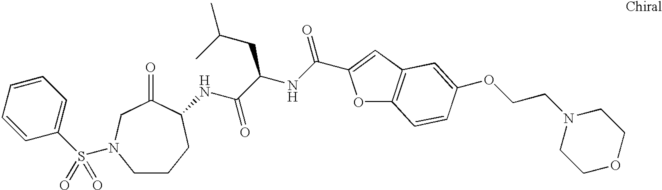 | | 21 | | | | | |
| 7 | 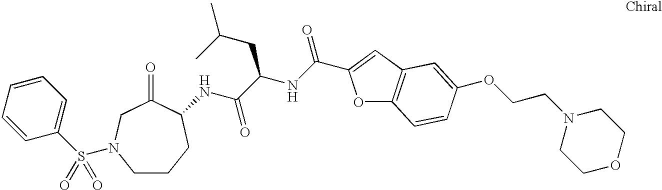 | | 23 | | | | | |
| 8 | 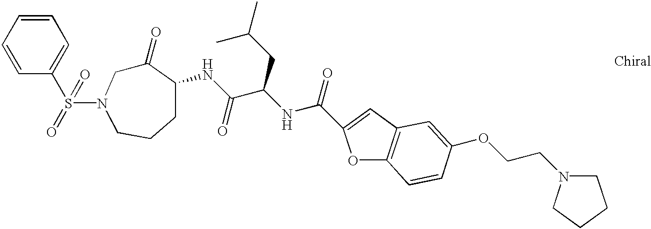 | | 8 | | | | | |
| 9 | 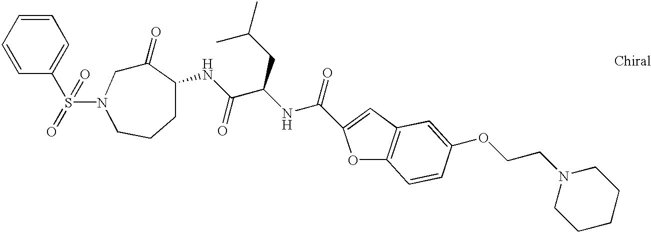 | 65 | | | | | | |
| 10 | 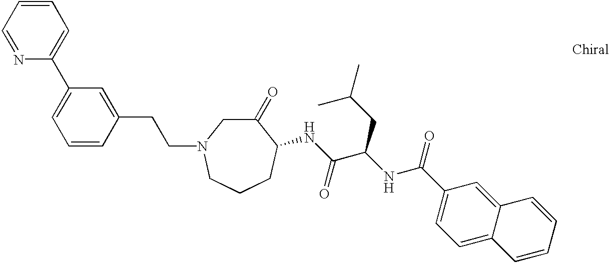 | | | | 82 | | | |
| 11 | 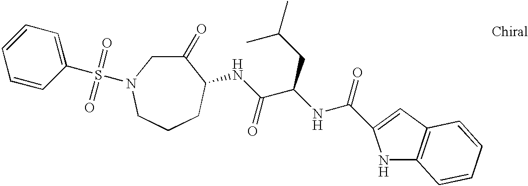 | | 100 | | | | | |
| 12 | 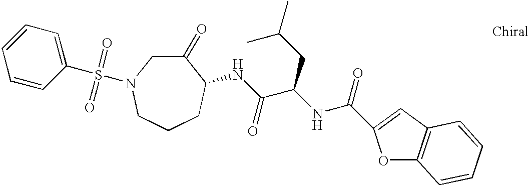 | | 16 | | | | | |
| 13 | 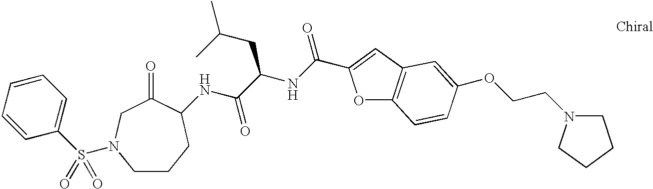 | | 32 | | | | | |
| 14 | 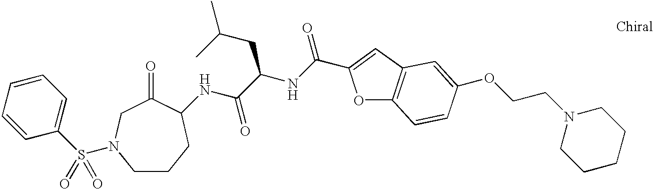 | | 67 | | | | | |
| 15 | 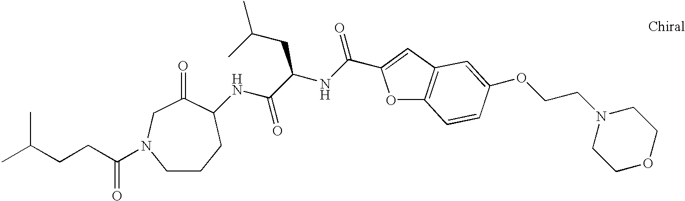 | | 24 | | | | | |
| 16 | 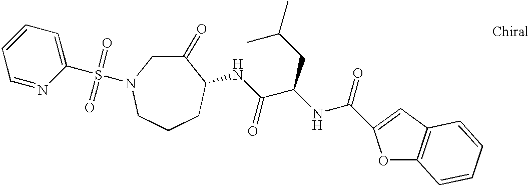 | | 6.1 | 5.5 | | | | |
| 17 | 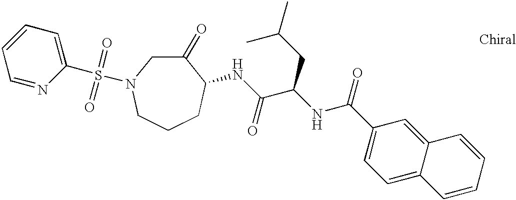 | | | 8.3 | | | | |
| 18 | 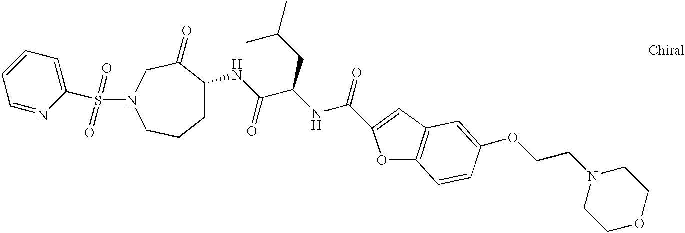 | | 6.3 | | | | | |
| 19 | 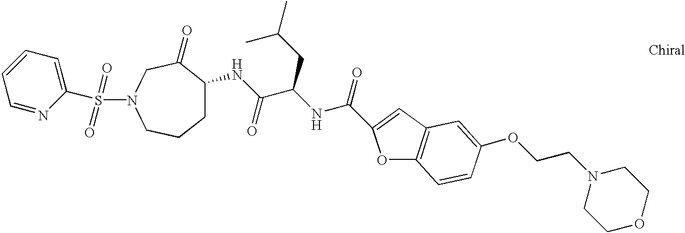 | | 2 | | | | | |
| 20 | 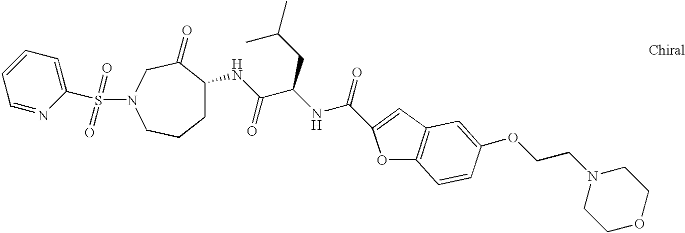 | | 8 | | | | | |
| 21 | 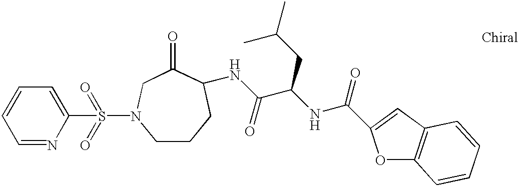 | 10 | 3.8 | 3.2 | | | | |
| 22 | 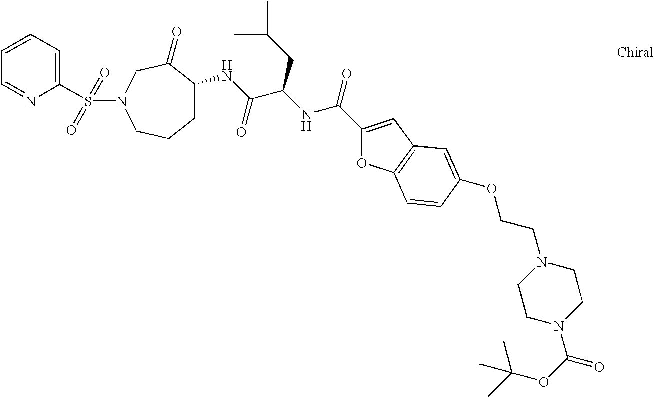 | | 14 | | | | | |
| 23 | 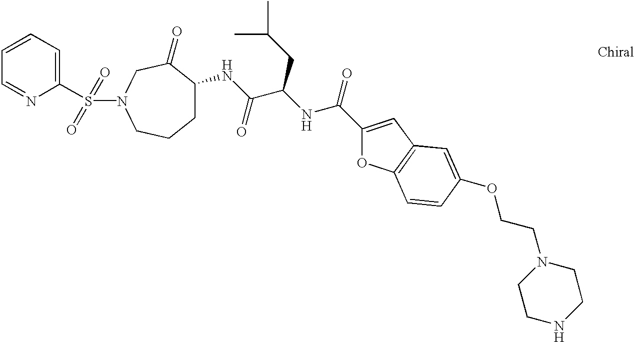 | 14 | | | | | | |
| 24 | 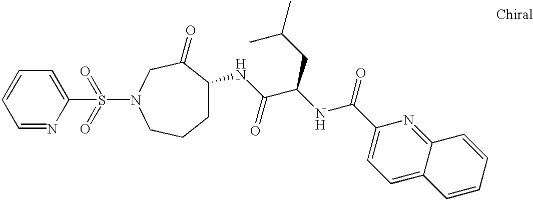 | | 23 | | | | | |
| 25 | 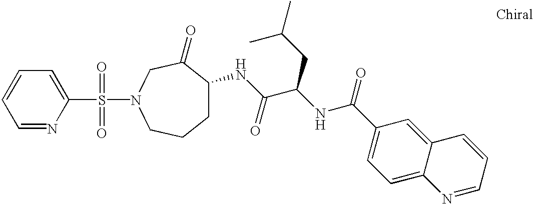 | | 8.3 | 8 | | | | |
| 26 | 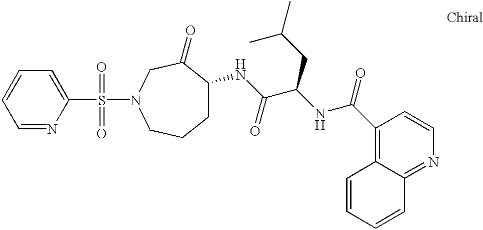 | | 9.4 | 8.1 | | | | |
| 27 | 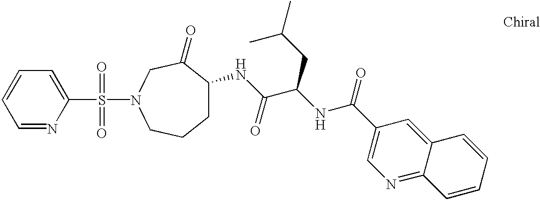 | | | 13 | | | | |
| 28 | 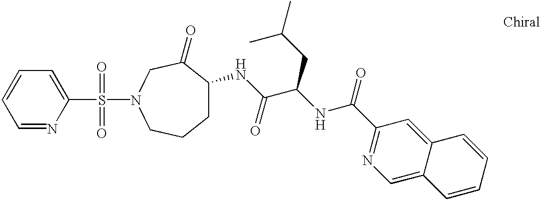 | | 24 | | | | | |
| 29 | 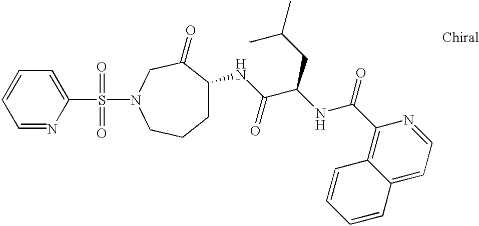 | | 11 | | | | | |
| 30 | 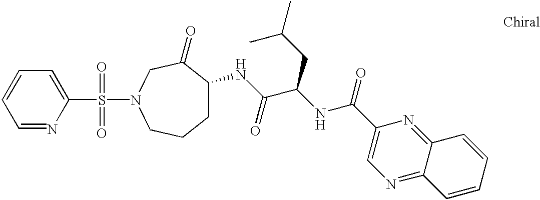 | | 17 | 10 | | | | |
| 31 | 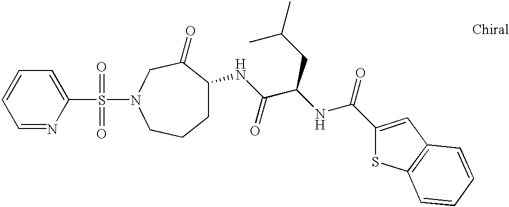 | | 7.2 | 5 | | | | |
| 32 | 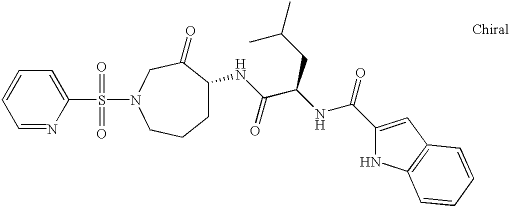 | | 17 | | | | | |
| 33 | 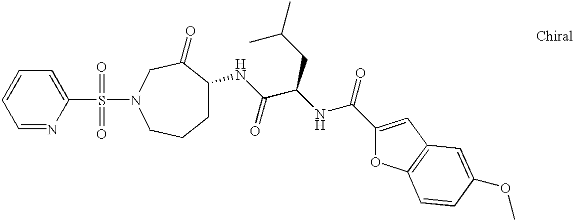 | | 14 | 6 2 | | | | |
| 34 | 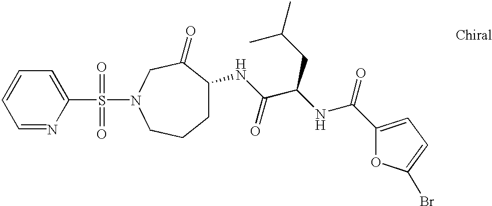 | | 12 | 2.6 | | | | |
| 35 | 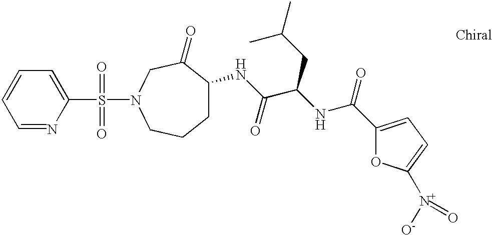 | 96 | | | | | | |
| 36 | 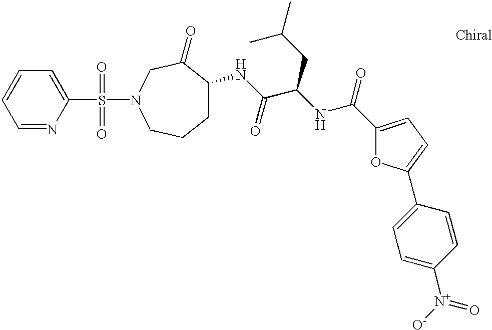 | 8 5 | | 9.1 | | | | |
| 37 | 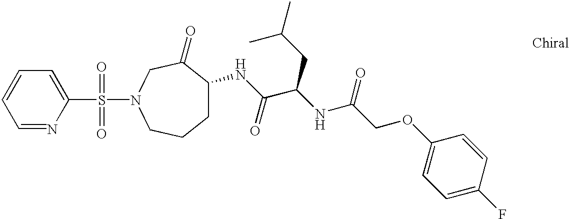 | | 33 | | | | | |
| 38 | 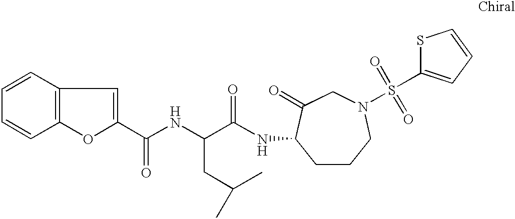 | | 4.5 | | | | | |
| 39 | 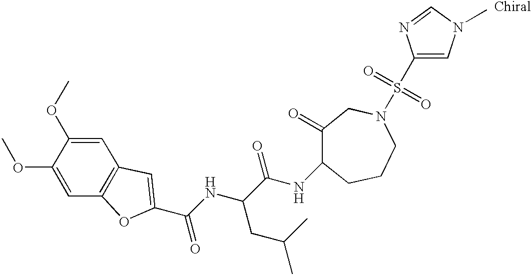 | 3.9 | 7.4 | 2.2 | | | | |
| 40 | 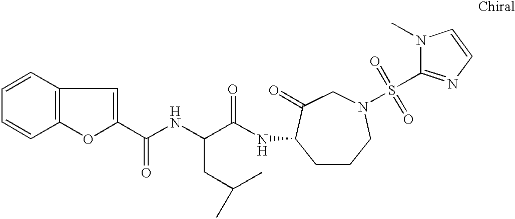 | 4.5 | 2.3 | 2 | | | | |
| 41 | 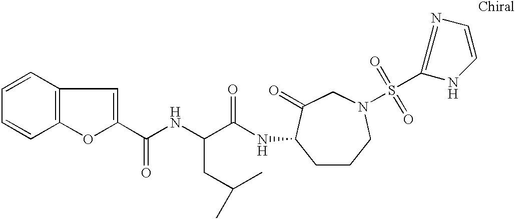 | | | 2.9 | | | | |
| 42 | 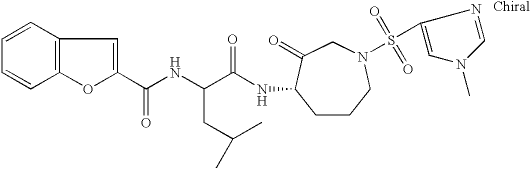 | 5.3 | 2 | 1.8 | | | | |
| 43 | 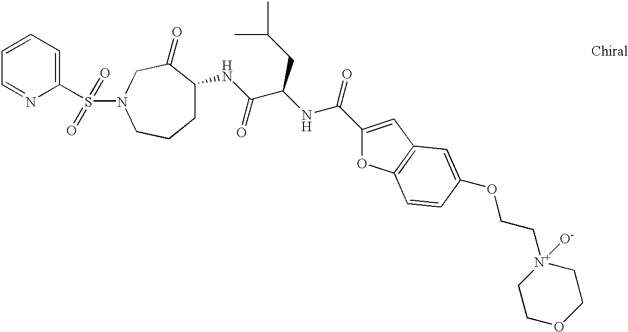 | | 3.6 | 4.9 | | | | |
| 44 | 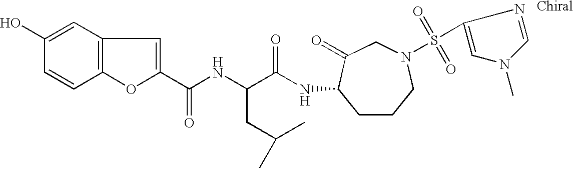 | | 11 | 7.2 | | | | |
| 45 | 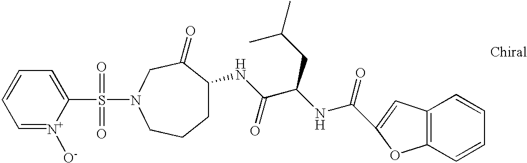 | 5.7 | 4.1 | 2.8 | | | | |
| 46 | 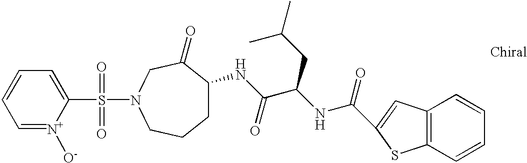 | 5.3 | 6.1 | 3 6 | | | | |
| 47 | 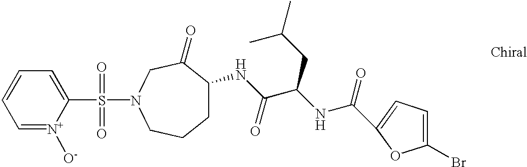 | | 13 | 2.7 | | | | |
| 48 | 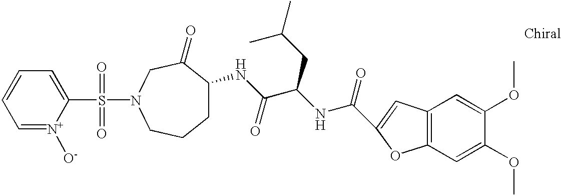 | | 19 | 4.2 | | | | |
| 49 | 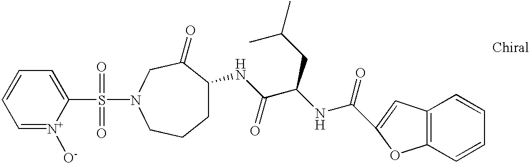 | | 5.6 | 3.1 | | | | |
| 50 | 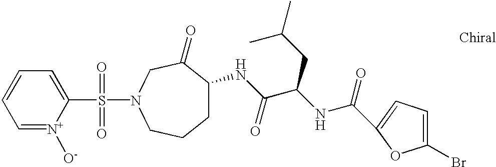 | | 10 | 2.1 | | | | |
| 51 | 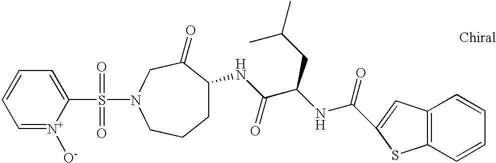 | 5.1 | 6.4 | 3 | | | | |
| 52 | 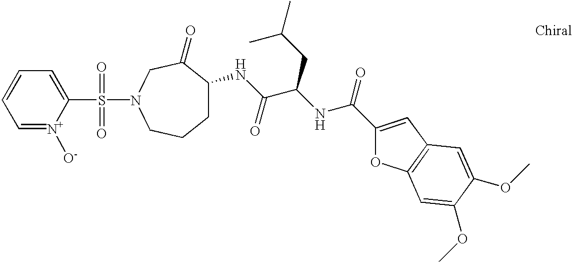 | 6 1 | 48 | 3.3 | | | | |
| 53 | 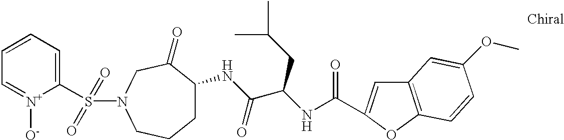 | | 10 | 6 8 | | | | |
| 54 | 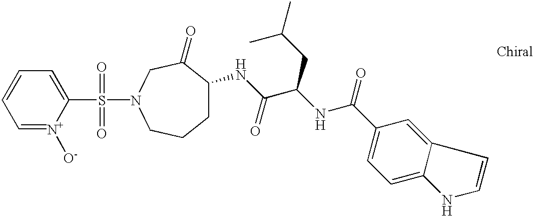 | 7.8 | 5.2 | 4.1 | | | | |
| 55 | 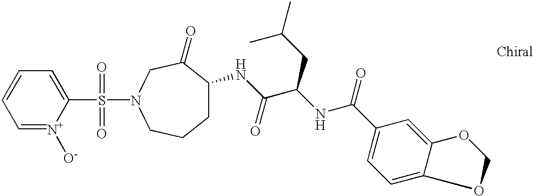 | | 12 | 7.3 | | | | |
| 56 | 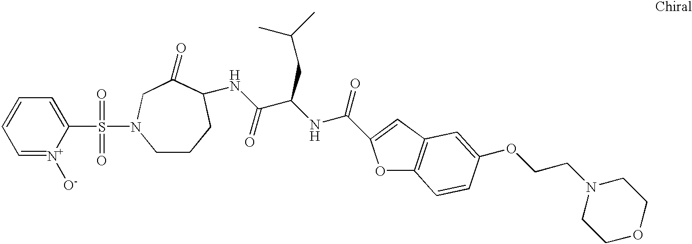 | | 7.8 | | | | | |
| 57 | 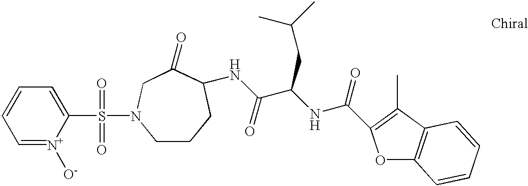 | | 10 | 5.4 | | | | |
| 58 | 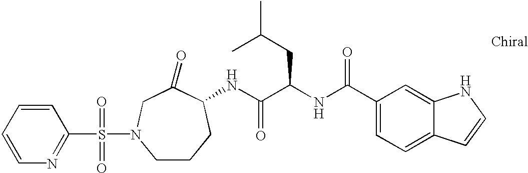 | 7.1 | 3 6 | 3.1 | | | | |
| 59 | 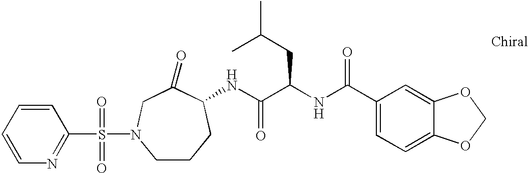 | 3.4 | 3.4 | 2.4 | | | | |
| 60 | 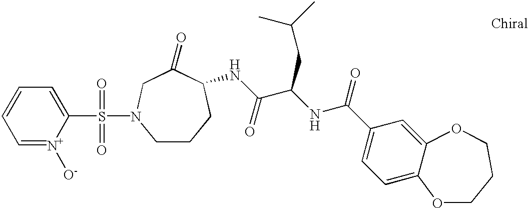 | | 9.8 | | | | | |
| 61 | 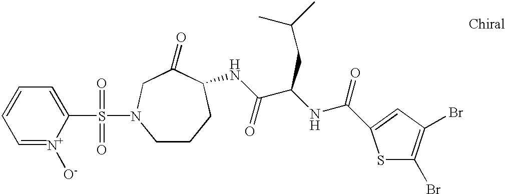 | | 40 | | | | | |
| 62 | 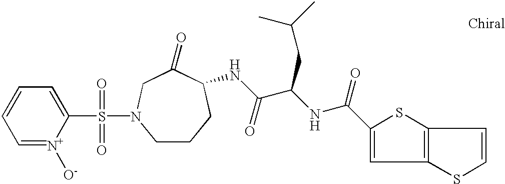 | | 1 1 | 5.3 | | | | |
| 63 | 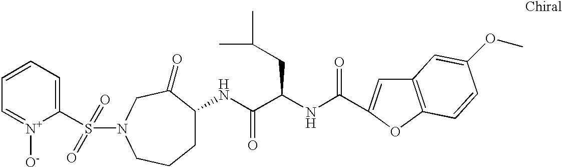 | | 9.8 | 5.9 | | | | |
| 64 | 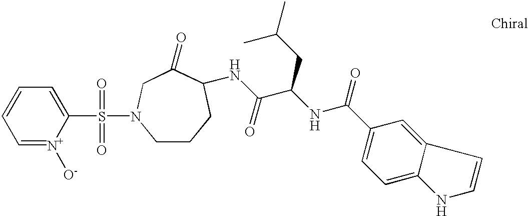 | 6.2 | 4.1 | 2.7 | | | | |
| 65 | 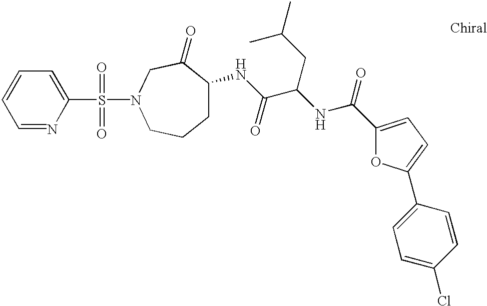 | | | 8.1 | | | | |
| 66 | 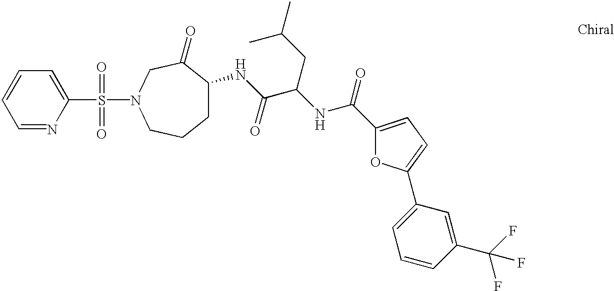 | 10 | | 5.1 | | | | |
| 67 | 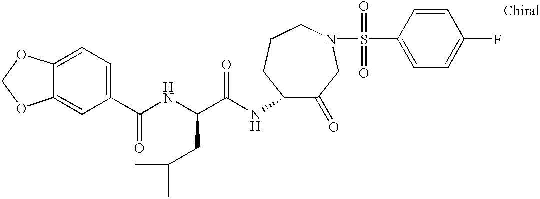 | | 11 | | | | 45 | 330 |
| 68 | 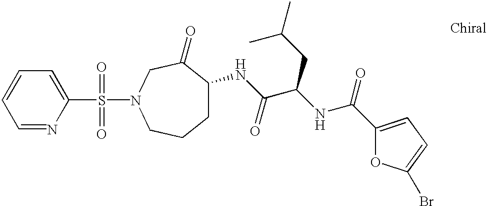 | 3 1 | 3.3 | 0.93 | | | | |
| 69 | 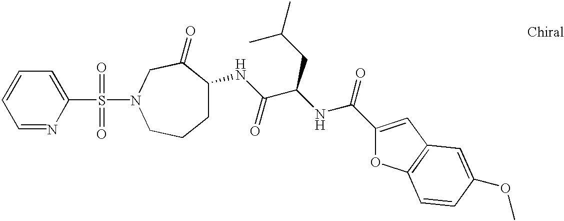 | 5.6 | 3 3 | 2.2 | | | | |
| 70 | 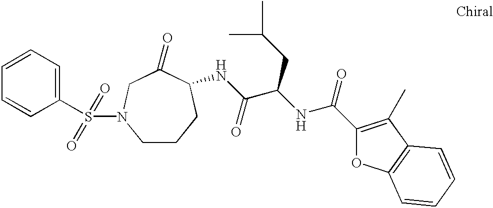 | | 6.6 | 5.5 | | | | |
| 71 | 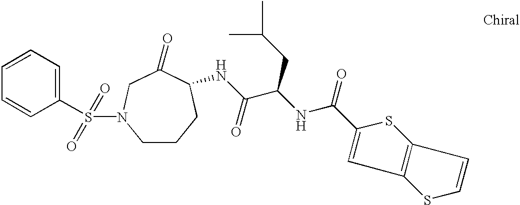 | | 9 | 5.9 | | | | |
| 72 | 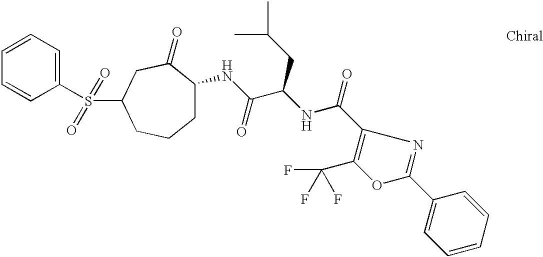 | | | 9.9 | | | | |
| 73 | 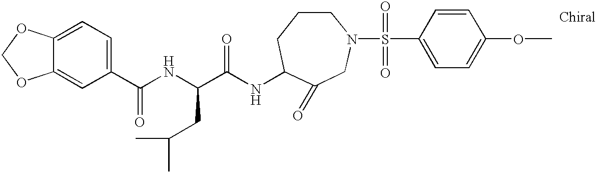 | 5.2 | | | | | | |
| 74 | 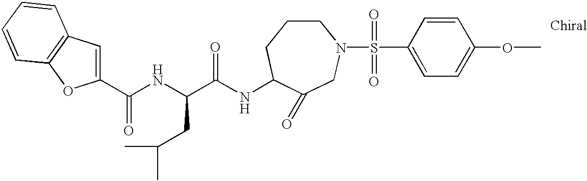 | | 24 | | | | | |
| 75 | 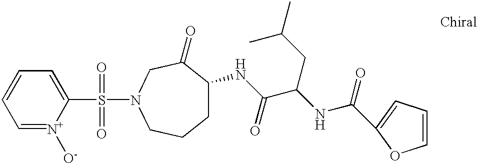 | | | 7.5 | | | | |
| 76 | 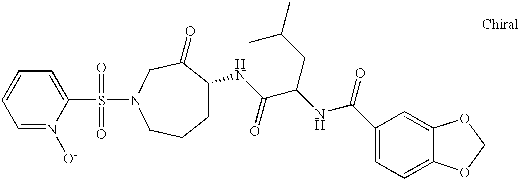 | 7.8 | 14 | 5.4 | | | | |
| 77 | 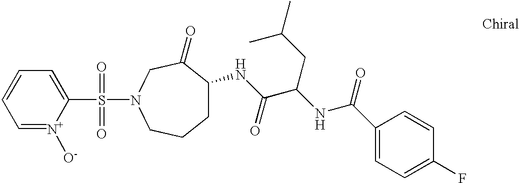 | | 15 | 7.9 | | | | |
| 78 | 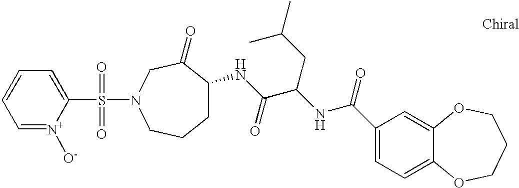 | 7.1 | 4.4 | 4.6 | | | | |
| 79 | 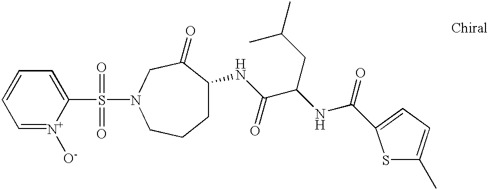 | | 16 | 8 | | | | |
| 80 | 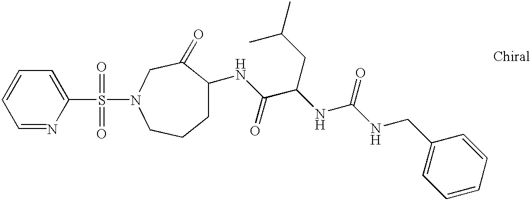 | | 15 | | | | | |
| 81 | 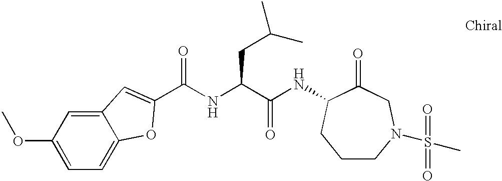 | | | 7.2 | | | | |
| 82 | 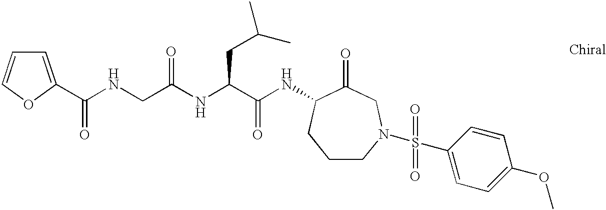 | | 17 | | | | | |
| 83 | 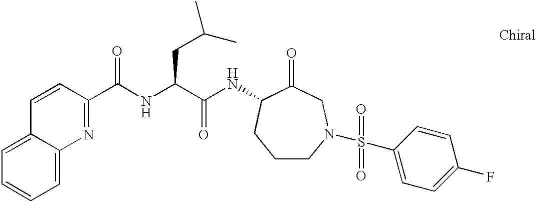 | | | | | | 61 | 230 |
| 84 | 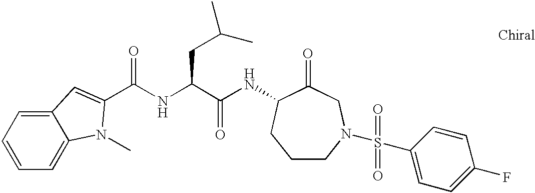 | | | | | | 160 | 1100 |
| 85 | 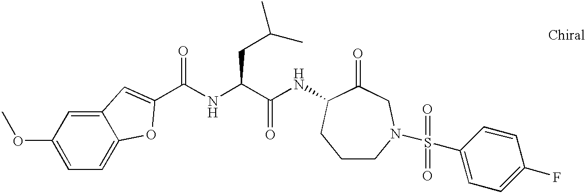 | | | | | | 45 | 130 |
| 86 | 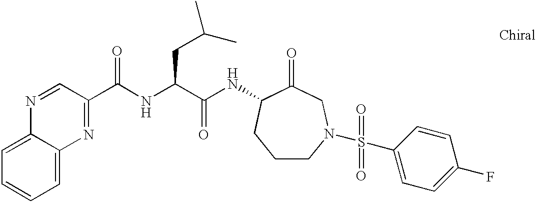 | | 23 | | | | 44 | 260 |
| 87 | 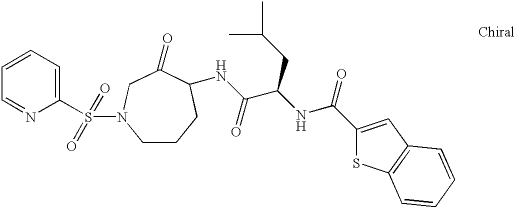 | 42 | 3 | 1.9 | | | 44 | 260 |
| 88 | 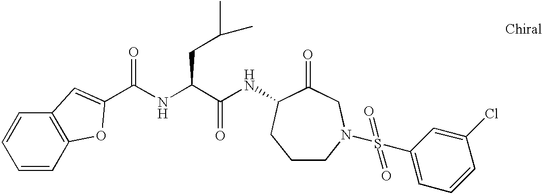 | | | | | | 77 | 140 |
| 89 | 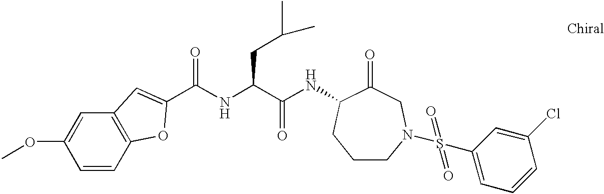 | | 11 | | | 24 | 34 | 130 |
| 90 | 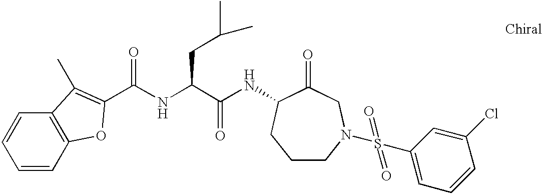 | | | | | 3.3 | 48 | 71 |
| 91 | 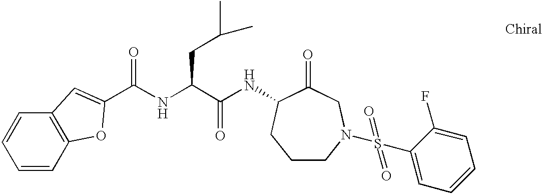 | | 6.2 | | | | | |
| 92 | 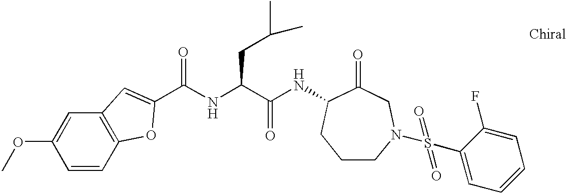 | | 49 | 6.3 | | | | |
| 93 | 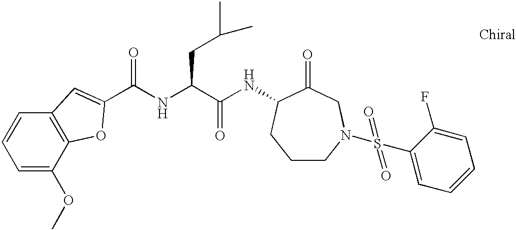 | 7 | 11 | 11 | | | | |
| 94 | 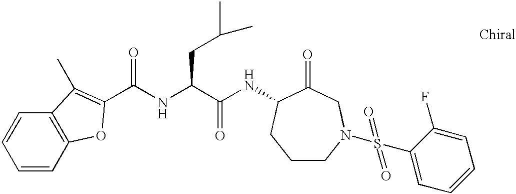 | | 8.6 | 9.4 | | | | |
| 95 | 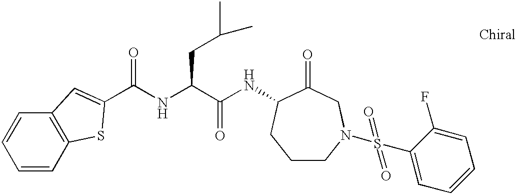 | | 11 | | | | | |
| 96 | 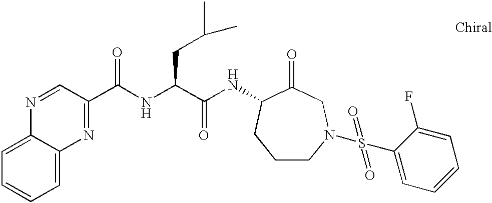 | | 8.7 | | | | | |
| 97 | 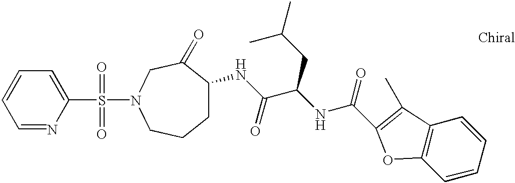 | | 4 | 2.7 | | | | |
| 98 | 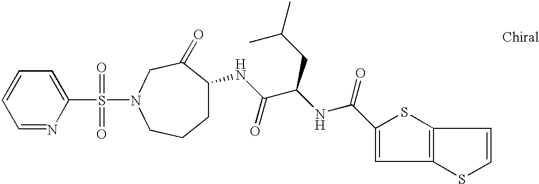 | 5 8 | 5.2 | 3.9 | | | | |
| 99 | 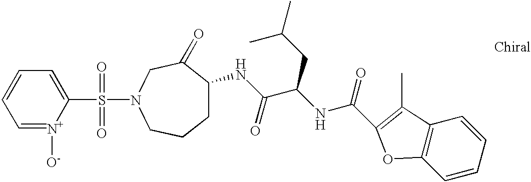 | | 7.8 | 5.1 | | | | |
| 100 | 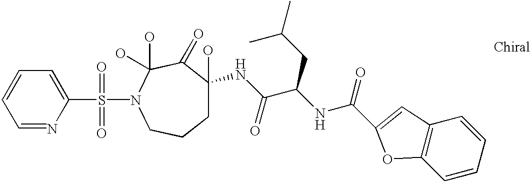 | 6.4 | 2 5 | 2.5 | | | | |
| 101 | 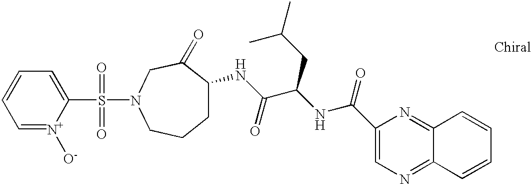 | | | 11 | | | | |
| 102 | 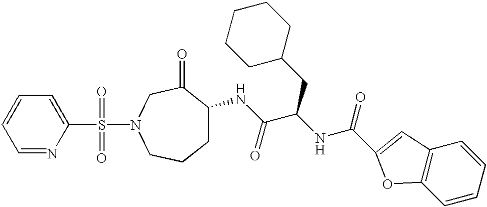 | | | 3.7 | | | | |
| 103 | 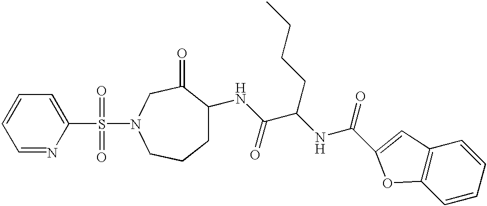 | | 60 | | | | | |
| 104 | 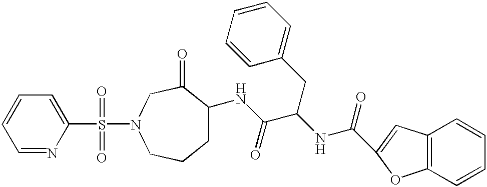 | | 13 | 3.2 | | | 76 | 270 |
| 105 | 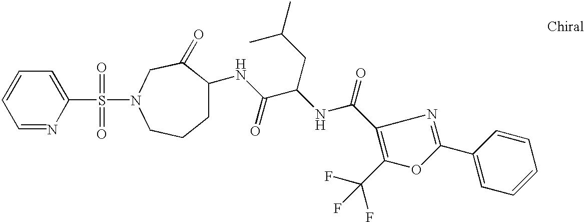 | 84 | 13 | 5.3 | | | | |
| 106 | 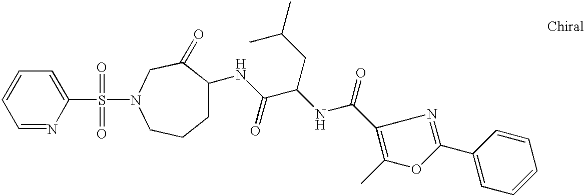 | | 16 | 5.8 | | | | |
| 107 | 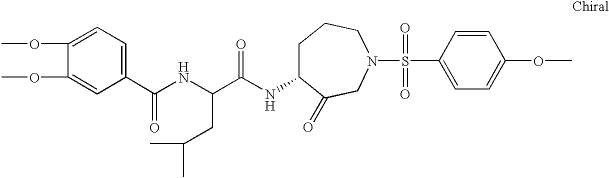 | 5.9 | | | | | | |
| 108 | 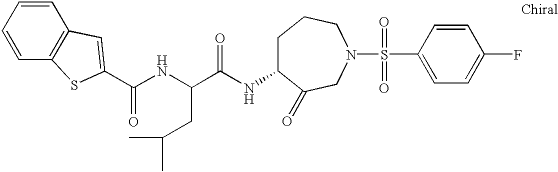 | | 20 | | | 9.4 | 34 | 110 |
| 109 | 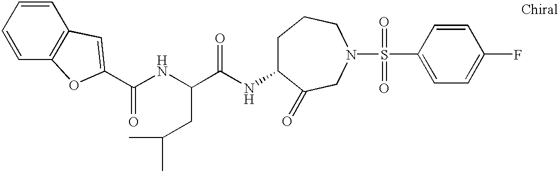 | | | | | | 31 | 120 |
| 110 | 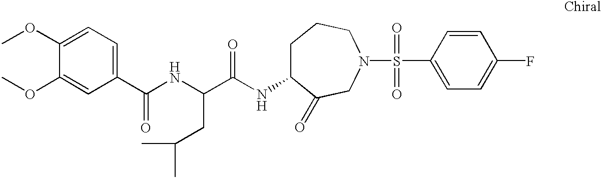 | 96 | 7.4 | 8 3 | | | 38 | 280 |
| 111 | 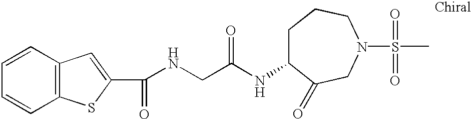 | | 18 | | | | | |
| 112 | 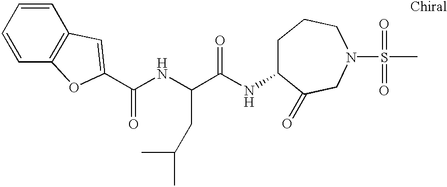 | | | 8.5 | | | | |
| 113 | 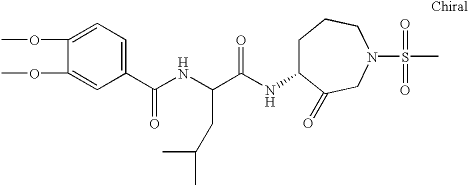 | | | 7.8 | | | | |
| 114 | 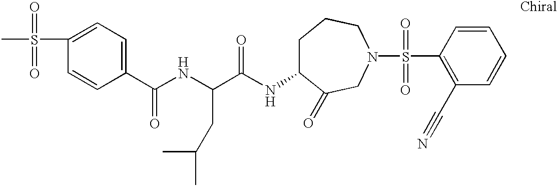 | | 14 | | | | | |
| 115 | 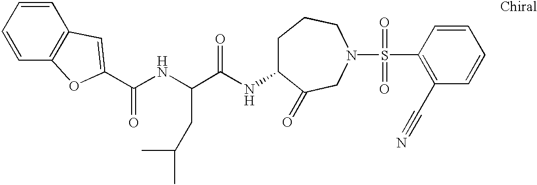 | | 14 | | | | | |
| 116 | 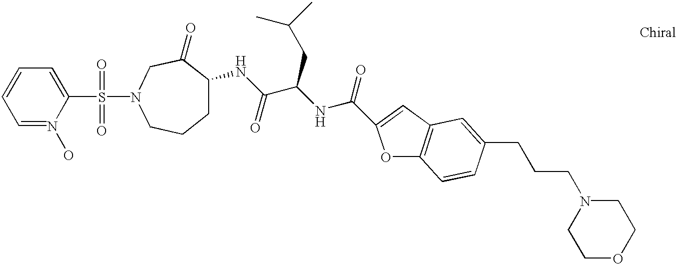 | | 4.4 | 3.2 | | | | |
| 117 | 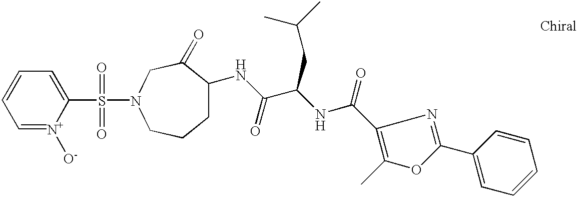 | 6.8 | 14 | 5 2 | | | | |
| 118 | 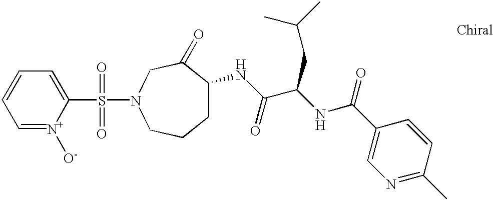 | | 18 | | | | | |
| 119 | 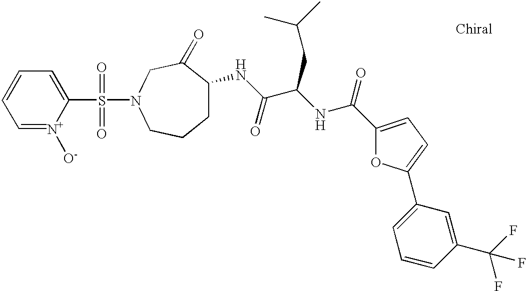 | 8.1 | | 5.1 | | | | |
| 120 | 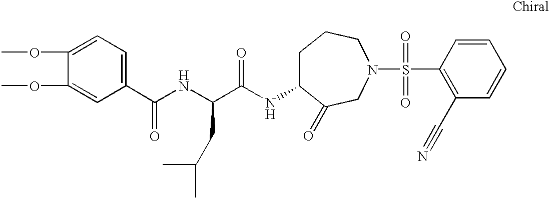 | | 9.9 | 11 | | | | |
| 121 | 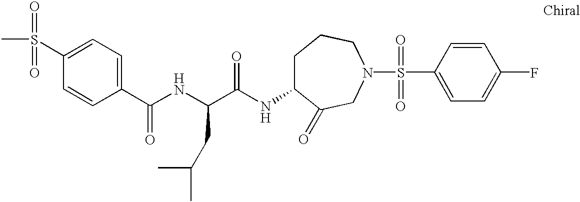 | | 11 | | | | | |
| 122 | 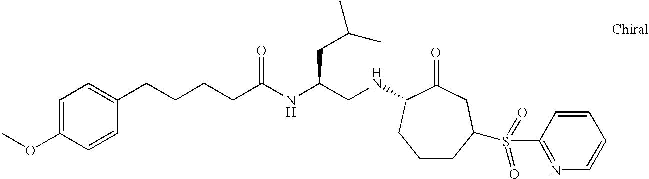 | | | 8.8 | | | | |
| 123 | 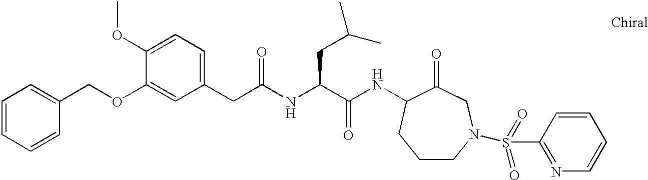 | | | 31 | | | | |
| 124 | 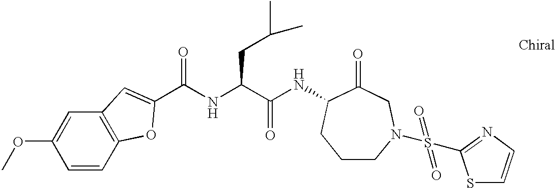 | 4.9 | 2 | 2.2 | | | | |
| 125 | 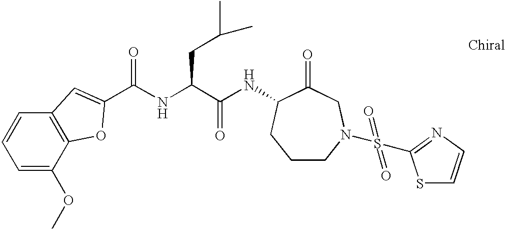 | 1 9 | 4 | 4.1 | | | | |
| 126 | 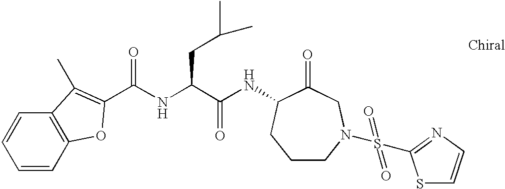 | | 4.3 | 3.8 | | | | |
| 127 | 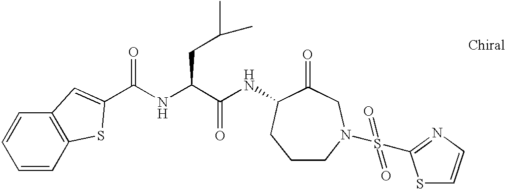 | 8.1 | 2.8 | 4.7 | | | | |
| 128 | 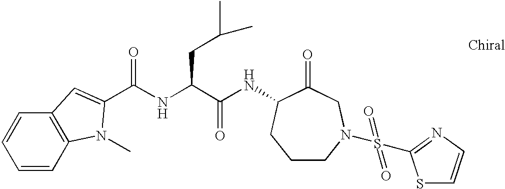 | | 17 | 8.2 | | | | |
| 129 | 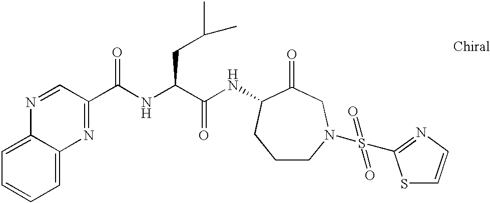 | | | 5.6 | | | | |
| 130 | 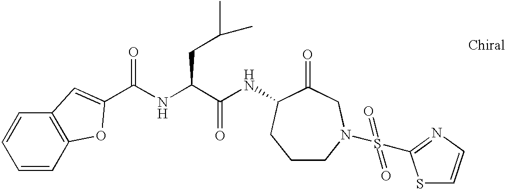 | | 2.5 | 2.6 | | | | |
| 131 | 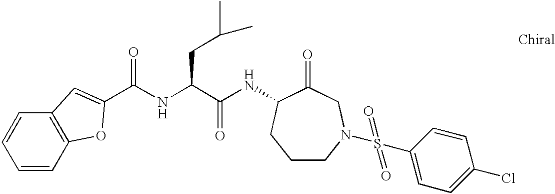 | | | | | 11 | 75 | 300 |
| 132 | 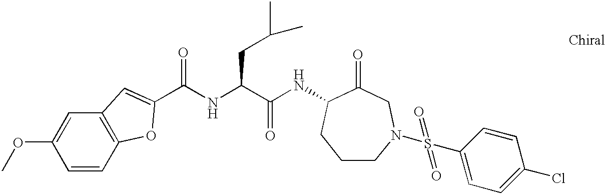 | 6 8 | | | | 8.1 | 150 | 880 |
| 133 | 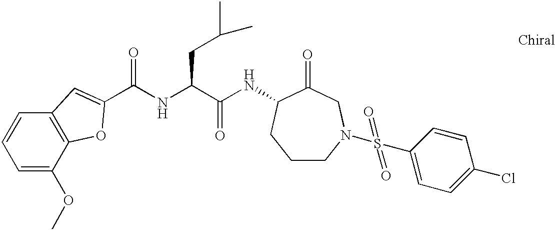 | 3.4 | | | | | | |
| 134 | 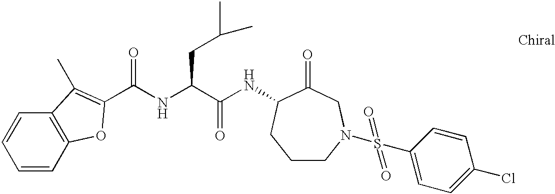 | | | | | | 78 | 360 |
| 135 | 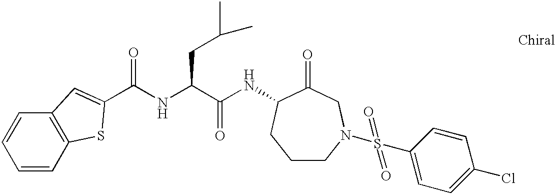 | | | | | 8 | 75 | 270 |
| 136 | 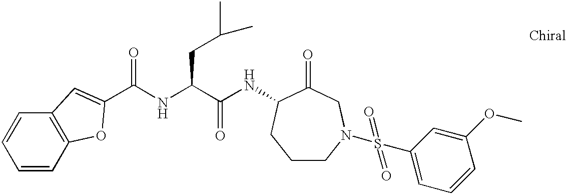 | | 12 | | | | 41 | 170 |
| 137 | 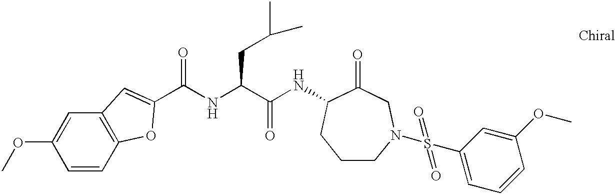 | | 19 | | | | 67 | 380 |
| 138 | 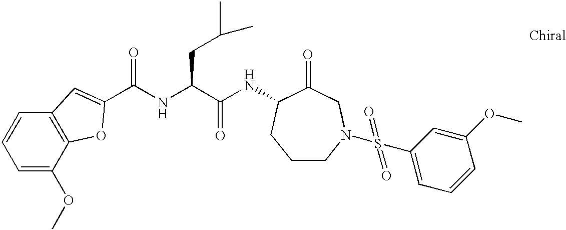 | 5.6 | | | | | | |
| 139 | 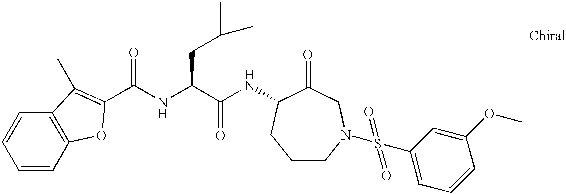 | | | | | | 64 | 230 |
| 140 | 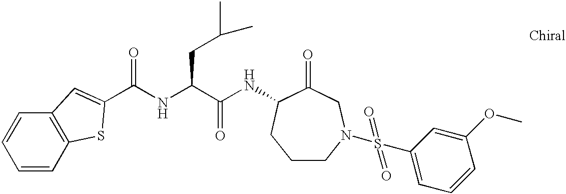 | | | | | | 68 | 280 |
| 141 | 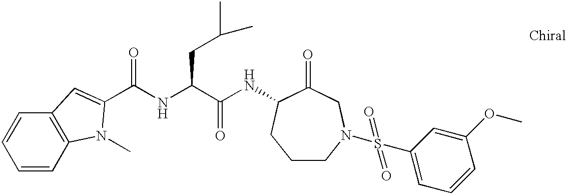 | | | | | | 50 | 400 |
| 142 | 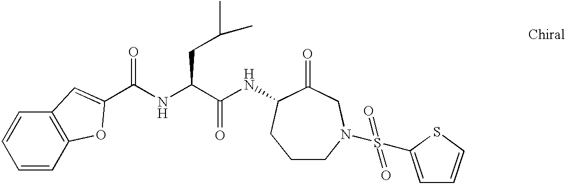 | | 2.4 | 5.2 | | | | |
| 143 | 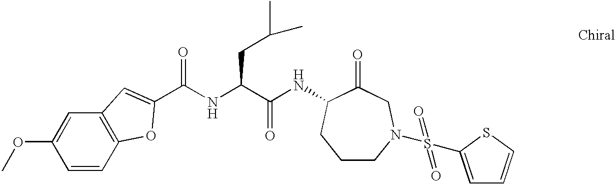 | | 5.7 | 6.5 | | | 55 | 110 |
| 144 | 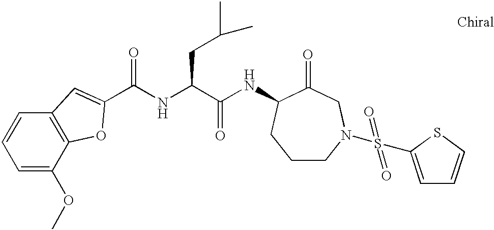 | 6.2 | 5.5 | 8.6 | | | | |
| 145 | 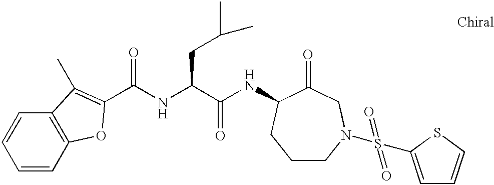 | | 6.2 | 9.4 | | | | |
| 146 | 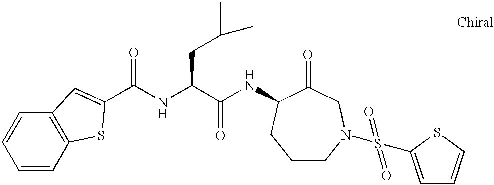 | | 6.7 | | | | | |
| 147 | 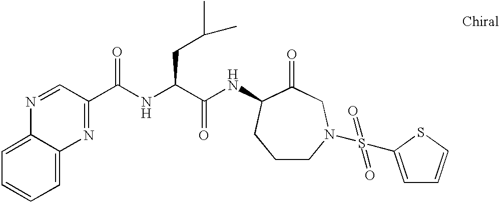 | | 7.5 | | | | | |
| 148 | 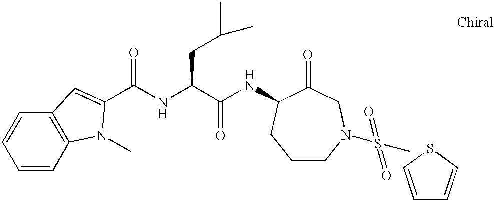 | | 8.2 | 23 | | | | |
| 149 | 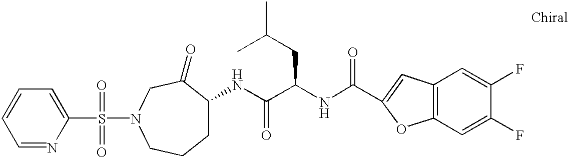 | | 9.1 | 6.3 | | | | |
| 150 | 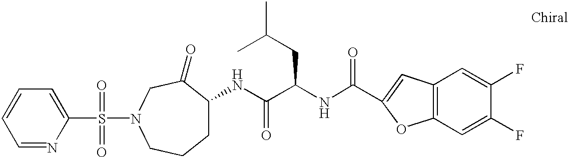 | | 5.4 | 3.7 | | | | |
| 151 | 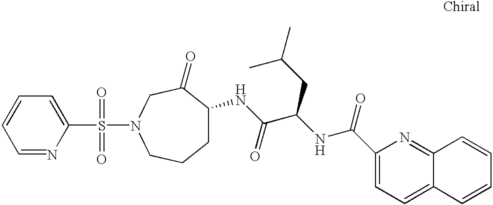 | | 7.1 | 6.1 | | | | |
| 152 | 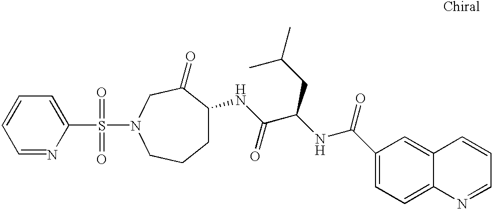 | 3.1 | 2 | 1.6 | | | | |
| 153 | 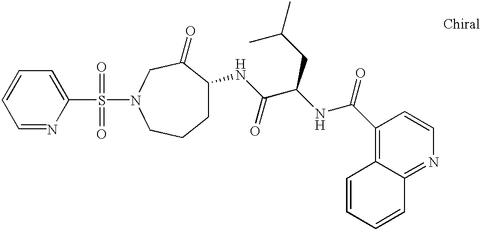 | 4.5 | 2.4 | 1.6 | | | | |
| 154 | 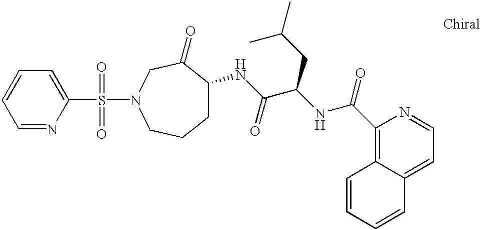 | | 4.4 | 4.4 | | | | |
| 155 | 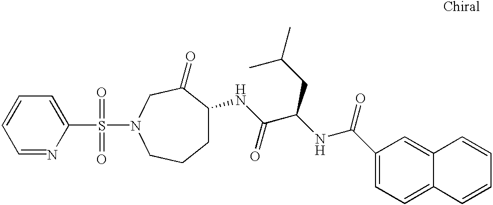 | 6.1 | 4.1 | 3 | | | | |
| 156 | 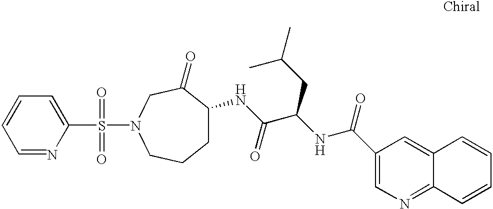 | 6.2 | 4.1 | 2.6 | | | | |
| 157 | 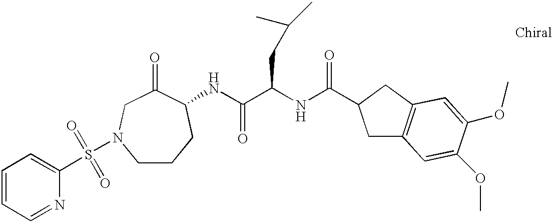 | 3.5 | 8.3 | 3.8 | | | | |
| 158 | 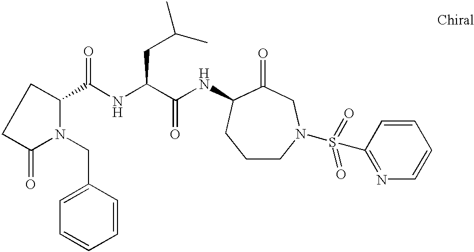 | | 6.8 | | | | | |
| 159 | 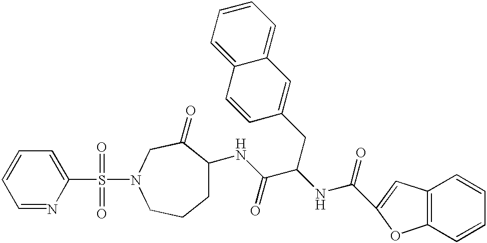 | | | 3.4 | 4.5 | 1.4 | 47 | 2 8 |
| 160 | 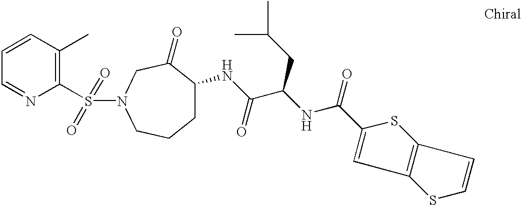 | | 7.4 | 6.7 | | | | |
| 161 | 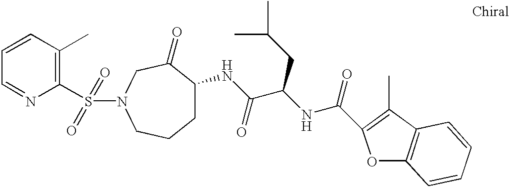 | | 7.8 | 6.4 | | | | |
| 162 | 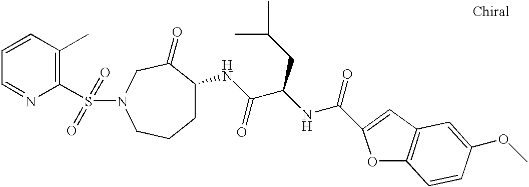 | 6.1 | 2.7 | 2.5 | | | | |
| 163 | 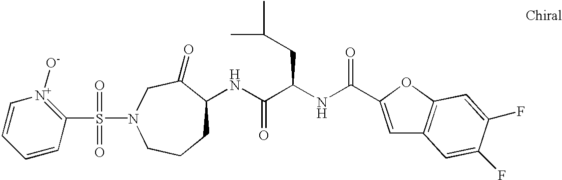 | | 13 | | | | | |
| 164 | 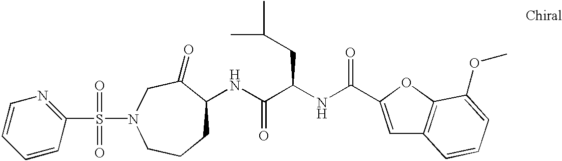 | 2.2 | 4.2 | 5.1 | | | | |
| 165 | 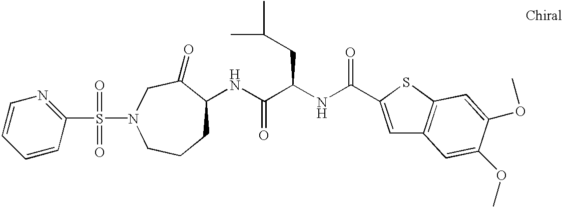 | 2.5 | 5.5 | 2.1 | | | | |
| 166 | 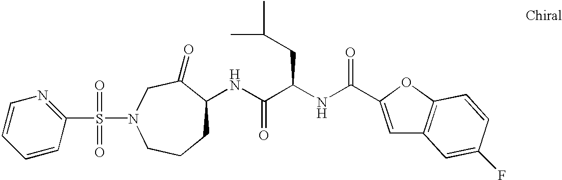 | | | 6 | | | | |
| 167 | 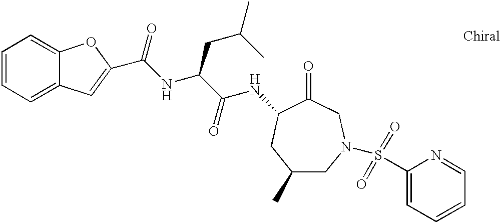 | | 3.5 | 4 | | | | |
| 168 | 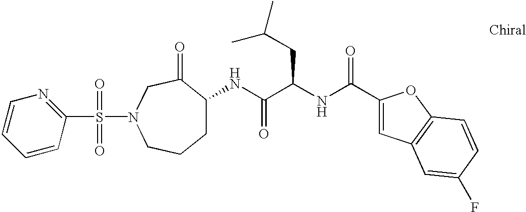 | | 3 4 | 2.7 | | | | |
| 169 | 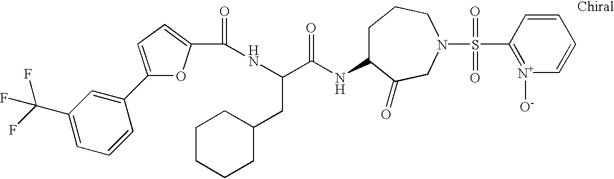 | 43 | | | | | | |
| 170 | 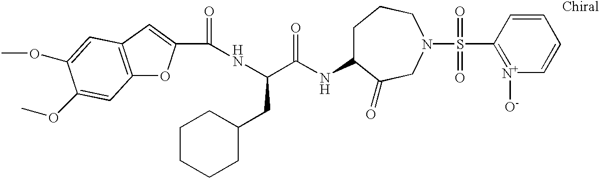 | | | 4.7 | | | 49 | 82 |
| 171 | 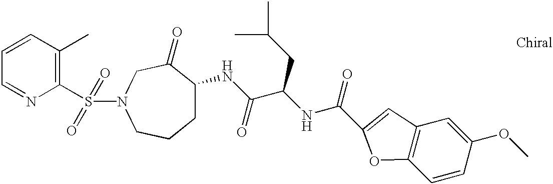 | 4.9 | 2.5 | 3.2 | | | | |
| 172 | 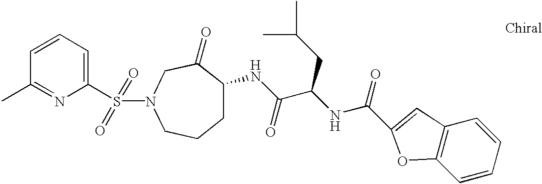 | | 7.4 | | | | | |
| 173 | 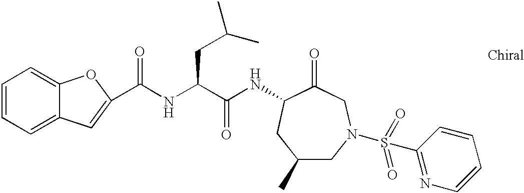 | | 2 | 2.2 | | | | |
| 174 | 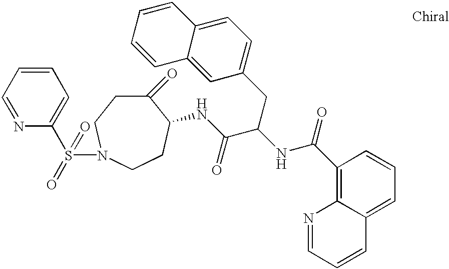 | | | 4.7 | | 1.7 | 27 | 8.3 |
| 175 | 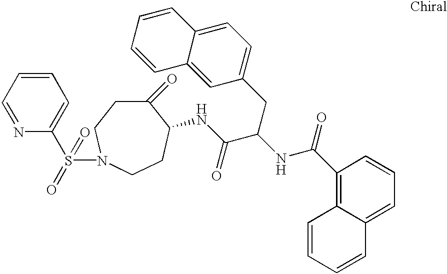 | | | 5.6 | | 1.7 | 37 | 21 |
| 176 | 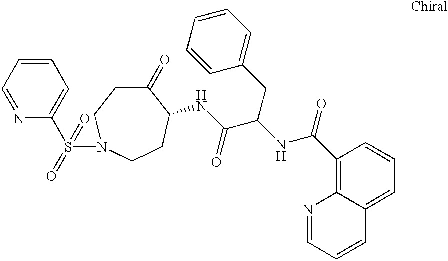 | | | 5.9 | | | | |
| 177 | 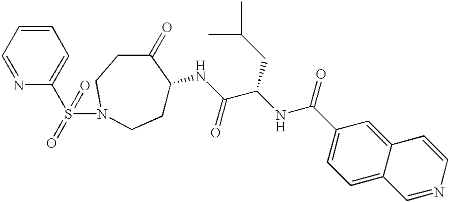 | | | 9 | | | | |
| 178 | 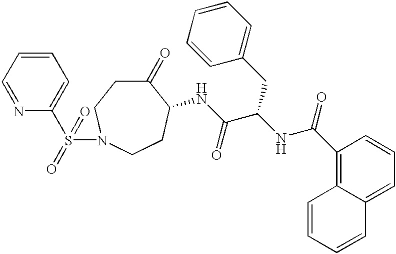 | | | 5.7 | | | | |
| 179 | 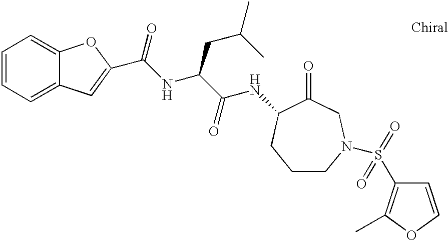 | | 2.2 | 5.1 | | | 11 | 110 |
| 180 | 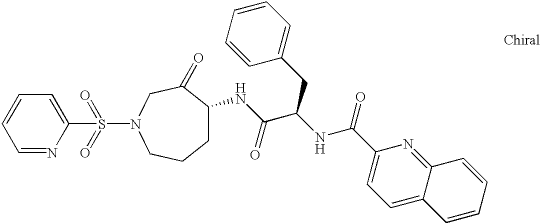 | | | 9.3 | | | | |
| 181 | 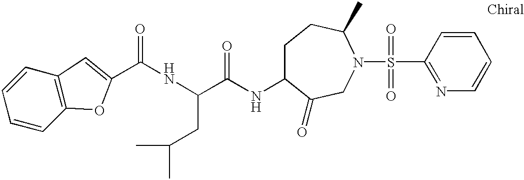 | | 15 | | | | | |
| 182 | 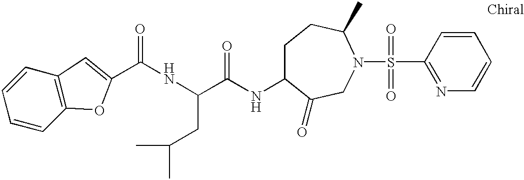 | | | 1.1 | 6.6 | 4.3 | 61 | 45 |
| 183 | 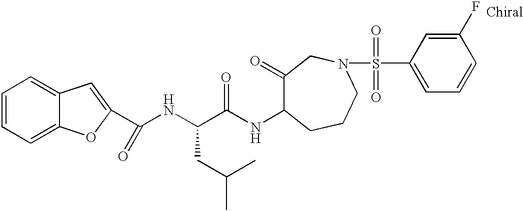 | | 7.1 | 6.5 | | | | |
| 184 | 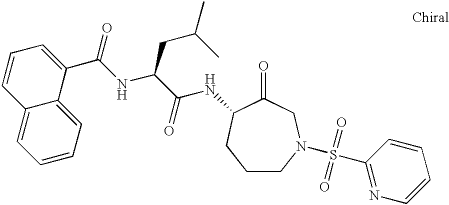 | 4 7 | 2.1 | 1.8 | | | | |
| 185 | 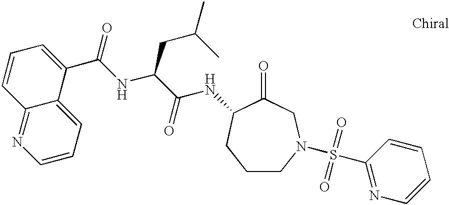 | 4.4 | 1.8 | 2.1 | | | | |
| 186 | 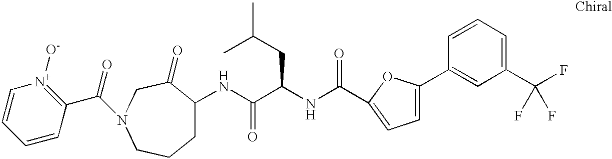 | | | 11 | | | | |
| 187 | 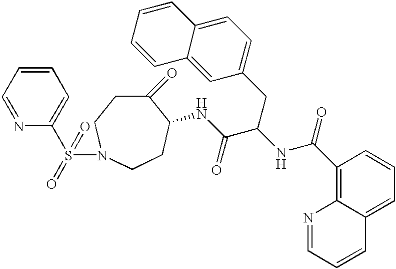 | | 6.8 | 2.5 | 5.1 | 2.1 | 8.4 | 5 1 |
| 188 | 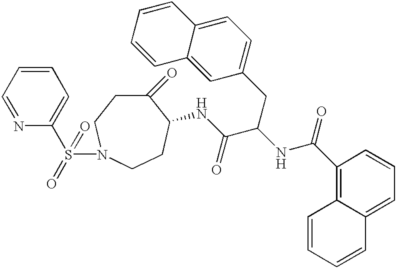 | | | 4 | 4.7 | 2.8 | 24 | 22 |
| 189 | 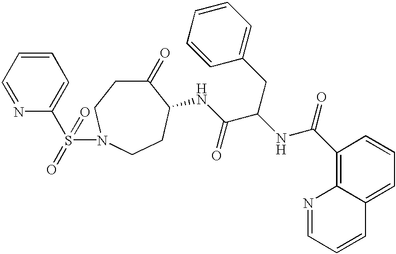 | | | 3.1 | | 3.9 | | |
| 190 | 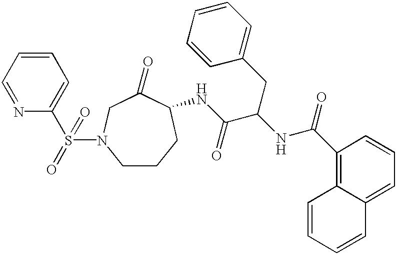 | | 8.4 | 2.5 | 6.4 | 4 6 | 38 | 68 |
| 191 | 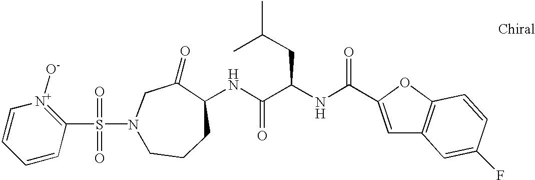 | | 12 | 8.3 | | | | |
| 192 | 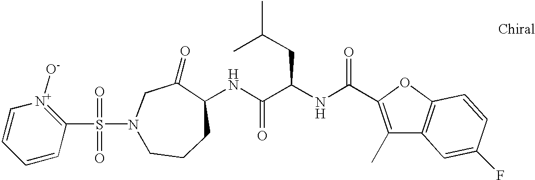 | | 14 | | | | | |
| 193 | 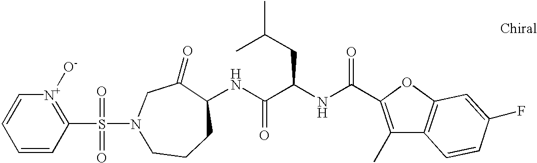 | | | 8.1 | | | | |
| 194 | 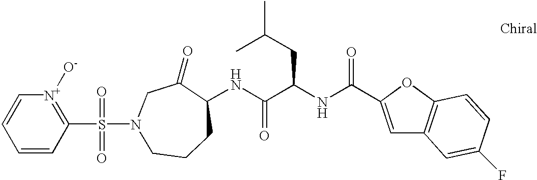 | | 8.7 | 6.7 | | | | |
| 195 | 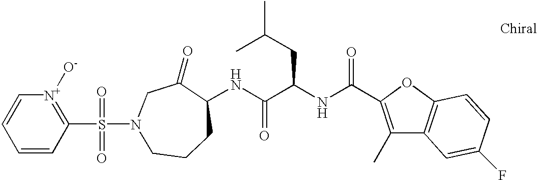 | | 9.4 | 6.6 | | | | |
| 196 | 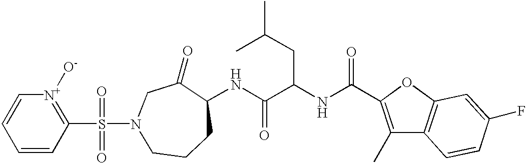 | | 11 | 6.8 | | | | |
| 197 | 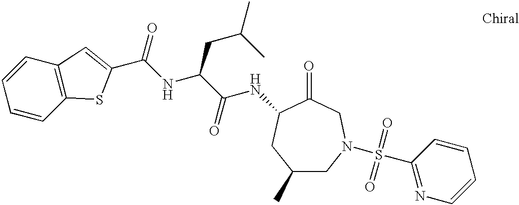 | 4.1 | 3.1 | 2.3 | 8.4 | | | |
| 198 | 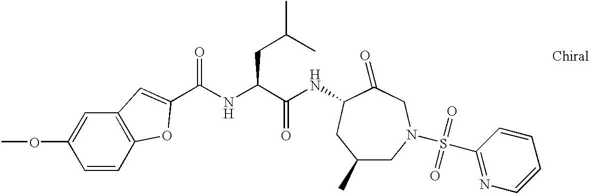 | 3.6 | 1.9 | 1.6 | | | | |
| 199 | 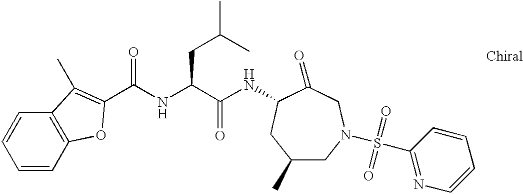 | 5.6 | 2.3 | 2 | | | | |
| 200 | 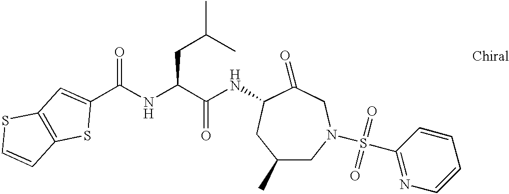 | 4.1 | 2.7 | 2.3 | | 4.7 | 63 | 65 |
| 201 | 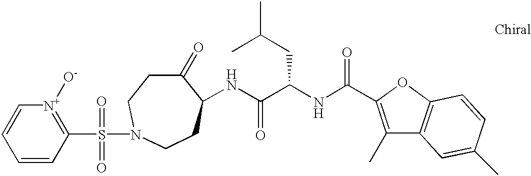 | | 10 | 7.6 | | | | |
| 202 | 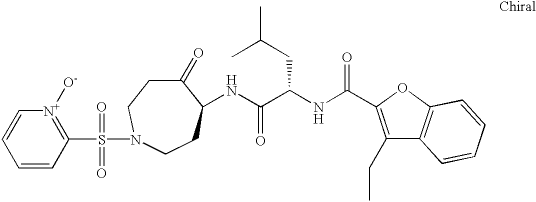 | | | 8.7 | | | | |
| 203 | 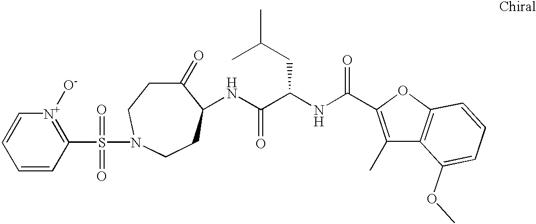 | | 20 | | | | | |
| 204 | 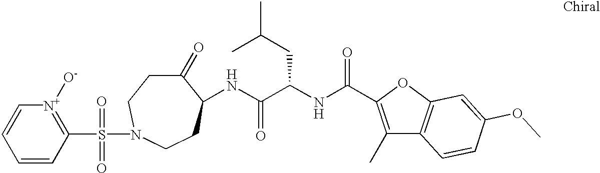 | | | 4.5 | | | | |
| 205 | 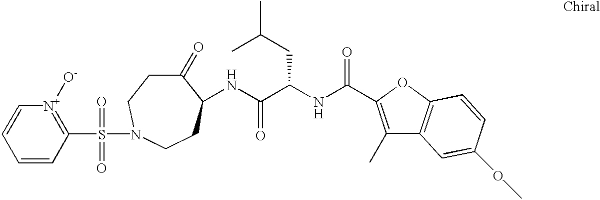 | | 5.2 | 3.2 | | | | |
| 206 | 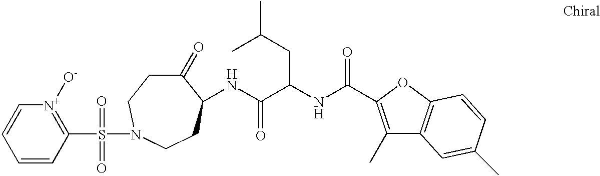 | | 6.6 | 4.9 | | | | |
| 207 | 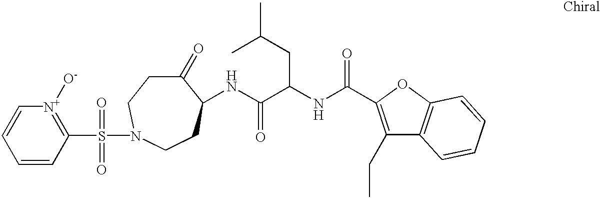 | | 10 | 5.4 | | | | |
| 208 | 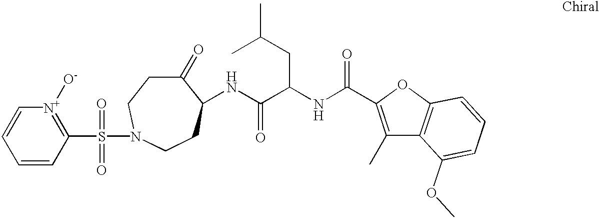 | | 10 | 4.2 | | | | |
| 209 | 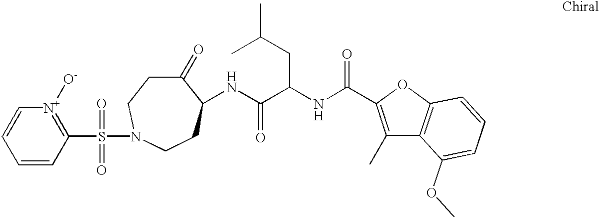 | | 8.9 | 4.1 | | | | |
| 210 | 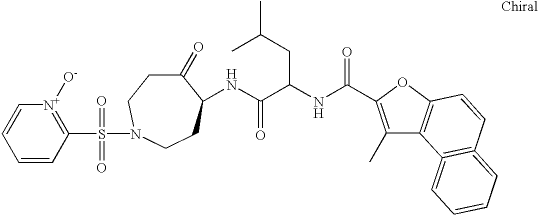 | | 6.6 | | | | | |
| 211 | 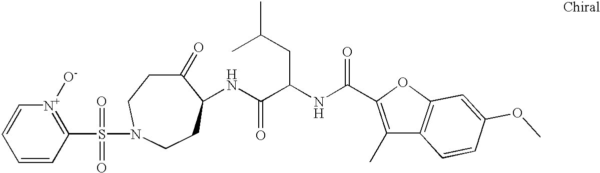 | | 10 | 2.8 | | | | |
| 212 | 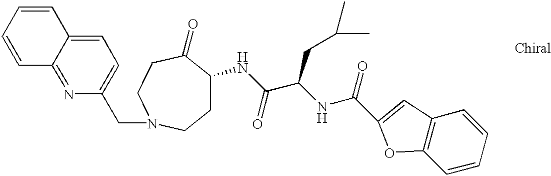 | | 7.7 | | | 1.7 | | |
| 213 | 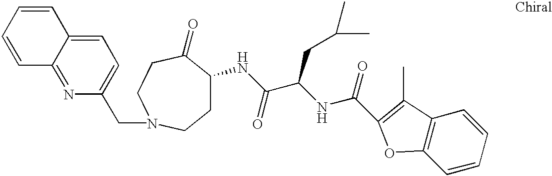 | | 7.1 | | | 0.77 | | |
| 214 | 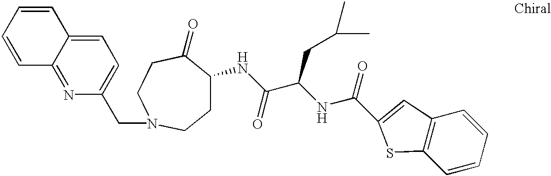 | | 7.4 | | | 1 | | |
| 215 | 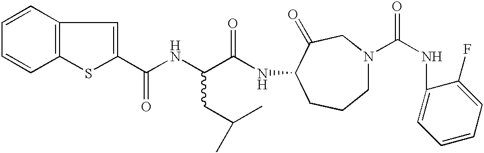 | | 6.4 | 3.6 | | | | |
| 216 | 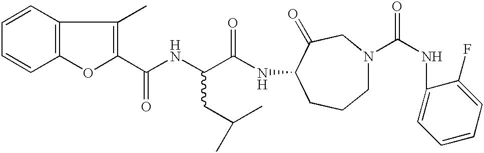 | | 6.9 | 3.4 | | | | |
| 217 | 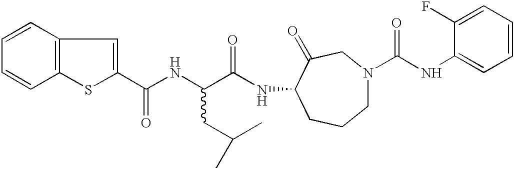 | | 11 | 6.7 | | | | |
| 218 | 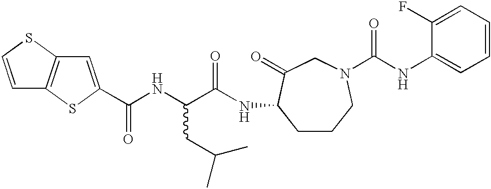 | 3.1 | 5.4 | 2.3 | 3 | | | |
| 219 | 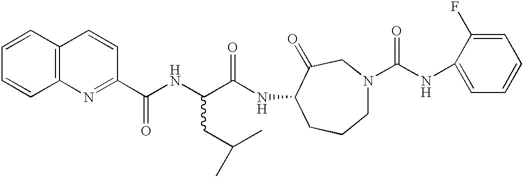 | | 8.4 | 5.5 | | | | |
| 220 | 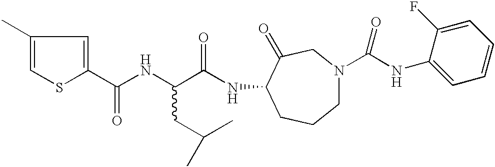 | 3.9 | 8.7 | 4.2 | | | | |
| 221 | 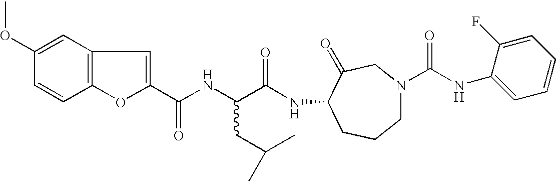 | 2 | 3 | 1.2 | 0.45 | | | |
| 222 | 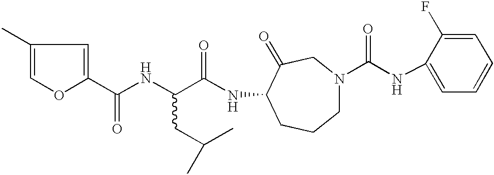 | | | 4.9 | | | | |

We claim:

1. A method of inhibiting a cysteine protease which comprises administering to a patient in need thereof an effective amount of a compound of Formula I: 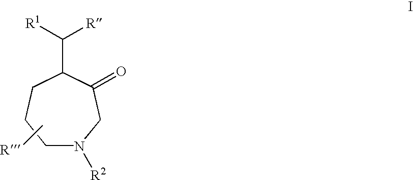
Wherein:
R¹ is selected from the group consisting of: 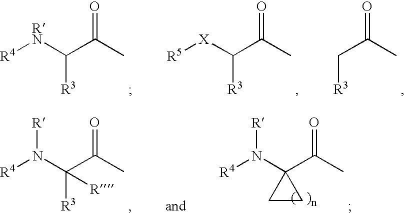
R² is selected from the group consisting of: H, C₁₋₆alkyl, C₃₋₆cycloalkyl-C₀₋₆alkyl, Ar—C₀₋₆alkyl, Het-C₀₋₆alkyl, R⁹C(O)—, R⁹C(S)—, R⁹SO₂—, R⁹OC(O)—, R⁹R¹¹NC(O)—, R⁹R¹¹ NC(S)—, 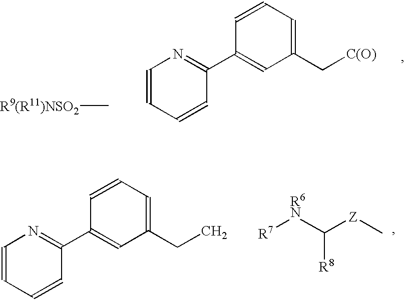
and R⁹SO₂R¹¹NC(O)—;
R³ is selected from the group consisting of: H, C₁₋₆alkyl, C₃₋₆cycloalkyl-C₀₋₆alkyl, C₂₋₆alkenyl, C₂₋₆alkynyl, HetC₀₋₆alkyl and ArC₀₋₆alkyl;
R³ and R′ may be connected to form a pyrrolidine, piperidine or morpholine ring;
R⁴ is selected from the group consisting of: H, C₁₋₆alkyl, C₃₋₆cycloalkyl-C₀₋₆alkyl, Ar—C₀₋₆alkyl, Het-C₀₋₆alkyl, R⁵C(O)—, R⁵C(S)—, R⁵SO₂—, R⁵OC(O)—, R⁵R¹²NC(O)—, and R⁵R¹²NC(S)—;
R⁵ is selected from the group consisting of: H, C₁₋₆alkyl, C₂₋₆alkenyl, C₂₋₆alkynyl, C₃₋₆cycloalkyl-C₀₋₆alkyl, Ar—C₀₋₆alkyl and Het-C₀₋₆alkyl;
R⁶ is selected from the group consisting of: H, C₁₋₆alkyl, Ar—C₀₋₆alkyl, and Het-C₀₋₆alkyl;
R⁷ is selected from the group consisting of: H, C₁₋₆alkyl, C₃₋₆cycloalkyl-C₀₋₆alkyl, Ar—C₀₋₆alkyl, Het-C₀₋₆alkyl, R¹⁰C(O)—, R¹⁰C(S)—, R¹⁰SO₂—, R¹⁰OC(O)—, R¹⁰R¹³NC(O)—, and R¹⁰R¹³NC(S)—;
R⁸ is selected from the group consisting of: H, C₁₋₆alkyl, C₂₋₆alkenyl, C₂₋₆alkynyl, HetC₀₋₆alkyl and ArC₀₋₆alkyl;
R⁹ is selected from the group consisting of: C₁₋₆alkyl, C₃₋₆cycloalkyl-C₀₋₆alkyl, Ar—C₀₋₆alkyl and Het-C₀₋₆alkyl;
R¹⁰ is selected from the group consisting of: C₁₋₆alkyl, C₃₋₆cycloalkyl-C₀₋₆alkyl, Ar—C₀₋₆alkyl and Het-C₀₋₆alkyl;
R¹¹ is selected from the group consisting of: H, C₁₋₆alkyl, Ar—C₀₋₆alky, and Het-C₀₋₆alkyl;
R¹² is selected from the group consisting of: H, C₁₋₆alkyl, Ar—C₀₋₆alkyl, and Het-C₀₋₆alkyl;
R¹³ is selected from the group consisting of: H, C₁₋₆alkyl, Ar—C₀₋₆alkyl, and Het-C-₀₋₆alkyl;
R′ is selected from the group consisting of: H, C₁₋₆alkyl, Ar—C₀₋₆alkyl, and Het-C₀₋₆alkyl;
R″ is selected from the group consisting of: H, C₁₋₆alkyl, Ar—C₀₋₆alkyl, or Het-C₀₋₆alkyl;
R″′ is selected from the group consisting of: H, C₁₋₆alkyl, C₃₋₆cycloalkyl-C₀₋₆alkyl, Ar—C₀₋₆alkyl, and Het-C₀₋₆alkyl;
R″″ is selected from the group consisting of: C₁₋₆alkyl, C₃₋₆cycloalkyl-C₀₋₆alkyl C₂₋₆alkenyl, C_{2 6}alkynyl, HetC₀₋₆alkyl and ArC₀₋₆alkyl;
X is selected from the group consisting of: CH₂, S, and O;
Z is selected from the group consisting of: C(O) and CH₂;
n is an integer from 1 to 5;
and pharmaceutically acceptable salts, hydrates and solvates thereof.

2. A method according to claim 1 wherein said compound is selected from the group consisting of:
Chemical Name
Benzo[1,3]dioxole-5-carboxylic acid [(S)-1-(1-benzyl-3-oxoazepan-4-ylcarbamoyl)-3-methyl-butyl]amide;
Quinoline-2-carboxylic acid [(S)-1-(1-benzyl-3-oxo-azepan-4-ylcarbamoyl)-3-methyl-butyl]amide;
5-(2-Morpholino-4-yl-ethoxy)-benzofuran-2-carboxylic acid ((S)-3-methyl-1-{3-oxo-1-[2-(3-pyridin-2-yl-phenyl)acetyl]-azepan-4-ylcarbamoyl}-butyl)amide;
5-(2-Morpholino-4-yl-ethoxy)-benzofuran-2-carboxylic acid ((S)-3-methyl-1-{3-oxo-1-[2-(3-pyridin-2-yl-phenyl)acetyl]-azepan-4-ylcarbamoyl}-butyl)amide;
4-((S)-4-Methyl-2-{[5-(2-morpholino-4-yl-ethoxy)-benzofuran-2-carbonyl]amino}-pentanoylamino)-3-oxo-azepane-1-carboxylic acid phenylamide;
5-(2-Morpholin-4-yl-ethoxy)-benzofuran-2-carboxylic acid [(S)-1-(1-benzenesulfonyl-3-oxo-azepan-4-ylcarbamoyl)-3-methyl-butyl]amide;
5-(2-Morpholin-4-yl-ethoxy)-benzofuran-2-carboxylic acid [(S)-1-(1-benzenesulfonyl-3-oxo-azepan-4-ylcarbamoyl)-3-methyl-butyl]amide;
5-(2-Pyrrolidin-1-yl-ethoxy)-benzofuran-2-carboxylic acid [(S)-1-(1-benzenesulfonyl-3-oxo-azepan-4-ylcarbamoyl)-3-methyl-butyl]amide;
5-(2-Piperidin-1-yl-ethoxy)-benzofuran-2-carboxylic acid [(S)-1-(1-benzenesulfonyl-3-oxo-azepan-4-ylcarbamoyl)-3-methyl-butyl]amide;
Naphthlene-2-carboxylic acid ((S)-3-methyl-1-{3-oxo-1-[2-(3-pyridin-2-yl-phenyl)ethyl]-azepan-4-ylcarbamoyl}-butyl)amide;
1H-Indole-2-carboxylic acid [(S)-1-(1-benzenesulfonyl-3-oxo-azepan-4-ylcarbamoyl)-3-methyl-butyl]amide;
Benzofuran-2-carboxylic acid [(S)-1-(1-benzenesulfonyl-3-oxo-azepan-4-ylcarbamoyl)-3-methyl-butyl]amide;
5-(2-Pyrrolidin-1-yl-ethoxy)-benzofuran-2-carboxylic acid [(S)-1-(1-benzenesulfonyl-3-oxo-azepan-4-ylcarbamoyl)-3-methyl-butyl]amide;
5-(2-Piperidin-1-yl-ethoxy)-benzofuran-2-carboxylic acid [(S)-1-(1-benzenesulfonyl-3-oxo-azepan-4-ylcarbamoyl)-3-methyl-butyl]amide;
5-(2-Morpholino-4-yl-ethoxy)-benzofuran-2-carboxylic acid {(S)-3-methyl-1-[1-(4-methyl-pentanoyl)-3-oxo-azepan-4-ylcarbamoyl]-butyl}-amide;
Benzofuran-2-carboxylic acid {(S)-3-methyl-1-[3-oxo-1-(pyridine-2-sulfonyl)-azepan-4-ylcarbamoyl]-butyl}-amide;
Naphthalene-2-carboxylic acid {(S)-3-methyl-1-[3-oxo-1-(pyridine-2-sulfonyl)-azepan-4-ylcarbamoyl]-butyl}-amide;
5-(2-Morpholino-4-yl-ethoxy)-benzofuran-2-carboxylic acid {(S)-3-methyl-1-[3-oxo-1-(pyridine-2-sulfonyl)-azepan-4-ylcarbamoyl]-butyl}-amide;
5-(2-Morpholino-4-yl-ethoxy)-benzofuran-2-carboxylic acid {(S)-3-methyl-1-[3-oxo-1-(pyridine-2-sulfonyl)-azepan-4-ylcarbamoyl]-butyl}-amide;
5-(2-Morpholino-4-yl-ethoxy)-benzofuran-2-carboxylic acid {(S)-3-methyl-1-[3-oxo-1-(pyridine-2-sulfonyl)-azepan-4-ylcarbamoyl]-butyl}-amide;
Benzofuran-2-carboxylic acid {(S)-3-methyl-1-[3-oxo-1-(pyridine-2-sulfonyl)-azepan-4-ylcarbamoyl]-butyl}-amide;
4-[2-(2-{(S)-3-Methyl-1-[3-oxo-1-(pyridine-2-sulfonyl)-azepan-4-ylcarbamoyl]-butylcarbamoyl}-benzofuran-5-yloxy)-ethyl]-piperazine-1-carboxylic acid tert-butyl ester;
5-(2-Piperizin-1-yl-ethoxy)-benzofuran-2-carboxylic acid {(S)-3-methyl-1-[3-oxo-1-(pyridine-2-sulfonyl)-azepan-4-ylcarbamoyl]-3-butyl}-amide;
Quinoline-2-carboxylic acid {(S)-3-methyl-1-[3-oxo-1-(pyridine-2-sulfonyl)-azepan-4-ylcarbamoyl]-butyl}amide;
Quinoline-6-carboxylic acid {(S)-3-methyl-1-[3-oxo-1-(pyridine-2-sulfonyl)-azepan-4-ylcarbamoyl]-butyl}amide;
Quinoline-4-carboxylic acid {(S)-3-methyl-1-[3-oxo-1-(pyridine-2-sulfonyl)-azepan-4-ylcarbamoyl]-butyl}amide;
Quinoline-3-carboxylic acid {(S)-3-methyl-1-[3-oxo-1-(pyridine-2-sulfonyl)-azepan-4-ylcarbamoyl]-butyl}amide;
Isoquinoline-3-carboxylic acid {(S)-3-methyl-1-[3-oxo-1-(pyridine-2-sulfonyl)-azepan-4-ylcarbamoyl]-butyl}amide;
Isoquinoline-1-carboxylic acid {(S)-3-methyl-1-[3-oxo-1-(pyridine-2-sulfonyl)-azepan-4-ylcarbamoyl]-butyl}amide;
Quinoxaline-2-carboxylic acid {(S)-3-methyl-1-[3-oxo-1-(pyridine-2-sulfonyl)-azepan-4-ylcarbamoyl]-butyl}amide;
Benzo[b]thiophene-2-carboxylic acid {(S)-3-methyl-1-[3-oxo-1-(pyridine-2-sulfonyl)-azepan-4-ylcarbamoyl]-butyl}amide;
1H-Indole-2-carboxylic acid {(S)-3-methyl-1-[3-oxo-1-(pyridine-2-sulfonyl)-azepan-4-ylcarbamoyl]-butyl}amide;
5-Methoxy-benzofuran-2-carboxylic acid {(S)-3-methyl-1-[3-oxo-1-(pyridine-2-sulfonyl)-azepan-4-ylcarbamoyl]-butyl}amide;
5-Bromo-furan-2-carboxylic acid {(S)-3-methyl-1-[3-oxo-1-(pyridine-2-sulfonyl)-azepan-4-ylcarbamoyl]-butyl}amide;
5-Nitro-furan-2-carboxylic acid {(S)-3-methyl-1-[3-oxo-1-(pyridine-2-sulfonyl)-azepan-4-ylcarbamoyl]-butyl}amide;
5-(4-Nitro-phenyl)-furan-2-carboxylic acid {(S)-3-methyl-1-[3-oxo-1-(pyridine-2-sulfonyl)-azepan-4-ylcarbamoyl]-butyl}amide;
(S)-2-[2-(4-Fluoro-phenoxy)-acetylamino]-4-methyl-pentanoic acid [3-oxo-(pyridine-2-sulfonyl)-azepan-4-yl]-amide;
Benzofuran-2-carboxylic acid {(S)-3-methyl-1-[3-oxo-1-(thiophene-2-sulfonyl)-azepan-4-ylcarbamoyl]-butyl}-amide;
5,6-Dimethoxy-benzofuran-2-carboxylic acid {(S)-3-methyl-1-[3-oxo-1-(1-methyl-1H-imidazole-4-sulfonyl)-azepan-4-ylcarbamoyl]-butyl}amide;
Benzofuran-2-carboxylic acid {(S)-3-methyl-1-[1-(1-methyl-1H-imidazole-3-sulfonyl)-3-oxo-azepan-4-ylcarbamoyl]-butyl}amide;
Benzofuran-2-carboxylic acid {(S)-3-methyl-1-[1-(1H-imidazole-2-sulfonyl)-3-oxo-azepan-4-ylcarbamoyl]-butyl}amide;
Benzofuran-2-carboxylic acid {(S)-3-methyl-1-[1-(1-methyl-1H-imidazole-4-sulfonyl)-3-oxo-azepan-4-ylcarbamoyl]-butyl}amide;
5-(4-Oxy-morpholino-4-yl-ethoxy)-benzofuran-2-carboxylic acid {(S)-3-methyl-1-[3-oxo-1-(pyridine-2-sulfonyl)-azepan-4-ylcarbamoyl]-butyl}amide;
5-Hydroxy-benzofuran-2-carboxylic acid {(S)-3-methyl-1-[1-(1-methyl-1H-imidazole-4-sulfonyl)-3-oxo-azepan-4-ylcarbamoyl]-butyl}amide;
Benzofuran-2-carboxylic acid {(S)-3-methyl-1-[3-oxo-1-(1-oxy-pyridine-2-sulfonyl)-azepan-4-ylcarbamoyl)]-3-methyl-butyl}-amide;
Benzo[b]thiophene-2-carboxylic acid {(S)-3-methyl-1-[3-oxo-1-(1-oxy-pyridine-2-sulfonyl)-azepan-4-ylcarbamoyl]-butyl}amide;
5-Bromo-furan-2-carboxylic acid {(S)-3-methyl-1-[3-oxo-1-(1-oxy-pyridine-2-sulfonyl)azepan-4-ylcarbamoyl]-butyl}amide;
5,6-Dimethoxy-benzofuran-2-carboxylic acid {(S)-3-methyl-1-[3-oxo-1-(1-oxy-pyridine-2-sulfonyl)-azepan-4-ylcarbamoyl]-butyl}amide;
Benzofuran-2-carboxylic acid {(S)-3-methyl-1-[3-oxo-1-(1-oxy-pyridine-2-sulfonyl)-azepan-4-ylcarbamoyl)]-3-methyl-butyl}-amide;
5-Bromo-furan-2-carboxylic acid {(S)-3-methyl-1-[3-oxo-1-(1-oxy-pyridine-2-sulfonyl)-azepan-4-ylcarbamoyl]-butyl}amide;
Benzo[b]thiophene-2-carboxylic acid {(S)-3-methyl-1-[3-oxo-1-(1-oxy-pyridine-2-sulfonyl)-azepan-4-ylcarbamoyl]-butyl}amide;
5,6-Dimethoxy-benzofuran-2-carboxylic acid {(S)-3-methyl-1-[3-oxo-1-(1-oxy-pyridine-2-sulfonyl)-azepan-4-ylcarbamoyl]-butyl}amide;
5-Methoxy-benzofuran-2-carboxylic acid {(S)-3-methyl-1-[3-oxo-1-(1-oxy-pyridine-2-sulfonyl)-azepan-4-ylcarbamoyl]-butyl}amide;
1H-Indole-5-carboxylic acid {(S)-3-methyl-1-[3-oxo-1-(1-oxy-pyridine-2-sulfonyl)-azepan-4-ylcarbamoyl]-butyl}amide;
Benzo[1,3]dioxole-5-carboxylic acid {(S)-3-methyl-1-[3-oxo-1-(1-oxy-pyridine-2-sulfonyl)-azepan-4-ylcarbamoyl]-butyl}amide;
5-(2-Morpholin-4-yl-ethoxy)-benzofuran-2-carboxylic acid {(S)-3-methyl-1-[3-oxo-(1-oxy-pyridine2-sulfonyl)-azepan-4-ylcarbamoyl]-buty}-amide;
3-Methyl-benzofuran-2-carboxylic acid {(S)-3-methyl-1-[3-oxo-1-(1-oxy-pyridine-2-sulfonyl)-azepan-4-ylcarbamoyl]-butyl}amide;
1H-Indole-6-carboxylic acid {(S)-3-methyl-1-[3-oxo-1-(pyridine-2-sulfonyl)-azepan-4-ylcarbamoyl]-butyl}amide;
Benzo[1,3]dioxole-5-carboxylic acid {(S)-3-methyl-1-[3-oxo-1-(pyridine-2-sulfonyl)-azepan-4-ylcarbamoyl]-butyl}amide;
3,4-Dihydro-2H-benzo[b][1,4]dioxepine-7-carboxylic acid {(S)-3-methyl-1-[3-oxo-1-(1-oxy-pyridine-2-sulfonyl)-azepan-4-ylcarbamoyl]butyl}amide;
4,5-Dibromo-thiophene-2-carboxylic acid {(S)-3-methyl-1-[3-oxo-1-(1-oxy-pyridine-2-sulfonyl)-azepan-4-ylcarbamoyl]-butyl}amide;
Thieno[3,2-b]thiophene-2-carboxylic acid {(S)-3-methyl-1-[3-oxo-1-(1-oxy-pyridine-2-sulfonyl)-azepan-4-ylcarbamoyl]-butyl}amide;
5-Methoxy-benzofuran-2-carboxylic acid {(S)-3-methyl-1-[3-oxo-1-(1-oxy-pyridine-2-sulfonyl)-azepan-4-ylcarbamoyl]-butyl}amide;
1H-Indole-5-carboxylic acid {(S)-3-methyl-1-[3-oxo-1-(1-oxy-pyridine-2-sulfonyl)-azepan-4-ylcarbamoyl]-butyl}amide;
5-(4-Chloro-phenyl)-furan-2-carboxylic acid {(S)-3-methyl-1-[3-oxo-1-(pyridine-2-sulfonyl)-azepan-4-ylcarbamoyl]-butyl}amide;
5-(3-Trifluoromethyl-phenyl)-furan-2-carboxylic acid {(S)-3-methyl-1-[3-oxo-1-(pyridine-2-sulfonyl)-azepan-4-ylcarbamoyl]-butyl}amide;
Benzo[1,3]dioxole-5-carboxylic acid {(S)-1-[1-(4-fluoro-benzenesulfonyl)-3-oxo-azepan-4-ylcarbamoyl]-3methyl-butyl}-amide;
5-Bromo-furan-2-carboxylic acid {(S)-3-methyl-1-[3-oxo-1-(pyridine-2-sulfonyl)-azepan-4-ylcarbamoyl]-butyl}amide;
5-Methoxy-benzofuran-2-carboxylic acid {(S)-3-methyl-1-[3-oxo-1-(pyridine-2-sulfonyl)-azepan-4-ylcarbamoyl]-butyl}amide;
3-Methyl-benzofuran-2-carboxylic acid {(S)-3-methyl-1-[3-oxo-1-(pyridine-2-sulfonyl)-azepan-4-ylcarbamoyl]-butyl}amide;
Thieno[3,2-b]thiophene-2-carboxylic acid {(S)-3-methyl-1-[3-oxo-1-(pyridine-2-sulfonyl)-azepan-4-ylcarbamoyl]-butyl}amide;
2-Phenyl-5-trifluoromethyl-oxazole-4-carboxylic acid {(S)-3-methyl-1-[3-oxo-1-(pyridine-2-sulfonyl)-azepan-4-ylcarbamoyl]-butyl}amide;
Benzo[1,3]dioxole-5-carboxylic acid {(S)-1-[1-(4-methoxy-benzenesulfonyl)-3-oxo-azepan-4-ylcarbamoyl]-3-methyl-butyl}-amide;
Benzofuran-2-carboxylic acid {(S)-1-[1-(4-methoxy-benzenesulfonyl)-3-oxo-azepan-4-ylcarbamoyl]-3-methyl-butyl}-amide;
Furan-2-carboxylic acid {(S)-3-methyl-1-[3-oxo-1-(1-oxy-pyridine-2-sulfonyl)-azepan-4-ylcarbamoyl]-butyl}amide;
Benzo[1,3]dioxole-5-carboxylic acid {(S)-3-methyl-1-[3-oxo-1-(1-oxy-pyridine-2-sulfonyl)-azepan-4-ylcarbamoyl]-butyl}amide;
4-Fluoro-{(S)-3-methyl-1-[3-oxo-1-(1-oxy-pyridine-2-sulfonyl)-azepan-4-carbamoyl]-butyl}-benzamide;
3,4-Dihydro-2H-benzo[b][1,4]dioxepine-7-carboxylic acid {(S)-3-methyl-1-[3-oxo-1-(1-oxy-pyridine-2-sulfonyl)-azepan-4-ylcarbamoyl]butyl}amide;
5-Methyl-thiophene-2-carboxylic acid {(S)-3-methyl-1-[3-oxo-1-(1-oxy-pyridine-2-sulfonyl)-azepan-4-ylcarbamoyl]-butyl}amide;
(S)-2-(3-Benzyl-ureido)-4-methyl-pentanoic acid [3-oxo-1-(pyridine-2-sulfonyl)-azepan-4-yl]-amide;
5-Methoxy-benzofuran-2-carboxylic acid [(S)-1-(1-methanesulfonyl-3-oxo-azepan-4-ylcarbamoyl)-3-methyl-butyl]-amide;
Furan-2-carboxylic acid ({(S)-1-[1-(4-methoxy-benzenesulfonyl)-3-oxo-azepan-4-ylcarbamoyl]-3-methyl-butylcarbamoyl}-methyl)-amide;
Quinoline-2-carboxylic acid {[(S)-1-[1-(4-fluoro-benzenesulfonyl)-3-oxo-azepan-4-ylcarbamoyl]-3-methyl-butyl}-amide;
1-Methyl-1H-indole-2-carboxylic acid {[(S)-1-[1-(4-fluoro-benzenesulfonyl)-3-oxo-azepan-4-ylcarbamoyl]-3-methyl-butyl}-amide;
5-Methoxy-benzofuran-2-carboxylic acid {[(S)-1-[1-(4-fluoro-benzenesulfonyl)-3-oxo-azepan-4-ylcarbamoyl]-3-methyl-butyl}-amide;
Quinoxaline-2-carboxylic acid {[(S)-1-[1-(4-fluoro-benzenesulfonyl)-3-oxo-azepan-4-ylcarbamoyl]-3-methyl-butyl}-amide;
Benzo[b]thiophene-2-carboxylic acid {(S)-3-methyl-1-[3-oxo-1-(pyridine-2-sulfonyl)-azepan-4-ylcarbamoyl]-butyl}amide;
Benzofuran-2-carboxylic acid-{(S)-1-[1-(3-chloro-benzenesulfonyl)-3-oxo-azepan-4-ylcarbamoyl]-3-methyl-butyl}-amide;
5-Methoxy-benzofuran-2-carboxylic acid-{(S)-1-[1-(3-chloro-benzenesulfonyl)-3-oxo-azepan-4-ylcarbamoyl]-3-methyl-butyl}-amide;
3-Methyl-benzofuran-2-carboxylic acid-{(S)-1-[1-(3-chloro-benzenesulfonyl)-3-oxo-azepan-4-ylcarbamoyl]-3-methyl-butyl}-amide;
Benzofuran-2-carboxylic acid-{(S)-1-[1-(2-fluoro-benzenesulfonyl)-3-oxo-azepan-4-ylcarbamoyl]-3-methyl-butyl}-amide;
5-Methoxy-benzofuran-2-carboxylic acid-{(S)-1-[1-(2-fluoro-benzenesulfonyl)-3-oxo-azepan-4-ylcarbamoyl]-3-methyl-butyl}-amide;
7-Methoxy-benzofuran-2-carboxylic acid-{(S)-1-[1-(2-fluoro-benzenesulfonyl)-3-oxo-azepan-4-ylcarbamoyl]-3-methyl-butyl}-amide;
3-methylbenzofuran-2-carboxylic acid-{(S)-1-[1-(2-fluoro-benzenesulfonyl)-3-oxo-azepan-4-ylcarbamoyl]-3-methyl-butyl}-amide;
Benzo[b]thiophene-2-carboxylic acid-{(S)-1-[1-(2-fluoro-benzenesulfonyl)-3-oxo-azepan-4-ylcarbamoy]-3-methyl-butyl}-amide;
Quinoxaline-2-carboxylic acid-{(S)-1-[1-(2-fluoro-benzenesulfonyl)-3-oxo-azepan-4-ylcarbamoyl]-3-methyl-butyl}-amide;
3-Methyl-benzofuran-2-carboxylic acid {(S)-3-methyl-1-[3-oxo-1-(pyridine-2-sulfonyl)-azepan-4-ylcarbamoyl]-butyl}amide;
Thieno[3,2-b]thiophene-2-carboxylic acid {(S)-3-methyl-1-[3-oxo-1-(pyridine-2-sulfonyl)-azepan-4-ylcarbamoyl]-butyl}amide;
3-Methyl-benzofuran-2-carboxylic acid {(S)-3-methyl-1-[3-oxo-1-(1-oxy-pyridine-2-sulfonyl)-azepan-4-ylcarbamoyl]-butyl}amide;
Benzofuran-2-carboxylic acid {(S)-3-methyl-1-[(2,2′,4-tridueterio)-3-oxo-1-(pyridine-2-sulfonyl)-azepan-4-ylcarbamoyl]-butyl}amide;
Quinoxaline-2-carboxylic acid {(S)-3-methyl-1-[3-oxo-1-(1-oxy-pyridine-2-sulfonyl)-azepan-4-ylcarbamoyl]-butyl}amide;
Benzofuran-2-carboxylic acid {(S)-2-cyclohexyl-1-[3-oxo-1-(pyridine-2-sulfonyl)-azepan-4-ylcarbamoyl]-ethyl}-amide;
Benzofuran-2-carboxylic acid {(S)-1-[3-oxo-1-(pyridine-2-sulfonyl)-azepan-4-ylcarbamoyl]-pentyl}-amide;
Benzofuran-2-carboxylic acid {(S)-1-[3-oxo-1-(pyridine-2-sulfonyl)-azepan-4-ylcarbamoyl]-2-phenyl-ethyl}-amide;
2-Phenyl-5-trifluoromethyl-oxazole-4-carboxylic acid {(S)-3-methyl-1-[3-oxo-1-(pyridine-2-sulfonyl)-azepan-4-ylcarbamoyl]-butyl}amide;
5-Methyl-2-phenyl-oxazole-4-carboxylic acid {(S)-3-methyl-1-[3-oxo-1-(pyridine-2-sulfonyl)-azepan-4-ylcarbamoyl]-butyl}amide;
3,4-Dimethoxy-N—{(S)-1-[1-(4-methoxy-benzenesulfonyl)-3-oxo-azepan-4-ylcarbamoyl]-3-methyl-butyl}-benzamide;
Benzo[b]thiophene-2-carboxylic acid-{(S)-1-[1-(4-fluoro-benzenesulfonyl)-3-oxo-azepan-4-ylcarbamoyl]-3-methyl-butyl}-amide;
Benzofuran-2-carboxylic acid-{(S)-1-[1-(4-fluoro-benzenesulfonyl)-3-oxo-azepan-4-ylcarbamoyl]-3-methyl-butyl}-amide;
N—{(S)-1-[1-(4-Fluoro-benzenesulfonyl)-3-oxo-azepan-4-ylcarbamoyl}-3-methyl-butyl}-3,4-dimethoxy-benzamide;
Benzo[b]thiophene-2-carboxylic acid-{(S)-1-(1-methanesulfonyl-3-oxo-azepan-4-ylcarbamoyl)-3-methyl-butyl]-amide;
Benzofuran-2-carboxylic acid-{(S)-1-(1-methanesulfonyl-3-oxo-azepan-4-yl carbamoyl)-3-methyl-butyl]-amide;
N—[(S)-1-(1-Methanesulfonyl)-3-oxo-azepan-4-ylcarbamoyl}-3-methyl-butyl}-3,4-dimethoxy-benzamide;
N—{(S)-1-[1-(2-Cyano-benzenesulfonyl)-3-oxo-azepan-4-ylcarbamoyl}-3-methyl-butyl}-4-methanesulfonyl-benzamide;
Benzofuran-2-carboxylic acid {(S)-1-[1-(2-cyano-benzenesulfonyl)-3-oxo-azepan-4-ylcarbamoyl]-3-methyl-butyl}-amide;
5-(2-Morpholin-4-yl-ethoxy)-benzofuran-2-carboxylic acid {(S)-3-methyl-1-[3-oxo-(1-oxy-pyridine2-sulfonyl)-azepan-4-ylcarbamoyl]-buty}-amide;
5-Methyl-2-phenyl-oxazole-4-carboxylic acid {(S)-3-methyl-1-[3-oxo-1-(1-oxy-pyridine-2-sulfonyl)-azepan-4-ylcarbamoyl]-butyl}amide;
6-Methyl-N—{(S)-3-methyl-1-[3-oxo-1-(1-oxy-pyridine-2-sulfonyl)-azepan-4-ylcarbamoyl]-butyl}-nicotinamide;
5-(3-Trifluoromethyl-phenyl)-furan-2-carboxylic acid {(S)-3-methyl-1-[3-oxo-1-(1-oxy-pyridine-2-sulfonyl)-azepan-4-ylcarbamoyl]-butyl}amide;
N—{(S)-1-[(1-(2-cyano-benzenesulfonyl)-3-oxo-azepan-4-ylcarbamoyl}-3-methyl-butyl}-3,4-dimethoxy-benzamide;
4-Methansulfonyl-N—{(S)-1-[4-fluoro-benzenesulfonyl)-3-oxo-azepan-4-carbamoyl]-3-methyl-butyl-benzamide;
(S)-2-[5-(4-Methoxy-phenyl)-pentanoylamino]-4-methyl-pentanoic acid [3-oxo-1-(pyridine-2-sulfonyl)-azepan-4-yl]-amide;
(S)-2-[2-(3-Benzyloxy-4-methoxy-phenyl)-acetylamnio]-4-methylpentanoic acid [3-oxo-1-(pyridine-2-sulfonyl)-azepan-4-yl]-amide;
5-Methoxybenzofuran-2-carboxylic acid {(S)-3-methyl-1-[3-oxo-1-(thiazole-2-sulfonyl)-azepan-4-ylcarbamoyl]-butyl}amide;
7-Methoxybenzofuran-2-carboxylic acid {(S)-3-methyl-1-[3-oxo-1-(thiazole-2-sulfonyl)-azepan-4-ylcarbamoyl]-butyl}amide;
3-Methylbenzofuran-2-carboxylic acid {(S)-3-methyl-1-[3-oxo-1-(thiazole-2-sulfonyl)-azepan-4-ylcarbamoyl]-butyl}amide;
Benzo[b]thiophene-2-carboxylic acid {(S)-3-methyl-1-[3-oxo-1-(thiazole-2-sulfonyl)-azepan-4-ylcarbamoyl]-butyl}amide;
1-Methyl-1H-indole-2-carboxylic acid {(S)-3-methyl-1-[3-oxo-1-(thiazole-2-sulfonyl)-azepan-4-ylcarbamoyl]-butyl}amide;
Quinoxaline-2-carboxylic acid {(S)-3-methyl-1-[3-oxo-1-(thiazole-2-sulfonyl)-azepan-4-ylcarbamoyl]-butyl}amide;
Benzofuran-2-carboxylic acid {(S)-3-methyl-1-[3-oxo-1-(thiazole-2-sulfonyl)-azepan-4-ylcarbamoyl]-butyl}amide;
Benzofuran-2-carboxylic acid {(S)-1-[1-(4-chloro-benzenesulfonyl)-3-oxo-azepan-4-ylcarbamoyl]-3-methyl-butyl}-amide;
5-Methoxy-benzofuran-2-carboxylic acid-{(S)-1-[1-(4-chloro-benzenesulfonyl)-3-oxo-azepan-4-ylcarbamoyl]-3-methyl-butyl}-amide;
7-Methoxy-benzofuran-2-carboxylic acid-{(S)-1-[1-(4-chloro-benzenesulfonyl)-3-oxo-azepan-4-ylcarbamoyl]-3-methyl-butyl}-amide;
3-Methyl-benzofuran-2-carboxylic acid-{(S)-1-[1-(4-chloro-benzenesulfonyl)-3-oxo-azepan-4-ylcarbamoyl]-3-methyl-butyl}-amide;
Benzo[b]thiophene-2-carboxylic acid-{(S)-1-[1-(4-chloro-benzenesulfonyl)-3-oxo-azepan-4-ylcarbamoyl]-3-methyl-butyl}-amide;
Benzofuran-2-carboxylic acid-{(S)-1-[1-(3-methoxy-benzenesulfonyl)-3-oxo-azepan-4-ylcarbamoyl]-3-methyl-butyl}-amide;
5-Methoxy-benzofuran-2-carboxylic acid-{(S)-1-[1-(3-methoxy-benzenesulfonyl)-3-oxo-azepan-4-ylcarbamoyl]-3-methyl-butyl}-amide;
7-Methoxy-benzofuran-2-carboxylic acid-{(S)-1-[1-(3-methoxy-benzenesulfonyl)-3-oxo-azepan-4-ylcarbamoyl]-3-methyl-butyl}-amide;
3-Methyl-benzofuran-2-carboxylic acid-{(S)-1-[1-(3-methoxy-benzenesulfonyl)-3-oxo-azepan-4-ylcarbamoyl]-3-methyl-butyl}-amide;
Benzo[b]thiophene-2-carboxylic acid-{(S)-1-[1-(3-methoxy-benzenesulfonyl)-3-oxo-azepan-4-ylcarbamoyl]-3-methyl-butyl}-amide;
1-Methyl-1H-indole-2-carboxylic acid-{(S)-1-[1-(3-methoxy-benzenesulfonyl)-3-oxo-azepan-4-ylcarbamoyl]-3-methyl-butyl}-amide;
Benzofuran-2-carboxylic acid-{(S)-3-methyl-1-[3-oxo-1-(thiophene-2-sulfonyl)-azepan-4-ylcarbamoyl]-butyl}-amide;
5-Methoxy-benzofuran-2-carboxylic acid-{(S)-3-methyl-1-[3-oxo-1-(thiophene-2-sulfonyl)-azepan-4-ylcarbamoyl]-butyl}-amide;
7-Methoxy-benzofuran-2-carboxylic acid-{(S)-3-methyl-1-[3-oxo-1-(thiophene-2-sulfonyl)-azepan-4-ylcarbamoyl]-butyl}-amide;
3-Methyl-benzofuran-2-carboxylic acid-{(S)-3-methyl-1-[3-oxo-1-(thiophene-2-sulfonyl)-azepan-4-ylcarbamoyl]-butyl}-amide;
Benzo[b]thiophene-2-carboxylic acid-{(S)-3-methyl-1-[3-oxo-1-(thiophene-2-sulfonyl)-azepan-4-ylcarbamoyl]-butyl}-amide;
Quinoxaline-2-carboxylic acid-{(S)-3-methyl-1-[3-oxo-1-(thiophene-2-sulfonyl)-azepan-4-ylcarbamoyl]-butyl}-amide;
1-Methyl-1-H-indole-2-carboxylic acid-{(S)-3-methyl-1-[3-oxo-1-(thiophene-2-sulfonyl)-azepan-4-ylcarbamoyl]-butyl}-amide;
5,6-Difluoro-benzofuran-2-carboxylic acid {(S)-3-methyl-1-[1-(pyridine-2-sulfonyl)-3-oxo-azepan-4-ylcarbamoyl]-butyl)}amide;
5,6-Difluoro-benzofuran-2-carboxylic acid {(S)-3-methyl-1-[1-(pyridine-2-sulfonyl)-3-oxo-azepan-4-ylcarbamoyl]-butyl}amide;
Quinoline-2-carboxylic acid {(S)-3-methyl-1-[3-oxo-1-(pyridine-2-sulfonyl)-azepan-4-ylcarbamoyl]-butyl}amide;
Quinoline-6-carboxylic acid {(S)-3-methyl-1-[3-oxo-1-(pyridine-2-sulfonyl)-azepan-4-ylcarbamoyl]-butyl}amide;
Quinoline-4-carboxylic acid {(S)-3-methyl-1-[3-oxo-1-(pyridine-2-sulfonyl)-azepan-4-ylcarbamoyl]-butyl}amide;
Isoquinoline-1-carboxylic acid {(S)-3-methyl-1-[3-oxo-1-(pyridine-2-sulfonyl)-azepan-4-ylcarbamoyl]-butyl}amide;
Naphthalene-2-carboxylic acid {(S)-3-methyl-1-[3-oxo-1-(pyridine-2-sulfonyl)-azepan-4-ylcarbamoyl]-butyl}-amide;
Quinoline-3-carboxylic acid {(S)-3-methyl-1-[3-oxo-1-(pyridine-2-sulfonyl)-azepan-4-ylcarbamoyl]-butyl}amide;
5,6-Dimethoxy-benzo[b]thiophene-2-carboxylic acid {(S)-3-methyl-1-[1-(6-methyl-pyridine-2-sulfonyl)3-oxo-azepan-4-ylcarbamoyl]-butyl}amide;
(R)-1-Benzyl-5-oxo-pyrrolidine-2-carboxylic acid {(S)-3-methyl-1-{3-oxo-(pyridine-2-sulfonyl)-azepan-4-ylcarbamoyl]-butyl}amide;
Benzofuran-2-carboxylic acid {(S)-2-naphthalen-2-yl-1-[3-oxo-1-(pyridine-2-sulfonyl)-azepan-4-ylcarbamoyl)-ethyl]-amide;
Thieno[3,2-b]thiophene-2-carboxylic acid {(S)-3-methyl-1-[1-(3-methyl-pyridine-2-sulfonyl)-3-oxo-azepan-4-ylcarbamoyl]-butyl}amide;
3-Methyl-benzofuran-2-carboxylic acid {(S)-3-methyl-1-[1-(3-methyl-pyridine-2-sulfonyl)-3-oxo-azepan-4-ylcarbamoyl]-butyl}amide;
5-Methoxy-benzofuran-2-carboxylic acid {(S)-3-methyl-1-[1-(3-methyl-pyridine-2-sulfonyl)-3-oxo-azepan-4-ylcarbamoyl]-butyl}amide;
5,6-Difluoro-benzofuran-2-carboxylic acid {(S)-3-methyl-1-[3-oxo-1-(1-oxy-pyridine-2-sulfonyl)-azepan-4-ylcarbamoyl]-butyl}amide;
7-Methoxy-benzofuran-2-carboxylic acid {(S)-3-methyl-1-[1-(6-methyl-pyridine-2-sulfonyl)-3-oxo-azepan-4-ylcarbamoyl]-butyl)}amide;
5,6-Dimethoxy-benzo[b]thiophene-2-carboxylic acid {(S)-3-methyl-1-[1-(6-methyl-pyridine-2-sulfonyl)3-oxo-azepan-4-ylcarbamoyl]-butyl}amide;
5-Fluoro-benzofuran-2-carboxylic acid {(S)-3-methyl-1-[3-oxo-1-(pyridine-2-sulfonyl)-azepan-4-ylcarbamoyl]-butyl}-amide;
Benzofuran-2-carboxylic acid {(S)-3-methyl-1-[6-methyl-3-oxo-1-(pyridine-sulfonyl)-azepan-4-ylcarbamoyl]-butyl}-amide;
5-Fluoro-benzofuran-2-carboxylic acid {(S)-3-methyl-1-[3-oxo-1-(pyridine-2-sulfonyl)-azepan-4-ylcarbamoyl]-butyl}-amide;
5-(3-Trifluoromethyl-phenyl)-furan-2-carboxylic acid {(S)-2-cyclohexyl-1-[3-oxo-1-(1-oxy-pyridine-2-sulfonyl)-azepan-4-ylcarbamoyl]-ethyl}-amide;
5,6-Dimethoxy-benzofuran-2-carboxylic acid (S)-2-cyclohexyl-1-[3-oxo-1-(1-oxy-pyridine-2-sulfonyl)-azepan-4-ylcarbamoyl]-ethyl}-amide;
5-Methoxy-benzofuran-2-carboxylic acid {(S)-3-methyl-1-[1-(3-methyl-pyridine-2-sulfonyl)-3-oxo-azepan-4-ylcarbamoyl]-butyl}amide;
Benzofuran-2-carboxylic acid {(S)-3-methyl-1-[1-(6-methyl-pyridine-2-sulfonyl)-3-oxo-azepan-4-ylcarbamoyl]-butyl}amide;
Benzofuran-2-carboxylic acid {(S)-3-methyl-1-[6-methyl-3-oxo-1-(pyridine-sulfonyl)-azepan-4-ylcarbamoyl]-butyl}-amide;
Quinoline-8-carboxylic acid {(S)-2-naphthalen-2-yl-1-[3-oxo-1-(pyridine-2-sulfonyl)-azepan-4-ylcarbamoyl)-ethyl]-amide;
Naphthalene-1-carboxylic acid {(S)-2-naphthalen-2-yl-1-[3-oxo-1-(pyridine-2-sulfonyl)-azepan-4-ylcarbamoyl)-ethyl]-amide;
Quinoline-8-carboxylic acid {(S)-1-[3-oxo-1-(pyridine-2-sulfonyl)-azepan-4-ylcarbamoyl]-2-phenyl-ethyl}-amide;
Naphthyridine-2-carboxylic acid {(S)-3-methyl-1-[3-oxo-1-(pyridine-2-sulfonyl)-azepan-4-ylcarbamoyl]-butyl}-amide;
Naphthalene-1-carboxylic acid {(S)-1-[3-oxo-1-(pyridine-2-sulfonyl)-azepan-4-ylcarbamoyl]-2-phenyl-ethyl}-amide;
Benzofuran-2-carboxylic acid {(S)-3-methyl-1-[1-(2-methyl-furan-3-sulfonyl)-3-oxo-azepan-4-ylcarbamoyl]-butyl}-amide;
Quinoline-2-carboxylic acid {(S)-1-[3-oxo-1-(pyridine-2-sulfonyl)-azepan-4-ylcarbamoyl]-2-phenyl-ethyl}-amide;
Benzofuran-2-carboxylic acid {(S)-3-methyl-1-[(4S ,7S)-7-methyl-3-oxo-1-(pyridine-2-sulfonyl)-azepan-4-ylcarbamoyl]-butyl}-amide;
Benzofuran-2-carboxylic acid {(S)-3-methyl-1-[(4R,7R)-7-methyl-3-oxo-1-(pyridine-2-sulfonyl)-azepan-4-ylcarbamoyl]-butyl}-amide;
Benzofuran-2-carboxylic acid {(S)-1-[-(3-fluoro-benzensulfonyl)-3-oxo-azepan-4-ylcarbamoyl]-3-methyl-1-butyl}-amide;
Naphthalene-1-carboxylic acid {(S)-3-methyl-1-[3-oxo-1-(pyridine-2-sulfonyl)-azepan-4-ylcarbamoyl]-butyl}-amide;
Quinoline-5-carboxylic acid {(S)-3-methyl-1-[3-oxo-1-(pyridine-2-sulfonyl)-azepan-4-ylcarbamoyl]-butyl}-amide;
5-(3-Trifluoromethyl-phenyl)-furan-2-carboxylic acid ((S)-3-methyl-1-{3-oxo-1-[1-(1-oxy-pyridin-2-yl)-methanoyl]-azepan-4-ylcarbamoyl}-butyl)-amide;
Quinoline-8-carboxylic acid {(S)-2-naphthalen-2-yl-1-[3-oxo-1-(pyridine-2-sulfonyl)-azepan-4-ylcarbamoyl]-ethyl}-amide;
Naphthalene-1-carboxylic acid {(S)-2-naphthalen-2-yl-1-[3-oxo-1-(pyridine-2-sulfonyl)-azepan-4-ylcarbamoyl]-ethyl}-amide;
Quinoline-8-carboxylic acid {(S)-1-[3-oxo-1-(pyridine-2-sulfonyl)-azepan-4-ylcarbamoyl]-2-phenyl-ethyl}-amide;
Naphthalene-1-carboxylic acid {(S)-1-[3-oxo-1-(pyridine-2-sulfonyl)-azepan-4-ylcarbamoyl]-2-phenyl-ethyl}-amide;
5-Fluoro-benzofuran-2-carboxylic acid {(S)-3-methyl-1-[3-oxo-1-(1-oxy-pyridine-2-sulfonyl)-azepan-4-ylcarbamoyl]-butyl}-amide;
5-Fluoro-3-methyl-benzofuran-2-carboxylic acid {(S)-3-methyl-1-[3-oxo-1-(1-oxy-pyridine-2-sulfonyl)-azepan-4-ylcarbamoyl]-butyl}-amide;
6-Fluoro-3-methyl-benzofuran-2-carboxylic acid {(S)-3-methyl-1-[3-oxo-1-(1-oxy-pyridine-2-sulfonyl)-azepan-4-ylcarbamoyl]-butyl}-amide;
5-Fluoro-benzofuran-2-carboxylic acid {(S)-3-methyl-1-[3-oxo-1-(1-oxy-pyridine-2-sulfonyl)-azepan-4-ylcarbamoyl]-butyl}-amide;
5-Fluoro-3-methyl-benzofuran-2-carboxylic acid {(S)-3-methyl-1-[3-oxo-1-(1-oxy-pyridine-2-sulfonyl)-azepan-4-ylcarbamoyl]-butyl}-amide;
6-Fluoro-3-methyl-benzofuran-2-carboxylic acid {(S)-3-methyl-1-[3-oxo-1-(1-oxy-pyridine-2-sulfonyl)-azepan-4-ylcarbamoyl]-butyl}-amide;
Benzo[b]thiophene-2-carboxylic acid {(S)-3-methyl-1-[6-methyl-3-oxo-1-(pyridine-2-sulfonyl)-azepan-4-ylcarbamoyl]-butyl}-amide;
5-Methoxy-benzofuran-2-carboxylic acid {(S)-3-methyl-1-[6-methyl-3-oxo-1-(pyridine-2-sulfonyl)-azepan-4-ylcarbamoyl]-butyl}-amide;
3-Methyl-benzofuran-2-carboxylic acid {(S)-3-methyl-1-[6-methyl-3-oxo-1-(pyridine-2-sulfonyl)-azepan-4-ylcarbamoyl]-butyl}-amide;
Thieno[3,2-b]thiophene-2-carboxylic acid {(S)-3-methyl-1-[6-methyl-3-oxo-1-(pyridine-2-sulfonyl)-azepan-4-ylcarbamoyl]-butyl}-amide;
3,5-Dimethyl-benzofuran-2-carboxylic acid {(S)-3-methyl-1-[3-oxo-1-(1-oxy-pyridine-2-sulfonyl)-azepan-4-ylcarbamoyl]-butyl}-amide;
3-Ethyl-benzofuran-2-carboxylic acid {(S)-3-methyl-1-[3-oxo-1-(1-oxy-pyridine-2-sulfonyl)-azepan-4-ylcarbamoyl]-butyl}-amide;
4-Methoxy-3-methyl-benzofuran-2-carboxylic acid {(S)-3-methyl-1-[3-oxo-1-(1-oxy-pyridine-2-sulfonyl)-azepan-4-ylcarbamoyl]-butyl}-amide;
6-Methoxy-3-methyl-benzofuran-2-carboxylic acid {(S)-3-methyl-1-[3-oxo-1-(1-oxy-pyridine-2-sulfonyl)-azepan-4-ylcarbamoyl]-butyl}-amide;
5-Methoxy-3-methyl-benzofuran-2-carboxylic acid {(S)-3-methyl-1-[3-oxo-1-(1-oxy-pyridine-2-sulfonyl)-azepan-4-ylcarbamoyl]-butyl}-amide;
3,5-Dimethyl-benzofuran-2-carboxylic acid {(S)-3-methyl-1-[3-oxo-1-(1-oxy-pyridine-2-sulfonyl)-azepan-4-ylcarbamoyl]-butyl}-amide;
3-Ethyl-benzofuran-2-carboxylic acid {(S)-3-methyl-1-[3-oxo-1-(1-oxy-pyridine-2-sulfonyl)-azepan-4-ylcarbamoyl]-butyl}-amide;
4-Methoxy-3-methylbenzofuran-2-carboxylic acid {(S)-3-methyl-1-[3-oxo-1-(1-oxy-pyridine-2-sulfonyl)-azepan-4-ylcarbamoyl]-butyl}-amide;
4-Methoxy-3-methyl-benzofuran-2-carboxylic acid {(S)-3-methyl-1-[3-oxo-1-(1-oxy-pyridine-2-sulfonyl)-azepan-4-ylcarbamoyl]-butyl}-amide;
1-methyl-naphtho[2,1-b]-furan-2-carboxylic acid {(S)-3-methyl-1-[3-oxo-1-(1-oxy-pyridine-2-sulfonyl)-azepan-4-ylcarbamoyl]-butyl}-amide;
6-Methoxy-3-methyl-benzofuran-2-carboxylic acid {(S)-3-methyl-1-[3-oxo-1-(1-oxy-pyridine-2-sulfonyl)-azepan-4-ylcarbamoyl]-butyl}-amide;
Benzofuran-2-carboxylic acid [(S)-3-methyl-1-[3-oxo-1-quinolin-2-ylmethyl-azepan-4-ylcarbamoyl]-butyl}-amide;
3-Methyl-benzofuran-2-carboxylic acid [(S)-3-methyl-1-[3-oxo-1-quinolin-2-ylmethyl-azepan-4-ylcarbamoyl]-butyl}-amide;
Benzo[b]thiophene-2-carboxylic acid [(S)-3-methyl-1-[3-oxo-1-quinolin-2-ylmethyl-azepan-4-ylcarbamoyl]-butyl}-amide;
Benzo[b]thiophene-2-carboxylic acid {(S)-1-[1-(2-fluoro-phenylcarbamoyl)-3-oxo-azepan-4-ylcarbamoyl]-3-methyl-butyl}-amide;
3-Methyl-benzofuran-2-carboxylic acid {(S)-1-[1-(2-fluoro-phenylcarbamoyl)-3-oxo-azepan-4-ylcarbamoyl]-3-methyl-butyl}-amide;
Quinoxaline-2-carboxylic acid {(S)-1-(2-fluoro-phenylcarbamoyl)-3-oxo-azepan-4-ylcarbamoyl]-3-methyl-butyl}-amide;
Thieno[3,2-b]thiophene-2-carboxylic acid {(S)-1-[1-(2-fluoro-phenylcarbamoyl)-3-oxo-azepan-4-ylcarbamoyl]-3-methyl-butyl}-amide;
Quinoline-2-carboxylic acid {(S)-1-[1-(2-fluoro-phenylcarbamoyl)-3-oxo-azepan-4-ylcarbamoyl]-3-methyl-butyl}-amide;
4-Methyl-2-carboxylic acid {(S)-1-[1-(2-fluoro-phenylcarbamoyl)-3-oxo-azepan-4-ylcarbamoyl]-3-methyl-butyl}-amide;
5-Methoxy-benzofuran-2-carboxylic acid {(S)-1-[1-(2-fluoro-phenylcarbamoyl)-3-oxo-azepan-4-ylcarbamoyl]-3-methyl-butyl}-amide; and
4-Methyl-furan-2-carboxylic acid {(S)-1-[1-(2-fluoro-phenylcarbamoyl)-3-oxo-azepan-4-ylcarbamoyl]-3-methyl-butyl}-amide.

3. A method of treating a parasitic disease mediated by a cysteine protease by administering to a patient in need thereof one or more of the compounds of Formula I: 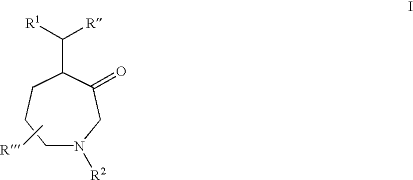
Wherein:
R¹ is selected from the group consisting of: 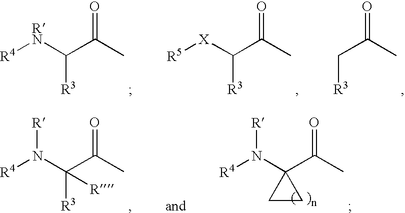
R² is selected from the group consisting of: H, C₁₋₆alkyl, C₃₋₆cycloalkyl-C₀₋₆alkyl, Ar—C₀₋₆alkyl, Het-C₀₋₆alkyl, R⁹C(O)—, R⁹C(S)—, R⁹SO₂—, R⁹OC(O)—,
R⁹R¹¹NC(O)—, R⁹R¹¹NC(S)—, 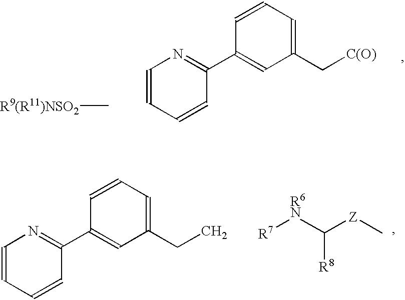
and R⁹SO₂R¹¹NC(O)—;
R³ is selected from the group consisting of: H, C₁₋₆alkyl, C₃₋₆cycloalkyl-C₀₋₆alkyl, C₂₋₆alkenyl, C₂₋₆alkynyl, HetC₀₋₆alkyl and ArC₀₋₆alkyl;
R³ and R′ may be connected to form a pyrrolidine, piperidine or morpholine ring;
R⁴ is selected from the group consisting of: H, C₁₋₆alkyl, C₃₋₆cycloalkyl-C₀₋₆alkyl, Ar—C₀₋₆alkyl, Het-C₀₋₆alkyl, R⁵C(O)—, R⁵C(S)—, R⁵SO₂—, R⁵OC(O)—, R⁵R¹²NC(O)—, and R⁵R¹²NC(S)—;
R⁵ is selected from the group consisting of: H, C₁₋₆alkyl, C₂₋₆alkenyl, C₂₋₆alkynyl, C₃₋₆cycloalkyl-C₀₋₆alkyl, Ar—C₀₋₆alkyl and Het-C₀₋₆alkyl;
R⁶ is selected from the group consisting of: H, C₁₋₆alkyl, Ar—C₀₋₆alkyl, and Het-C₀₋₆alkyl;
R⁷ is selected from the group consisting of: H, C₁₋₆alkyl, C₃₋₆cycloalkyl-C₀₋₆alkyl, Ar—C₀₋₆alkyl, Het-C₀₋₆alkyl, R¹⁰C(O)—, R¹⁰C(S)—, R¹⁰SO₂—, R¹⁰OC(O)—, R¹⁰R¹³NC(O)—, and R¹⁰R¹³NC(S)—;
R⁸ is selected from the group consisting of: H, C₁₋₆alkyl, C₂₋₆alkenyl, C₂₋₆alkynyl, HetC₀₋₆alkyl and ArC₀₋₆alkyl;
R⁹ is selected from the group consisting of: C₁₋₆alkyl, C₃₋₆cycloalkyl-C₀₋₆alkyl, Ar—C₀₋₆alkyl and Het-C₀₋₆alkyl;
R¹⁰ is selected from the group consisting of: C₁₋₆alkyl, C₃₋₆cycloalkyl-C₀₋₆alkyl, Ar—C₀₋₆alkyl and Het-C₀₋₆alkyl;
R¹¹ is selected from the group consisting of: H, C₁₋₆alkyl, Ar—C₀₋₆alkyl, and Het-C₀₋₆alkyl;
R¹² is selected from the group consisting of: H, C₁₋₆alkyl, Ar—C₀₋₆alkyl, and Het-C₀₋₆alkyl;
R¹³ is selected from the group consisting of: H, C₁₋₆alkyl, Ar—CO6alkyl and Het-C₀₋₆alkyl;
R′ is selected from the group consisting of: H, C₁₋₆alkyl, Ar—C₀₋₆alkyl, and Het-C₀₋₆alkyl;
R″ is selected from the group consisting of: H, C₁₋₆alkyl, Ar—C₀₋₆alkyl, or Het-C₀₋₆alkyl;
R′″ is selected from the group consisting of: H, C₁₋₆alkyl, C₃₋₆cycloalkyl-C₀₋₆alkyl, Ar—C₀₋₆alkyl, and Het-C₀₋₆alkyl;
R″″ is selected from the group consisting of: C₁₋₆alkyl, C₃₋₆cycloalkyl-C₀₋₆alkyl C₂₋₆alkenyl, C₂₋₆alkynyl, HetC₀₋₆alkyl and ArC₀₋₆alkyl;
X is selected from the group consisting of: CH₂, S, and O;
Z is selected from the group consisting of: C(O) and CH₂;
n is an integer from 1 to 5;
and pharmaceutically acceptable salts, hydrates and solvates thereof.

4. A method of treating a parasitic disease mediated by a cysteine protease by administering to an animal in need thereof an effective amount of a compound selected from the group consisting of:
Chemical Name
Benzo[1,3]dioxole-5-carboxylic acid [(S)-1-(1-benzyl-3-oxoazepan-4-ylcarbamoyl)-3-methyl-butyl]amide;
Quinoline-2-carboxylic acid [(S)-1-(1-benzyl-3-oxo-azepan-4-ylcarbamoyl)-3-methyl-butyl]amide;
5-(2-Morpholino-4-yl-ethoxy)-benzofuran-2-carboxylic acid ((S)-3-methyl-1-{3-oxo-1-[2-(3-pyridin-2-yl-phenyl)acetyl]-azepan-4-ylcarbamoyl}-butyl)amide;
5-(2-Morpholino-4-yl-ethoxy)-benzofuran-2-carboxylic acid ((S)-3-methyl-1-{3-oxo-1-[2-(3-pyridin-2-yl-phenyl)acetyl]-azepan-4-ylcarbamoyl}-butyl)amide;
4-((S)-4-Methyl-2-{[5-(2-morpholino-4-yl-ethoxy)-benzofuran-2-carbonyl]amino}-pentanoylamino)-3-oxo-azepane-1-carboxylic acid phenylamide;
5-(2-Morpholin-4-yl-ethoxy)-benzofuran-2-carboxylic acid [(S)-1-(1-benzenesulfonyl-3-oxo-azepan-4-ylcarbamoyl)-3-methyl-butyl]amide;
5-(2-Morpholin-4-yl-ethoxy)-benzofuran-2-carboxylic acid [(S)-1-(1-benzenesulfonyl-3-oxo-azepan-4-ylcarbamoyl)-3-methyl-butyl]amide;
5-(2-Pyrrolidin-1-yl-ethoxy)-benzofuran-2-carboxylic acid [(S)-1-(1-benzenesulfonyl-3-oxo-azepan-4-ylcarbamoyl)-3-methyl-butyl]amide;
5-(2-Piperidin-1-yl-ethoxy)-benzofuran-2-carboxylic acid [(S)-1-(1-benzenesulfonyl-3-oxo-azepan-4-ylcarbamoyl)-3-methyl-butyl]amide;
Naphthlene-2-carboxylic acid ((S)-3-methyl-1-{3-oxo-1-[2-(3-pyridin-2-yl-phenyl)ethyl]-azepan-4-ylcarbamoyl}-butyl)amide;
1H-Indole-2-carboxylic acid [(S)-1-(1-benzenesulfonyl-3-oxo-azepan-4-ylcarbamoyl)-3-methyl-butyl]amide;
Benzofuran-2-carboxylic acid [(S)-1-(1-benzenesulfonyl-3-oxo-azepan-4-ylcarbamoyl)-3-methyl-butyl]amide;
5-(2-Pyrrolidin-1-yl-ethoxy)-benzofuran-2-carboxylic acid [(S)-1-(1-benzenesulfonyl-3-oxo-azepan-4-ylcarbamoyl)-3-methyl-butyl]amide;
5-(2-Piperidin-1-yl-ethoxy)-benzofuran-2-carboxylic acid [(S)-1-(1-benzenesulfonyl-3-oxo-azepan-4-ylcarbamoyl)-3-methyl-butyl]amide;
5-(2-Morpholino-4-yl-ethoxy)-benzofuran-2-carboxylic acid {(S)-3-methyl-1-[1-(4-methyl-pentanoyl)-3-oxo-azepan-4-ylcarbamoyl]-butyl}-amide;
Benzofuran-2-carboxylic acid {(S)-3-methyl-1-[3-oxo-1-(pyridine-2-sulfonyl)-azepan-4-ylcarbamoyl]-butyl}-amide;
Naphthalene-2-carboxylic acid {(S)-3-methyl-1-[3-oxo-1-(pyridine-2-sulfonyl)-azepan-4-ylcarbamoyl]-butyl}-amide;
5-(2-Morpholino-4-yl-ethoxy)-benzofuran-2-carboxylic acid {(S)-3-methyl-1-[3-oxo-1-(pyridine-2-sulfonyl)-azepan-4-ylcarbamoyl]-butyl}-amide;
5-(2-Morpholino-4-yl-ethoxy)-benzofuran-2-carboxylic acid {(S)-3-methyl-1-[3-oxo-1-(pyridine-2-sulfonyl)-azepan-4-ylcarbamoyl]-butyl}-amide;
5-(2-Morpholino-4-yl-ethoxy)-benzofuran-2-carboxylic acid {(S)-3-methyl-1-[3-oxo-1-(pyridine-2-sulfonyl)-azepan-4-ylcarbamoyl]-butyl}-amide;
Benzofuran-2-carboxylic acid {(S)-3-methyl-1-[3-oxo-1-(pyridine-2-sulfonyl)-azepan-4-ylcarbamoyl]-butyl}-amide;
4-[2-(2-{(S)-3-Methyl-1-[3-oxo-1-(pyridine-2-sulfonyl)-azepan-4-ylcarbamoyl]-butylcarbamoyl}-benzofuran-5-yloxy)-ethyl]-piperazine-1-carboxylic acid tert-butyl ester;
5-(2-Piperizin-1-yl-ethoxy)-benzofuran-2-carboxylic acid {(S)-3-methyl-1-[3-oxo-1-(pyridine-2-sulfonyl)-azepan-4-ylcarbamoyl]-3-butyl}-amide;
Quinoline-2-carboxylic acid {(S)-3-methyl-1-[3-oxo-1-(pyridine-2-sulfonyl)-azepan-4-ylcarbamoyl]-butyl}amide;
Quinoline-6-carboxylic acid {(S)-3-methyl-1-[3-oxo-1-(pyridine-2-sulfonyl)-azepan-4-ylcarbamoyl]-butyl}amide;
Quinoline-4-carboxylic acid {(S)-3-methyl-1-[3-oxo-1-(pyridine-2-sulfonyl)-azepan-4-ylcarbamoyl]-butyl}amide;
Quinoline-3-carboxylic acid {(S)-3-methyl-1-[3-oxo-1-(pyridine-2-sulfonyl)-azepan-4-ylcarbamoyl]-butyl}amide;
Isoquinoline-3-carboxylic acid {(S)-3-methyl-1-[3-oxo-1-(pyridine-2-sulfonyl)-azepan-4-ylcarbamoyl]-butyl}amide;
Isoquinoline-1-carboxylic acid {(S)-3-methyl-1-[3-oxo-1-(pyridine-2-sulfonyl)-azepan-4-ylcarbamoyl]-butyl}amide;
Quinoxaline-2-carboxylic acid {(S)-3-methyl-1-[3-oxo-1-(pyridine-2-sulfonyl)-azepan-4-ylcarbamoyl]-butyl}amide;
Benzo[b]thiophene-2-carboxylic acid {(S)-3-methyl-1-[3-oxo-1-(pyridine-2-sulfonyl)-azepan-4-ylcarbamoyl]-butyl}amide;
1H-Indole-2-carboxylic acid {(S)-3-methyl-1-[3-oxo-1-(pyridine-2-sulfonyl)-azepan-4-ylcarbamoyl]-butyl}amide;
5-Methoxy-benzofuran-2-carboxylic acid {(S)-3-methyl-1-[3-oxo-1-(pyridine-2-sulfonyl)-azepan-4-ylcarbamoyl]-butyl}amide;
5-Bromo-furan-2-carboxylic acid {(S)-3-methyl-1-[3-oxo-1-(pyridine-2-sulfonyl)-azepan-4-ylcarbamoyl]-butyl}amide;
5-Nitro-furan-2-carboxylic acid {(S)-3-methyl-1-[3-oxo-1-(pyridine-2-sulfonyl)-azepan-4-ylcarbamoyl]-butyl}amide;
5-(4-Nitro-phenyl)-furan-2-carboxylic acid {(S)-3-methyl-1-[3-oxo-1-(pyridine-2-sulfonyl)-azepan-4-ylcarbamoyl]-butyl}amide;
(S)-2-[2-(4-Fluoro-phenoxy)-acetylamino]-4-methyl-pentanoic acid [3-oxo-(pyridine-2-sulfonyl)-azepan-4-yl]-amide;
Benzofuran-2-carboxylic acid {(S)-3-methyl-1-[3-oxo-1-(thiophene-2-sulfonyl)-azepan-4-ylcarbamoyl]-butyl}-amide;
5,6-Dimethoxy-benzofuran-2-carboxylic acid {(S)-3-methyl-1-[3-oxo-1-(1-methyl-1H-imidazole-4-sulfonyl)-azepan-4-ylcarbamoyl]-butyl}amide;
Benzofuran-2-carboxylic acid {(S)-3-methyl-1-[1-(1-methyl-1H-imidazole-3-sulfonyl)-3-oxo-azepan-4-ylcarbamoyl]-butyl}amide;
Benzofuran-2-carboxylic acid {(S)-3-methyl-1-[1-(1H-imidazole-2-sulfonyl)-3-oxo-azepan-4-ylcarbamoyl]-butyl}amide;
Benzofuran-2-carboxylic acid {(S)-3-methyl-1-[1-(1-methyl-1H-imidazole-4-sulfonyl)-3-oxo-azepan-4-ylcarbamoyl]-butyl}amide;
5-(4-Oxy-morpholino-4-yl-ethoxy)-benzofuran-2-carboxylic acid {(S)-3-methyl-1-[3-oxo-1-(pyridine-2-sulfonyl)-azepan-4-ylcarbamoyl]-butyl}amide;
5-Hydroxy-benzofuran-2-carboxylic acid {(S)-3-methyl-1-[1-(1-methyl-1H-imidazole-4-sulfonyl)-3-oxo-azepan-4-ylcarbamoyl]-butyl}amide;
Benzofuran-2-carboxylic acid {(S)-3-methyl-1-[3-oxo-1-(1-oxy-pyridine-2-sulfonyl)-azepan-4-ylcarbamoyl)]-3-methyl-butyl}-amide;
Benzo[b]thiophene-2-carboxylic acid {(S)-3-methyl-1-[3-oxo-1-(1-oxy-pyridine-2-sulfonyl)-azepan-4-ylcarbamoyl]-butyl}amide;
5-Bromo-furan-2-carboxylic acid {(S)-3-methyl-1-[3-oxo-1-(1-oxy-pyridine-2-sulfonyl)azepan-4-ylcarbamoyl]-butyl}amide;
5,6-Dimethoxy-benzofuran-2-carboxylic acid {(S)-3-methyl-1-[3-oxo-1-(1-oxy-pyridine-sulfonyl)-azepan-4-ylcarbamoyl]-butyl}amide;
Benzofuran-2-carboxylic acid {(S)-3-methyl-1-[3-oxo-1-(1-oxy-pyridine-2-sulfonyl)-azepan-4-ylcarbamoyl)]-3-methyl-butyl}-amide;
5-Bromo-furan-2-carboxylic acid {(S)-3-methyl-1-[3-oxo-1-(1-oxy-pyridine-2-sulfonyl)azepan-4-ylcarbamoyl]-butyl}amide;
Benzo[b]thiophene-2-carboxylic acid {(S)-3-methyl-1-[3-oxo-1-(1-oxy-pyridine-2-sulfonyl)-azepan-4-ylcarbamoyl]-butyl}amide;
5,6-Dimethoxy-benzofuran-2-carboxylic acid {(S)-3-methyl-1-[3-oxo-1-(1-oxy-pyridine-2-sulfonyl)-azepan-4-ylcarbamoyl]-butyl}amide;
5-Methoxy-benzofuran-2-carboxylic acid {(S)-3-methyl-1-[3-oxo-1-(1-oxy-pyridine-2-sulfonyl)-azepan-4-ylcarbamoyl]-butyl}amide;
1H-Indole-5-carboxylic acid {(S)-3-methyl-1-[3-oxo-1-(1-oxy-pyridine-2-sulfonyl)-azepan-4-ylcarbamoyl]-butyl}amide;
Benzo[1,3]dioxole-5-carboxylic acid {(S)-3-methyl-1-[3-oxo-1-(1-oxy-pyridine-2-sulfonyl)-azepan-4-ylcarbamoyl]-butyl}amide;
5-(2-Morpholin-4-yl-ethoxy)-benzofuran-2-carboxylic acid {(S)-3-methyl-1-[3-oxo-(1-oxy-pyridine2-sulfonyl)-azepan-4-ylcarbamoyl]-buty}-amide;
3-Methyl-benzofuran-2-carboxylic acid {(S)-3-methyl-1-[3-oxo-1-(1-oxy-pyridine-2-sulfonyl)-azepan-4-ylcarbamoyl]-butyl}amide;
1H-Indole-6-carboxylic acid {(S)-3-methyl-1-[3-oxo-1-(pyridine-2-sulfonyl)-azepan-4-ylcarbamoyl]-butyl}amide;
Benzo[1,3]dioxole-5-carboxylic acid {(S)-3-methyl-1-[3-oxo-1-(pyridine-2-sulfonyl)-azepan-4-ylcarbamoyl]-butyl}amide;
3,4-Dihydro-2H-benzo[b][1,4]dioxepine-7-carboxylic acid {(S)-3-methyl-1-[3-oxo-1-(1-oxy-pyridine-2-sulfonyl)-azepan-4-ylcarbamoyl]butyl}amide;
4,5-Dibromo-thiophene-2-carboxylic acid {(S)-3-methyl-1-[3-oxo-1-(1-oxy-pyridine-2-sulfonyl)-azepan-4-ylcarbamoyl]-butyl}amide;
Thieno[3,2-b]thiophene-2-carboxylic acid {(S)-3-methyl-1-[3-oxo-1-(1-oxy-pyridine-2-sulfonyl)-azepan-4-ylcarbamoyl]-butyl}amide;
5-Methoxy-benzofuran-2-carboxylic acid {(S)-3-methyl-1-[3-oxo-1-(1-oxy-pyridine-2-sulfonyl)-azepan-4-ylcarbamoyl]-butyl}amide;
1H-Indole-5-carboxylic acid {(S)-3-methyl-1-[3-oxo-1-(1-oxy-pyridine-2-sulfonyl)-azepan-4-ylcarbamoyl]-butyl}amide;
5-(4-Chloro-phenyl)-furan-2-carboxylic acid {(S)-3-methyl-1-[3-oxo-1-(pyridine-2-sulfonyl)-azepan-4-ylcarbamoyl]-butyl}amide;
5-(3-Trifluoromethyl-phenyl)-furan-2-carboxylic acid {(S)-3-methyl-1-[3-oxo-1-(pyridine-2-sulfonyl)-azepan-4-ylcarbamoyl]-butyl}amide;
Benzo[1,3]dioxole-5-carboxylic acid {(S)-1-[1-(4-fluoro-benzenesulfonyl)-3-oxo-azepan-4-ylcarbamoyl]-3methyl-butyl}-amide;
5-Bromo-furan-2-carboxylic acid {(S)-3-methyl-1-[3-oxo-1-(pyridine-2-sulfonyl)-azepan-4-ylcarbamoyl]-butyl}amide;
5-Methoxy-benzofuran-2-carboxylic acid {(S)-3-methyl-1-[3-oxo-1-(pyridine-2-sulfonyl)-azepan-4-ylcarbamoyl]-butyl}amide;
3-Methyl-benzofuran-2-carboxylic acid {(S)-3-methyl-1-[3-oxo-1-(pyridine-2-sulfonyl)-azepan-4-ylcarbamoyl]-butyl}amide;
Thieno[3,2-b]thiophene-2-carboxylic acid {(S)-3-methyl-1-[3-oxo-1-(pyridine-2-sulfonyl)-azepan-4-ylcarbamoyl]-butyl}amide;
2-Phenyl-5-trifluoromethyl-oxazole-4-carboxylic acid {(S)-3-methyl-1-[3-oxo-1-(pyridine-2-sulfonyl)-azepan-4-ylcarbamoyl]-butyl}amide;
Benzo[1,3]dioxole-5-carboxylic acid {(S)-1-[1-(4-methoxy-benzenesulfonyl)-3-oxo-azepan-4-ylcarbamoyl]-3-methyl-butyl}-amide;
Benzofuran-2-carboxylic acid {(S)-1-[1-(4-methoxy-benzenesulfonyl)-3-oxo-azepan-4-ylcarbamoyl]-3-methyl-butyl}-amide;
Furan-2-carboxylic acid {(S)-3-methyl-1-[3-oxo-1-(1-oxy-pyridine-2-sulfonyl)-azepan-4-ylcarbamoyl]-butyl}amide;
Benzo[1,3]dioxole-5-carboxylic acid {(S)-3-methyl-1-[3-oxo-1-(1-oxy-pyridine-2-sulfonyl)-azepan-4-ylcarbamoyl]-butyl}amide;
4-Fluoro-{(S)-3-methyl-1-[3-oxo-1-(1-oxy-pyridine-2-sulfonyl)-azepan-4-carbamoyl]-butyl}-benzamide;
3,4-Dihydro-2H-benzo[b][1,4]dioxepine-7-carboxylic acid {(S)-3-methyl-1-[3-oxo-1-(1-oxy-pyridine-2-sulfonyl)-azepan-4-ylcarbamoyl]butyl}amide;
5-Methyl-thiophene-2-carboxylic acid {(S)-3-methyl-1-[3-oxo-1-(1-oxy-pyridine-2-sulfonyl)-azepan-4-ylcarbamoyl]-butyl}amide;
(S)-2-(3-Benzyl-ureido)-4-methyl-pentanoic acid [3-oxo-1-(pyridine-2-sulfonyl)-azepan-4-yl]-amide;
5-Methoxy-benzofuran-2-carboxylic acid [(S)-1-(1-methanesulfonyl-3-oxo-azepan-4-ylcarbamoyl)-3-methyl-butyl]-amide;
Furan-2-carboxylic acid ({(S)-1-[1-(4-methoxy-benzenesulfonyl)-3-oxo-azepan-4-ylcarbamoyl]-3-methyl-butylcarbamoyl}-methyl)-amide;
Quinoline-2-carboxylic acid {[(S)-1-[1-(4-fluoro-benzenesulfonyl)-3-oxo-azepan-4-ylcarbamoyl]-3-methyl-butyl}-amide;
1-Methyl-1H-indole-2-carboxylic acid {[(S)-1-[1-(4-fluoro-benzenesulfonyl)-3-oxo-azepan-4-ylcarbamoyl]-3-methyl-butyl}-amide;
5-Methoxy-benzofuran-2-carboxylic acid {[(S)-1-[1-(4-fluoro-benzenesulfonyl)-3-oxo-azepan-4-ylcarbamoyl]-3-methyl-butyl}-amide;
Quinoxaline-2-carboxylic acid {[(S)-1-[1-(4-fluoro-benzenesulfonyl)-3-oxo-azepan-4-ylcarbamoyl]-3-methyl-butyl}-amide;
Benzo[b]thiophene-2-carboxylic acid {(S)-3-methyl-1-[3-oxo-1-(pyridine-2-sulfonyl)-azepan-4-ylcarbamoyl]-butyl}amide;
Benzofuran-2-carboxylic acid-{(S)-1-[1-(3-chloro-benzenesulfonyl)-3-oxo-azepan-4-ylcarbamoyl]-3-methyl-butyl}-amide;
5-Methoxy-benzofuran-2-carboxylic acid-{(S)-1-[1-(3-chloro-benzenesulfonyl)-3-oxo-azepan-4-ylcarbamoyl]-3-methyl-butyl}-amide;
3-Methyl-benzofuran-2-carboxylic acid-{(S)-1-[1-(3-chloro-benzenesulfonyl)-3-oxo-azepan-4-ylcarbamoyl]-3-methyl-butyl}-amide;
Benzofuran-2-carboxylic acid-{(S)-1-[1-(2-fluoro-benzenesulfonyl)-3-oxo-azepan-4-ylcarbamoyl]-3-methyl-butyl}-amide;
5-Methoxy-benzofuran-2-carboxylic acid-{(S)-1-[1-(2-fluoro-benzenesulfonyl)-3-oxo-azepan-4-ylcarbamoyl]-3-methyl-butyl}-amide;
7-Methoxy-benzofuran-2-carboxylic acid-{(S)-1-[1-(2-fluoro-benzenesulfonyl)-3-oxo-azepan-4-ylcarbamoyl]-3-methyl-butyl}-amide;
3-methylbenzofuran-2-carboxylic acid-{(S)-1-[1-(2-fluoro-benzenesulfonyl)-3-oxo-azepan-4-ylcarbamoyl]-3-methyl-butyl}-amide;
Benzo[b]thiophene-2-carboxylic acid-{(S)-1-[1-(2-fluoro-benzenesulfonyl)-3-oxo-azepan-4-ylcarbamoy]-3-methyl-butyl}-amide;
Quinoxaline-2-carboxylic acid-{(S)-1-[1-(2-fluoro-benzenesulfonyl)-3-oxo-azepan-4-ylcarbamoyl]-3-methyl-butyl}-amide;
3-Methyl-benzofuran-2-carboxylic acid {(S)-3-methyl-1-[3-oxo-1-(pyridine-2-sulfonyl)-azepan-4-ylcarbamoyl]-butyl}amide;
Thieno[3,2-b]thiophene-2-carboxylic acid {(S)-3-methyl-1-[3-oxo-1-(pyridine-2-sulfonyl)-azepan-4-ylcarbamoyl3-butyl}amide;
3-Methyl-benzofuran-2-carboxylic acid {(S)-3-methyl-1-[3-oxo-1-(1-oxy-pyridine-2-sulfonyl)-azepan-4-ylcarbamoyl]-butyl}amide;
Benzofuran-2-carboxylic acid {(S)-3-methyl-1-[(2,2′,4-tridueterio)-3-oxo-1-(pyridine-2-sulfonyl)-azepan-4-ylcarbamoyl]-butyl}amide;
Quinoxaline-2-carboxylic acid {(S)-3-methyl-1-[3-oxo-1-(1-oxy-pyridine-2-sulfonyl)-azepan-4-ylcarbamoyl]-butyl}amide;
Benzofuran-2-carboxylic acid {(S)-2-cyclohexyl-1-[3-oxo-1-(pyridine-2-sulfonyl)-azepan-4-ylcarbamoyl]-ethyl}-amide;
Benzofuran-2-carboxylic acid {(S)-1-[3-oxo-1-(pyridine-2-sulfonyl)-azepan-4-ylcarbamoyl]-pentyl}-amide;
Benzofuran-2-carboxylic acid {(S)-1-[3-oxo-1-(pyridine-2-sulfonyl)-azepan-4-ylcarbamoyl]-2-phenyl-ethyl}-amide;
2-Phenyl-5-trifluoromethyl-oxazole-4-carboxylic acid {(S)-3-methyl-1-[3-oxo-1-(pyridine-2-sulfonyl)-azepan-4-ylcarbamoyl]-butyl}amide;
5-Methyl-2-phenyl-oxazole-4-carboxylic acid {(S)-3-methyl-1-[3-oxo-1-(pyridine-2-sulfonyl)-azepan-4-ylcarbamoyl]-butyl}amide;
3,4-Dimethoxy-N—{(S)-1-[1-(4-methoxy-benzenesulfonyl)-3-oxo-azepan-4-ylcarbamoyl]-3-methyl-butyl}-benzamide;
Benzo[b]thiophene-2-carboxylic acid-{(S)-1-[1-(4-fluoro-benzenesulfonyl)-3-oxo-azepan-4-yl carbamoyl]-3-methyl-butyl}-amide;
Benzofuran-2-carboxylic acid-{(S)-1-[1-(4-fluoro-benzenesulfonyl)-3-oxo-azepan-4-ylcarbamoyl]-3-methyl-butyl}-amide;
N—{(S)-1-[1-(4-Fluoro-benzenesulfonyl)-3-oxo-azepan-4-ylcarbamoyl}-3-methyl-butyl}-3,4-dimethoxy-benzamide;
Benzo[b]thiophene-2-carboxylic acid-{(S)-1-(1-methanesulfonyl-3-oxo-azepan-4-ylcarbamoyl)-3-methyl-butyl]-amide;
Benzofuran-2-carboxylic acid-{(S)-1-(1-methanesulfonyl-3-oxo-azepan-4-yl carbamoyl)-3-methyl-butyl]-amide;
N—[(S)-1-(1-Methanesulfonyl)-3-oxo-azepan-4-ylcarbamoyl}-3-methyl-butyl}-3,4-dimethoxy-benzamide;
N—{(S)-1-[1-(2-Cyano-benzenesulfonyl)-3-oxo-azepan-4-ylcarbamoyl}-3-methyl-butyl}-4-methanesulfonyl-benzamide;
Benzofuran-2-carboxylic acid {(S)-1-[1-(2-cyano-benzenesulfonyl)-3-oxo-azepan-4-ylcarbamoyl]-3-methyl-butyl}-amide;
5-(2-Morpholin-4-yl-ethoxy)-benzofuran-2-carboxylic acid {(S)-3-methyl-1-[3-oxo-(1-oxy-pyridine2-sulfonyl)-azepan-4-ylcarbamoyl]-buty}-amide;
5-Methyl-2-phenyl-oxazole-4-carboxylic acid {(S)-3-methyl-1-[3-oxo-1-(1-oxy-pyridine-2-sulfonyl)-azepan-4-ylcarbamoyl]-butyl}amide;
6-Methyl-N—{(S)-3-methyl-1-[3-oxo-1-(1-oxy-pyridine-2-sulfonyl)-azepan-4-ylcarbamoyl]-butyl}-nicotinamide;
5-(3-Trifluoromethyl-phenyl)-furan-2-carboxylic acid {(S)-3-methyl-1-[3-oxo-1-(1-oxy-pyridine-2-sulfonyl)-azepan-4-ylcarbamoyl]-butyl}amide;
N—{(S)-1-[(1-(2-cyano-benzenesulfonyl)-3-oxo-azepan-4-ylcarbamoyl]-3-methyl-butyl}-3,4-dimethoxy-benzamide;
4-Methansulfonyl-N—{(S)-1-[4-fluoro-benzenesulfonyl)-3-oxo-azepan-4-carbamoyl]-3-methyl-butyl-benzamide;
(S)-2-[5-(4-Methoxy-phenyl)-pentanoylamino]-4-methyl-pentanoic acid [3-oxo-1-(pyridine-2-sulfonyl)-azepan-4-yl]-amide;
(S)-2-[2-(3-Benzyloxy-4-methoxy-phenyl)-acetylamnio]-4-methylpentanoic acid [3-oxo-1-(pyridine-2-sulfonyl)-azepan-4-yl]-amide;
5-Methoxybenzofuran-2-carboxylic acid {(S)-3-methyl-1-[3-oxo-1-(thiazole-2-sulfonyl)-azepan-4-ylcarbamoyl]-butyl}amide;
7-Methoxybenzofuran-2-carboxylic acid {(S)-3-methyl-1-[3-oxo-1-(thiazole-2-sulfonyl)-azepan-4-ylcarbamoyl]-butyl}amide;
3-Methylbenzofuran-2-carboxylic acid {(S)-3-methyl-1-[3-oxo-1-(thiazole-2-sulfonyl)-azepan-4-ylcarbamoyl]-butyl}amide;
Benzo[b]thiophene-2-carboxylic acid {(S)-3-methyl-1-[3-oxo-1-(thiazole-2-sulfonyl)-azepan-4-ylcarbamoyl]-butyl}amide;
1-Methyl-1H-indole-2-carboxylic acid {(S)-3-methyl-1-[3-oxo-1-(thiazole-2-sulfonyl)-azepan-4-ylcarbamoyl]-butyl}amide;
Quinoxaline-2-carboxylic acid {(S)-3-methyl-1-[3-oxo-1-(thiazole-2-sulfonyl)-azepan-4-ylcarbamoyl]-butyl}amide;
Benzofuran-2-carboxylic acid {(S)-3-methyl-1-[3-oxo-1-(thiazole-2-sulfonyl)-azepan-4-ylcarbamoyl]-butyl}amide;
Benzofuran-2-carboxylic acid {(S)-1-[1-(4-chloro-benzenesulfonyl)-3-oxo-azepan-4-ylcarbamoyl]-3-methyl-butyl}-amide;
5-Methoxy-benzofuran-2-carboxylic acid-{(S)-1-[1-(4-chloro-benzenesulfonyl)-3-oxo-azepan-4-ylcarbamoyl]-3-methyl-butyl}-amide;
7-Methoxy-benzofuran-2-carboxylic acid-{(S)-1-[1-(4-chloro-benzenesulfonyl)-3-oxo-azepan-4-ylcarbamoyl]-3-methyl-butyl}-amide;
3-Methyl-benzofuran-2-carboxylic acid-{(S)-1-[1-(4-chloro-benzenesulfonyl)-3-oxo-azepan-4-ylcarbamoyl]-3-methyl-butyl}-amide;
Benzo[b]thiophene-2-carboxylic acid-{(S)-1-[1-(4-chloro-benzenesulfonyl)-3-oxo-azepan-4-ylcarbamoyl]-3-methyl-butyl}-amide;
Benzofuran-2-carboxylic acid-{(S)-1-[1-(3-methoxy-benzenesulfonyl)-3-oxo-azepan-4-ylcarbamoyl]-3-methyl-butyl}-amide;
5-Methoxy-benzofuran-2-carboxylic acid-{(S)-1-[1-(3-methoxy-benzenesulfonyl)-3-oxo-azepan-4-ylcarbamoyl]-3-methyl-butyl}-amide;
7-Methoxy-benzofuran-2-carboxylic acid-{(S)-1-[1-(3-methoxy-benzenesulfonyl)-3-oxo-azepan-4-ylcarbamoyl]-3-methyl-butyl}-amide;
3-Methyl-benzofuran-2-carboxylic acid-{(S)-1-[1-(3-methoxy-benzenesulfonyl)-3-oxo-azepan-4-ylcarbamoyl]-3-methyl-butyl}-amide;
Benzo[b]thiophene-2-carboxylic acid-{(S)-1-[1-(3-methoxy-benzenesulfonyl)-3-oxo-azepan-4-ylcarbamoyl]-3-methyl-butyl}-amide;
1-Methyl-1H-indole-2-carboxylic acid-{(S)-1-[1-(3-methoxy-benzenesulfonyl)-3-oxo-azepan-4-ylcarbamoyl]-3-methyl-butyl}-amide;
Benzofuran-2-carboxylic acid-{(S)-3-methyl-1-[3-oxo-1-(thiophene-2-sulfonyl)-azepan-4-ylcarbamoyl]-butyl}-amide;
5-Methoxy-benzofuran-2-carboxylic acid-{(S)-3-methyl-1-[3-oxo-1-(thiophene-2-sulfonyl)-azepan-4-ylcarbamoyl]-butyl}-amide;
7-Methoxy-benzofuran-2-carboxylic acid-{(S)-3-methyl-1-[3-oxo-1-(thiophene-2-sulfonyl)-azepan-4-ylcarbamoyl]-butyl}-amide;
3-Methyl-benzofuran-2-carboxylic acid-{(S)-3-methyl-1-[3-oxo-1-(thiophene-2-sulfonyl)-azepan-4-ylcarbamoyl]-butyl}-amide;
Benzo[b]thiophene-2-carboxylic acid-{(S)-3-methyl-1-[3-oxo-1-(thiophene-2-sulfonyl)-azepan-4-ylcarbamoyl]-butyl}-amide;
Quinoxaline-2-carboxylic acid-{(S)-3-methyl-1-[3-oxo-1-(thiophene-2-sulfonyl)-azepan-4-ylcarbamoyl]-butyl}-amide;
1-Methyl-1-H-indole-2-carboxylic acid-{(S)-3-methyl-1-[3-oxo-1-(thiophene-2-sulfonyl)-azepan-4-ylcarbamoyl]-butyl}-amide;
5,6-Difluoro-benzofuran-2-carboxylic acid {(S)-3-methyl-1-[1-(pyridine-2-sulfonyl)-3-oxo-azepan-4-ylcarbamoyl]-butyl)}amide;
5,6-Difluoro-benzofuran-2-carboxylic acid {(S)-3-methyl-1-[1-(pyridine-2-sulfonyl)-3-oxo-azepan-4-ylcarbamoyl]-butyl}amide;
Quinoline-2-carboxylic acid {(S)-3-methyl-1-[3-oxo-1-(pyridine-2-sulfonyl)-azepan-4-ylcarbamoyl]-butyl}amide;
Quinoline-6-carboxylic acid {(S)-3-methyl-1-[3-oxo-1-(pyridine-2-sulfonyl)-azepan-4-ylcarbamoyl]-butyl}amide;
Quinoline-4-carboxylic acid {(S)-3-methyl-1-[3-oxo-1-(pyridine-2-sulfonyl)-azepan-4-ylcarbamoyl]-butyl}amide;
Isoquinoline-1-carboxylic acid {(S)-3-methyl-1-[3-oxo-1-(pyridine-2-sulfonyl)-azepan-4-ylcarbamoyl]-butyl}amide;
Naphthalene-2-carboxylic acid {(S)-3-methyl 1-1-[3-oxo-1-(pyridine-2-sulfonyl)-azepan-4-ylcarbamoyl]-butyl}-amide;
Quinoline-3-carboxylic acid {(S)-3-methyl-1-[3-oxo-1-(pyridine-2-sulfonyl)-azepan-4-ylcarbamoyl]-butyl}amide;
5,6-Dimethoxy-benzo[b]thiophene-2-carboxylic acid {(S)-3-methyl-1-[1-(6-methyl-pyridine-2-sulfonyl)3-oxo-azepan-4-ylcarbamoyl]-butyl}amide;
(R)-1-Benzyl-5-oxo-pyrrolidine-2-carboxylic acid {(S)-3-methyl-1-{3-oxo-(pyridine-2-sulfonyl)-azepan-4-ylcarbamoyl]-butyl}amide;
Benzofuran-2-carboxylic acid {(S)-2-naphthalen-2-yl-1-[3-oxo-1-(pyridine-2-sulfonyl)-azepan-4-ylcarbamoyl)-ethyl]-amide;
Thieno[3,2-b]thiophene-2-carboxylic acid {(S)-3-methyl-1-[1-(3-methyl-pyridine-2-sulfonyl)-3-oxo-azepan-4-ylcarbamoyl]-butyl}amide;
3-Methyl-benzofuran-2-carboxylic acid {(S)-3-methyl-1-[1-(3-methyl-pyridine-2-sulfonyl)-3-oxo-azepan-4-ylcarbamoyl]-butyl}amide;
5-Methoxy-benzofuran-2-carboxylic acid {(S)-3-methyl-1-[1-(3-methyl-pyridine-2-sulfonyl)-3-oxo-azepan-4-ylcarbamoyl]-butyl}amide;
5,6-Difluoro-benzofuran-2-carboxylic acid {(S)-3-methyl-1-[3-oxo-1-(1-oxy-pyridine-2-sulfonyl)-azepan-4-ylcarbamoyl]-butyl}amide;
7-Methoxy-benzofuran-2-carboxylic acid {(S)-3-methyl-1-[1-(6-methyl-pyridine-2-sulfonyl)-3-oxo-azepan-4-ylcarbamoyl]-butyl)}amide;
5,6-Dimethoxy-benzo[b]thiophene-2-carboxylic acid {(S)-3-methyl-1-[1-(6-methyl-pyridine-2-sulfonyl)3-oxo-azepan-4-ylcarbamoyl]-butyl}amide;
5-Fluoro-benzofuran-2-carboxylic acid {(S)-3-methyl-1-[3-oxo-1-(pyridine-2-sulfonyl)-azepan-4-ylcarbamoyl]-butyl}-amide;
Benzofuran-2-carboxylic acid {(S)-3-methyl-1-[6-methyl-3-oxo-1-(pyridine-sulfonyl)-azepan-4-ylcarbamoyl]-butyl}-amide;
5-Fluoro-benzofuran-2-carboxylic acid {(S)-3-methyl-1-[3-oxo-1-(pyridine-2-sulfonyl)-azepan-4-ylcarbamoyl]-butyl}-amide;
5-(3-Trifluoromethyl-phenyl)-furan-2-carboxylic acid {(S)-2-cyclohexyl-1-[3-oxo-1-(1-oxy-pyridine-2-sulfonyl)-azepan-4-ylcarbamoyl]-ethyl}-amide;
5,6-Dimethoxy-benzofuran-2-carboxylic acid (S)-2-cyclohexyl-1-[3-oxo-1-(1-oxy-pyridine-2-sulfonyl)-azepan-4-ylcarbamoyl]-ethyl}-amide;
5-Methoxy-benzofuran-2-carboxylic acid {(S)-3-methyl-1-[1-(3-methyl-pyridine-2-sulfonyl)-3-oxo-azepan-4-ylcarbamoyl]-butyl}amide;
Benzofuran-2-carboxylic acid {(S)-3-methyl-1-[1-(6-methyl-pyridine-2-sulfonyl)-3-oxo-azepan-4-ylcarbamoyl]-butyl}amide;
Benzofuran-2-carboxylic acid {(S)-3-methyl-1-[6-methyl-3-oxo-1-(pyridine-sulfonyl)-azepan-4-ylcarbamoyl]-butyl}-amide;
Quinoline-8-carboxylic acid {(S)-2-naphthalen-2-yl-1-[3-oxo-1-(pyridine-2-sulfonyl)-azepan-4-ylcarbamoyl)-ethyl]-amide;
Naphthalene-1-carboxylic acid {(S)-2-naphthalen-2-yl-1-[3-oxo-1-(pyridine-2-sulfonyl)-azepan-4-ylcarbamoyl)-ethyl]-amide;
Quinoline-8-carboxylic acid {(S)-1-[3-oxo-1-(pyridine-2-sulfonyl)-azepan-4-ylcarbamoyl]-2-phenyl-ethyl}-amide;
Naphthyridine-2-carboxylic acid {(S)-3-methyl-1-[3-oxo-1-(pyridine-2-sulfonyl)-azepan-4-ylcarbamoyl]-butyl}-amide;
Naphthalene-1-carboxylic acid {(S)-1-[3-oxo-1-(pyridine-2-sulfonyl)-azepan-4-ylcarbamoyl]-2-phenyl-ethyl}-amide;
Benzofuran-2-carboxylic acid {(S)-3-methyl-1-[1-(2-methyl-furan-3-sulfonyl)-3-oxo-azepan-4-ylcarbamoyl]-butyl}-amide;
Quinoline-2-carboxylic acid {(S)-1-[3-oxo-1-(pyridine-2-sulfonyl)-azepan-4-ylcarbamoyl]-2-phenyl-ethyl}-amide;
Benzofuran-2-carboxylic acid {(S)-3-methyl-1-[(4S ,7S)-7-methyl-3-oxo-1-(pyridine-2-sulfonyl)-azepan-4-ylcarbamoyl]-butyl}-amide;
Benzofuran-2-carboxylic acid {(S)-3-methyl-1-[(4R,7R)-7-methyl-3-oxo-1-(pyridine-2-sulfonyl)-azepan-4-ylcarbamoyl]-butyl}-amide;
Benzofuran-2-carboxylic acid {(S)-1-[-(3-fluoro-benzensulfonyl)-3-oxo-azepan-4-ylcarbamoyl]-3-methyl-1-butyl}-amide;
Naphthalene-1-carboxylic acid {(S)-3-methyl-1-[3-oxo-1-(pyridine-2-sulfonyl)-azepan-4-ylcarbamoyl]-butyl}-amide;
Quinoline-5-carboxylic acid {(S)-3-methyl-1-[3-oxo-1-(pyridine-2-sulfonyl)-azepan-4-ylcarbamoyl]-butyl}-amide;
5-(3-Trifluoromethyl-phenyl)-furan-2-carboxylic acid ((S)-3-methyl-1-{3-oxo-1-[1-(1-oxy-pyridin-2-yl)-methanoyl]-azepan-4-ylcarbamoyl}-butyl)-amide;
Quinoline-8-carboxylic acid {(S)-2-naphthalen-2-yl-1-[3-oxo-1-(pyridine-2-sulfonyl)-azepan-4-ylcarbamoyl]-ethyl}-amide;
Naphthalene-1-carboxylic acid {(S)-2-naphthalen-2-yl-1-[3-oxo-1-(pyridine-2-sulfonyl)-azepan-4-ylcarbamoyl]-ethyl}-amide;
Quinoline-8-carboxylic acid {(S)-1-[3-oxo-1-(pyridine-2-sulfonyl)-azepan-4-ylcarbamoyl]-2-phenyl-ethyl}-amide;
Naphthalene-1-carboxylic acid {(S)-1-[3-oxo-1-(pyridine-2-sulfonyl)-azepan-4-ylcarbamoyl]-2-phenyl-ethyl}-amide;
5-Fluoro-benzofuran-2-carboxylic acid {(S)-3-methyl-1-[3-oxo-1-(1-oxy-pyridine-2-sulfonyl)-azepan-4-ylcarbamoyl]-butyl}-amide;
5-Fluoro-3-methyl-benzofuran-2-carboxylic acid {(S)-3-methyl-1-[3-oxo-1-(1-oxy-pyridine-2-sulfonyl)-azepan-4-ylcarbamoyl]-butyl}-amide;
6-Fluoro-3-methyl-benzofuran-2-carboxylic acid {(S)-3-methyl-1-[3-oxo-1-(1-oxy-pyridine-2-sulfonyl)-azepan-4-ylcarbamoyl]-butyl}-amide;
5-Fluoro-benzofuran-2-carboxylic acid {(S)-3-methyl-1-[3-oxo-1-(1-oxy-pyridine-2-sulfonyl)-azepan-4-ylcarbamoyl]-butyl}-amide;
5-Fluoro-3-methyl-benzofuran-2-carboxylic acid {(S)-3-methyl-1-[3-oxo-1-(1-oxy-pyridine-2-sulfonyl)-azepan-4-ylcarbamoyl]-butyl}-amide;
6-Fluoro-3-methyl-benzofuran-2-carboxylic acid {(S)-3-methyl-1-[3-oxo-1-(1-oxy-pyridine-2-sulfonyl)-azepan-4-ylcarbamoyl]-butyl}-amide;
Benzo[b]thiophene-2-carboxylic acid {(S)-3-methyl-1-[6-methyl-3-oxo-1-(pyridine-2-sulfonyl)-azepan-4-ylcarbamoyl]-butyl}-amide;
5-Methoxy-benzofuran-2-carboxylic acid {(S)-3-methyl-1-[6-methyl-3-oxo-1-(pyridine-2-sulfonyl)-azepan-4-ylcarbamoyl]-butyl}-amide;
3-Methyl-benzofuran-2-carboxylic acid {(S)-3-methyl-1-[6-methyl-3-oxo-1-(pyridine-2-sulfonyl)-azepan-4-ylcarbamoyl]-butyl}-amide;
Thieno[3,2-b]thiophene-2-carboxylic acid {(S)-3-methyl-1-[6-methyl-3-oxo-1-(pyridine-2-sulfonyl)-azepan-4-ylcarbamoyl]-butyl}-amide;
3,5-Dimethyl-benzofuran-2-carboxylic acid {(S)-3-methyl-1-[3-oxo-1-(1-oxy-pyridine-2-sulfonyl)-azepan-4-ylcarbamoyl]-butyl}-amide;
3-Ethyl-benzofuran-2-carboxylic acid {(S)-3-methyl-1-[3-oxo-1-(1-oxy-pyridine-2-sulfonyl)-azepan-4-ylcarbamoyl]-butyl}-amide;
4-Methoxy-3-methyl-benzofuran-2-carboxylic acid {(S)-3-methyl-1-[3-oxo-1-(1-oxy-pyridine-2-sulfonyl)-azepan-4-ylcarbamoyl]-butyl}-amide;
6-Methoxy-3-methyl-benzofuran-2-carboxylic acid {(S)-3-methyl-1-[3-oxo-1-(1-oxy-pyridine-2-sulfonyl)-azepan-4-ylcarbamoyl]-butyl}-amide;
5-Methoxy-3-methyl-benzofuran-2-carboxylic acid {(S)-3-methyl-1-[3-oxo-1-(1-oxy-pyridine-2-sulfonyl)-azepan-4-ylcarbamoyl]-butyl}-amide;
3,5-Dimethyl-benzofuran-2-carboxylic acid {(S)-3-methyl-1-[3-oxo-1-(1-oxy-pyridine-2-sulfonyl)-azepan-4-ylcarbamoyl]-butyl}-amide;
3-Ethyl-benzofuran-2-carboxylic acid {(S)-3-methyl-1-[3-oxo-1-(1-oxy-pyridine-2-sulfonyl)-azepan-4-ylcarbamoyl]-butyl}-amide;
4-Methoxy-3-methylbenzofuran-2-carboxylic acid {(S)-3-methyl-1-[3-oxo-1-(1-oxy-pyridine-2-sulfonyl)-azepan-4-ylcarbamoyl]-butyl}-amide;
4-Methoxy-3-methyl-benzofuran-2-carboxylic acid {(S)-3-methyl-1-[3-oxo-1-(1-oxy-pyridine-2-sulfonyl)-azepan-4-ylcarbamoyl]-butyl}-amide;
1-methyl-naphtho[2,1-b]-furan-2-carboxylic acid {(S)-3-methyl-1-[3-oxo-1-(1-oxy-pyridine-2-sulfonyl)-azepan-4-ylcarbamoyl]-butyl}-amide;
6-Methoxy-3-methyl-benzofuran-2-carboxylic acid {(S)-3-methyl-1-[3-oxo-1-(1-oxy-pyridine-2-sulfonyl)-azepan-4-ylcarbamoyl]-butyl}-amide;
Benzofuran-2-carboxylic acid [(S)-3-methyl-1-[3-oxo-1-quinolin-2-ylmethyl-azepan-4-ylcarbamoyl]-butyl}-amide;
3-Methyl-benzofuran-2-carboxylic acid [(S)-3-methyl-1-[3-oxo-1-quinolin-2-ylmethyl-azepan-4-ylcarbamoyl]-butyl}-amide;
Benzo[b]thiophene-2-carboxylic acid [(S)-3-methyl-1-[3-oxo-1-quinolin-2-ylmethyl-azepan-4-ylcarbamoyl]-butyl}-amide;
Benzo[b]thiophene-2-carboxylic acid {(S)-1-[1-(2-fluoro-phenylcarbamoyl)-3-oxo-azepan-4-ylcarbamoyl]-3-methyl-butyl}-amide;
3-Methyl-benzofuran-2-carboxylic acid {(S)-1-[1-(2-fluoro-phenylcarbamoyl)-3-oxo-azepan-4-ylcarbamoyl]-3-methyl-butyl}-amide;
Quinoxaline-2-carboxylic acid {(S)-1-(2-fluoro-phenylcarbamoyl)-3-oxo-azepan-4-ylcarbamoyl]-3-methyl-butyl}-amide;
Thieno[3,2-b]thiophene-2-carboxylic acid {(S)-1-[1-(2-fluoro-phenylcarbamoyl)-3-oxo-azepan-4-ylcarbamoyl]-3-methyl-butyl}-amide;
Quinoline-2-carboxylic acid {(S)-1-[1-(2-fluoro-phenylcarbamoyl)-3-oxo-azepan-4-ylcarbamoyl]-3-methyl-butyl}-amide;
4-Methyl-2-carboxylic acid {(S)-1-[1-(2-fluoro-phenylcarbamoyl)-3-oxo-azepan-4-ylcarbamoyl]-3-methyl-butyl}-amide;
5-Methoxy-benzofuran-2-carboxylic acid {(S)-1-[1-(2-fluoro-phenylcarbamoyl)-3-oxo-azepan-4-ylcarbamoyl]-3-methyl-butyl}-amide; and
4-Methyl-furan-2-carboxylic acid {(S)-1-[1-(2-fluoro-phenylcarbamoyl)-3-oxo-azepan-4-ylcarbamoyl]-3-methyl-butyl}-amide.

5. A method according to either claim 3 or claim 4 wherein said disease is selected from a group consisting of: malaria, trypanosomiasis (African sleeping sickness, Chagas disease), leishmaniasis, schistosomiasis, onchocerciasis (river blindness) and giardiasis.

6. A method according to claim 5 wherein said disease is malaria.
